(19) United States
(12) Patent Application Publication
(10) Pub. No.: US 2019/0031599 A1
(43) Pub. Date: Jan. 31, 2019

(54) INDANE DERIVATIVES AS MGLUR7 MODULATORS

(71) Applicant: Takeda Pharmaceutical Company Limited, Osaka-shi (JP)

(72) Inventors: Anne Goldby, Cambridge (GB); Gemma Liwicki, Cambridge (GB); Stephen Mack, Cambridge (GB); Martin Teall, Cambridge (GB); Katy White, Cambridge (GB)

(21) Appl. No.: 16/072,296

(22) PCT Filed: Jan. 24, 2017
(86) PCT No.: PCT/JP2017/003078

§ 371 Date: Jul. 24, 2018

(30) Foreign Application Priority Data
Jan. 25, 2016 (GB) 1601301.3

(51) Int. Cl. C07C 233/41; C07C 233/23; A61P 25/30; A61P 25/18; A61P 27/16

(52) U.S. Cl. CPC C07C 233/41; C07C 233/23; A61P 25/30; C07D 239/42; A61P 27/16; C07C 2602/08; A61P 25/18

(57) ABSTRACT

The present invention provides compounds of formula (I) and pharmaceutically acceptable salts thereof,

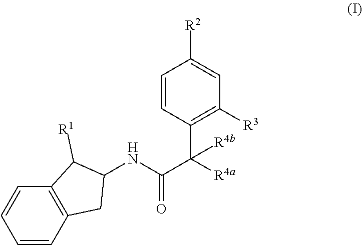

wherein R¹, R², R³, R^{4a} and R^{4b} are as defined in the specification, processes for their preparation, pharmaceutical compositions containing them and their use in therapy.

The present invention relates to indane derivatives, processes for their preparation, pharmaceutical compositions containing them and their use in therapy, particularly for use in treating disorders associated with changes in one or both of the glutamatergic and GABAergic signalling pathways regulated in full or in part by metabotropic glutamate receptor 7 (mGluR7).

L-Glutamate is the major neurotransmitter in the mammalian central nervous system and activates both ionotropic and metabotropic glutamate receptors. L-Glutamate plays a central role in numerous physiological functions such as learning and memory (1), sensory perception, development of synaptic plasticity, motor control, respiration and regulation of cardiovascular function. Thus an imbalance in glutamatergic neurotransmission often underlies many neuropathological conditions.

The metabotropic glutamate receptors are a family of G protein-coupled receptors that have been divided into three groups on the basis of sequence homology, putative signal transduction mechanisms and pharmacologic properties. Group I includes mGluR1 and mGluR5 and these receptors have been shown to activate phospholipase C. Group II includes mGluR2 and mGluR3 whilst Group III includes mGluR4, mGluR6, mGluR7 and mGluR8. Group II and III receptors are linked to the inhibition of the cyclic AMP cascade but differ in their agonist selectivities.

mGluR7 is an inhibitory GPCR expressed pre-synaptically at the synaptic cleft on GABAergic and glutamatergic neurons. Depending on the location it can inhibit or disinhibit synaptic activity and can therefore be seen as a modulator of neuronal function. Therefore, mGluR7 modulators would be expected to be useful in treating a wide variety of neurological and psychiatric disorders such as Parkinson's disease (2, 3); dementia associated with Parkinson's disease (3, 4); Alzheimer's disease (5); Huntington's Chorea (6); amyotrophic lateral sclerosis and multiple sclerosis; bipolar disorder (6, 7); psychiatric diseases such as schizophrenia, post-traumatic stress disorder, anxiety disorders and depression (1, 4, 6, 8-11); addiction; and age-related hearing loss/tinnitus. WO 01/02342 discloses compounds which are modulators of mGlurRs.

The compound N-[(1S,2S)-1-amino-2,3-dihydro-1H-inden-2-yl]-1-(4-fluorophenyl)cyclopropane-1-carboxamide is a chemical library compound (CAS Registry No. 1434131-28-8) commercially available from ChemBridge Corporation with no known pharmaceutical or other use except as a chemical reagent.

There is a need for treatment of the above conditions and others described herein with compounds that are mGluR7 modulators. The present invention provides modulators of mGluR7.

In accordance with the present invention, there is provided a compound of formula (I)

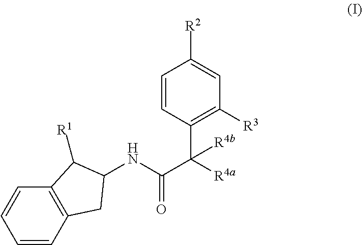

wherein

R¹ represents hydroxyl, —CH₂OH, cyano, —SO₂R^{1a}, —(CH₂)ₘ—(O)ₙ—R⁵ or —(CH₂)ₚNR⁶R⁷;

m is 0 or 1;

n is 0 or 1;

p is 0 or 1;

R^{1a} represents C₁-C₆ alkyl, C₃-C₆ cycloalkyl or C₃-C₆ cycloalkylmethyl;

R² and R³ each independently represent hydrogen, halogen, fluoromethyl, difluoromethyl, trifluoromethyl, methoxy, fluoromethoxy, difluoromethoxy or trifluoromethoxy;

either R^{4a} represents (X)ₜ—(CH₂)ᵥ—R¹⁶ or —CH₂O—R¹⁷ and R^{4b} represents hydrogen, methyl or fluorine, or

R^{4a} and R^{4b} together with the carbon atom to which they are attached form a saturated 3- to 6-membered carbocyclic or heterocyclic ring, the heterocyclic ring comprising at least one ring heteroatom selected from nitrogen and oxygen atoms, wherein the carbocyclic or heterocyclic ring is unsubstituted or substituted with at least one substituent selected from halogen, oxo, C₁-C₃ alkyl, C₁-C₃ alkoxy, amino (NH₂), methylamino, dimethylamino and C₁-C₃ haloalkyl;

R⁵ represents a C₃-C₆ cycloalkyl group, a saturated 4- to 6-membered heterocyclic ring containing a single ring heteroatom being a nitrogen atom wherein the heterocyclic ring is unsubstituted or substituted with at least one substituent selected from halogen, C₁-C₃ alkyl and C₁-C₃ haloalkyl, or a C₁-C₆ alkyl group which is unsubstituted or substituted with at least one substituent selected from C₃-C₆ cycloalkyl, —NR²²R²³ and a saturated 4- to 6-membered heterocyclic ring comprising at least one ring heteroatom selected from nitrogen and oxygen atoms, which heterocyclic ring is unsubstituted or substituted by halogen;

R⁶ and R⁷ each independently represent hydrogen, —(CH₂)_{q}—R⁸, —SO₂R⁹, C₁-C₆ alkyl, C₁-C₆ alkylcarbonyl, C₃-C₆ cycloalkylcarbonyl or C₁-C₆ alkoxycarbonyl, wherein each of the alkyl, cycloalkyl or alkoxy moieties in the latter four substituents is unsubstituted or substituted with at least one substituent selected from halogen, C₁-C₄ alkoxy and —NR¹⁰R¹¹, or

R⁶ and R⁷ together with the nitrogen atom to which they are attached form a saturated or unsaturated 4- to 7-membered heterocyclic ring optionally comprising a further ring heteroatom selected from nitrogen, oxygen and sulphur, the heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹²R¹³;

q is 0, 1 or 2;

R⁸ represents a saturated or unsaturated 3- to 6-membered carbocyclic or heterocyclic ring wherein the heterocyclic ring comprises from 1 to 4 ring heteroatoms independently selected from nitrogen, oxygen and sulphur, the carbocyclic or heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹⁴R¹⁵;

R⁹ represents C₁-C₆ alkyl, C₃-C₆ cycloalkyl or C₃-C₆ cycloalkylmethyl, each of which is unsubstituted or substituted with at least one halogen atom;

R¹⁰ and R¹¹ each independently represent hydrogen, C₁-C₆ alkyl, C₃-C₆ cycloalkyl or

C₃-C₆ cycloalkylmethyl, or

R¹⁰ and R¹¹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

R¹² and R¹³ each independently represent hydrogen, C₁-C₆ alkyl, C₃-C₆ cycloalkyl or C₃-C₆ cycloalkylmethyl, or

R¹² and R¹³ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

R¹⁴ and R¹⁵ each independently represent hydrogen, C₁-C₆ alkyl, C₃-C₆ cycloalkyl or C₃-C₆ cycloalkylmethyl, or

R¹⁴ and R¹⁵ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

t is 0 or 1;

v is 0, 1 or 2;

R¹⁶ represents —R¹⁷, —NR¹⁸R¹⁹ or a saturated or unsaturated 4- to 6-membered heterocyclic ring comprising from 1 to 4 ring heteroatoms independently selected from nitrogen, oxygen and sulphur, the heterocyclic ring being unsubstituted or substituted with at least one substituent selected from oxo, halogen, cyano, fluoromethoxy, difluoromethoxy, trifluoromethoxy, C₁-C₆ alkyl, C₁-C₆ alkoxy and C₁-C₆ haloalkyl;

X is O, NH, —NHC(O)—, —NHC(O)—, —NHC(O)O—, —C(O)NH—, —NHSO₂— or —SO₂NH—, provided that when X is O, NH, —C(O)NH— or —SO₂NH— and R¹⁶ represents —NR¹⁸R¹⁹, then v is 2;

R¹⁷ represents C₁-C₆ alkyl, C₃-C₆ cycloalkyl or C₃-C₆ cycloalkylmethyl, each of which is unsubstituted or substituted with at least one substituent selected from hydroxyl, halogen and —NR²⁰R²¹;

R¹⁸ and R¹⁹ each independently represent hydrogen, C₁-C₆ alkyl, C₁-C₆ alkylcarbonyl, C₃-C₆ cycloalkylcarbonyl, C₁-C₆ alkylsulphonyl or C₃-C₆ cycloalkylsulphonyl, wherein each of the alkyl or cycloalkyl moieties in the latter five substituents is unsubstituted or substituted with at least one substituent selected from halogen and C₁-C₄ alkoxy, or

R¹⁸ and R¹⁹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

R²⁰ and R²¹ each independently represent hydrogen, C₁-C₆ alkyl, C₃-C₆ cycloalkyl or C₃-C₆ cycloalkylmethyl, or

R²⁰ and R²¹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl; and

R²² and R²³ each independently represent hydrogen, C₁-C₆ alkyl, C₃-C₆ cycloalkyl or C₃-C₆ cycloalkylmethyl, or

R²² and R²³ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

provided that the compound of formula (I) is not N-[(1S,2S)-1-amino-2,3-dihydro-1H-inden-2-yl]-1-(4-fluorophenyl)cyclopropane-1-carboxamide;

or a pharmaceutically acceptable salt thereof.

In the context of the present specification, unless otherwise stated, an “alkyl” substituent group or an “alkyl” moiety in a substituent group (such as an alkoxy group) may be linear or branched.

Examples of C₁-C₆ alkyl groups/moieties include methyl, ethyl, propyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, tert-butyl, n-pentyl, and n-hexyl.

A “cycloalkyl” substituent group or a “cycloalkyl” moiety in a substituent group refers to a saturated hydrocarbyl ring containing, for example, from 3 to 8 carbon atoms, examples of which include cyclopropyl, cyclobutyl, cyclopentyl and cyclohexyl.

A “haloalkyl” substituent group or a “haloalkyl” moiety in a substituent group refers to an alkyl group or moiety in which one or more, e.g. one, two, three, four or five, hydrogen atoms are replaced independently by halogen atoms, i.e. by fluorine, chlorine, bromine or iodine atoms. Examples of haloalkyl groups/moieties include fluoromethyl, difluoromethyl, trifluoromethyl and 2,2,2-trifluoroethyl.

The term “oxo” refers to an oxygen atom doubly bonded to the carbon atom to which it is attached to form the carbonyl of a ketone or aldehyde.

The term “halogen” includes fluorine, chlorine, bromine and iodine.

When any of R¹⁰ and R^{1l}, or R¹² and R¹³, or R¹⁴ and R¹⁵, or R¹⁸ and R¹⁹, or R²⁰ and R²¹, or R²² and R²³, together with the nitrogen atom to which they are attached, form a saturated 4- to 6-membered heterocyclic ring, the heterocyclic ring may contain one further ring heteroatom selected from nitrogen and oxygen atoms, in addition to the nitrogen atom to which R¹⁰ and R¹¹, or R¹² and R¹³, or R¹⁴ and R¹⁵, or R¹⁸ and R¹⁹, or R²⁰ and R²¹, or R²² and R²³, are attached. If a substituent is present on the ring, it may be attached to any suitable ring atom. Examples of such heterocyclic rings include azetidinyl, pyrrolidinyl, piperidinyl, morpholinyl and piperazinyl.

When a group or moiety is described as being ‘unsaturated’, it should be understood that the group or moiety may be partially or fully unsaturated and thus may have aliphatic or aromatic properties.

For the purposes of the present invention, where a combination of moieties is referred to as one group, for example, alkylcarbonyl or alkoxycarbonyl, the last mentioned moiety contains the atom by which the group is attached to the rest of the molecule.

When any chemical group or moiety in formula (I) is described as substituted, it will be appreciated that the number and nature of substituents will be selected so as to avoid sterically undesirable combinations.

Further, it will be appreciated that the invention does not encompass any unstable ring or other structures (e.g. >NCH₂N<, >NCH₂O— or aminal groupings of the type >C(NRₐR_{b})(NR_{c}R_{d})) or any O—O or S—S bonds.

R¹ represents hydroxyl, —CH₂OH, cyano, —SO₂R^{1a}, —(CH₂)ₘ—(O)ₙ—R⁵ or —(CH₂)ₚNR⁶R⁷.

In one embodiment, R¹ represents hydroxyl, —(CH₂)ₘ—(O)ₙ—R⁵ or —(CH₂)ₚNR⁶R⁷.

In another embodiment, R¹ represents —(CH₂)ₘ—(O)ₙ—R⁵ or —(CH₂)ₚNR⁶R⁷.

In a further embodiment, R¹ represents —(CH₂)ₚNR⁶R⁷.

When R¹ represents —SO₂R^{1a}, then R^{1a} represents C₁-C₆, or C₁-C₄, or C₁-C₂ alkyl, C₃-C₆, or C₄-C₆, or C₅-C₆ cycloalkyl or C₃-C₆, or C₄-C₆, or C₅-C₆ cycloalkylmethyl.

In one embodiment, R^{1a} represents C₁-C₄, or C₁-C₃, or C₁-C₂ alkyl, C₃-C₆ or C₃-C₅ cycloalkyl or C₃-C₆ or C₃-C₅ cycloalkylmethyl.

In another embodiment, R^{1a} represents methyl, ethyl, cyclopropyl or cyclopropylmethyl, in particular methyl.

When R¹ represents —(CH₂)ₘ—(O)ₙ—R⁵, then m is 0 or 1, n is 0 or 1 and R⁵ represents a C₃-C₆, or C₄-C₆, or C₅-C₆ cycloalkyl group, a saturated 4- to 6-membered heterocyclic ring containing a single ring heteroatom being a nitrogen atom (e.g. azetidinyl) wherein the heterocyclic ring is unsubstituted or substituted with at least one substituent, e.g. one, two, three or four substituents independently, selected from halogen (e.g. fluorine or chlorine), C₁-C₃ alkyl (e.g. methyl or ethyl) and C₁-C₃ haloalkyl (e.g. trifluoromethyl), or R⁵ represents a C₁-C₆, or C₁-C₄, or C₁-C₂ alkyl group which is unsubstituted or substituted with at least one substituent, e.g. one, two, three or four substituents independently, selected from C₃-C₆, or C₄-C₆, or 22-23 C₅-C₆ cycloalkyl, —NR²²R²³ and a saturated 4- to 6-membered heterocyclic ring comprising at least one ring heteroatom, e.g. one or two ring heteroatoms independently, selected from nitrogen and oxygen atoms, which heterocyclic ring is unsubstituted or substituted by halogen, e.g. one, two, three or four halogen (such as fluorine or chlorine) atoms.

Examples of R⁵ saturated 4- to 6-membered heterocyclic rings include azetidinyl, pyrrolidinyl, piperidinyl, piperazinyl, morpholinyl, oxazolidinyl, oxetanyl, oxolanyl (tetrahydrofuranyl) and oxanyl (tetrahydropyranyl).

In one embodiment, R²² and R²³ each independently represent hydrogen, C₁-C₆, or C₁-C₄, or C₁-C₂ alkyl, C₃-C₆, or C₄-C₆, or C₅-C₆ cycloalkyl or C₃-C₆, or C₄-C₆, or C₅-C₆ cycloalkylmethyl.

In another embodiment, R²² and R²³ each independently represent hydrogen, C₁-C₂ alkyl, C₃-C₄ cycloalkyl or C₃-C₄ cycloalkylmethyl.

In a further embodiment, R²² and R²³ each independently represent hydrogen or methyl.

Alternatively, R²² and R²³ may together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent, e.g. one, two, three or four substituents independently, selected from halogen (such as fluorine or chlorine) and C₁-C₃ alkyl, e.g methyl or ethyl.

In one aspect, the saturated heterocyclic ring may contain a single ring heteroatom (being the nitrogen atom to which R²² and R²³ are attached).

In a second aspect, the saturated heterocyclic ring may contain a second ring heteroatom selected from nitrogen or oxygen.

In a third aspect, R²² and R²³ together with the nitrogen atom to which they are attached form an azetidinyl or pyrrolidinyl ring which is unsubstituted or substituted by one or two substituents independently selected from fluorine, chlorine and methyl.

In an embodiment of the invention, m is 0 and n is 0; or m is 0 and n is 1; or m is 1 and n is 0; or m is 1 and n is 1; and R⁵ is as defined above.

In a further embodiment, m is 0; n is 0 or 1; and R⁵ represents a saturated 4- to 6-membered heterocyclic ring containing a single ring heteroatom being a nitrogen atom (e.g. azetidinyl) wherein the heterocyclic ring is unsubstituted or substituted with at least one substituent, e.g. one, two, three or four substituents independently, selected from halogen (e.g. fluorine or chlorine), C₁-C₃ alkyl (e.g. methyl or ethyl) and C₁-C₃ haloalkyl (e.g. trifluoromethyl), or R⁵ represents a C₁-C₆, or C₁-C₄, or C₁-C₂ alkyl group which is unsubstituted or substituted with at least one —NR²²R²³.

In a still further embodiment, m is 0; n is 0 or 1; and R⁵ represents a C₁-C₆, or C₁-C₄, or C₁-C₂ alkyl which is unsubstituted or substituted as defined above, in particular an unsubstituted C₁-C₂ alkyl group.

When R¹ represents —(CH₂)ₚNR⁶R⁷, R⁶ and R⁷ may each independently represent hydrogen, —(CH₂)_{q}—R⁸, —SO₂R⁹, C₁-C₆, or C₁-C₄, or C₁-C₂ alkyl, C₁-C₆, or C₁-C₄, or C₁-C₂ alkylcarbonyl, C₃-C₆, or C₄-C₆, or C₅-C₆ cycloalkylcarbonyl or C₁-C₆, or C₁-C₄, or C₁-C₂ alkoxycarbonyl, wherein each of the alkyl, cycloalkyl or alkoxy moieties in the latter four substituents is unsubstituted or substituted with at least one substituent, e.g. one, two, three or four substituents independently, selected from halogen (e.g. fluorine or chlorine), C₁-C₄ or C₁-C₂ alkoxy and —NR¹⁰R¹¹.

R⁸ represents a saturated or unsaturated 3- to 6-membered carbocyclic or heterocyclic ring wherein the heterocyclic ring comprises from 1 to 4 ring heteroatoms independently selected from nitrogen, oxygen and sulphur, the carbocyclic or heterocyclic ring being unsubstituted or substituted with at least one substituent, e.g. one, two, three or four substituents independently, selected from halogen (e.g. fluorine or chlorine), cyano, C₁-C₆, or C₁-C₄, or C₁-C₂ alkyl, C₃-C₆ or C₃-C₅ cycloalkyl (e.g. cyclopropyl or cyclobutyl), C₃-C₆ or C₃-C₅ cycloalkylmethyl (e.g. cyclopropylmethyl or cyclobutylmethyl), C₁-C₆, or C₁-C₄, or C₁-C₂ haloalkyl (e.g. fluoromethyl, difluoromethyl or trifluoromethyl), C₁-C₆, or C₁-C₄, or C₁-C₂ alkoxy, C₃-C₆ or C₃-C₅ cycloalkyloxy (e.g. cyclopropyloxy or cyclobutyloxy), C₃-C₆ or C₃-C₅ cycloalkylmethyloxy (e.g. cyclopropylmethyloxy or cyclobutylmethyloxy) and —NR¹⁴R¹⁵.

Examples of R⁸ saturated or unsaturated 3- to 6-membered carbocyclic or heterocyclic rings include cyclopropyl, cyclobutyl, cylcopentyl, cyclohexyl, cyclopentene, cyclohexene, phenyl, azetidinyl, pyrrolidinyl, piperidinyl, piperazinyl, morpholinyl, thiomorpholinyl, oxazolidinyl, oxetanyl, oxolanyl (tetrahydrofuranyl), oxanyl (tetrahydropyranyl), pyrazolidinyl, oxazolidinyl, imidazolidinyl, thiazolidinyl, dioxolanyl, 1,4-dioxanyl, pyrrolyl, imidazolyl, pyrazolyl, triazolyl, tetrazolyl, pyridinyl, pyrazinyl, pyrimidinyl, pyridazinyl, triazinyl, thienyl, furyl, furazanyl, oxazolyl, thiazolyl, oxadiazolyl, isothiazolyl, isoxazolyl, thiadiazolyl and tetrazinyl. Preferred rings include cyclopropyl, cyclobutyl, oxanyl, pyrrolidinyl, morpholinyl and pyridinyl.

R¹⁴ and R¹⁵ are defined as for R²² and R²³ above.

In one aspect, R⁸ represents a saturated or unsaturated 3-, 4-, 5- or 6-membered carbocyclic ring (e.g. cyclopropyl or cyclobutyl) or a saturated or unsaturated 4-, 5- or 6-membered heterocyclic ring comprising one or two ring heteroatoms independently selected from nitrogen and oxygen (e.g. oxanyl, pyrrolidinyl, morpholinyl or pyridinyl), the carbocyclic or heterocyclic ring being unsubstituted or substituted with at least one substituent, e.g. one, two, three or four substituents independently, selected from halogen (e.g. fluorine or chlorine), cyano, C₁-C₂ alkyl, C₃-C₆ cycloalkyl (e.g. cyclopropyl or cyclobutyl), C₃-C₆ cycloalkylmethyl (e.g. cyclopropylmethyl or cyclobutylmethyl), C₁-C₂ haloalkyl (e.g. fluoromethyl, difluoromethyl or trifluoromethyl), C₁-C₂ alkoxy, C₃-C₆ cycloalkyloxy (e.g. cyclopropyloxy or cyclobutyloxy), C₃-C₆ cycloalkylmethyloxy (e.g. cyclopropylmethyloxy or cyclobutylmethyloxy) and —NR¹⁴R¹⁵.

In another aspect, q is 0 or 1 and R⁸ represents a saturated 3- to 6-membered carbocyclic ring (e.g. cyclopropyl or cyclobutyl) or a saturated 4- to 6-membered heterocyclic ring comprising one or two ring heteroatoms independently selected from nitrogen and oxygen (e.g. oxanyl, pyrrolidinyl or morpholinyl), the carbocyclic or heterocyclic ring being unsubstituted or substituted with one, two, three or four substituents independently, selected from fluorine, chlorine, cyano, C₁-C₂ alkyl, cyclopropyl, cyclobutyl, cyclopropylmethyl, cyclobutylmethyl, fluoromethyl, difluoromethyl, trifluoromethyl, C₁-C₂ alkoxy, cyclopropyloxy, cyclobutyloxy, cyclopropylmethyloxy, cyclobutylmethyloxy and —NR¹⁴R¹⁵.

In still another aspect, q is 0 and R⁸ represents a saturated 3- to 6-membered carbocyclic ring (e.g. cyclopropyl or cyclobutyl) or a saturated or unsaturated 5- to 6-membered heterocyclic ring comprising one or two ring heteroatoms independently selected from nitrogen and oxygen (e.g. oxanyl, pyrrolidinyl, morpholinyl, pyridinyl, oxazolyl or pyrimidinyl), the carbocyclic or heterocyclic ring being unsubstituted or substituted with at least one halogen atom, particularly a fluorine atom.

In still another aspect, q is 1 and R⁸ represents a saturated 3- to 6-membered carbocyclic ring (e.g. cyclopropyl or cyclobutyl) or a saturated or unsaturated 5- to 6-membered heterocyclic ring comprising one or two ring heteroatoms independently selected from nitrogen and oxygen (e.g. oxanyl, pyrrolidinyl, morpholinyl, pyridinyl, oxazolyl or pyrimidinyl, or e.g. oxanyl, pyrrolidinyl, morpholinyl or pyridinyl), the carbocyclic or heterocyclic ring being unsubstituted or substituted with at least one halogen atom, particularly a fluorine atom.

R⁹ represents C₁-C₆, or C₁-C₄, or C₁-C₂ alkyl, C₃-C₆ or C₃-C₅ cycloalkyl (e.g. cyclopropyl or cyclobutyl), C₃-C₆ or C₃-C₅ cycloalkylmethyl (e.g. cyclopropylmethyl or cyclobutylmethyl), each of which is unsubstituted or substituted with at least one, e.g. one, two, three, four or five, halogen (e.g. fluorine or chlorine) atoms.

In one aspect, R⁹ represents C₁-C₄, or C₁-C₃, or C₁-C₂ alkyl, C₃-C₅ cycloalkyl (e.g. cyclopropyl or cyclobutyl), C₃-C₅ cycloalkylmethyl (e.g. cyclopropylmethyl or cyclobutylmethyl), each of which is unsubstituted or substituted with one, two, three, four or five fluorine atoms.

In another aspect, R⁹ represents C₁-C₄, or C₁-C₃, or C₁-C₂ alkyl (e.g. methyl or ethyl) which is unsubstituted or substituted with one, two, three, four or five fluorine atoms.

R¹⁰ and R¹¹ are defined as for R²² and R²³ above.

In one embodiment, R⁶ and R⁷ each independently represent hydrogen, —(CH₂)_{q}—R⁸, C₁-C₂ alkyl (e.g. methyl), C₁-C₂ alkylcarbonyl (e.g. methylcarbonyl) or C₁-C₄ alkoxycarbonyl (e.g. methoxycarbonyl, ethoxycarbonyl, n-propoxycarbonyl, n-butoxycarbonyl or t-butoxycarbonyl), wherein each of the alkyl or alkoxy moieties in the latter three substituents is unsubstituted or substituted with at least one substituent, e.g. one, two, three or four substituents independently, selected from fluorine, chlorine, C₁-C₂ alkoxy and —NR¹⁰R¹¹.

In a further embodiment, R⁶ and R⁷ each independently represent hydrogen, —(CH₂)_{q}—R⁸, or a methyl, ethyl, methylcarbonyl or t-butoxycarbonyl group, wherein each of the latter four groups is unsubstituted or substituted with from one to three fluorine atoms.

In an alternative embodiment of the invention, when R¹ represents —(CH₂)ₚNR⁶R⁷, R⁶ and R⁷ together with the nitrogen atom to which they are attached form a saturated or unsaturated 4- or 5- to 6- or 7-membered heterocyclic ring optionally comprising a further ring heteroatom selected from nitrogen, oxygen and sulphur, the heterocyclic ring being unsubstituted or substituted with at least one substituent, e.g. one, two, three or four substituents independently, selected from halogen (e.g. fluorine or chlorine), cyano, C₁-C₆, or C₁-C₄, or C₁-C₂ alkyl, C₃-C₆ or C₃-C₅ cycloalkyl (e.g. cyclopropyl or cyclobutyl), C₃-C₆ or C₃-C₅ cycloalkylmethyl (e.g. cyclopropylmethyl or cyclobutylmethyl), C₁-C₆, or C₁-C₄, or C₁-C₂ haloalkyl (e.g. fluoromethyl, difluoromethyl or trifluoromethyl), C₁-C₆, or C₁-C₄, or C₁-C₂ alkoxy, C₃-C₆ or C₃-C₅ cycloalkyloxy (e.g. cyclopropyloxy or cyclobutyloxy), C₃-C₆ or C₃-C₅ cycloalkylmethyloxy (e.g. cyclopropylmethyloxy or cyclobutylmethyloxy) and —NR¹²R¹³.

Examples of such heterocyclic rings include azetidinyl, pyrrolidinyl, piperidinyl, morpholinyl, thiomorpholinyl, piperazinyl, azepanyl, 1,4-oxaazepanyl, pyrrolyl, imidazolyl, pyrazolyl, thiazolyl, isothiazolyl, oxazolyl, isoxazolyl, pyridinyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl and triazinyl. Preferred rings include azetidinyl, pyrrolidinyl, piperidinyl and morpholinyl.

R¹² and R¹³ are defined as for R²² and R²³ above.

In one embodiment, R⁶ and R⁷ together with the nitrogen atom to which they are attached form a saturated or unsaturated 5- to 6-membered heterocyclic ring optionally comprising a further ring heteroatom selected from nitrogen and oxygen, the heterocyclic ring being unsubstituted or substituted with at least one substituent, e.g. one, two, three or four substituents independently, selected from halogen (e.g. fluorine or chlorine), cyano, C₁-C₆, or C₁-C₄, or C₁-C₂ alkyl, C₃-C₆ or C₃-C₅ cycloalkyl (e.g. cyclopropyl or cyclobutyl), C₃-C₆ or C₃-C₅ cycloalkylmethyl (e.g. cyclopropylmethyl or cyclobutylmethyl), C₁-C₆, or C₁-C₄, or C₁-C₂ haloalkyl (e.g. fluoromethyl, difluoromethyl or trifluoromethyl), C₁-C₆, or C₁-C₄, or C₁-C₂ alkoxy, C₃-C₆ or C₃-C₅ cycloalkyloxy (e.g. cyclopropyloxy or cyclobutyloxy), C₃-C₆ or C₃-C₅ cycloalkylmethyloxy (e.g. cyclopropylmethyloxy or cyclobutylmethyloxy) and —NR¹²R¹³.

In a further embodiment, R⁶ and R⁷ together with the nitrogen atom to which they are attached form a saturated 5- to 6-membered heterocyclic ring optionally comprising a further ring heteroatom selected from nitrogen and oxygen (e.g. pyrrolidinyl or morpholinyl), the heterocyclic ring being unsubstituted or substituted with at least one substituent, e.g. one, two, three or four substituents independently, selected from fluorine, chlorine, cyano, C₁-C₂ alkyl, C₃-C₆ cycloalkyl (e.g. cyclopropyl or cyclobutyl), C₃-C₆ cycloalkylmethyl (e.g. cyclopropylmethyl or cyclobutylmethyl), C₁-C₂ haloalkyl (e.g. fluoromethyl, difluoromethyl or trifluoromethyl), C₁-C₂ alkoxy, C₃-C₆ cycloalkyloxy (e.g. cyclopropyloxy or cyclobutyloxy), C₃-C₆ cycloalkylmethyloxy (e.g. cyclopropylmethyloxy or cyclobutylmethyloxy) and —NR¹²R¹³.

In a still further embodiment, R⁶ and R⁷ together with the nitrogen atom to which they are attached form a pyrrolidinyl, morpholinyl or azetidinyl ring which is unsubstituted or substituted as defined above.

In a still further embodiment, R⁶ and R⁷ together with the nitrogen atom to which they are attached form a pyrrolidinyl or morpholinyl ring which is unsubstituted or substituted as defined above.

In a particular embodiment of the invention, R¹ represents any one of the following moieties or is selected from a group containing any two or more of such moieties:

(i) hydroxyl

(ii) methoxy

(iii) ethoxy

(iv) methyl

(v) amino (NH₂)

(vi) methylamino

(vii) dimethylamino

(viii) methylcarbonylamino

(ix) t-butoxycarbonylamino

(x) pyrrolidinyl

(xi) morpholinyl

(xii) 2,2,2-trifluoroethylamino

(xiii) (oxan-4-ylmethyl)amino

(xiv) (cyclopropylmethyl)amino

(xv) ethylamino

(xvi) 2,2-difluoroethylamino

(xvii) (cyclobutylmethyl)amino

(xviii) (3-fluoropyridin-2-ylmethyl)amino

(xix) (cyclobutyl)amino

(xx) (pyrimidin-2-yl)amino

(xxi) bis[(1,3-oxazol-5-yl)methyl]amino

(xxii) 3-fluoroazetidin-1-yl

(xxiii) methanesulphonamido

(xxiv) methanesulfonyl

(xxv) ethanesulfonyl.

In another particular embodiment of the invention, R¹ represents any one of the following moieties or is selected from a group containing any two or more of such moieties:

(i) hydroxyl

(ii) methoxy

(iii) ethoxy

(iv) methyl

(v) amino (NH₂)

(vi) methylamino

(vii) dimethylamino

(viii) methylcarbonylamino

(ix) t-butoxycarbonylamino

(x) pyrrolidinyl

(xi) morpholinyl

(xii) 2,2,2-trifluoroethylamino

(xiii) (oxan-4-ylmethyl)amino

(xiv) (cyclopropylmethyl)amino.

R² and R³ each independently represent hydrogen, halogen (e.g. fluorine or chlorine), fluoromethyl, difluoromethyl, trifluoromethyl, methoxy, fluoromethoxy, difluoromethoxy or trifluoromethoxy.

In one embodiment, R² and R³ each independently represent hydrogen, halogen (e.g. fluorine or chlorine), trifluoromethyl, methoxy or difluoromethoxy.

In another embodiment, R² and R³ each independently represent hydrogen, halogen (e.g. fluorine or chlorine), trifluoromethyl or methoxy.

In another embodiment, R² represents hydrogen, fluorine, chlorine, trifluoromethyl or methoxy and R³ represents hydrogen, fluorine or chlorine.

In still another embodiment, R² and R³ each independently represent hydrogen or fluorine.

In one aspect of the invention, R^{4a} represents (X)ₜ—(CH₂)ᵥ—R¹⁶ or —CH₂O—R¹⁷ (in particular (X)ₜ—(CH₂)ᵥ—R¹⁶) and R^{4b} represents hydrogen, methyl or fluorine, in particular hydrogen.

In one embodiment, R^{4a} represents (X)ₜ—(CH₂)ᵥ—R¹⁶ where t is 0 or 1 and v is 0 or 1.

In another embodiment, R^{4a} represents (X)ₜ—(CH₂)ᵥ—R¹⁶ where t is 1, v is 0 or 1 and X is O, NH, —NHC(O)—, —NHC(O)O— or —NHSO₂—.

In still another embodiment, R^{4a} represents (X)ₜ—(CH₂)ᵥ—R¹⁶ where t is 0, v is 0 and R¹⁶ represents —NR¹⁸R¹⁹.

R¹⁶ represents —R¹⁷, —NR¹⁸R¹⁹ or a saturated or unsaturated 4- to 6-membered heterocyclic ring comprising from 1 to 4 ring heteroatoms independently selected from nitrogen, oxygen and sulphur, the heterocyclic ring being unsubstituted or substituted with at least one substituent, e.g. one, two, three or four substituents independently, selected from oxo, halogen (e.g. fluorine or chlorine), cyano, fluoromethoxy, difluoromethoxy, trifluoromethoxy, C₁-C₆, or C₁-C₄, or C₁-C₂ alkyl, C₁-C₆, or C₁-C₄, or C₁-C₂ alkoxy and C₁-C₆, or C₁-C₄, or C₁-C₂ haloalkyl (e.g. fluoromethyl, difluoromethyl or trifluoromethyl).

Examples of R¹⁶ saturated or unsaturated 4- to 6-membered heterocyclic rings include azetidinyl, pyrrolidinyl, piperidinyl, piperazinyl, morpholinyl, oxazolidinyl, oxetanyl, oxolanyl (tetrahydrofuranyl), oxanyl (tetrahydropyranyl), thienyl, furyl, pyrrolyl, imidazolyl, pyrazolyl, thiazolyl, isothiazolyl, oxazolyl, isoxazolyl, pyridinyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl and triazinyl. In particular the heterocyclic ring is azetidinyl, imidazolyl, pyridinyl, thiazolyl, oxazolyl, pyrazinyl or pyrazolyl.

In one embodiment, R¹⁶ represents azetidinyl, pyrrolidinyl, oxanyl (tetrahydropyranyl), imidazolyl, pyrazolyl, thiazolyl, oxazolyl, pyridinyl or pyridazinyl, each of which is unsubstituted or substituted as defined above.

R¹⁷ represents C₁-C₆, or C₁-C₄, or C₁-C₂ alkyl, C₃-C₆ or C₃-C₅ cycloalkyl (e.g. cyclopropyl or cyclobutyl) or C₃-C₆ or C₃-C₅ cycloalkylmethyl (e.g. cyclopropylmethyl or cyclobutylmethyl), each of which is unsubstituted or substituted with at least one substituent, e.g. one, two, three or four substituents independently, selected from hydroxyl, halogen (e.g. fluorine or chlorine) and —NR²⁰R²¹.

R²⁰ and R²¹ are defined as for R²² and R²³ above.

In an embodiment, R¹⁷ represents C₁-C₄, or C₁-C₃, or C₁-C₂ alkyl or C₃-C₆ cycloalkyl, each of which is unsubstituted or substituted with at least one substituent, e.g. one, two, three or four substituents independently, selected from hydroxyl, halogen (e.g. fluorine or chlorine) and —NR²⁰R²¹.

In another embodiment, R¹⁷ represents methyl, ethyl, isopropyl, t-butyl or cyclopropyl, in particular methyl.

When R¹⁶ represents —NR¹⁸R¹⁹, R¹⁸ and R¹⁹ may each independently represent hydrogen, C₁-C₆, or C₁-C₄, or C₁-C₂ alkyl, C₁-C₆, or C₁-C₄, or C₁-C₂ alkylcarbonyl, C₃-C₆, or C₄-C₆, or C₅-C₆ cycloalkylcarbonyl, C₁-C₆, or C₁-C₄, or C₁-C₂ alkylsulphonyl or C₃-C₆, or C₄-C₆, or C₅-C₆ cycloalkylsulphonyl, wherein each of the alkyl or cycloalkyl moieties in the latter five substituents is unsubstituted or substituted with at least one substituent, e.g. one, two, three or four substituents independently, selected from halogen (e.g. fluorine or chlorine) and C₁-C₄ or C₁-C₂ alkoxy.

In one embodiment, R¹⁸ and R¹⁹ each independently represent hydrogen, C₁-C₄, or C₁-C₃, or C₁-C₂ alkyl, C₁-C₂ alkylcarbonyl, C₃-C₄ cycloalkylcarbonyl, C₁-C₄, or C₁-C₃, or C₁-C₂ alkylsulphonyl or C₃-C₄ cycloalkylsulphonyl, wherein each of the alkyl or cycloalkyl moieties in the latter five substituents is unsubstituted or substituted with at least one substituent, e.g. one, two, three or four substituents independently, selected from halogen (e.g. fluorine or chlorine) and C₁-C₂ alkoxy.

In a further embodiment, R¹⁸ and R¹⁹ each independently represent hydrogen, C₁-C₂ alkyl, C₁-C₂ alkylcarbonyl, cyclopropylcarbonyl, C₁-C₂ alkylsulphonyl or cyclopropylsulphonyl, wherein each of the alkyl or cyclopropyl moieties in the latter five substituents is unsubstituted or substituted with at least one substituent, e.g. one, two, three or four substituents independently, selected from fluorine and methoxy.

In a still further embodiment, R¹⁸ and R¹⁹ both represent hydrogen.

Alternatively, when R¹⁶ represents —NR¹⁸R¹⁹, R¹⁸ and R¹⁹ may together with the nitrogen atom to which they are attached form a saturated 4-, 5- or 6-membered heterocyclic ring optionally comprising a further ring heteroatom selected from nitrogen and oxygen atoms, the heterocyclic ring being unsubstituted or substituted by at least one substituent, e.g. one, two, three or four substituents independently, selected from halogen (e.g. fluorine or chlorine) and C₁-C₃ alkyl.

In one embodiment, R¹⁸ and R¹⁹ together with the nitrogen atom to which they are attached form a saturated 4- or 5-membered heterocyclic ring (e.g. azetidinyl), the heterocyclic ring being unsubstituted or substituted by at least one substituent, e.g. one, two, three or four substituents independently, selected from fluorine and methyl.

In one embodiment, R¹⁶ represents —R¹⁷, —NR¹⁸R¹⁹ or a saturated or unsaturated 5- to 6-membered heterocyclic ring comprising from 1 to 4 ring heteroatoms independently selected from nitrogen, oxygen and sulphur, the heterocyclic ring being unsubstituted or substituted with at least one substituent, e.g. one, two, three or four substituents independently, selected from oxo, halogen (e.g. fluorine or chlorine), cyano, fluoromethoxy, difluoromethoxy, trifluoromethoxy, C₁-C₄, or C₁-C₃, or C₁-C₂ alkyl, C₁-C₄, or C₁-C₃, or C₁-C₂ alkoxy and C₁-C₄, or C₁-C₃, or C₁-C₂ haloalkyl (e.g. fluoromethyl, difluoromethyl or trifluoromethyl).

In another embodiment, R¹⁶ represents a saturated or unsaturated 4-, 5- or 6-membered heterocyclic ring comprising one or two ring heteroatoms independently selected from nitrogen, oxygen and sulphur, the heterocyclic ring being unsubstituted or substituted with one, two or three substituents independently selected from oxo, fluorine, chlorine, fluoromethoxy, difluoromethoxy, trifluoromethoxy, methyl, ethyl, methoxy, ethoxy, fluoromethyl, difluoromethyl or trifluoromethyl.

In another embodiment, R¹⁶ represents C₁-C₄ alkyl, cyclopropyl, NH₂ or an unsaturated 5- to 6-membered heterocyclic ring comprising from 1 to 4 ring heteroatoms independently selected from nitrogen, oxygen and sulphur (e.g. imidazolyl, pyridinyl, thiazolyl or pyrazolyl), the heterocyclic ring being unsubstituted or substituted with at least one substituent, e.g. one, two, three or four substituents independently, selected from oxo, fluorine, chlorine, cyano, fluoromethoxy, difluoromethoxy, trifluoromethoxy, C₁-C₂ alkyl, C₁-C₂ alkoxy and C₁-C₂ haloalkyl (e.g. fluoromethyl, difluoromethyl or trifluoromethyl).

In yet another embodiment, R¹⁶ represents C₁-C₄ alkyl, cyclopropyl, NH₂ or an unsaturated 5- to 6-membered heterocyclic ring system comprising one or two ring heteroatoms independently selected from nitrogen, oxygen and sulphur (e.g. imidazolyl, pyridinyl, thiazolyl or pyrazolyl), the ring system being unsubstituted or substituted with one or two substituents independently selected from oxo and C₁-C₂ alkyl (particularly methyl).

In a particular embodiment of the invention, R^{4a} represents any one of the following moieties or is selected from a group containing any two or more of such moieties:

(i) methyl

(ii) ethyl

(iii) propyl

(iv) isopropyl

(v) methoxy

(vi) 2-oxo-1,2-dihydropyridin-1-yl

(vii) amino (NH₂)

(viii) (cyclopropylmethyl)amino

(ix) [(2-methyl-1,3-thiazol-4-yl)methyl]amino

(x) cyclopropylformamido

(xi) (1-methyl-1H-pyrazol-4-yl)formamido

(xii) t-butoxycarbonylamino

(xiii) methanesulphonamido

(xiv) (pyrrolidin-1-yl)methyl

(xv) (cyclopropyl)methoxy

(xvi) (oxan-4-yl)formamido

(xvii) (3,5-dimethyl-1,2-isoxazol-4-yl)sulphonamido

(xviii) cyclopropyl

(xix) pyrazol-1-yl

(xx) 2-methyl-imidazol-1-yl

(xxi) azetidin-1-yl

(xxii) 3-fluoroazetidin-1-yl

(xxiii) 3,3-difluoroazetidin-1-yl

(xxiv) 3-methoxyazetidin-1-yl

(xxv) 3-(difluoromethoxy)azetidin-1-yl

(xxvi) 6-oxo-1,6-dihydropyridazin-1-yl

and R^{4b} is as defined above, in particular hydrogen or fluorine.

In another particular embodiment of the invention, R^{4a} represents any one of the following moieties or is selected from a group containing any two or more of such moieties:

(i) methyl

(ii) ethyl

(iii) propyl

(iv) isopropyl

(v) methoxy

(vi) 2-oxo-1,2-dihydropyridin-1-yl

(vii) amino (NH₂)

(viii) (cyclopropylmethyl)amino

(ix) [(2-methyl-1,3-thiazol-4-yl)methyl]amino

(x) cyclopropylformamido

(xi) (1-methyl-1H-pyrazol-4-yl)formamido

(xii) t-butoxycarbonylamino

(xiii) methanesulphonamido

and R^{4b} is as defined above, in particular hydrogen or fluorine.

In an alternative aspect of the invention, R^{4a} and R^{4b} together with the carbon atom to which they are attached form a saturated 3- to 6-membered carbocyclic or heterocyclic ring, the heterocyclic ring comprising at least one ring heteroatom, e.g. one or two ring heteroatoms independently, selected from nitrogen and oxygen atoms, wherein the carbocyclic or heterocyclic ring is unsubstituted or substituted with at least one substituent, e.g. one, two or three substituents independently, selected from halogen (e.g. fluorine or chlorine), oxo, C₁-C₃ alkyl (e.g. methyl), C₁-C₃ alkoxy (e.g. methoxy), amino (NH₂), methylamino, dimethylamino and C₁-C₃ haloalkyl (e.g. trifluoromethyl).

Examples of such carbocyclic and heterocyclic rings include cyclopropyl, cyclobutyl, cyclopentyl, cyclohexyl, oxetanyl, oxanyl, pyrrolidinyl and piperidinyl.

In a particular embodiment, R^{4a} and R^{4b} together with the carbon atom to which they are attached form a cyclopropyl ring.

In another particular embodiment of the invention, R^{4a} and R^{4b} together represent any one of the following moieties or are selected from a group containing any two or more of such moieties:

(i) —CH₂CH₂CH₂—

(ii) —CH₂—C(O)—CH₂—

(iii) —CH₂OCH₂—

(iv) —CH₂CH₂—NH—CH₂—

(v) —CH₂—C(O)—NH—CH₂—

(vi) —CH₂CH₂OCH₂CH₂—.

In a preferred embodiment of the invention, the compounds of formula (I) are those in which

R¹ represents hydroxyl, —(CH₂)ₘ—(O)ₙ—R⁵ or —(CH₂)ₚNR⁶R⁷;

m is 0 or 1;

n is 0 or 1;

p is 0 or 1;

R² and R³ each independently represent hydrogen or halogen;

either R^{4a} represents (X)ₜ—(CH₂)ᵥ—R¹⁶ and R^{4b} represents hydrogen or fluorine, or

R^{4a} and R^{4b} together with the carbon atom to which they are attached form a saturated 3- to 6-membered carbocyclic ring;

R⁵ represents a C₁-C₆ alkyl group;

R⁶ and R⁷ each independently represent hydrogen, —(CH₂)_{q}—R⁸, or a methyl, ethyl, methylcarbonyl or t-butoxycarbonyl group, wherein each of the latter four groups is unsubstituted or substituted with from one to three fluorine atoms, or

R⁶ and R⁷ together with the nitrogen atom to which they are attached form a saturated 5- to 6-membered heterocyclic ring optionally comprising a further ring heteroatom selected from nitrogen and oxygen;

q is 0, 1 or 2;

R⁸ represents a saturated 3- to 6-membered carbocyclic ring or a saturated 5- to 6-membered heterocyclic ring comprising one or two ring heteroatoms independently selected from nitrogen and oxygen;

t is 0 or 1;

v is 0, 1 or 2;

R¹⁶ represents —R¹⁷, —NR¹⁸R¹⁹ or a saturated or unsaturated 4- to 6-membered heterocyclic ring comprising from 1 to 4 ring heteroatoms independently selected from nitrogen, oxygen and sulphur, the heterocyclic ring being unsubstituted or substituted with at least one substituent selected from oxo, halogen, cyano, fluoromethoxy, difluoromethoxy, trifluoromethoxy, C₁-C₆ alkyl, C₁-C₆ alkoxy and C₁-C₆ haloalkyl;

X is O, NH, —NHC(O)—, —NHC(O)O— or —NHSO₂—, provided that when X is O or NH and R¹⁶ represents —NR¹⁸R¹⁹, then v is 2;

R¹⁷ represents C₁-C₆ alkyl or C₃-C₆ cycloalkyl; and

R¹⁸ and R¹⁹ both represent hydrogen.

In another preferred embodiment, the compounds of formula (I) are those in which

R¹ represents —(CH₂)ₚNR⁶R⁷;

p is 0 or 1;

R² and R³ each independently represent hydrogen or halogen;

R^{4a} represents (X)ₜ—(CH₂)ᵥ—R¹⁶;

R^{4b} represents hydrogen, methyl or fluorine;

R⁶ and R⁷ each independently represent hydrogen, —(CH₂)_{q}—R⁸, —SO₂R⁹, C₁-C₆ alkyl, C₁-C₆ alkylcarbonyl, C₃-C₆ cycloalkylcarbonyl or C₁-C₆ alkoxycarbonyl, wherein each of the alkyl, cycloalkyl or alkoxy moieties in the latter four substituents is unsubstituted or substituted with at least one substituent selected from halogen, C₁-C₄ alkoxy and —NR¹⁰R¹¹, or

R⁶ and R⁷ together with the nitrogen atom to which they are attached form a saturated or unsaturated 4- to 7-membered heterocyclic ring optionally comprising a further ring heteroatom selected from nitrogen, oxygen and sulphur, the heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹²R¹³;

q is 0, 1 or 2;

R⁸ represents a saturated or unsaturated 3- to 6-membered carbocyclic or heterocyclic ring wherein the heterocyclic ring comprises from 1 to 4 ring heteroatoms independently selected from nitrogen, oxygen and sulphur, the carbocyclic or heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹⁴R¹⁵;

R⁹ represents C₁-C₆ alkyl, C₃-C₆ cycloalkyl or C₃-C₆ cycloalkylmethyl, each of which is unsubstituted or substituted with at least one halogen atom;

R¹⁰ and R¹¹ each independently represent hydrogen, C₁-C₆ alkyl, C₃-C₆ cycloalkyl or C₃-C₆ cycloalkylmethyl, or

R¹⁰ and R¹¹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

R¹² and R¹³ each independently represent hydrogen, C₁-C₆ alkyl, C₃-C₆ cycloalkyl or C₃-C₆ cycloalkylmethyl, or

R¹² and R¹³ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

R¹⁴ and R¹⁵ each independently represent hydrogen, C₁-C₆ alkyl, C₃-C₆ cycloalkyl or C₃-C₆ cycloalkylmethyl, or

R¹⁴ and R¹⁵ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

t is 0 or 1;

v is 0, 1 or 2;

R¹⁶ represents —R¹⁷;

X is NH, —NHC(O)—, or —NHSO₂—;

R¹⁷ represents C₁-C₆ alkyl, C₃-C₆ cycloalkyl or C₃-C₆ cycloalkylmethyl, each of which is unsubstituted or substituted with at least one substituent selected from hydroxyl, halogen and —NR²⁰R²¹; and

R²⁰ and R²¹ each independently represent hydrogen, C₁-C₆ alkyl, C₃-C₆ cycloalkyl or C₃-C₆ cycloalkylmethyl, or

R²⁰ and R²¹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl.

In still another preferred embodiment, the compounds of formula (I) are those in which

R¹ represents —(CH₂)ₚNR⁶R⁷;

p is 0;

R² and R³ each independently represent hydrogen or halogen;

R^{4a} represents (X)ₜ—(CH₂)ᵥ—R¹⁶;

R^{4b} represents hydrogen;

R⁶ and R⁷ each independently represent hydrogen, or

R⁶ and R⁷ together with the nitrogen atom to which they are attached form a saturated or unsaturated 4- to 7-membered heterocyclic ring optionally comprising a further ring heteroatom selected from nitrogen, oxygen and sulphur, the heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹²R¹³;

R¹² and R¹³ each independently represent hydrogen, C₁-C₆ alkyl, C₃-C₆ cycloalkyl or C₃-C₆ cycloalkylmethyl, or

R¹² and R¹³ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;

t is 0 or 1;

v is 0, 1 or 2;

R¹⁶ represents —R¹⁷;

X is NH, —NHC(O)—, or —NHSO₂—; and

R¹⁷ represents C₁-C₆ alkyl or C₃-C₆ cycloalkyl.

In yet another preferred embodiment, the compounds of formula (I) are those in which

R¹ represents —(CH₂)ₚNR⁶R⁷;

p is 0;

R² and R³ each independently represent hydrogen or halogen;

R^{4a} represents (X)ₜ—(CH₂)ᵥ—R¹⁶;

R^{4b} represents hydrogen;

R⁶ and R⁷ each independently represent hydrogen;

t is 0 or 1;

v is 0, 1 or 2;

R¹⁶ represents —R¹⁷;

X is NH, —NHC(O)—, or —NHSO₂—; and

R¹⁷ represents C₁-C₆ alkyl or C₃-C₆ cycloalkyl.

In yet another embodiment, R¹, R², R³, R^{4a} and R^{4b} are not all simultaneously:

R¹ this: —NR⁶R⁷, wherein R⁶ and R⁷ each independently represent hydrogen or C₁-C₅ alkyl, or R⁶ and R⁷ together with the nitrogen atom to which they are attached form a saturated or unsaturated 5- or 6-membered heterocyclic ring optionally comprising a further ring heteroatom selected from nitrogen, oxygen and sulphur, the heterocyclic ring being unsubstituted or substituted with one substituent selected from C₁-C₄ alkyl;

R² this: hydrogen, halogen, trifluoromethyl or methoxy;

R³ this: hydrogen, halogen, trifluoromethyl or methoxy;

R^{4a} this: C₁-C₇ alkyl; and

R^{4b} this: hydrogen or methyl.

In yet another embodiment, R¹, R², R³, R^{4a} and R^{4b} are not all simultaneously:

R¹ this: —NR⁶R⁷;

R² this: hydrogen, halogen, trifluoromethyl or methoxy;

R³ this: hydrogen, halogen, trifluoromethyl or methoxy;

R^{4a} this: alkyl; and

R^{4b} this: hydrogen or methyl.

In yet another embodiment, when R¹ represents —NR⁶R⁷, wherein R⁶ and R⁷ each independently represent hydrogen or C₁-C₅ alkyl, or R⁶ and R⁷ together with the nitrogen atom to which they are attached form a saturated or unsaturated 5- or 6-membered heterocyclic ring optionally comprising a further ring heteroatom selected from nitrogen, oxygen and sulphur, the heterocyclic ring being unsubstituted or substituted with one substituent selected from C₁-C₄ alkyl, and R^{4b} represents hydrogen or methyl, then R^{4a} does not represent C₁-C₇ alkyl.

In yet another embodiment, when R¹ represents —NR⁶R⁷, wherein R⁶ and R⁷ each independently represent hydrogen or C₁-C₅ alkyl, or R⁶ and R⁷ together with the nitrogen atom to which they are attached form a saturated or unsaturated 5- or 6-membered heterocyclic ring optionally comprising a further ring heteroatom selected from nitrogen, oxygen and sulphur, the heterocyclic ring being unsubstituted or substituted with one substituent selected from C₁-C₄ alkyl, and R^{4a} represents C₁-C₇ alkyl, then R^{4b} does not represent hydrogen or methyl.

In yet another embodiment, when R^{4a} represents C₁-C₇ alkyl and R^{4b} represents hydrogen or methyl, then R¹ does not represent —NR⁶R⁷, wherein R⁶ and R⁷ each independently represent hydrogen or C₁-C₅ alkyl, or R⁶ and R⁷ together with the nitrogen atom to which they are attached form a saturated or unsaturated 5- or 6-membered heterocyclic ring optionally comprising a further ring heteroatom selected from nitrogen, oxygen and sulphur, the heterocyclic ring being unsubstituted or substituted with one substituent selected from C₁-C₄ alkyl.

Examples of compounds of the invention include:
(2R)—N-((trans)-1-hydroxy-2,3-dihydro-1H-inden-2-yl)-2-phenylpropanamide;
(2 S)—N-((trans)-1-hydroxy-2,3-dihydro-1H-inden-2-yl)-2-phenylpropanamide;
(2 S)—N-((cis)-1-hydroxy-2,3-dihydro-1H-inden-2-yl)-2-phenylpropanamide;
(2 S)—N-((trans)-1-methoxy-2,3-dihydro-1H-inden-2-yl)-2-phenylpropanamide;
(2 S)—N-((cis)-1-methoxy-2,3-dihydro-1H-inden-2-yl)-2-phenylpropanamide;
(2S)—N-[(1S,2S)-1-amino-2,3-dihydro-1H-inden-2-yl]-2-phenylpropanamide;
(2S)—N-[(1R,2R)-1-amino-2,3-dihydro-1H-inden-2-yl]-2-phenylpropanamide;
(2S)—N-[(1S,2S)-1-acetamido-2,3-dihydro-1H-inden-2-yl]-2-phenylpropanamide;
N-[(1S,2S)-1-amino-2,3-dihydro-1H-inden-2-yl]-2-(2,4-difluorophenyl)butanamide;
N-[(1R,2R)-1-amino-2,3-dihydro-1H-inden-2-yl]-2-(2,4-difluorophenyl)butanamide;
tert-butyl N-[(1R,2R)-2-[(2S)-2-phenylpropanamido]-2,3-dihydro-1H-inden-1-yl]carbamate;
(2S)—N-[(1S,2S)-1-amino-2,3-dihydro-1H-inden-2-yl]-2-methoxy-2-phenylacetamide;
N-[(1S,2S)-1-amino-2,3-dihydro-1H-inden-2-yl]-2-(4-chlorophenyl)-3-methylbutanamide;
tert-butyl N-[(1S,2S)-2-[(2S)-2-(4-fluorophenyl)propanamido]-2,3-dihydro-1H-inden-1-yl]carbamate;
(2S)—N-[(1S,2S)-1-(methylamino)-2,3-dihydro-1H-inden-2-yl]-2-phenylpropanamide;
tert-butyl N-[(1S,2S)-2-[(2S)-2-(2,4-difluorophenyl)propanamido]-2,3-dihydro-1H-inden-1-yl]carbamate;
(2 S)—N-[(1S,2S)-1-amino-2,3-dihydro-1H-inden-2-yl]-2-(4-fluorophenyl)propanamide;
(2S)-2-(2,4-difluorophenyl)-N-((trans)-1-methoxy-2,3-dihydro-1H-inden-2-yl)propanamide;
(2S)-2-(2,4-difluorophenyl)-N-((trans)-1-ethoxy-2,3-dihydro-1H-inden-2-yl)propanamide;
(2S)—N-((trans)-1-ethoxy-2,3-dihydro-1H-inden-2-yl)-2-(4-fluorophenyl)propanamide;
(2 S)—N-[(1S,2S)-1-amino-2,3-dihydro-1H-inden-2-yl]-2-(2,4-difluorophenyl)propanamide;
(2 S)—N-[(1R,2R)-1-amino-2,3-dihydro-1H-inden-2-yl]-2-(2,4-difluorophenyl)propanamide;
(2S)-2-(4-fluorophenyl)-N-((trans)-1-methoxy-2,3-dihydro-1H-inden-2-yl)propanamide;
(2S)-2-(4-fluorophenyl)-N-(cis)-(1-methoxy-2,3-dihydro-1H-inden-2-yl)propanamide;
(2S)-2-(2,4-difluorophenyl)-N-[(1S,2S)-1-{[(oxan-4-yl)methyl]amino}-2,3-dihydro-1H-inden-2-yl]propanamide;
tert-butyl N-[(1R,2R)-2-[2-(2,4-difluorophenyl)butanamido]-2,3-dihydro-1H-inden-1-yl]carbamate;
tert-butyl N-[(1S,2S)-2-[(2S)-2-(4-fluorophenyl)butanamido]-2,3-dihydro-1H-inden-1-yl]carbamate;
tert-butyl N-[(1R,2R)-2-[(2S)-2-(4-fluorophenyl)butanamido]-2,3-dihydro-1H-inden-1-yl]carbamate;
(2S)-2-(4-fluorophenyl)-N-((trans)-1-methoxy-2,3-dihydro-1H-inden-2-yl)butanamide;
(2 S)—N-[(1S,2S)-1-[(cyclopropylmethyl)amino]-2,3-dihydro-1H-inden-2-yl]-2-(4-fluorophenyl)propanamide;
tert-butyl N-[(1S,2S)-2-[2-(2,4-difluorophenyl)-2-(2-oxo-1,2-dihydropyridin-1-yl)acetamido]-2,3-dihydro-1H-inden-1-yl]carbamate;
N-[(1S,2S)-1-amino-2,3-dihydro-1H-inden-2-yl]-2-(2,4-difluorophenyl)-2-(2-oxo-1,2-dihydropyridin-1-yl)acetamide;
(2 S)—N-[(1S,2S)-1-amino-2,3-dihydro-1H-inden-2-yl]-2-(4-fluorophenyl)butanamide;
(2 S)—N-[(1R,2R)-1-amino-2,3-dihydro-1H-inden-2-yl]-2-(4-fluorophenyl)butanamide;
(2S)-2-amino-2-(4-fluorophenyl)-N-(trans)-(1-methoxy-2,3-dihydro-1H-inden-2-yl)acetamide;
(2S)-2-[(cyclopropylmethyl)amino]-2-(4-fluorophenyl)-N-(trans)-(1-methoxy-2,3-dihydro-1H-inden-2-yl)acetamide;
N-[(1S,2S)-1-amino-2,3-dihydro-1H-inden-2-yl]-2-(4-methoxyphenyl)propanamide;
N-[(1S,2S)-1-amino-2,3-dihydro-1H-inden-2-yl]-2-[4-(trifluoromethyl)phenyl]propanamide;
(2S)-2-(2,4-difluorophenyl)-N-[(1S,2S)-1-[(2,2,2-trifluoroethyl)amino]-2,3-dihydro-1H-inden-2-yl]propanamide;
(2S)—N-(trans)-(1-ethoxy-2,3-dihydro-1H-inden-2-yl)-2-(4-fluorophenyl)-2-{[(2-methyl-1,3-thiazol-4-yl)methyl]amino}acetamide;
(2S)-2-(4-fluorophenyl)-N-(trans)-(1-hydroxy-2,3-dihydro-1H-inden-2-yl)propanamide;
(2S)-2-(4-fluorophenyl)-N-(cis)-(1-hydroxy-2,3-dihydro-1H-inden-2-yl)propanamide;
(2S)-2-(4-fluorophenyl)-N-[(1S,2S)-1-(methylamino)-2,3-dihydro-1H-inden-2-yl]propanamide;
(2S)-2-(4-fluorophenyl)-N-(trans)-(1-methoxy-2,3-dihydro-1H-inden-2-yl)-2-[(1-methyl-1H-pyrazol-4-yl)formamido]acetamide;
(2S)-2-(cyclopropylformamido)-2-(4-fluorophenyl)-N-(trans)-(1-methoxy-2,3-dihydro-1H-inden-2-yl)acetamide;
(2S)-2-(4-fluorophenyl)-N-(trans)-[1-(pyrrolidin-1-yl)-2,3-dihydro-1H-inden-2-yl]propanamide;
N-[(1S,2S)-1-amino-2,3-dihydro-1H-inden-2-yl]-2-(4-chlorophenyl)propanamide;
tert-butyl N—[(S)-(4-fluorophenyl)[(trans)-(1-methyl-2,3-dihydro-1H-inden-2-yl)carbamoyl]methyl]carbamate;
(2S)-2-(4-fluorophenyl)-2-methanesulphonamido-N-(trans)-(1-methoxy-2,3-dihydro-1H-inden-2-yl)acetamide;
(2S)-2-(4-fluorophenyl)-N-(trans)-[1-(morpholin-4-yl)-2,3-dihydro-1H-inden-2-yl]propanamide;
(2S)—N-(trans)-[1-(dimethylamino)-2,3-dihydro-1H-inden-2-yl]-2-(4-fluorophenyl)propanamide;
N-[(1S,2S)-1-amino-2,3-dihydro-1H-inden-2-yl]-2-fluoro-2-(4-fluorophenyl)propanamide;
(2S)-2-phenyl-N-(trans)-[1-(pyrrolidin-1-yl)-2,3-dihydro-1H-inden-2-yl]propanamide;
N-[(1S,2S)-1-amino-2,3-dihydro-1H-inden-2-yl]-1-(2-chlorophenyl)cyclopropane-1-carboxamide;
(2S)-2-(2,4-difluorophenyl)-N-[(1S,2S)-1-acetamido-2,3-dihydro-1H-inden-2-yl]propanamide;
tert-butyl N-[(1S,2S)-2-[2-(2,4-difluorophenyl)-2-(1H-pyrazol-1-yl)acetamido]-2,3-dihydro-1H-inden-1-yl]carbamate;
tert-butyl N-[(1S,2S)-2-[2-(2,4-difluorophenyl)-2-(2-methyl-1H-imidazol-1-yl)acetamido]-2,3-dihydro-1H-inden-1-yl]carbamate;
(2S)-2-(3,5-dimethyl-1,2-isoxazole-4-sulfonamido)-2-(4-fluorophenyl)-N-((trans)-1-methoxy-2,3-dihydro-1H-inden-2-yl)acetamide;
(2S)—N-{(trans)-1-[(2,2-difluoroethyl)amino]-2,3-dihydro-1H-inden-2-yl}-2-(4-fluorophenyl)propanamide;
(2S)-2-(4-fluorophenyl)-2-methanesulfonamido-N-((trans)-1-methyl-2,3-dihydro-1H-inden-2-yl)acetamide;
N-[(1S,2S)-1-amino-2,3-dihydro-1H-inden-2-yl]-2-cyclopropyl-2-(4-fluorophenyl)acetamide;
tert-butyl N-[(1S,2S)-2-[2-(4-fluorophenyl)-2-methylpropanamido]-2,3-dihydro-1H-inden-1-yl]carbamate;
tert-butyl N-[(1S,2S)-2-(3-phenyloxetane-3-amido)-2,3-dihydro-1H-inden-1-yl]carbamate;
(2S)-2-(2,4-difluorophenyl)-N-[(1S,2S)-1-methanesulfonamido-2,3-dihydro-1H-inden-2-yl]propanamide;
(2S)—N-[(1S,2S)-1-[(cyclobutylmethyl)amino]-2,3-dihydro-1H-inden-2-yl]-2-(2,4-difluorophenyl)propanamide;
(2S)—N-[(1S,2S)-1-(cyclobutylamino)-2,3-dihydro-1H-inden-2-yl]-2-(2,4-difluorophenyl)propanamide;
(2S)-2-(2,4-difluorophenyl)-N-[(1S,2S)-1-{[(3-fluoropyridin-2-yl)methyl]amino}-2,3-dihydro-1H-inden-2-yl]propanamide;
tert-butyl N-[(1S,2S)-2-[2-(2,4-difluorophenyl)-2-(3-fluoroazetidin-1-yl)acetamido]-2,3-dihydro-1H-inden-1-yl]carbamate;
tert-butyl N-[(1S,2S)-2-[4-(4-fluorophenyl)oxane-4-amido]-2,3-dihydro-1H-inden-1-yl]carbamate;
(2S)-2-(4-fluorophenyl)-N-[(1R,2R)-1-(methylamino)-2,3-dihydro-1H-inden-2-yl]propanamide;
(2S)-2-(4-fluorophenyl)-N-(trans)-(1-methoxy-2,3-dihydro-1H-inden-2-yl)-2-[(oxan-4-yl)formamido]acetamide;
N-[(1S,2S)-1-amino-2,3-dihydro-1H-inden-2-yl]-2-(4-fluorophenyl)-2-methylpropanamide;
tert-butyl N-[(1S,2S)-2-[2-(azetidin-1-yl)-2-(2,4-difluorophenyl)acetamido]-2,3-dihydro-1H-inden-1-yl]carbamate;
N-[(1S,2S)-1-amino-2,3-dihydro-1H-inden-2-yl]-2-(3,3-difluoroazetidin-1-yl)-2-(2,4-difluorophenyl)acetamide;
tert-butyl N-[(1S,2S)-2-[2-(2,4-difluorophenyl)-2-(3-methoxyazetidin-1-yl)acetamido]-2,3-dihydro-1H-inden-1-yl]carbamate;
N-[(1S,2S)-1-amino-2,3-dihydro-1H-inden-2-yl]-2-(2,4-difluorophenyl)-2-(3-fluoroazetidin-1-yl)acetamide;
N-[(1S,2S)-1-amino-2,3-dihydro-1H-inden-2-yl]-2-(azetidin-1-yl)-2-(2,4-difluorophenyl)acetamide;
N-[(1S,2S)-1-amino-2,3-dihydro-1H-inden-2-yl]-2-(2,4-difluorophenyl)-2-(6-oxo-1,6-dihydropyridazin-1-yl)acetamide;
N-[(1S,2S)-1-amino-2,3-dihydro-1H-inden-2-yl]-2-(2,4-difluorophenyl)-2-(3-methoxyazetidin-1-yl)acetamide;
(2 S)—N-[(1R,2R)-1-(3-fluoroazetidin-1-yl)-2,3-dihydro-1H-inden-2-yl]-2-(4-fluorophenyl)propanamide;
(2S)-2-(4-fluorophenyl)-N-[(1R,2R)-1-methanesulfonyl-2,3-dihydro-1H-inden-2-yl]propanamide;
(2S)—N-[(1S,2S)-1-{bis[(1,3-oxazol-2-yl)methyl]amino}-2,3-dihydro-1H-inden-2-yl]-2-(2,4-difluorophenyl)propanamide;
N-[(1S,2S)-1-amino-2,3-dihydro-1H-inden-2-yl]-2-(3-fluoroazetidin-1-yl)-2-(4-fluorophenyl)acetamide;
N-[(1S,2S)-1-amino-2,3-dihydro-1H-inden-2-yl]-2-(3-fluoroazetidin-1-yl)-2-(4-fluorophenyl)acetamide;
N-[(1S,2S)-1-amino-2,3-dihydro-1H-inden-2-yl]-2-[3-(difluoromethoxy)azetidin-1-yl]-2-(2,4-difluorophenyl)acetamide;
N-[(1S,2S)-1-amino-2,3-dihydro-1H-inden-2-yl]-3-phenylpyrrolidine-3-carboxamide;
tert-butyl N-[(1S,2S)-2-(5-oxo-3-phenylpyrrolidine-3-amido)-2,3-dihydro-1H-inden-1-yl]carbamate;
N-[(1S,2S)-1-amino-2,3-dihydro-1H-inden-2-yl]-5-oxo-3-phenylpyrrolidine-3-carboxamide;
tert-butyl N-[(1S,2S)-2-(3-oxo-1-phenylcyclobutaneamido)-2,3-dihydro-1H-inden-1-yl]carbamate;
2-(2,4-difluorophenyl)-N-(trans)-(1-methoxy-2,3-dihydro-1H-inden-2-yl)-2-(6-oxo-1,6-dihydropyridazin-1-yl)acetamide;
2-(4-fluorophenyl)-N-(trans)-(1-methoxy-2,3-dihydro-1H-inden-2-yl)-2-(6-oxo-1,6-dihydropyridazin-1-yl)acetamide;
(2S)-2-(4-fluorophenyl)-N-[(1S,2S)-1-methanesulfonyl-2,3-dihydro-1H-inden-2-yl]propanamide;
(2S)-2-(2,4-difluorophenyl)-N-[(1S,2S)-1-[(pyrimidin-2-yl)amino]-2,3-dihydro-1H-inden-2-yl]propanamide;
(2S)—N-[(1S,2S)-1-(ethylamino)-2,3-dihydro-1H-inden-2-yl]-2-(4-fluorophenyl)propanamide;
2-(cyclopropylmethoxy)-N-(trans)-(1-methanesulfonyl-2,3-dihydro-1H-inden-2-yl)-2-phenylacetamide;
2-(2,4-difluorophenyl)-2-(3-fluoroazetidin-1-yl)-N-(trans)-(1-methoxy-2,3-dihydro-1H-inden-2-yl)acetamide;
2-(4-fluorophenyl)-2-(3-fluoroazetidin-1-yl)-N-(trans)-(1-methoxy-2,3-dihydro-1H-inden-2-yl)acetamide;
(2R)-2-(cyclopropylformamido)-2-(4-fluorophenyl)-N-(trans)-(1-methoxy-2,3-dihydro-1H-inden-2-yl)acetamide;
(2 S)—N-(trans)-(1-methanesulfonyl-2,3-dihydro-1H-inden-2-yl)-2-methoxy-2-phenylacetamide;
(2S)—N-(trans)-[1-(ethanesulfonyl)-2,3-dihydro-1H-inden-2-yl]-2-methoxy-2-phenylacetamide;
tert-butyl N-[(1S,2S)-2-{2-[4-(difluoromethoxy)phenyl]propanamido}-2,3-dihydro-1H-inden-1-yl]carbamate;
tert-butyl N-[(1S,2S)-2-[2-(4-fluoro-2-methoxyphenyl)propanamido]-2,3-dihydro-1H-inden-1-yl]carbamate;
tert-butyl N-[(1S,2S)-2-[2-(2-chloro-4-fluorophenyl)propanamido]-2,3-dihydro-1H-inden-1-yl]carbamate;
tert-butyl N-[(1S,2S)-2-{2-[4-fluoro-2-(trifluoromethyl)phenyl]propanamido}-2,3-dihydro-1H-inden-1-yl]carbamate;
(2R)-2-(4-fluorophenyl)-N-[(1R,2R)-1-(methylamino)-2,3-dihydro-1H-inden-2-yl]propanamide;
tert-butyl N-[(1S,2S)-2-[2-phenyl-3-(pyrrolidin-1-yl)propanamido]-2,3-dihydro-1H-inden-1-yl]carbamate;
(2R)-2-(4-fluorophenyl)-N-[(1S,2S)-1-(methylamino)-2,3-dihydro-1H-inden-2-yl]propanamide;
(2R)—N-(trans)-(1-methanesulfonyl-2,3-dihydro-1H-inden-2-yl)-2-methoxy-2-phenylacetamide;
(2 S)—N-(trans)-(1-methanesulfonyl-2,3-dihydro-1H-inden-2-yl)-2-methoxy-2-phenylacetamide;
(2S)-2-(4-fluorophenyl)-N-[(1R,2S)-1-(methylamino)-2,3-dihydro-1H-inden-2-yl]propanamide;
2-(cyclopropylmethoxy)-2-(4-fluorophenyl)-N-(trans)-(1-methanesulfonyl-2,3-dihydro-1H-inden-2-yl)acetamide;
2-(cyclopropylmethoxy)-2-(4-fluorophenyl)-N-(trans)-(1-methanesulfonyl-2,3-dihydro-1H-inden-2-yl)acetamide; and
   enantiomers, diastereoisomers and mixtures thereof; and
   pharmaceutically acceptable salts of any of the foregoing.

It should be noted that each of the chemical compounds listed above represents a particular and independent aspect of the invention.

The present invention further provides a process for the preparation of a compound of formula (I) or a pharmaceutically acceptable salt thereof as defined above which comprises reacting a compound of formula (II), or a salt (e.g. hydrochloride salt) thereof,

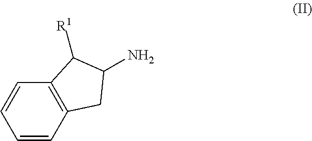

in which R¹ is as defined in formula (I) above, with a compound of formula (III), or a salt (e.g. lithium salt, or hydrochloride salt) thereof,

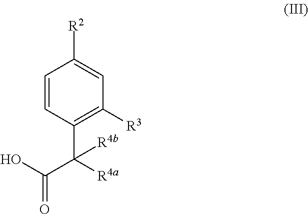

in which R², R³, R^{4a} and R^{4b} are as defined in formula (I) above;
and optionally thereafter carrying out one or more of the following procedures:

converting a compound of formula (I) into another compound of formula (I)

removing any protecting groups

forming a pharmaceutically acceptable salt.

The above process may conveniently be carried out by combining the amine of formula (II) with the carboxylic acid of formula (III) in the presence of a coupling reagent such as

(1) EDC (1-ethyl-3-(3-dimethylaminopropyl)carbodiimide) and HOAt (7-aza-1-hydroxybenzotriazole) with triethylamine in dichloromethane at room temperature, or
(2) HATU (1-[bis(dimethylamino)methylene]-1H-1,2,3-triazolo[4,5-b]pyridinium 3-oxid hexafluorophosphate) with triethylamine in dichloromethane at room temperature.

Compounds of formulae (II) and (III) are known compounds or may be prepared according to processes known in the art.

In one embodiment, a compound of formula (I) may be converted into another compound of formula (I). For example, a compound of formula (I) in which R¹ represents a hydroxyl group may be converted into a corresponding compound of formula (I) in which R¹ represents a —(CH₂)ₘ—(O)ₙ—R⁵ group in which m is 0 or 1, n is 1 and R⁵ is a C₁-C₆ alkyl, by reacting the former with silver oxide and a suitable halide (e.g an alkyl halide such as methyl iodide or ethyl iodide) in the presence of a polar solvent such as dimethylformamide or acetonitrile at a temperature in the range of from 18° C. to 100° C.

Alternatively, a compound of formula (I) in which R¹ represents a hydroxyl group cis to the NH of the NHC(O) in formula (I) may be converted into a corresponding compound of formula (I) in which R¹ represents a —(CH₂)ₚNR⁶R⁷ group trans to the NH of the NHC(O) in formula (I) and p is 0, by reacting the former with methanesulphonic anhydride and triethylamine in tetrahydrofuran at a temperature in the range of from −78° C. to 0° C., followed by reaction with an amine of formula, HNR⁶R⁷, where R⁶ and R⁷ are as defined above, at a temperature in the range of from 0° C. to room temperature.

A compound of formula (I) in which R¹ represents a —(CH₂)ₚNR⁶R⁷ group where p is 0, R⁶ is hydrogen and R⁷ is t-butoxycarbonyl may be converted into a corresponding compound of formula (I) in which R¹ represents a —(CH₂)ₚNR⁶R⁷ group where p is 0, R⁶ is hydrogen and R⁷ is hydrogen, by reacting the former with hydrochloric acid in methanol at room temperature or trifluoroacetic acid (TFA) in dichloromethane at room temperature.

A compound of formula (I) in which R¹ represents a —(CH₂)ₚNR⁶R⁷ group where p is 0, R⁶ is hydrogen and R⁷ is t-butoxycarbonyl may be converted into a corresponding compound of formula (I) in which R¹ represents a —(CH₂)ₚNR⁶R⁷ group where p is 0, R⁶ is hydrogen and R⁷ is methyl, by reacting the former with a reducing agent such as lithium aluminium hydride in tetrahydrofuran at a temperature in the range of from room temperature to the reflux temperature.

A compound of formula (I) in which R¹ represents a —(CH₂)ₚNR⁶R⁷ group where p is 0, R⁶ is hydrogen and R⁷ is t-butoxycarbonyl may be converted into a corresponding compound of formula (I) in which R¹ represents a —(CH₂)ₚNR⁶R⁷ group where p is 0, R⁶ is hydrogen and R⁷ is (CH₂)_{q}—R⁸ where q is 0, by reacting the former with lithium bis(trimethylsilyl)amide and a compound of formula R⁸-L¹, where L¹ represents a halogen atom or a leaving group such as mesyl (methanesulphonyl) or tosyl (toluenesulphonyl) and R⁸ is as defined above, in dimethylformamide at room temperature, followed by reaction with hydrochloric acid.

A compound of formula (I) in which R¹ represents a —(CH₂)ₚNR⁶R⁷ group where p is 0, R⁶ is hydrogen and R⁷ is hydrogen may be converted into a corresponding compound of formula (I) in which R¹ represents a —(CH₂)ₚNR⁶R⁷ group where p is 0, R⁶ is hydrogen and R⁷ is —SO₂R⁹ (where R⁹ is as defined above) or C₁-C₆ alkylcarbonyl or C₃-C₆ cycloalkylcarbonyl, by reacting the former with a suitable sulphonyl chloride (e.g. methylsulphonyl chloride) or acid chloride (e.g. acetyl chloride) with triethylamine in dichloromethane at a temperature in the range of from room temperature to 40° C.

A compound of formula (I) in which R¹ represents a —(CH₂)ₚNR⁶R⁷ group where p is 0, R⁶ is hydrogen and R⁷ is hydrogen may be converted into a corresponding compound of formula (I) in which R¹ represents a —(CH₂)ₚNR⁶R⁷ group where p is 0, R⁶ is hydrogen and R⁷ is (CH₂)_{q}—R⁸ where q is 0, by reacting the former with a compound of formula R⁸-L¹, where L¹ represents a halogen atom or a leaving group such as mesyl (methanesulphonyl) or tosyl (toluenesulphonyl) and R⁸ is as defined above, in the presence of diisopropylethylamine (DIPEA) in ethanol at room temperature.

A compound of formula (I) in which R¹ represents a —(CH₂)ₚNR⁶R⁷ group where p is 0, R⁶ is hydrogen and R⁷ is hydrogen may be converted into a corresponding compound of formula (I) in which R¹ represents a —(CH₂)ₚNR⁶R⁷ group where p is 0, R⁶ is hydrogen and R⁷ is (CH₂)_{q}—R⁸ where q is 0 or 1, by reacting the former with a suitable aldehyde (such as cyclopropane carbaldehyde) or ketone (such as cyclobutanone), in the presence of sodium triacetoxyborohydride and glacial acetic acid in dichloromethane at a temperature in the range of from room temperature to 40° C.

A compound of formula (I) in which R¹ represents a —(CH₂)ₚNR⁶R⁷ group where p is 0, R⁶ is hydrogen and R⁷ is hydrogen may be converted into a corresponding compound of formula (I) in which R¹ represents a —(CH₂)ₚNR⁶R⁷ group where p is 0, and R⁶ and R⁷ are —(CH₂)_{q}—R⁸ where q is 0 or 1, by reacting the former with a suitable aldehyde (such as cyclopropane carbaldehyde) or ketone (such as cyclobutanone), in the presence of sodium triacetoxyborohydride and glacial acetic acid in dichloromethane at a temperature in the range of from room temperature to 40° C.

A compound of formula (I) in which R¹ represents a —(CH₂)ₚNR⁶R⁷ group where p is 0, R⁶ is hydrogen and R⁷ is hydrogen may be converted into a corresponding compound of formula (I) in which R¹ represents a —(CH₂)ₚNR⁶R⁷ group where p is 0, R⁶ is hydrogen and R⁷ is —CH₂CF₃, by reacting the former with 2,2,2-trifluoroethyl trichloromethanesulphonate in the presence of potassium carbonate in acetone at a temperature in the range of from room temperature to the reflux temperature, or under microwave irradiation up to 250° C.

A compound of formula (I) in which R¹ represents a —(CH₂)ₚNR⁶R⁷ group where p is 0, and R⁶ and R⁷ together with the nitrogen atom to which they are attached form a saturated 4- to 7-membered heterocylic ring substituted with —OSi(R′)₃ where R′ is C₁-C₆ alkyl, may be converted into a corresponding compound of formula (I) in which R¹ represents a —(CH₂)ₚNR⁶R⁷ group where p is 0, and R⁶ and R⁷ together with the nitrogen atom to which they are attached form a saturated 4- to 7-membered heterocylic ring substituted with OH, by reacting the former with tetra-n-butylammonium fluoride (TBAF).

A compound of formula (I) in which R¹ represents a —(CH₂)ₚNR⁶R⁷ group where p is 0, and R⁶ and R⁷ together with the nitrogen atom to which they are attached form a saturated 4- to 7-membered heterocylic ring substituted with OH, may be converted into a corresponding compound of formula (I) in which R¹ represents a —(CH₂)ₚNR⁶R⁷ group where p is 0, and R⁶ and R⁷ together with the nitrogen atom to which they are attached form a saturated 4- to 7-membered heterocylic ring substituted with F, by reacting the former with diethylaminosulfur trifluoride (DAST).

A compound of formula (I) in which R^{4a} represents (X)ₜ—(CH₂)ᵥ—R¹⁶ where t is 1, v is 0, X is NHC(O)O and R¹⁶═R¹⁷=t-butyl can be converted into a corresponding compound of formula (I) in which R^{4a} represents (X)ₜ—(CH₂)ᵥ—R¹⁶ where t is 0, v is 0 and R¹⁶═NR¹⁸R¹⁹═NH₂, by reacting the former with hydrochloric acid in methanol at room temperature.

A compound of formula (I) in which R^{4a} represents (X)ₜ—(CH₂)ᵥ—R¹⁶ where t is 0, v is 0 and R¹⁶═NR¹⁸R¹⁹═NH₂ can be converted into a corresponding compound of formula (I) in which R^{4a} represents (X)ₜ—(CH₂)ᵥ—R¹⁶ where t is 0, v is 0 and R¹⁶═NR¹⁸R¹⁹═NH(C₁-C₆ alkyl), by reacting the former with a suitable aldehyde, e.g. acetaldehyde, in the presence of sodium triacetoxyborohydride and glacial acetic acid in dichloromethane at a temperature in the range of from room temperature to 40° C.

A compound of formula (I) in which R^{4a} represents (X)ₜ—(CH₂)ᵥ—R¹⁶ where t is 0, v is 0 and R¹⁶═NR¹⁸R¹⁹═NH₂ can be converted into a corresponding compound of formula (I) in which R^{4a} represents (X)ₜ—(CH₂)ᵥ—R¹⁶ where t is 0, v is 0 and R¹⁶═NR¹⁸R¹⁹═NHC(O)C₁-C₆ alkyl or NHC(O)C₃-C₆ cycloalkyl or NHSO₂C₁-C₆ alkyl, by reacting the former with a suitable acid chloride (e.g. acetyl chloride) or sulphonyl chloride/sulphonyl anhydride (e.g. cyclopropanesulphonyl chloride or methanesulphonyl methanesulphonate) in the presence of triethylamine in dichloromethane at a temperature in the range of from room temperature to 40° C.

A compound of formula (I) in which R^{4a} represents (X)ₜ—(CH₂)ᵥ—R¹⁶ where t is 0, v is 0 and R¹⁶═NR¹⁸R¹⁹═NH₂ can be converted into a corresponding compound of formula (I) in which R^{4a} represents (X)ₜ—(CH₂)ᵥ—R¹⁶ where t is 1, X is NHSO₂, v is 0 and R¹⁶ is a saturated or unsaturated 4- to 6-membered optionally substituted heterocyclic ring, by reacting the former with a suitable sulphonyl chloride/sulphonyl anhydride (e.g. cyclopropanesulphonyl chloride or methanesulphonyl methanesulphonate) in the presence of triethylamine in dichloromethane at a temperature in the range of from room temperature to 40° C.

Further processes for converting a compound of formula (I) into another compound of formula (I) are shown in the following reaction schemes 1 to 3 in which R², R³, R^{4a} and R^{4b} are as defined above.

When R¹ represents a hydroxyl group cis to the NH of NHC(O), it can be converted to a cyano group which is trans to the NH of NH(CO) as illustrated below:

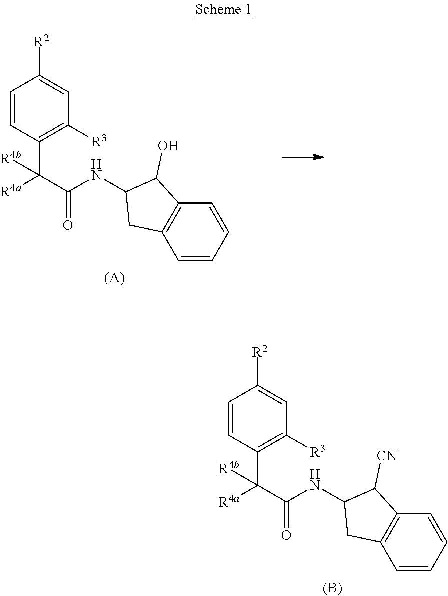

The reaction is carried out in two steps. Firstly compound (A) is reacted with methanesulphonic anhydride and triethylamine in tetrahydrofuran at a temperature of about −78° C., followed by reaction with sodium cyanide at a temperature in the range of from 0° C. to room temperature to yield compound (B).

When R¹ represents a hydroxyl group cis to the NH of NHC(O), it can be converted to a —SO₂R^{1a} group which is trans to the NH of NHC(O) as follows:

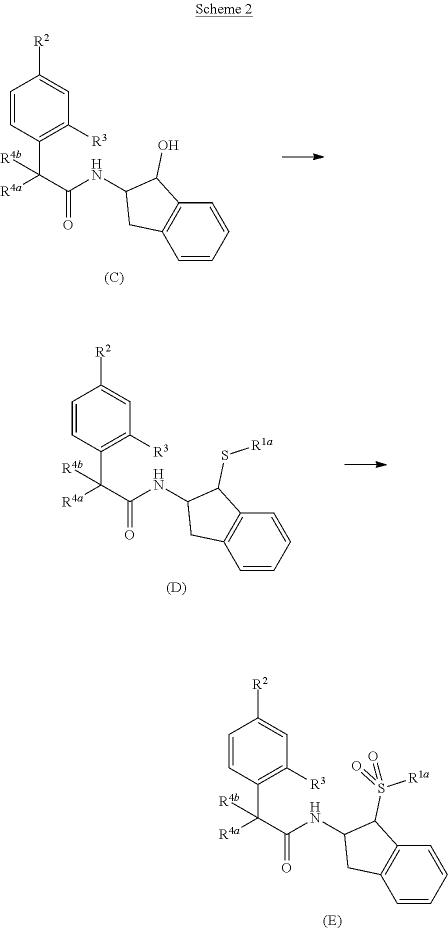

Compound (C) is reacted with methanesulphonic anhydride and triethylamine in tetrahydrofuran at a temperature of about −78° C., followed by reaction with a thiol of formula R^{1a}—SH in which R^{1a} is as defined above, at a temperature in the range of from 0° C. to room temperature to form compound (D). Compound (D) is then reacted with an oxidising agent such as meta-chloroperoxybenzoic acid at a temperature in the range of from 0° C. to 40° C. to yield compound (E).

When R¹ represents a cyano group, it can be converted to —CH₂OH or —CH₂NR⁶R⁷ as follows:

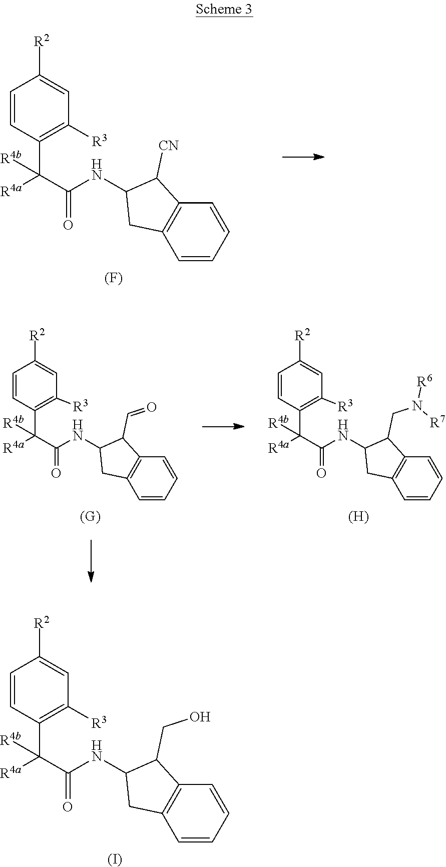

Compound (F) is reacted with a reducing agent such as diisobutylaluminium hydride in tetrahydrofuran at a temperature of about −78° C. to form compound (G). Compound (G) can be reacted (i) with an amine of formula HNR⁶R⁷ where R⁶ and R⁷ are as defined above, in the presence of acetic acid and sodium triacetoxyborohydride in dichloromethane and at a temperature in the range of from room temperature to 40° C. to form compound (H), or (ii) with a reducing agent such as sodium borohydride in methanol at a temperature in the range of from 0° C. to room temperature to form compound (I).

A compound of formula (I) in which R¹ represents NHBoc (Boc=t-butoxycarbonyl), 4a represents (X)ₜ—(CH₂)ᵥ—R¹⁶ where t is 0, v is 1 and R¹⁶═NR¹⁸R¹⁹ may be prepared as illustrated in the following reaction scheme 4 in which R² and R³ are as defined above:

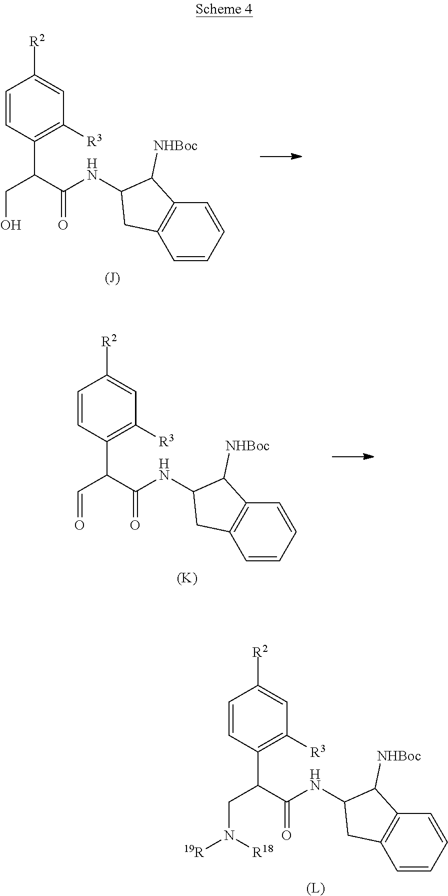

Compound (J) can be reacted with an oxidising agent such as Dess-Martin Periodinane in dichloromethane at a temperature in the range of from 0° C. to room temperature to form Compound (K) which in turn is reacted with an amine of formula HNR¹⁸R¹⁹ where R¹⁸ and R¹⁹ are as defined above, in the presence of acetic acid and sodium triacetoxyborohydride in dichloromethane and at a temperature in the range of from room temperature to 40° C. to form compound (L).

A compound of formula (I) in which R¹ represents NHBoc (Boc=t-butoxycarbonyl), and R^{4a} represents (X)ₜ—(CH₂)ᵥ—R¹⁶ where t is 0, v is 1 and R¹⁶═OH may be converted into a corresponding compound of formula (I) in which R¹ represents NHBoc (Boc=t-butoxycarbonyl), and R^{4a} represents (X)ₜ—(CH₂)ᵥ—R¹⁶ where t is 0, v is 1 and R¹⁶═NR¹⁸R¹⁹ wherein R¹⁸ and R¹⁹ are as defined above, by reacting the former with methanesulfonic anhydride and an amine HNR¹⁸R¹⁹.

It will be appreciated by those skilled in the art that in the processes of the present invention certain functional groups such as phenol, hydroxyl or amino groups in the reagents may need to be protected by protecting groups. Thus, the preparation of the compounds of formula (I) may involve, at an appropriate stage, the introduction and/or removal of one or more protecting groups.

The protection and deprotection of functional groups is described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3^{rd} edition, P. J. Kocienski, Thieme (2005).

The compounds of formula (I) above may be converted to a pharmaceutically acceptable salt thereof, preferably an acid addition salt such as a formate, hemi-formate, hydrochloride, hydrobromide, benzenesulphonate (besylate), saccharin (e.g. monosaccharin), trifluoroacetate, sulphate, nitrate, phosphate, acetate, fumarate, maleate, tartrate, lactate, citrate, pyruvate, succinate, valerate, propanoate, butanoate, malonate, oxalate, 1-hydroxy-2-napthoate (xinafoate), methanesulphonate or p-toluenesulphonate salt. In one embodiment of the invention, the compounds of formula (I) are in the form of a hydrochloride salt.

In one aspect of the invention, compounds of formula (I) may bear one or more radiolabels. Such radiolabels may be introduced by using radiolabel-containing reagents in the synthesis of the compounds of formula (I), or may be introduced by coupling the compounds of formula (I) to chelating moieties capable of binding to a radioactive metal atom. Such radiolabeled versions of the compounds may be used, for example, in diagnostic imaging studies.

Unless stated otherwise, any atom specified herein may also be an isotope of said atom. For example, the term “hydrogen” encompasses ¹H, ²H and ³H. Similarly carbon atoms are to be understood to include ¹²C, ¹³C and ¹⁴C, nitrogen atoms are to be understood to include ¹⁴N and ¹⁵N, and oxygen atoms are to be understood to include ¹⁶O, ¹⁷O and ¹⁸O.

In a further aspect of the invention, compounds of formula (I) may be isotopically labelled. As used herein, an “isotopically labelled” compound is one in which the abundance of a particular nuclide at a particular atomic position within the molecule is increased above the level at which it occurs in nature.

Compounds of formula (I) and their salts may be in the form of hydrates or solvates which form an aspect of the present invention. Such solvates may be formed with common organic solvents, including but not limited to, alcoholic solvents e.g. methanol, ethanol or isopropanol.

Where compounds of formula (I) are capable of existing in stereoisomeric forms, it will be understood that the invention encompasses the use of all geometric and optical isomers (including atropisomers) of the compounds of formula (I) and mixtures thereof including racemates. The use of tautomers and mixtures thereof also forms an aspect of the present invention. Enantiomerically pure forms are particularly desired. “Enantiomerically pure” denotes the presence of at least 75% w (percent by weight), in particular at least 90% w and, more particularly, at least 95% w of one of the two possible enantiomers of a compound.

Compounds of formula (I) and their salts may be amorphous or in a polymorphic form or a mixture of any of these, each of which forms an aspect of the present invention.

The compounds of formula (I) and their pharmaceutically acceptable salts have activity as pharmaceuticals and may be used in treating conditions or disorders associated with changes in one or both of the glutamatergic and GABAergic signalling pathways regulated in full or in part by metabotropic glutamate receptor 7.

Thus, the present invention provides a compound of formula (I) or a pharmaceutically acceptable salt thereof as hereinbefore defined for use in therapy, in particular for the treatment of conditions associated with metabotropic glutamate receptor 7.

The present invention also provides the use of a compound of formula (I) or a pharmaceutically acceptable salt thereof as hereinbefore defined for the preparation of a medicament for the treatment of conditions associated with metabotropic glutamate receptor 7.

The present invention still further provides a method of treating a condition associated with metabotropic glutamate receptor 7 which comprises administering to a patient in need thereof a therapeutically effective amount of a compound of formula (I) or a pharmaceutically acceptable salt thereof as hereinbefore defined.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

As used herein the terms “condition”, “disorder”, and “disease” relate to any unhealthy or abnormal state. The term “conditions associated with metabotropic glutamate receptor 7” includes conditions, disorders and diseases in which the modulation of mGluR7 may provide a therapeutic benefit, examples of which include:

(1) Nervous system disorders: Parkinson's disease, including dementia associated with Parkinson's disease; Alzheimer's disease; Huntington's Chorea; amyotrophic lateral sclerosis; multiple sclerosis; bipolar disorder; and psychiatric disorders such as schizophrenia, post-traumatic stress disorder, anxiety disorders and depression (e.g. major depressive disorder);
(2) Addiction disorders: alcohol, drug or nicotine addiction;
(3) Hearing disorders: hearing loss and/or tinnitus caused by age, noise or trauma; and
(4) Others: idiopathic autism; severe neonatal encephalopathy; autism spectrum disorder (ASD); X-linked intellectual disability (also known as X-linked mental retardation); epilepsy; cerebral ischemias; eye disorders; and pain (e.g. inflammatory pain or neuropathic pain).

Schizophrenia is a debilitating psychiatric disorder characterised by a combination of negative symptoms (such as social withdrawal, anhedonia, avolition and apathy) and positive symptoms (including hallucinations, delusions and paranoia) as well as marked cognitive deficits (such as impairment of executive function). The executive function (EF) has been defined as “a set of abilities, which allows us to invoke voluntary control of our behavioral responses. These functions enable human beings to develop and carry out plans, make up analogies, obey social rules, solve problems, adapt to unexpected circumstances, do many tasks simultaneously, and locate episodes in time and place. EF includes divided attention and sustained attention, working memory (WM), set-shifting, flexibility, planning, and the regulation of goal directed behavior and can be defined as a brain function underlying the human faculty to act or think not only in reaction to external events but also in relation with internal goals and states” (Orellana G. and Slachevsky A., 2013. Executive Functioning in Schizophrenia. Front. Psychiatry, 4, 35).

Accordingly, the present invention also provides a method of treating a negative symptom, a positive symptom and/or a cognitive deficit associated with a psychiatric disorder, especially schizophrenia, which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (m/kg) to 100 micrograms per kilogram body weight (m/kg). Alternatively, if the compound is administered orally, then the daily dosage of the compound of the invention may be in the range from 0.01 micrograms per kilogram body weight (m/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier.

Therefore the present invention further provides a pharmaceutical composition comprising a compound of formula (I) or a pharmaceutically acceptable salt thereof as hereinbefore defined, in association with a pharmaceutically acceptable adjuvant, diluent or carrier.

The invention still further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to, capsules, tablets, powders, granules, and aqueous suspensions and solutions. These dosage forms are prepared according to techniques well-known in the art of pharmaceutical formulation. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The compounds of the invention (that is, compounds of formula (I) and pharmaceutically acceptable salts thereof) may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. Such therapeutic agents may be selected from the following:

(i) anti-addiction drugs including, for example, acamprosate, disulfiram, naltrexone and nalmefene for alcohol dependency, and gabapentin, modafinil, topiramate, vigabatrin and baclofen for drug, particularly cocaine, addiction;
(ii) antidepressants such as amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robaizotan, sertraline, sibutramine, tianeptine, thionisoxetine, tranylcypromaine, trazodone, trimipramine, venlafaxine, vortioxetine and equivalents and pharmaceutically active isomer(s) and/or metabolite(s) thereof;
(iii) antipsychotics including, for example, amisulpride, aripiprazole, asenapine, benzisoxidil, bifeprunox, brexpiprazole, carbamazepine, cariprazine, clozapine, chlorpromazine, debenzapine, divalproex, duloxetine, eszopiclone, fluphenazine, haloperidol, iloperidone, lamotrigine, loxapine, lurasidone, mesoridazine, olanzapine, paliperidone, perlapine, perphenazine, phenothiazine, phenylbutlypiperidine, pimozide, prochlorperazine, quetiapine, risperidone, sertindole, sulpiride, suproclone, suriclone, thioridazine, trifluoperazine, trimetozine, valproate, valproic acid, zopiclone, zotepine, zicronapine, ziprasidone, and equivalents and pharmaceutically active isomer(s) and/or metabolite(s) thereof;
(iv) anxiolytics including, for example, alnespirone, azapirones, benzodiazepines, barbiturates, and equivalents and pharmaceutically active isomer(s) and/or metabolite(s) thereof. Example anxiolytics include adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, prazosin, quazepam, reclazepam, tracazolate, trepipam, temazepam, triazolam, uldazepam, and zolazepam; and equivalents and pharmaceutically active isomer(s) and/or metabolite(s) thereof;
(v) anticonvulsants including, for example, carbamazepine, valproate, lamotrigine, levetiracetam and gabapentin, and equivalents and pharmaceutically active isomer(s) and/or metabolite(s) thereof;
(vi) Alzheimer's therapies including, for example, donepezil, galantamine, memantine, rivastigmine, tacrine, and equivalents and pharmaceutically active isomer(s) and/or metabolite(s) thereof;
(vii) Parkinson's therapies including, for example, L-dopa, ropinirole, pramipexole, monoamine oxidase type B (MAO-B) inhibitors such as deprenyl, selegiline and rasagiline, catechol-O-methyl transferase (COMT) inhibitors such as entacapone or tolcapone, adenosine A-2 inhibitors, dopamine re-uptake inhibitors, NMDA antagonists, Nicotine agonists, and Dopamine agonists and inhibitors of neuronal nitric oxide synthase, and equivalents and pharmaceutically active isomer(s) and/or metabolite(s) thereof;
(viii) migraine therapies including, for example, almotriptan, amantadine, botulinum toxin A, bromocriptine, butalbital, cabergoline, dichloralphenazone, dihydroergotamine, eletriptan, frovatriptan, lisuride, naratriptan, pergolide, pramipexole, rizatriptan, ropinirole, sumatriptan, topiramate, zolmitriptan, and zomitriptan, and equivalents and pharmaceutically active isomer(s) and/or metabolite(s) thereof;
(ix) stroke therapies including, for example, abciximab, activase, citicoline, desmoteplase, and equivalents and pharmaceutically active isomer(s) and/or metabolite(s) thereof;
(x) urinary incontinence therapies including, for example, darafenacin, duloxetine, falvoxate, mirabegron, oxybutynin, propiverine, robalzotan, solifenacin, and tolterodine, and equivalents and pharmaceutically active isomer(s) and/or metabolite(s) thereof;
(xi) neuropathic pain therapies including, for example, capsaicin, gabapentin, lidoderm, and pregabalin, and equivalents and pharmaceutically active isomer(s) and/or metabolite(s) thereof;
(xii) nociceptive pain therapies such as celecoxib, etoricoxib, lumiracoxib, rofecoxib, valdecoxib, diclofenac, loxoprofen, naproxen, and paracetamol, and equivalents and pharmaceutically active isomer(s) and/or metabolite(s) thereof;
(xiii) insomnia therapies including, for example, allobarbital, alonimid, amobarbital, benzoctamine, butabarbital, capuride, chloral, cloperidone, clorethate, dexclamol, ethchlorvynol, eszopiclone, etomidate, glutethimide, halazepam, hydroxyzine, lorediplon, mecloqualone, melatonin, mephobarbital, methaqualone, midaflur, nisobamate, pentobarbital, phenobarbital, propofol, ralmeteon, roletamide, suvorexant, triclofos, secobarbital, zaleplon, and zolpidem, zopiclone and equivalents and pharmaceutically active isomer(s) and/or metabolite(s) thereof;
(xiv) mood stabilizers including, for example, carbamazepine, divalproex, gabapentin, lamotrigine, lithium, olanzapine, quetiapine, valproate, valproic acid, and verapamil, and equivalents and pharmaceutically active isomer(s) and/or metabolite(s) thereof;
(xv) 5HT1B ligands such as, for example, compounds disclosed in WO 99/05134 and WO 02/08212;
(xvi) mGluR2 agonists;
(xvii) alpha 7 nicotinic agonists such as, for example, compounds disclosed in WO 96/006098, WO 97/030998, WO 99/003859, WO 00/042044, WO 01/029034, WO 01/60821, WO 01/36417, WO 02/096912, WO 03/087102, WO 03/087103, WO 03/087104, WO 2004/016617, WO 2004/016616, and WO 2004/019947;
(xviii) chemokine receptor CCR1 inhibitors; and
(xix) delta opioid agonists such as, for example, compounds disclosed in WO 1997/23466 and WO 2002/094794.

Such combination products employ the compounds of this invention within the dosage range described herein and the other pharmaceutically active agent within approved dosage ranges.

The present invention will now be further explained by reference to the following illustrative examples, in which the starting materials and reagents used are available from commercial suppliers or prepared via literature procedures.

Nuclear magnetic resonance (NMR) spectra were recorded at 400 MHz or 300 MHz as stated and at 300.3K, 298.2K or 293K unless otherwise stated; the chemical shifts (6) are reported in parts per million. Spectra were recorded using a Bruker 400 AVANCE instrument fitted with a 5 mm BBFO probe with instrument controlled by Bruker TopSpin 2.1 software, or by a Bruker 400 AVANCE-III HD instrument fitted with a 5 mm BBFO smart probe or a 5 mm BBFO probe with instrument controlled by Bruker TopSpin 3.2 software, or by a Bruker 400 AVANCE-III instrument fitted with a 5 mm BBFO probe with instrument controlled by Bruker Topspin 3.0 software or by a Bruker 300 MHz AVANCE II instrument fitted with a 5 mm DUL probe with instrument controlled by Bruker TopSpin 1.3 software, or 5 mm BBFO probe controlled by Bruker Topspin 3.2 software.

Purity was assessed using one or more of the following:
UPLC with UV (photodiode array) detection over a wide range of wavelengths, normally 220-450 nm, using a Waters Acquity UPLC system equipped with Acquity UPLC BEH, HSS or HSS T3 C18 columns (2.1 mm id×50 mm long) operated at 50 or 60° C. Mobile phases typically consisted of acetonitrile mixed with water containing either 0.1% formic acid, 0.1% TFA or 0.025% ammonia. Mass spectra were recorded with a Waters SQD single quadrupole mass spectrometer using atmospheric pressure ionisation.
UPLC with UV (photodiode array) detection over a wide range of wavelengths, normally 220-450 nm, using Shimadzu Nexera X2 UPLC controlled by Lab Solution software equipped with Acquity UPLC BEH, HSS or HSS T3 C18 columns (2.1 mm id×50 mm long) operated at 50° C. Mobile phases typically consisted of acetonitrile mixed with water containing either 0.1% formic acid, 0.1% TFA or 0.025% ammonia. Mass spectra were recorded with a Shimadzu single quadrupole mass spectrometer using DUIS ionisation.

Compounds were purified using normal phase chromatography on silica, using Biotage KP-Sil cartridges, Interchim PuriFlash cartridges or Kinesis Telos Silica cartridges, or on basic silica using Biotage KP-NH cartridges, or by reverse phase chromatographic methods using Biotage KP-C18-HS cartridges or by Biotage Isolute SCX-2 or Phenomenex Strata ABW catch-release cartridges, or by preparative HPLC.

Preparative HPLC was performed using Agilent Technologies 1100 Series system or a Waters autopurification LC/MS system typically using Waters 19 mm id×250 mm long C18 columns such as)(Bridge or SunFire 5 μm materials at rt. Mobile phases typically consisted of acetonitrile mixed with water containing either 0.1% formic acid or 0.1% ammonia, unless stated otherwise.

SFC chiral separations were performed on a Waters prep30/MS system, using a flow rate of 30 mL/min, temperature of 40° C. and a pressure of 100 bar. Mobile phases typically consisted of supercritical CO₂ and a polar solvent such as methanol, ethanol or isopropanol. Column type and eluent are detailed for individual examples.

‘Room temperature’, as used in the present specification, means a temperature in the range from about 18° C. to about 25° C.

### ABBREVIATIONS

15-Crown-5: 1,4,7,10,13-Pentaoxacyclopentadecane
DAST: Diethylaminosulfur trifluoride
DCM: Dichloromethane
DIPEA: N,N-Diisopropylethylamine
DMF: Dimethylformamide
EDC: N-(3-Dimethylaminopropyl)-N′-ethylcarbodiimide hydrochloride
HATU: 1-[Bis(dimethylamino)methylene]-1H-1,2,3-triazolo[4,5-b]pyridinium 3-oxid hexafluorophosphate
HOAt: 1-Hydroxy-7-azabenzotriazole
HPLC: High-Performance Liquid Chromatography
IPA: Isopropyl alcohol
NBS: N-Bromosuccinimide
SFC: Super Critical Fluid Chromatography
TBAF: Tetrabutylammonium fluoride
THF: Tetrahydrofuran
T3P: 1-Propylphosphonic acid

### 1. INTERMEDIATES

### Intermediate 1: 2-(2,4-difluorophenyl)butanoic acid

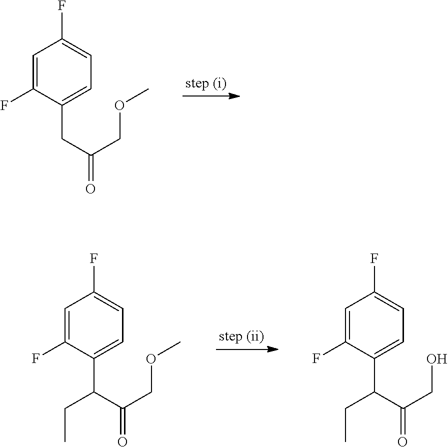 [SMILES: COCC(=O)Cc1ccc(F)cc1F ; CCC(C(=O)COC)c1ccc(F)cc1F ; CCC(C(=O)CO)c1ccc(F)cc1F]

### Step (i): ethyl 2-(2,4-difluorophenyl)butanoate

Sodium hydride (60% dispersion in mineral oil, 0.048 g, 1.199 mmol) was added to a solution of ethyl 2-(2,4-difluorophenyl)acetate (0.2 g, 0.999 mmol) in THF (5 mL) under nitrogen. The reaction was stirred at room temperature for 30 minutes. Iodoethane (0.129 mL, 1.599 mmol) and DMF (3 mL) were added and the reaction was stirred overnight. The mixture was partitioned between ethyl acetate and water. The phases were separated and the aqueous extracted with ethyl acetate. The combined organics were washed with half saturated brine, dried (phase separator) and concentrated in vacuo to afford the title compound.

¹H NMR (300 MHz, DMSO-d₆) δ ppm 0.77-0.86 (m, 3H) 1.13 (t, J=7.00 Hz, 3H) 1.62-1.79 (m, 1H) 1.94-2.10 (m, 1H) 3.71-3.80 (m, 1H) 4.08 (q, J=7.00 Hz, 2H) 7.03-7.14 (m, 1H) 7.17-7.29 (m, 1H) 7.35-7.47 (m, 1H)

### Step (ii): 2-(2,4-difluorophenyl)butanoic acid

Lithium hydroxide (0.044 g, 1.840 mmol) was added to a solution of ethyl 2-(2,4-difluorophenyl)butanoate (0.21 g, 0.920 mmol) in THF (2 mL) and water (2 mL). The reaction was stirred at room temperature overnight. Water was added and the reaction acidified to pH 2 with 2 M HCl then extracted with ethyl acetate. The combined organics were washed with saturated brine, dried (phase separator) and concentrated in vacuo to afford the title compound.

¹H NMR (400 MHz, DMSO-d₆) δ ppm 0.77-0.84 (m, 3H) 1.60-1.74 (m, 1H) 1.93-2.05 (m, 1H) 3.66 (t, J=7.61 Hz, 1H) 7.02-7.10 (m, 1H) 7.17-7.26 (m, 1H) 7.35-7.44 (m, 1H) 12.49 (s, 1H)

### Intermediate 2: (S)-2-(2,4-difluorophenyl)propanoic acid

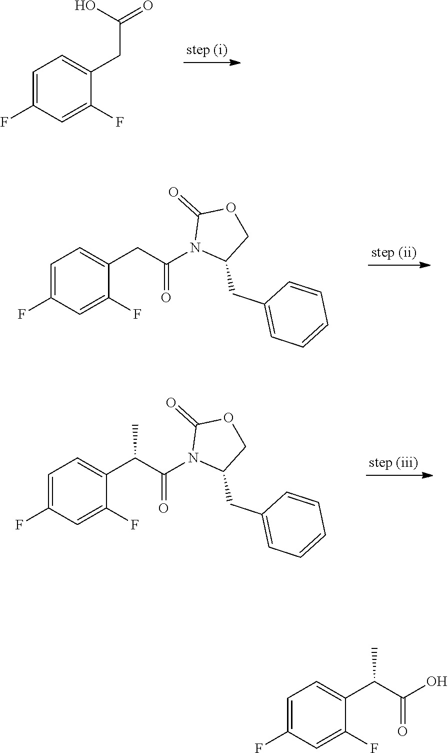 [SMILES: O=C(O)Cc1ccc(F)cc1F ; O=C(Cc1ccc(F)cc1F)N1C(=O)OC[C@@H]1Cc1ccccc1 ; C[C@H](C(=O)N1C(=O)OC[C@@H]1Cc1ccccc1)c1ccc(F)cc1F ; C[C@H](C(=O)O)c1ccc(F)cc1F]

### Step (i): (S)-4-benzyl-3-(2-(2,4-difluorophenyl)acetyl)oxazolidin-2-one

n-Butyl lithium (2.5 M solution in hexane, 34.75 mL, 87 mmol) was added slowly to a solution of (S)-4-benzyloxazolidin-2-one (14.0 g, 79.09 mmol) in THF (280 mL) under nitrogen at −70° C. The mixture was stirred at −70° C. for 30 minutes. Meanwhile, triethylamine (13.58 g, 134.46 mmol) was added to a solution of 2-(2,4-difluorophenyl)acetic acid (14.96 g, 86.9 mmol) in THF (280 mL) at 0° C. and stirred for 30 minutes. Pivaloyl chloride (12.44 g, 102.82 mmol) was added drop-wise over 30 minutes at 0° C. and then stirred for 1 hour at 0° C. The benzyloxazolidinone solution was then transferred by cannula to the anhydride solution at −70° C. and stirred for 30 minutes at −70° C. The mixture was quenched with saturated NH₄Cl solution, diluted with water and extracted with ethyl acetate. The combined organics were washed with brine, dried (sodium sulphate) and concentrated in vacuo. The crude product was purified by column chromatography on silica, eluted with 8-10% ethyl acetate/hexane to afford the title compound.

¹H NMR (400 MHz, DMSO-d₆) δ ppm 2.90-3.04 (m, 2H) 4.11-4.43 (m, 4H) 4.62-4.72 (m, 1H) 7.04-7.12 (m, 1H) 7.17-7.36 (m, 6H) 7.37-7.47 (m, 1H)

### Step (ii): (S)-4-benzyl-3-((S)-2-(2,4-difluorophenyl)propanoyl)oxazolidin-2-one

Sodium bis(trimethylsilyl)amide (1 M solution in THF, 68 mL, 68 mmol) was added slowly to a solution of (S)-4-benzyl-3-(2-(2,4-difluorophenyl)acetyl)oxazolidin-2-one (15 g, 45.31 mmol) in THF (180 mL) at −70° C. and stirred for 1 hour. Methyl iodide (32.18 g, 226.58 mmol) was then added at −70° C., the mixture was allowed to warm to 0° C. and stirred for 30 minutes. The mixture was quenched with saturated NH₄Cl solution, diluted with water and extracted by ethyl acetate. The combined organics were washed with brine, dried (sodium sulphate) and concentrated in vacuo. The crude product was purified by column chromatography on silica, eluted with 4-6% ethyl acetate/hexane to afford the title compound.

¹H NMR (400 MHz, DMSO-d₆) δ ppm 1.45 (d, J=7.09 Hz, 3H) 2.94-3.08 (m, 2H) 4.20-4.26 (m, 1H) 4.29-4.37 (m, 1H) 4.67-4.76 (m, 1H) 5.03-5.12 (m, 1H) 7.04-7.11 (m, 1H) 7.18-7.43 (m, 7H)

### Step (iii): (S)-2-(2,4-difluorophenyl)propanoic acid

Lithium hydroxide (2.37 g, 57.97 mmol) was added to a solution of (S)-4-benzyl-3-((S)-2-(2,4-difluorophenyl)propanoyl)oxazolidin-2-one (10 g, 28.98 mmol) in THF (360 mL) and water (120 mL). Hydrogen peroxide (26.28 mL, 231.88 mmol) was then added slowly at 0° C. and stirred for 3 hours at 0° C. The mixture was quenched with saturated sodium thiosulphate solution, diluted with water and extracted with ethyl acetate. The aqueous phase was acidified with glacial acetic acid to pH 5 then extracted with ethyl acetate to afford the product with traces of acetic acid. The compound was then lyophilized from acetonitrile to afford the title compound.

¹H NMR (400 MHz, DMSO-d₆) δ ppm 1.37 (d, J=7.32 Hz, 3H) 3.87 (q, J=7.32 Hz, 1H) 7.01-7.10 (m, 1H) 7.15-7.25 (m, 1H) 7.34-7.44 (m, 1H) 12.48 (br. s., 1H)

### Intermediate 3: (S)-2-(4-fluorophenyl)propanoic acid

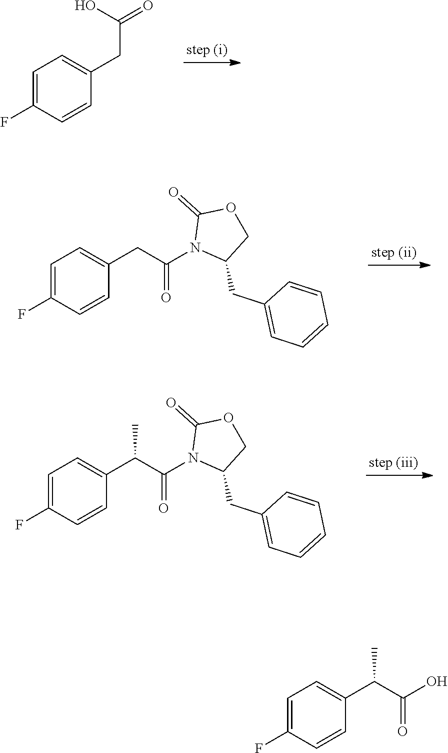 [SMILES: O=C(O)Cc1ccc(F)cc1 ; O=C(Cc1ccc(F)cc1)N1C(=O)OC[C@@H]1Cc1ccccc1 ; C[C@H](C(=O)N1C(=O)OC[C@@H]1Cc1ccccc1)c1ccc(F)cc1 ; C[C@H](C(=O)O)c1ccc(F)cc1]

### Step (i): (S)-4-benzyl-3-(2-(4-fluorophenyl)acetyl)oxazolidin-2-one

Prepared as described for Intermediate 2 step (i) using n-Butyl lithium (2.5 M solution in hexane, 34.75 mL, 87 mmol), (S)-4-benzyloxazolidin-2-one (14.0 g, 79.09 mmol), 2-(4-fluorophenyl)acetic acid (13.4 g, 86.9 mmol), triethylamine (20 g, 197.51 mmol) and pivaloyl chloride (18.96 g, 158.01 mmol). The crude product was purified by column chromatography on silica, eluted with 0-7% ethyl acetate/hexane to afford the title compound.

¹H NMR (400 MHz, DMSO-d₆) δ ppm 2.85-3.03 (m, 2H) 4.09-4.29 (m, 3H) 4.31-4.39 (m, 1H) 4.62-4.70 (m, 1H) 7.09-7.21 (m, 4H) 7.22-7.36 (m, 5H)

MS ES+: 314

### Step (ii): (S)-4-benzyl-3-((S)-2-(4-fluorophenyl)propanoyl)oxazolidin-2-one

Prepared as described for Intermediate 2 step (ii) using sodium bis(trimethylsilyl)amide (1 M solution in THF, 62.3 mL, 62.30 mmol), (S)-4-benzyl-3-(2-(4-fluorophenyl)acetyl)oxazolidin-2-one (13 g, 41.53 mmol) and methyl iodide (29.50 g, 207.60 mmol). The crude product was purified by column chromatography on silica, eluted with 0-4% ethyl acetate/hexane to afford the title compound.

¹H NMR (400 MHz, DMSO-d₆) δ ppm 1.44 (d, J=7.09 Hz, 3H) 2.93-3.07 (m, 2H) 4.16-4.23 (m, 1H) 4.24-4.32 (m, 1H) 4.62-4.69 (m, 1H) 4.93-5.01 (m, 1H) 7.09-7.18 (m, 2H) 7.19-7.38 (m, 7H)

MS ES+: 328

### Step (iii): (S)-2-(4-fluorophenyl)propanoic acid

Prepared as described for Intermediate 2 step (iii) using lithium hydroxide (2.15 g, 51.98 mmol), (S)-4-benzyl-3-((S)-2-(4-fluorophenyl)propanoyl)oxazolidin-2-one (8.5 g, 25.99 mmol) and hydrogen peroxide (24 mL, 207.9 mmol) to afford the title compound.

¹H NMR (400 MHz, DMSO-d₆) δ ppm 1.34 (d, J=7.02 Hz, 3H) 3.69 (q, J=7.02 Hz, 1H) 7.10-7.19 (m, 2H) 7.28-7.36 (m, 2H) 12.37 (br. s., 1H)

### Intermediate 4: (trans)-1-methoxy-2,3-dihydro-1H-inden-2-amine

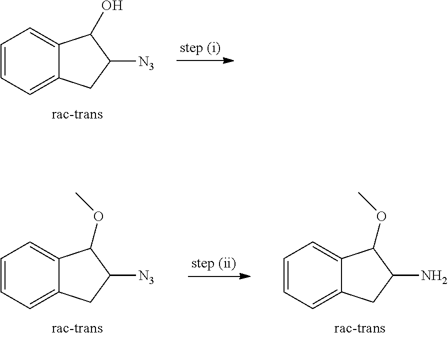

### Step (i): (trans)-2-azido-1-methoxy-2,3-dihydro-1H-indene

Methyl iodide (1.428 mL, 22.83 mmol) was added to a suspension of (trans)-2-azido-2,3-dihydro-1H-inden-1-ol ((synthesis described in Tetrahedron: Asymmetry, 1995, 6, 7, 1535, using racemic cis starting material) 1.6 g, 9.13 mmol) and silver oxide (2.54 g, 10.96 mmol) in acetonitrile (25 mL). The reaction was stirred at room temperature for 2 days in a sealed flask in the dark. The reaction was heated to 60° C. for 5 hours then stirred at room temperature overnight. The suspension was filtered through celite and concentrated in vacuo. The crude product was purified by column chromatography on silica, eluted with 0-10% ethyl acetate/petroleum ether to afford the title compound.

¹H NMR (400 MHz, CHLOROFORM-d) δ ppm 2.86-2.95 (m, 1H) 3.34-3.42 (m, 1H) 3.60 (s, 3H) 4.14-4.20 (m, 1H) 4.70-4.74 (m, 1H) 7.21-7.33 (m, 3H) 7.37-7.42 (m, 1H)

### Step (ii): (trans)-1-methoxy-2,3-dihydro-1H-inden-2-amine

A suspension of (trans)-2-azido-1-methoxy-2,3-dihydro-1H-indene (1.36 g, 7.19 mmol) and 10% palladium on activated carbon powder (0.765 g, 0.719 mmol) in ethanol (50 mL) was evacuated and purged with nitrogen three times, then stirred under an atmosphere of hydrogen overnight. The suspension was filtered through celite and concentrated in vacuo. The product was loaded onto a cation exchange cartridge, washed with methanol and eluted with 2 M ammonia/methanol solution then concentrated in vacuo to afford the title compound.

¹H NMR (400 MHz, CHLOROFORM-d) δ ppm 1.46 (br. s., 2H) 2.55-2.64 (m, 1H) 3.27-3.36 (m, 1H) 3.55 (s, 3H) 3.68-3.74 (m, 1H) 4.45-4.49 (m, 1H) 7.20-7.26 (m, 3H) 7.37-7.42 (m, 1H)

### Intermediate 5: (trans)-1-ethoxy-2,3-dihydro-1H-inden-2-amine

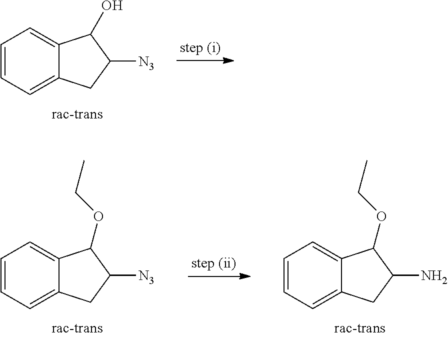

### Step (i): (trans)-2-azido-1-ethoxy-2,3-dihydro-1H-indene

Iodoethane (1.826 mL, 22.83 mmol) was added to a suspension of (trans)-2-azido-2,3-dihydro-1H-inden-1-ol ((synthesis described in Tetrahedron: Asymmetry, 1995, 6, 7, 1535, using racemic cis starting material) 1.6 g, 9.13 mmol) and silver oxide (2.54 g, 10.96 mmol) in acetonitrile (25 mL). The reaction was stirred at room temperature for 2 days in a sealed flask in the dark. The reaction was heated to 60° C. for 5 hours. Further portions of iodoethane (1.826 mL, 22.83 mmol) and silver oxide (2.54 g, 10.96 mmol) were added and the reaction stirred at room temperature overnight, then heated in a sealed flask at 70° C. for 4 hours and room temperature over the weekend. The suspension was heated to 70° C. for 4 hours. The suspension was filtered through celite and concentrated in vacuo. The crude product was purified by column chromatography on silica, eluted with 0-10% ethyl acetate/petroleum ether to afford the title compound.

¹H NMR (400 MHz, CHLOROFORM-d) δ ppm 1.28-1.34 (m, 3H) 2.84-2.93 (m, 1H) 3.32-3.40 (m, 1H) 3.74-3.89 (m, 2H) 4.13-4.19 (m, 1H) 4.81 (d, J=4.95 Hz, 1H) 7.20-7.31 (m, 3H) 7.35-7.41 (m, 1H)

### Step (ii): (trans)-1-ethoxy-2,3-dihydro-1H-inden-2-amine

A suspension of (trans)-2-azido-1-ethoxy-2,3-dihydro-1H-indene (1.40 g, 6.89 mmol) and 10% palladium on activated carbon powder (0.733 g, 0.689 mmol) in ethanol (50 mL) was evacuated and purged with nitrogen three times, then stirred under an atmosphere of hydrogen for 2 hours. The suspension was filtered through celite and concentrated in vacuo to afford the title compound.

¹H NMR (400 MHz, CHLOROFORM-d) δ ppm 1.29 (t, J=6.97 Hz, 3H) 1.43 (br. s., 2H) 2.53-2.64 (m, 1H) 3.26-3.35 (m, 1H) 3.64-3.74 (m, 1H) 3.74-3.83 (m, 2H) 4.55 (d, J=4.59 Hz, 1H) 7.18-7.26 (m, 3H) 7.33-7.41 (m, 1H)

### Intermediate 6: (S)-2-(4-fluorophenyl)butanoic acid

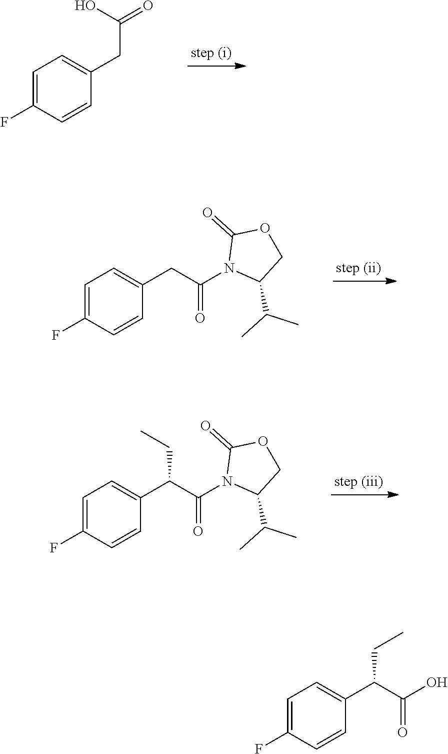 [SMILES: O=C(O)Cc1ccc(F)cc1 ; CC(C)[C@H]1COC(=O)N1C(=O)Cc1ccc(F)cc1 ; CC[C@H](C(=O)N1C(=O)OC[C@@H]1C(C)C)c1ccc(F)cc1 ; CC[C@H](C(=O)O)c1ccc(F)cc1]

### Step (i): (S)-3-(2-(4-fluorophenyl)acetyl)-4-isopropyloxazolidin-2-one

Prepared as described for Intermediate 2 step (i) using n-Butyl lithium (2.5 M solution in hexane, 71.62 mL, 179 mmol), (S)-4-isopropyloxazolidin-2-one (21.0 g, 162.79 mmol), 2-(4-fluorophenyl)acetic acid (27.57 g, 179 mmol), triethylamine (19.73 g, 195.34 mmol) and pivaloyl chloride (29.54 g, 244.18 mmol). The crude product was purified by column chromatography on silica, eluted with 5-7% ethyl acetate/hexane to afford the title compound.

¹H NMR (400 MHz, DMSO-d₆) δ ppm 0.70-0.87 (m, 6H) 2.07-2.20 (m, 1H) 4.05-4.15 (m, 1H) 4.25-4.40 (m, 4H) 7.09-7.17 (m, 2H) 7.24-7.31 (m, 2H)

### Step (ii): (S)-3-((S)-2-(4-fluorophenyl)butanoyl)-4-isopropyloxazolidin-2-one

Prepared as described for Intermediate 2 step (ii) using sodium bis(trimethylsilyl)amide (1 M solution in THF, 114 mL, 113.2 mmol), (S)-3-(2-(4-fluorophenyl)acetyl)-4-isopropyloxazolidin-2-one (20 g, 75.47 mmol) and ethyl iodide (58.56 g, 377.35 mmol). A second portion of ethyl iodide was added and the reaction stirred for another 30 minutes at 0° C. The crude product was purified by column chromatography on silica, eluted with 2-3% ethyl acetate/hexane to afford the title compound.

¹H NMR (400 MHz, DMSO-d₆) δ ppm 0.76-0.93 (m, 9H) 1.63-1.76 (m, 1H) 1.95-2.09 (m, 1H) 2.18-2.30 (m, 1H) 4.19-4.32 (m, 2H) 4.34-4.42 (m, 1H) 4.85-4.93 (m, 1H) 7.10-7.19 (m, 2H) 7.28-7.35 (m, 2H)

### Step (iii): (S)-2-(4-fluorophenyl)butanoic acid

Prepared as described for Intermediate 2 step (iii) using lithium hydroxide (3.15 g, 75.08 mmol), (S)-3-((S)-2-(4-fluorophenyl)butanoyl)-4-isopropyloxazolidin-2-one (11 g, 37.54 mmol) and hydrogen peroxide (33.9 mL, 299.31 mmol) to afford the title compound.

¹H NMR (400 MHz, DMSO-d₆) δ ppm 0.81 (t, J=6.72 Hz, 3H) 1.56-1.70 (m, 1H) 1.88-2.00 (m, 1H) 3.40-3.48 (m, 1H) 7.08-7.19 (m, 2H) 7.25-7.37 (m, 2H) 12.34 (br. s., 1H)

### Intermediate 7: 2-(2,4-difluorophenyl)-2-(2-oxopyridin-1(2H)-yl)acetic acid

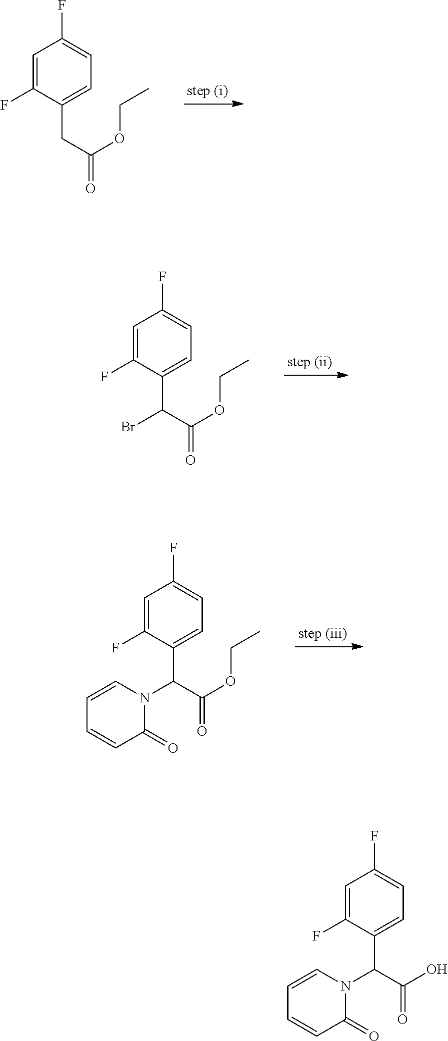 [SMILES: CCOC(=O)Cc1ccc(F)cc1F ; CCOC(=O)C(Br)c1ccc(F)cc1F ; CCOC(=O)C(c1ccc(F)cc1F)n1ccccc1=O ; O=C(O)C(c1ccc(F)cc1F)n1ccccc1=O]

### Step (i): ethyl 2-bromo-2-(2,4-difluorophenyl)acetate

1,1′-Azobis(cyclohexanecarbonitrile) (0.122 g, 0.500 mmol) was added to a suspension of NBS (0.898 g, 5.05 mmol) and ethyl 2-(2,4-difluorophenyl)acetate (1 g, 5.00 mmol) in chlorobenzene (20 mL) under nitrogen. The reaction was stirred at 76° C. for 10 hours. The mixture was partitioned between DCM and water. The phases were separated and the aqueous extracted with DCM. The combined organics were washed with water, dried (phase separator) and concentrated in vacuo. The crude product was purified by column chromatography on silica, eluted with 0-5% ethyl acetate/petroleum ether to afford the title compound.

¹H NMR (400 MHz, DMSO-d₆) δ ppm 1.18 (t, J=7.15 Hz, 3H) 4.20 (q, J=7.15 Hz, 2H) 6.13 (s, 1H) 7.11-7.18 (m, 1H) 7.28-7.36 (m, 1H) 7.60-7.70 (m, 1H)

### Step (ii): ethyl 2-(2,4-difluorophenyl)-2-(2-oxopyridin-1(2H)-yl)acetate

Cesium carbonate (1.374 g, 4.22 mmol) was added to a solution of ethyl 2-bromo-2-(2,4-difluorophenyl)acetate (1.07 g, 3.83 mmol) and pyridin-2-ol (0.413 g, 4.22 mmol) in DMF (20 mL) under nitrogen. The reaction was stirred at room temperature for 3 hours. The mixture was partitioned between ethyl acetate and half saturated brine. The phases were separated and the aqueous extracted with ethyl acetate. The combined organics were washed with half saturated brine, dried (phase separator) and concentrated in vacuo. The crude product was purified by column chromatography on silica, eluted with 0-50% ethyl acetate/petroleum ether to afford the title compound.

¹H NMR (400 MHz, DMSO-d₆) δ ppm 1.18 (t, J=7.15 Hz, 3H) 4.13-4.27 (m, 2H) 6.22-6.31 (m, 1H) 6.48 (d, J=8.99 Hz, 1H) 6.59 (s, 1H) 7.12-7.27 (m, 1H) 7.34-7.56 (m, 4H)

### Step (iii): 2-(2,4-difluorophenyl)-2-(2-oxopyridin-1(2H)-yl)acetic acid

Lithium hydroxide (90 mg, 3.75 mmol) was added to a solution of ethyl 2-(2,4-difluorophenyl)-2-(2-oxopyridin-1(2H)-yl)acetate (550 mg, 1.875 mmol) in water (5 mL) and THF (5 mL) under nitrogen. The reaction was stirred at room temperature overnight. Water was added and the reaction acidified to pH 2 with 2M HCl. The aqueous was extracted with ethyl acetate and the combined organics were washed with water, dried (phase separator) and concentrated in vacuo to afford the title compound.

¹H NMR (300 MHz, DMSO-d₆) δ ppm 6.16-6.28 (m, 1H) 6.46 (d, J=8.94 Hz, 1H) 6.58 (s, 1H) 7.14-7.28 (m, 1H) 7.31-7.61 (m, 4H) 13.61 (br. s., 1H)

### Intermediate 8: tert-butyl ((1S)-1-(4-fluorophenyl)-2-((trans)-(1-methoxy-2,3-dihydro-1H-inden-2-yl)amino)-2-oxoethyl)carbamate

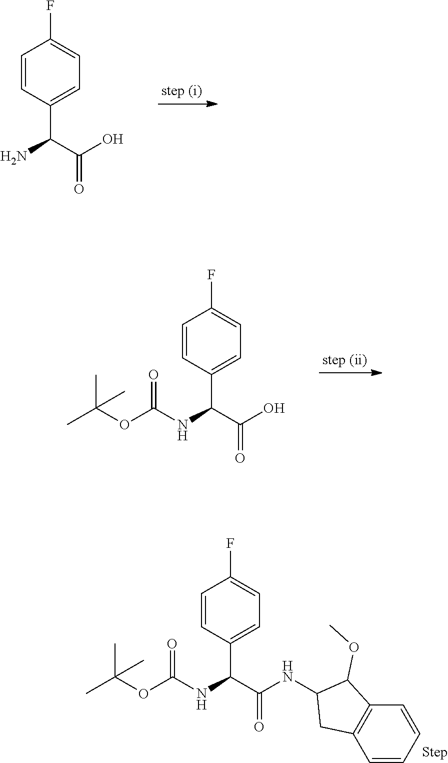 [SMILES: N[C@H](C(=O)O)c1ccc(F)cc1 ; CC(C)(C)OC(=O)N[C@H](C(=O)O)c1ccc(F)cc1 ; COC1c2ccccc2CC1NC(=O)[C@@H](NC(=O)OC(C)(C)C)c1ccc(F)cc1]

### (i): (S)-2-((tert-butoxycarbonyl)amino)-2-(4-fluorophenyl)acetic acid

Triethylamine (5.97 g, 59.171 mmol) was added dropwise to a suspension of (S)-2-amino-2-(4-fluorophenyl)acetic acid (5.0 g, 29.586 mmol) in acetonitrile:water (75 mL: 25 mL) at 0° C. and stirred for 30 minutes. Di-tert-butyl dicarbonate (7.74 g, 35.503 mmol) was added and the reaction was stirred at room temperature for 5 hours. The reaction mixture was diluted with ice cold water and the pH was adjusted to 5 by using 1M HCl solution. The aqueous was extracted with DCM and the combined organics washed with brine, dried over sodium sulphate and concentrated in vacuo to afford the title compound.

¹H NMR (400 MHz, DMSO-d₆) δ ppm 1.34-1.43 (m, 9H) 5.10 (d, J=8.24 Hz, 1H) 7.10-7.24 (m, 2H) 7.36-7.48 (m, 2H) 7.54-7.66 (m, 1H) 12.96 (br. s, 1H)

### Step (ii): tert-butyl ((1S)-1-(4-fluorophenyl)-2-((trans)-(1-methoxy-2,3-dihydro-1H-inden-2-yl)amino)-2-oxoethyl)carbamate

EDC (1.281 g, 6.68 mmol) was added to a solution of (S)-2-((tert-butoxycarbonyl)amino)-2-(4-fluorophenyl)acetic acid (1.5 g, 5.57 mmol), (trans)-1-methoxy-2,3-dihydro-1H-inden-2-amine (Intermediate 4, 1.000 g, 6.13 mmol), HOAt (0.910 g, 6.68 mmol) and 4-methylmorpholine (1.225 mL, 11.14 mmol) in DCM (25 mL) under nitrogen. The reaction was stirred at room temperature overnight. The mixture was partitioned between DCM and 5% citric acid, passed through a phase separator and concentrated in vacuo. The crude product was purified by column chromatography on silica, eluted with 5-40% ethyl acetate/petroleum ether to afford the title compound.

¹H NMR (300 MHz, DMSO-d₆) δ ppm 1.37 (s, 9H) 2.54-2.77 (m, 1H) 3.12-3.40 (m, 4H) 4.23-4.37 (m, 1H) 4.46-4.71 (m, 1H) 5.08-5.22 (m, 1H) 7.09-7.38 (m, 7H) 7.39-7.51 (m, 2H) 8.49-8.62 (m, 1H)

### Intermediate 9: (2S)-2-amino-N-(trans)-(1-ethoxy-2,3-dihydro-1H-inden-2-yl)-2-(4-fluorophenyl)acetamide

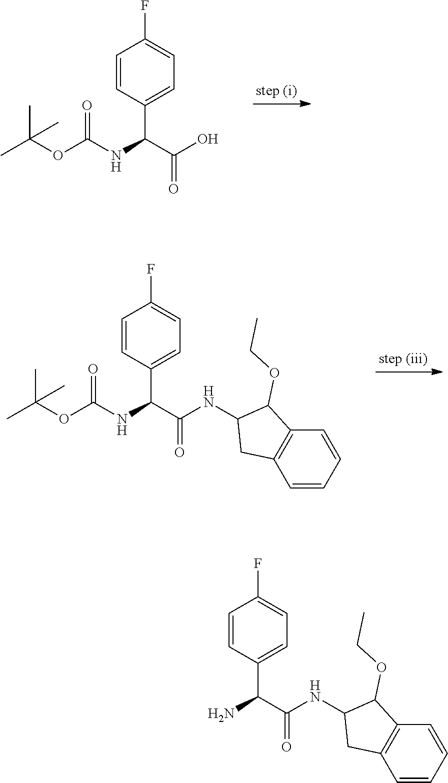 [SMILES: CC(C)(C)OC(=O)N[C@H](C(=O)O)c1ccc(F)cc1 ; CCOC1c2ccccc2CC1NC(=O)[C@@H](NC(=O)OC(C)(C)C)c1ccc(F)cc1 ; CCOC1c2ccccc2CC1NC(=O)[C@@H](N)c1ccc(F)cc1]

### Step (i): tert-butyl ((1S)-2-((trans)-(1-ethoxy-2,3-dihydro-1H-inden-2-yl)amino)-1-(4-fluorophenyl)-2-oxoethyl)carbamate

Prepared as described for tert-butyl ((1S)-1-(4-fluorophenyl)-2-((trans)-(1-methoxy-2,3-dihydro-1H-inden-2-yl)amino)-2-oxoethyl)carbamate (Intermediate 8, step (ii)) using EDC (1.281 g, 6.68 mmol), HOAt (0.910 g, 6.68 mmol), (S)-2-((tert-butoxycarbonyl)amino)-2-(4-fluorophenyl)acetic acid (Intermediate 8, step (i), 1.5 g, 5.57 mmol), (trans)-1-ethoxy-2,3-dihydro-1H-inden-2-amine (Intermediate 5 1.086 g, 6.13 mmol) and 4-methylmorpholine (1.127 g, 11.14 mmol). The crude product was purified by column chromatography on silica, eluted with 0-70% ethyl acetate/petroleum ether to afford the title compound.

¹H NMR (400 MHz, DMSO-d₆) δ ppm 0.87-1.14 (m, 3H) 1.38 (s, 9H) 2.53-2.77 (m, 1H) 3.07-3.27 (m, 1H) 3.35-3.73 (m, 2H) 4.16-4.36 (m, 1H) 4.53-4.79 (m, 1H) 5.06-5.22 (m, 1H) 7.04-7.53 (m, 9H) 8.56 (d, J=8.16 Hz, 1H)

### Step (ii): (2S)-2-amino-N-(trans)-(1-ethoxy-2,3-dihydro-1H-inden-2-yl)-2-(4-fluorophenyl)acetamide

HCl (4 M solution in dioxane, 13.24 mL, 53.0 mmol) was added to a solution of tert-butyl ((1S)-2-((trans)-(1-ethoxy-2,3-dihydro-1H-inden-2-yl)amino)-1-(4-fluorophenyl)-2-oxoethyl)carbamate (2.27 g, 5.30 mmol) in DCM (50 mL) and stirred overnight. The reaction mixture was partitioned between DCM and saturated NaHCO₃ and the organic phase concentrated in vacuo. The crude product was purified by reverse phase chromatography on C18 silica eluted with 5-95% methanol/water (with 0.05% ammonia) to afford the title compound.

MS ES⁺: 329

### Intermediate 10: 2-fluoro-2-(4-fluorophenyl)propanoic acid

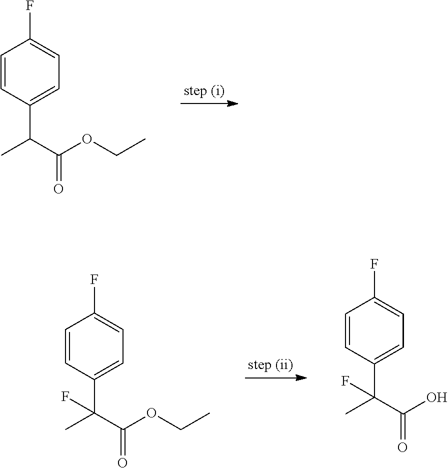

### Step (i): ethyl 2-fluoro-2-(4-fluorophenyl)propanoate

A solution of diisopropylamine (3.80 mL, 26.6 mmol) in THF (30 mL) was cooled in a dry ice/acetone bath. n-Butyl lithium (2.5 M solution in hexanes, 10.65 mL, 26.6 mmol) was added, followed by ethyl 2-(4-fluorophenyl)propanoate (4.02 g, 20.49 mmol) in THF (10 mL) (over ca. 15 minutes). The mixture was stirred cold for 30 minutes then in an ice/water bath for 30 minutes and then cooled to ca. −70° C. again. A solution of N-fluorobenzenesulphonimide (7.11 g, 22.54 mmol) in THF (20 mL) was added and the mixture stirred, warming to room temperature. Acetic acid (1.5 mL) was added and the mixture partitioned between water and ethyl acetate. The aqueous was extracted with ethyl acetate. The combined organics were dried and concentrated in vacuo. The crude product was purified by column chromatography on silica, eluted with 5-20% ethyl acetate/petroleum ether to afford the title compound.

¹H NMR (400 MHz, DMSO-d₆) δ ppm 1.10-1.23 (m, 3H) 1.82-1.96 (m, 3H) 4.09-4.25 (m, 2H) 7.20-7.35 (m, 2H) 7.44-7.59 (m, 2H)

### Step (ii): 2-fluoro-2-(4-fluorophenyl)propanoic acid

LiOH (1.372 g, 57.3 mmol) was added to a solution of ethyl 2-fluoro-2-(4-fluorophenyl)propanoate (4.09 g, 19.09 mmol) in THF (30 mL) and water (10 mL). The mixture was stirred at room temperature for 2 hours. The mixture was diluted with water and extracted with DCM. The aqueous phase was acidified with HCl (2 M) and extracted with DCM-THF (3:1). The combined organic phases were dried and concentrated in vacuo to afford the title compound.

¹H NMR (400 MHz, DMSO-d₆) δ ppm 1.80-1.95 (m, 3H) 7.16-7.40 (m, 2H) 7.44-7.64 (m, 2H) 13.53 (br. s., 1H)

### Intermediate 11: lithio 2-(2,4-difluorophenyl)-2-(1H-pyrazol-1-yl)acetate

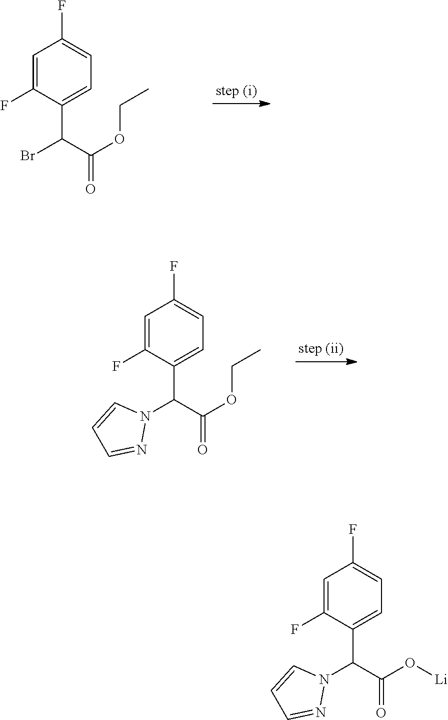

### Step (i): ethyl 2-(2,4-difluorophenyl)-2-(1H-pyrazol-1-yl)acetate

Caesium carbonate (0.321 g, 0.985 mmol) was added to a solution of ethyl 2-bromo-2-(2,4-difluorophenyl)acetate (Intermediate 7, step (i), 0.250 g, 0.896 mmol) and 1H-pyrazole (0.067 g, 0.985 mmol) in DMF (2.5 mL) under nitrogen. The reaction was stirred at room temperature for 22 hours. The mixture was partitioned between ethyl acetate and half saturated brine. The phases were separated and the aqueous extracted with ethyl acetate. The combined organics were washed with half saturated brine, dried (phase separator) and concentrated in vacuo. The crude product was purified by column chromatography on silica, eluted with 0-50% ethyl acetate/petroleum ether to afford the title compound.

¹H NMR (400 MHz, DICHLOROMETHANE-d₂) δ ppm 1.24 (t, J=7.15 Hz, 3H), 4.26 (q, J=7.12 Hz, 2H), 6.30-6.32 (m, 1H), 6.37 (s, 1H), 6.89-7.00 (m, 2H), 7.34-7.43 (m, 1H), 7.47-7.50 (m, 1H), 7.52-7.57 (m, 1H)

MS ES⁺: 267

### Step (ii): lithio 2-(2,4-difluorophenyl)-2-(1H-pyrazol-1-yl)acetate

Lithium hydroxide (1M aqueous) (1.0 mL, 1.00 mmol) was added to a solution of ethyl 2-(2,4-difluorophenyl)-2-(1H-pyrazol-1-yl)acetate (136 mg, 0.511 mmol) in THF (1.5 mL). The reaction was stirred at room temperature for 18 hours. The reaction mixture was concentrated in vacuo to afford the title compound.

MS ES⁺: 239

### Intermediate 12: lithio 2-(2,4-difluorophenyl)-2-(2-methyl-1H-imidazol-1-yl)acetate

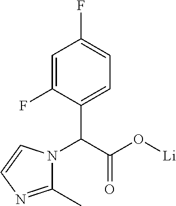

Prepared as described for Intermediate 11 using 2-methyl-1H-imidazole (81 mg, 0.985 mmol) and ethyl 2-bromo-2-(2,4-difluorophenyl)acetate (Intermediate 7, step (i), 0.250 g, 0.896 mmol) to afford the title compound.

MS ES⁺: 253

### Intermediate 13: (2S)-2-amino-2-(4-fluorophenyl)-N-(trans)-(1-methyl-2,3-dihydro-1H-inden-2-yl)acetamide hydrochloride

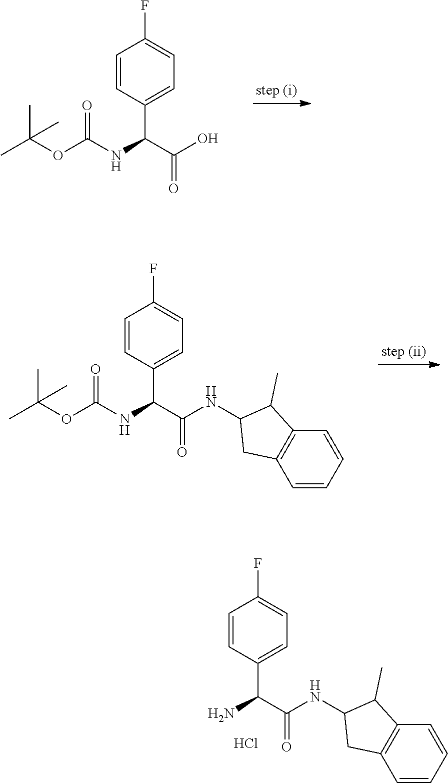 [SMILES: CC(C)(C)OC(=O)N[C@H](C(=O)O)c1ccc(F)cc1 ; CC1c2ccccc2CC1NC(=O)[C@@H](NC(=O)OC(C)(C)C)c1ccc(F)cc1 ; CC1c2ccccc2CC1NC(=O)[C@@H](N)c1ccc(F)cc1 ; Cl]

### Step (i): tert-butyl N—[(S)-(4-fluorophenyl)[((trans)-1-methyl-2,3-dihydro-1H-inden-2-yl)carbamoyl]methyl]carbamate

T3P (0.404 mL, 0.679 mmol) was added to a stirred solution of triethylamine (0.138 mL, 1.019 mmol), (S)-2-((tert-butoxycarbonyl)amino)-2-(4-fluorophenyl)acetic acid (Intermediate 8, step (i), 91 mg, 0.340 mmol) and 1-methyl-2,3-dihydro-1H-inden-2-amine (50 mg, 0.340 mmol) in DCM (2 mL) and stirred for 30 minutes. The reaction mixture was washed with saturated aq. NaHCO₃, dried (phase separator) and concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 0.65-1.12 (m, 3H), 1.27-1.46 (m, 9H), 2.65-2.84 (m, 1H), 2.99-3.31 (m, 2H), 4.40-4.59 (m, 1H), 5.15-5.30 (m, 1H), 7.07-7.38 (m, 7H), 7.42-7.52 (m, 2H), 8.20-8.31 (m, 1H)

MS ES⁺: 399

### Step (ii): (2S)-2-amino-2-(4-fluorophenyl)-N-(trans)-(1-methyl-2,3-dihydro-1H-inden-2-yl)acetamide hydrochloride

HCl (4N in dioxane, 0.816 mL, 3.26 mmol) was added to a solution of tert-butyl N—[(S)-(4-fluorophenyl)[((trans)-1-methyl-2,3-dihydro-1H-inden-2-yl)carbamoyl]methyl]carbamate (65 mg, 0.163 mmol) in DCM (2 mL) and stirred for 5 hours. Additional HCl (4N in dioxane, 0.5 mL) was added and the reaction stirred for 18 hours. The reaction was concentrated in vacuo to afford the title compound.

MS ES⁺: 299

### Intermediate 14: lithio 2-(2,4-difluorophenyl)-2-(3-fluoroazetidin-1-yl)acetate

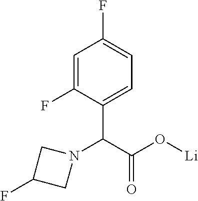

Prepared as described for Intermediate 11 using 3-fluoroazetidine hydrochloride (0.220 g, 1.971 mmol) and ethyl 2-bromo-2-(2,4-difluorophenyl)acetate (Intermediate 7, step (i), 500 mg, 1.792 mmol) to afford the title compound.

MS ES⁺: 246

### Intermediates 15 and 16: tert-butyl N-[(1S,2S)-2-(3-hydroxy-2-phenylpropanamido)-2,3-dihydro-1H-inden-1-yl]carbamate Stereoisomers A and B

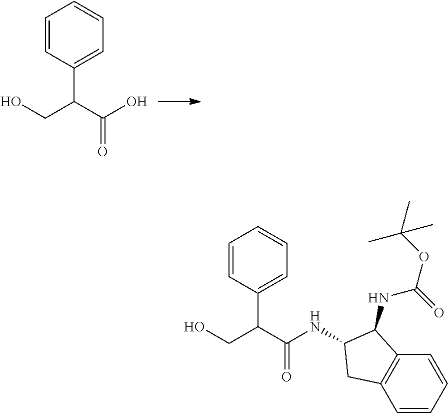 [SMILES: O=C(O)C(CO)c1ccccc1 ; CC(C)(C)OC(=O)N[C@H]1c2ccccc2C[C@@H]1NC(=O)C(CO)c1ccccc1]

COMU (1.993 g, 4.65 mmol) was added to a stirred solution of 3-hydroxy-2-phenylpropanoic acid (0.703 g, 4.23 mmol), tert-butyl ((1S,2S)-2-amino-2,3-dihydro-1H-inden-1-yl)carbamate (1.051 g, 4.23 mmol) and 2,2,6,6-tetramethylpiperidine (0.598 g, 4.23 mmol) in DCM (20 mL) and stirred for 1 hour. The reaction mixture was washed with water and concentrated in vacuo. The crude product was purified by column chromatography on silica, eluted with 0-70% ethyl acetate/petroleum ether to afford the title compounds as single stereoisomers.

### Intermediate 15—Stereoisomer A—First Eluting

¹H NMR (300 MHz, DMSO-d₆) δ ppm 1.44 (s, 9H) 2.42-2.48 (m, 1H) 2.96-3.18 (m, 1H) 3.45-3.68 (m, 2H) 3.86-4.03 (m, 1H) 4.19-4.43 (m, 1H) 4.62-4.79 (m, 1H) 4.93-5.06 (m, 1H) 6.94-7.41 (m, 10H) 8.34-8.51 (m, 1H)

MS ES⁺: 397

### Intermediate 16—Stereoisomer B—Second Eluting

¹H NMR (400 MHz, DMSO-d₆) δ ppm 1.42-1.62 (m, 4H) 2.53-2.75 (m, 2H) 3.11-3.40 (m, 6H) 3.77-3.90 (m, 2H) 4.22-4.36 (m, 1H) 4.46-4.69 (m, 1H) 5.44 (d, J=8.07 Hz, 1H) 7.09-7.38 (m, 6H) 7.40-7.50 (m, 2H) 8.42-8.54 (m, 1H) 8.63-8.74 (m, 1H)

MS ES⁺: 397

### Intermediate 17: lithio 2-(azetidin-1-yl)-2-(2,4-difluorophenyl)acetate

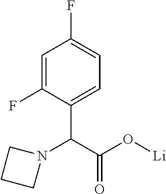

Prepared as described for Intermediate 11 using azetidine hydrochloride (184 mg, 1.971 mmol) and ethyl 2-bromo-2-(2,4-difluorophenyl)acetate (Intermediate 7, step (i), 500 mg, 1.792 mmol) to afford the title compound.

MS ES⁺: 228

### Intermediate 18: lithio 2-(azetidin-1-yl)-2-(2,4-difluorophenyl)acetate

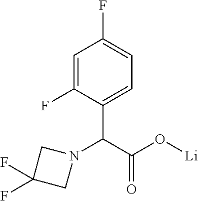

Prepared as described for Intermediate 11 using 3,3-difluoroazetidine hydrochloride (255 mg, 1.971 mmol) and ethyl 2-bromo-2-(2,4-difluorophenyl)acetate (Intermediate 7, step (i), 500 mg, 1.792 mmol) to afford the title compound.

MS ES⁺: 264

### Intermediate 19: lithio 2-(2,4-difluorophenyl)-2-(3-methoxyazetidin-1-yl)acetate

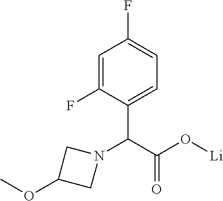

Prepared as described for Intermediate 11 using 3-methoxyazetidine hydrochloride (244 mg, 1.971 mmol) and ethyl 2-bromo-2-(2,4-difluorophenyl)acetate (Intermediate 7, step (i), 500 mg, 1.792 mmol) to afford the title compound.

MS ES⁺: 258

### Intermediate 20: (2S)—N-(trans)-(1-{3-[(tert-butyldimethylsilyl)oxy]azetidin-1-yl}-2,3-dihydro-1H-inden-2-yl)-2-(4-fluorophenyl)propanamide Single Stereoisomer

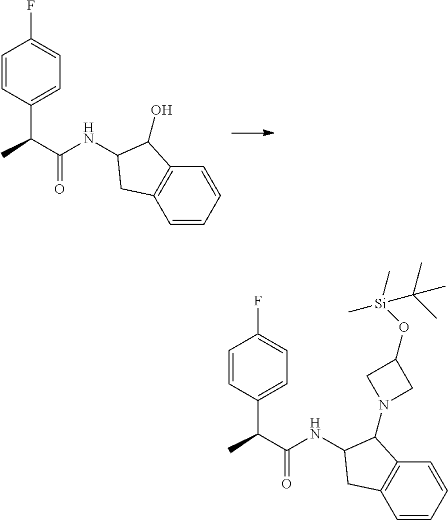 [SMILES: C[C@H](C(=O)NC1Cc2ccccc2C1O)c1ccc(F)cc1 ; C[C@H](C(=O)NC1Cc2ccccc2C1N1CC(O[Si](C)(C)C(C)(C)C)C1)c1ccc(F)cc1]

A solution of methanesulfonic anhydride (0.372 g, 2.135 mmol) in THF (2 mL) was added drop wise to a solution of (2S)-2-(4-fluorophenyl)-N-((cis)-1-hydroxy-2,3-dihydro-1H-inden-2-yl)propanamide (Example 63 (second eluting peak), 0.320 g, 1.067 mmol) and triethylamine (0.446 mL, 3.20 mmol) in THF (2 mL) at −78° C. under nitrogen. The reaction was stirred in a salt/ice bath for 15 minutes. 3-((tert-Butyldimethylsilyl)oxy)azetidine (1.00 g, 5.34 mmol) was added as a solution in THF (2 mL). The reaction was stirred in the ice bath and allowed to slowly warm to room temperature over 6.5 hours. The mixture was partitioned between ethyl acetate and water. The phases were separated and the aqueous extracted with ethyl acetate. The combined organics were washed with brine, dried (phase separator) and concentrated in vacuo. The crude product was purified by column chromatography on silica, eluted with 12-100% ethyl acetate/petroleum ether to afford the title compound.

¹H NMR (400 MHz, DMSO-d₆) δ −0.03-0.05 (m, 6H), 0.79-0.86 (m, 9H), 1.26 (d, J=7.06 Hz, 3H), 2.49-2.54 (m, 1H), 2.95-3.04 (m, 2H), 3.11-3.19 (m, 1H), 3.36-3.43 (m, 1H), 3.50-3.58 (m, 2H), 3.61-3.65 (m, 1H), 4.04-4.11 (m, 1H), 4.23-4.32 (m, 1H), 7.02-7.23 (m, 5H), 7.25-7.34 (m, 3H), 8.16-8.23 (m, 1H)

MS ES⁺: 469

### Intermediate 21: lithio 2-(2,4-difluorophenyl)-2-(6-oxo-1,6-dihydropyridazin-1-yl)acetate

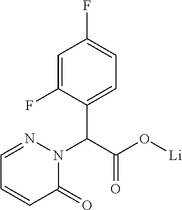

Prepared as described for Intermediate 11, using pyridazin-3-ol (189 mg, 1.971 mmol) and ethyl 2-bromo-2-(2,4-difluorophenyl)acetate (500 mg, 1.792 mmol) to afford the title compound.

MS ES⁺: 267

### Intermediate 22: tert-butyl N-[(1S,2S)-2-[2-(2,4-difluorophenyl)-2-(6-oxo-1,6-dihydropyridazin-1-yl)acetamido]-2,3-dihydro-1H-inden-1-yl]carbamate

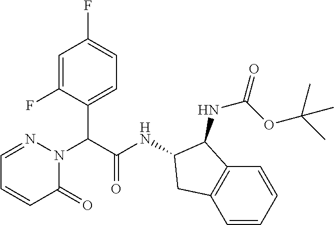 [SMILES: CC(C)(C)OC(=O)N[C@H]1c2ccccc2C[C@@H]1NC(=O)C(c1ccc(F)cc1F)n1ncccc1=O]

HATU (539 mg, 1.419 mmol) was added to a solution of lithio 2-(2,4-difluorophenyl)-2-(6-oxo-1,6-dihydropyridazin-1-yl)acetate (Intermediate 21, 351 mg, 1.290 mmol) and DIPEA (0.473 mL, 2.71 mmol) in DMF (5 mL). The reaction was stirred at room temperature for 5 minutes. tert-Butyl ((1S,2S)-2-amino-2,3-dihydro-1H-inden-1-yl)carbamate (352 mg, 1.419 mmol) was added to the reaction mixture. The reaction was stirred at room temperature for 4 days. The mixture was partitioned between DCM and saturated NaHCO₃. The organic layer was concentrated in vacuo. The crude product was purified by column chromatography on silica, eluted with 0-100% ethyl acetate/petroleum ether, then dissolved in diethyl ether and concentrated in vacuo to afford the title compound.

¹H NMR (400 MHz, DICHLOROMETHANE-d₂) δ ppm 1.38-1.51 (m, 9H), 2.68-2.87 (m, 1H), 3.38-3.64 (m, 2H), 4.15-4.36 (m, 1H), 4.98-5.18 (m, 2H), 6.80-7.03 (m, 4H), 7.08-7.18 (m, 1H), 7.19-7.36 (m, 4H), 7.56-7.66 (m, 1H), 7.74-7.86 (m, 1H)

MS ES⁺: 497

### Intermediate 23: (2S)-2-(4-fluorophenyl)-N-(trans)-[1-(methylsulfanyl)-2,3-dihydro-1H-inden-2-yl]propanamide Single Stereoisomer

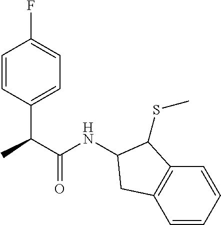 [SMILES: CSC1c2ccccc2CC1NC(=O)[C@@H](C)c1ccc(F)cc1]

Prepared as described for Intermediate 20, using (2S)-2-(4-fluorophenyl)-N-(1-hydroxy-2,3-dihydro-1H-inden-2-yl)propanamide (Example 63 (second eluting peak), 0.4 g, 0.334 mmol) and sodium methanethiolate (0.117 g, 1.670 mmol) to afford the title compound.

¹H NMR (400 MHz, DMSO-d₆) δ ppm 1.31-1.33 (m, 3H), 2.07 (s, 3H), 2.58-2.71 (m, 1H), 3.16-3.28 (m, 1H), 3.55-3.63 (m, 1H), 4.09-4.15 (m, 1H), 4.32-4.43 (m, 1H), 6.93-7.01 (m, 1H), 7.09-7.42 (m, 7H), 8.34-8.49 (m, 1H)

MS ES⁻: 328

### Intermediate 24: lithio 2-(3-fluoroazetidin-1-yl)-2-(4-fluorophenyl)acetate

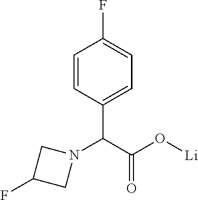

Prepared as described for Intermediate 11, using 3-fluoroazetidine hydrochloride (0.940 g, 8.43 mmol) and ethyl 2-bromo-2-(4-fluorophenyl)acetate (2.00 g, 7.66 mmol) to afford the title compound.

MS ES⁺: 228

### Intermediates 25 and 26: tert-butyl N-[(1S,2S)-2-[2-(3-fluoroazetidin-1-yl)-2-(4-fluorophenyl)acetamido]-2,3-dihydro-1H-inden-1-yl]carbamate Stereoisomers A and B

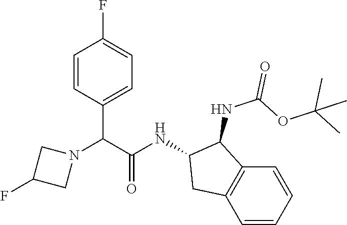 [SMILES: CC(C)(C)OC(=O)N[C@H]1c2ccccc2C[C@@H]1NC(=O)C(c1ccc(F)cc1)N1CC(F)C1]

HATU (485 mg, 1.276 mmol) was added to a solution of lithio 2-(3-fluoroazetidin-1-yl)-2-(4-fluorophenyl)acetate (Intermediate 24, 248 mg, 1.064 mmol) and DIPEA (0.372 mL, 2.127 mmol) in DMF (5 mL). The reaction was stirred at room temperature for 2 minutes. tert-butyl ((1S,2S)-2-amino-2,3-dihydro-1H-inden-1-yl)carbamate (317 mg, 1.276 mmol) was added to the reaction mixture. The reaction was stirred at room temperature for 6 hours. The mixture was partitioned between DCM and saturated NaHCO₃. The phases were separated and the aqueous extracted with DCM. The combined organics were concentrated in vacuo. The crude product was purified by column chromatography on silica, eluted with 0-25% ethyl acetate/petroleum ether to afford the two stereoisomers.

### Intermediate 25 Stereoisomer A—First Eluting

¹H NMR (400 MHz, DICHLOROMETHANE-d₂) δ ppm 1.42-1.52 (m, 9H), 2.45-2.68 (m, 1H), 3.08-3.34 (m, 3H), 3.43 (br. s., 1H), 3.76-3.99 (m, 2H), 4.15-4.28 (m, 1H), 4.97-5.28 (m, 3H), 7.06 (t, J=8.48 Hz, 2H), 7.16 (d, J=3.85 Hz, 1H), 7.20-7.29 (m, 3H), 7.39 (br. s., 2H), 7.59 (br. s., 1H)

MS ES⁺: 458

### Intermediate 26—Stereoisomer B—Second Eluting

¹H NMR (400 MHz, DICHLOROMETHANE-d₂) δ ppm 1.51 (s, 9H), 2.66-2.80 (m, 1H), 3.11-3.39 (m, 2H), 3.42-3.58 (m, 2H), 3.70-3.86 (m, 1H), 3.88-4.01 (m, 1H), 4.97-5.22 (m, 2H), 5.35-5.37 (m, 1H), 7.01-7.11 (m, 2H), 7.19-7.34 (m, 5H), 7.40-7.50 (m, 2H), 7.64-7.77 (m, 1H)

MS ES⁺: 458

### Intermediate 27: lithio 2-[3-(difluoromethoxy)azetidin-1-yl]-2-(2,4-difluorophenyl)acetate

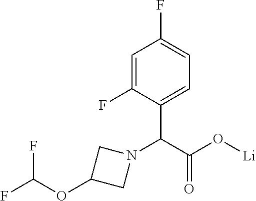

Prepared as described for Intermediate 11, using 3-(difluoromethoxy)azetidine hydrochloride (157 mg, 0.985 mmol) and ethyl 2-bromo-2-(2,4-difluorophenyl)acetate (Intermediate 7, step (i), 0.250 g, 0.896 mmol) to afford the title compound.

MS ES⁺: 294

### Intermediate 28: tert-butyl N-[(1S,2S)-2-{2-[3-(difluoromethoxy)azetidin-1-yl]-2-(2,4-difluorophenyl)acetamido}-2,3-dihydro-1H-inden-1-yl]carbamate

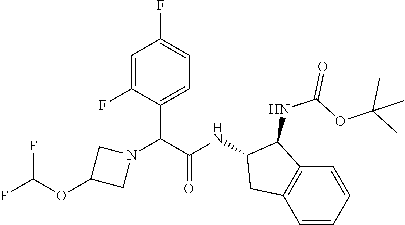 [SMILES: CC(C)(C)OC(=O)N[C@H]1c2ccccc2C[C@@H]1NC(=O)C(c1ccc(F)cc1F)N1CC(OC(F)F)C1]

Prepared as described for Intermediate 22 using of lithio 2-[3-(difluoromethoxy)azetidin-1-yl]-2-(2,4-difluorophenyl)acetate (Intermediate 27, 184 mg, 0.615 mmol) and tert-butyl ((1S,2S)-2-amino-2,3-dihydro-1H-inden-1-yl)carbamate (183 mg, 0.738 mmol). The crude material was purified by column chromatography on silica, eluted with 0-50% ethyl acetate/petroleum ether to afford the title compound.

¹H NMR (400 MHz, DICHLOROMETHANE-d₂) δ ppm 1.42-1.49 (m, 9H), 2.55-2.75 (m, 1H), 2.96-3.25 (m, 2H), 3.29-3.55 (m, 2H), 3.70-4.00 (m, 1H), 4.12-4.38 (m, 2H), 4.71-4.84 (m, 1H), 4.94-5.05 (m, 1H), 5.08-5.21 (m, 1H), 5.96-6.40 (m, 1H), 6.79-6.97 (m, 2H), 7.15-7.25 (m, 4H), 7.32-7.48 (m, 1H), 7.64-7.84 (m, 1H)

MS ES⁺: 524

### Intermediate 29: lithio 3-[(oxan-4-yl)formamido]-2-phenylpropanoate

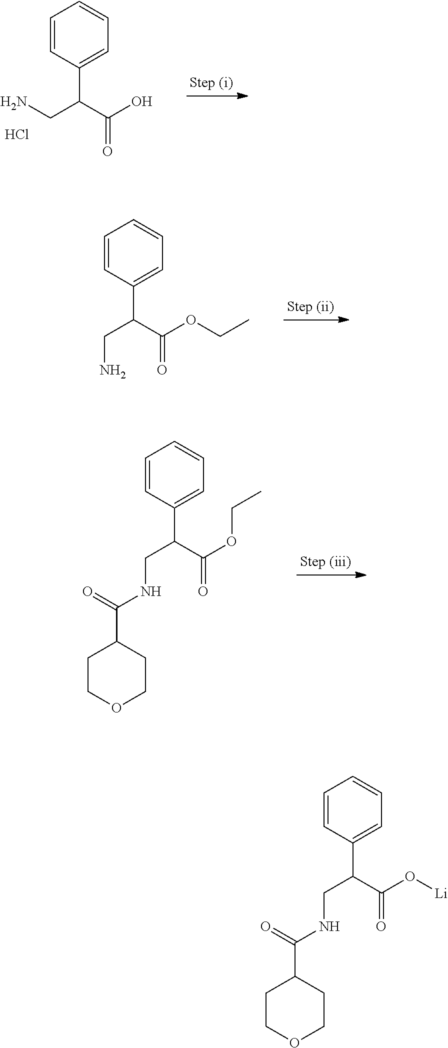

### Step (i): ethyl 3-amino-2-phenylpropanoate

Sulfuric acid (0.013 mL, 0.248 mmol) was added to a suspension of 3-amino-2-phenylpropanoic acid hydrochloride (0.5 g, 2.480 mmol) in EtOH (10 mL). The reaction was heated to 70° C. for 4 hours. The solution was concentrated in vacuo. The mixture was partitioned between ethyl acetate and saturated NaHCO₃. The phases were separated and the aqueous extracted with EtOAc. The combined organics were dried (phase separator) and concentrated in vacuo to afford the title compound.

¹H NMR (400 MHz, DMSO-d₆) δ ppm 1.14 (t, J=7.11 Hz, 3H), 1.45 (br. s., 2H), 2.74-2.84 (m, 1H), 3.02-3.14 (m, 1H), 3.59-3.68 (m, 1H), 4.01-4.13 (m, 2H), 7.22-7.29 (m, 3H), 7.30-7.38 (m, 2H)

### Step (ii): ethyl 3-[(oxan-4-yl)formamido]-2-phenylpropanoate

T3P (50% in ethyl acetate) (0.844 mL, 0.963 mmol) was added to a solution of ethyl 3-amino-2-phenylpropanoate (0.124 g, 0.642 mmol), tetrahydro-2H-pyran-4-carboxylic acid (0.092 g, 0.706 mmol) and triethylamine (0.134 mL, 0.963 mmol) in DCM (5 mL). The reaction was stirred at room temperature for 1 hour. The mixture was partitioned between DCM and saturated aq. NaHCO₃, dried (phase separator) and concentrated in vacuo. The crude product was purified by column chromatography on silica, eluted with 0-100% ethyl acetate/petroleum ether to afford the title compound.

¹H NMR (400 MHz, DICHLOROMETHANE-d₂) δ ppm 1.20 (t, J=7.15 Hz, 3H), 1.55-1.70 (m, 4H), 2.17-2.31 (m, 1H), 3.28-3.40 (m, 2H), 3.55-3.73 (m, 2H), 3.82-3.95 (m, 3H), 4.06-4.21 (m, 2H), 5.83-5.96 (m, 1H), 7.21-7.40 (m, 5H)

MS ES⁺: 306

### Step (iii): lithio 3-[(oxan-4-yl)formamido]-2-phenylpropanoate

Lithium hydroxide (0.016 g, 0.654 mmol) was added to a solution of ethyl 3-[(oxan-4-yl)formamido]-2-phenylpropanoate (0.130 g, 0.436 mmol) in THF (2 mL) and water (1 mL). The reaction was stirred at room temperature for 72 hours. The reaction was concentrated in vacuo to afford the title compound.

MS ES⁺: 277

### Intermediate 30: lithio 2-phenyl-3-[(pyridin-2-yl)formamido]propanoate

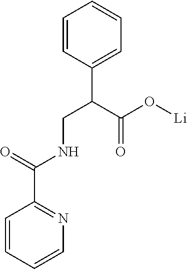

Prepared as described for Intermediate 29 using picolinic acid (0.087 g, 0.706 mmol) in step (ii) to afford the title compound.

MS ES⁺: 277

### Intermediate 31: lithio 2-(4-fluorophenyl)-2-(6-oxo-1,6-dihydropyridazin-1-yl)acetate

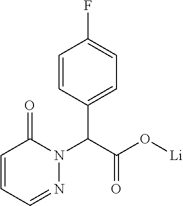

Prepared as described for Intermediate 7 using ethyl 2-(4-fluorophenyl)acetate (15 g, 82 mmol) in step (i) and pyridazin-3(2H)-one (184 mg, 1.915 mmol) in step (ii) to afford the title compound.

MS ES⁺: 249

### Intermediate 32: cis-2-azido-2,3-dihydro-1H-inden-1-ol

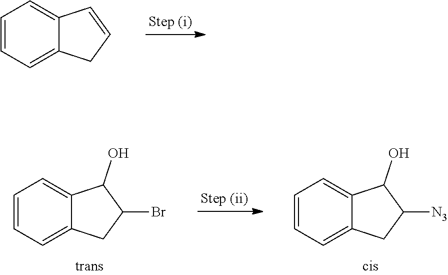 [SMILES: C1=Cc2ccccc2C1 ; OC1c2ccccc2CC1Br ; [N-]=[N+]=NC1Cc2ccccc2C1O]

### Step (i): trans-2-bromo-2,3-dihydro-1H-inden-1-ol

NBS (25.2 g, 141 mmol) was added portion wise to a solution of 1H-indene (15.0 mL, 129 mmol) in THF (150 mL) and water (150 mL). The reaction was stirred at room temperature over 4 days open to the air. The mixture concentrated in vacuo then partitioned between EtOAc and water. The phases were separated and the aqueous extracted twice with EtOAc. The combined organics were washed with saturated Na₂S₂O₃, brine, dried (MgSO₄) and concentrated in vacuo. The crude material was triturated with diethyl ether to afford the title compound.

¹H NMR (400 MHz, DMSO-d₆) δ ppm 3.04-3.16 (m, 1H), 3.50-3.63 (m, 1H), 4.27-4.36 (m, 1H), 5.06-5.13 (m, 1H), 5.94-6.00 (m, 1H), 7.20-7.31 (m, 3H), 7.32-7.40 (m, 1H)

### Step (ii): cis-2-azido-2,3-dihydro-1H-inden-1-ol

A suspension of trans-2-bromo-2,3-dihydro-1H-inden-1-ol (10.0 g, 46.9 mmol) and sodium azide (3.36 g, 51.6 mmol) in DMSO (100 mL) was heated to 60° C. for 1.5 h. The mixture was partitioned between diethyl ether and water. The phases were separated and the aqueous extracted three times with diethyl ether. The combined organics were washed with water, half saturated brine, and brine, dried (MgSO₄) and concentrated in vacuo to afford a pale yellow solid. A suspension of the solid in DMSO (100 mL) was treated with sodium azide (2.288 g, 35.2 mmol) and heated to 60° C. for 2 hours. The reaction was cooled and partitioned between diethyl ether and water. The phases were separated and the aqueous extracted three times with diethyl ether. The combined organics were washed with water, half saturated brine, and brine, dried (MgSO₄) and concentrated in vacuo to afford the title compound.

¹H NMR (300 MHz, CDCl₃) δ ppm 2.31-2.37 (m, 1H), 3.08-3.29 (m, 2H), 4.31-4.41 (m, 1H), 5.12-5.23 (m, 1H), 7.27-7.34 (m, 3H), 7.40-7.52 (m, 1H)

### Intermediate 33: cis-2-amino-2,3-dihydro-1H-inden-1-ol

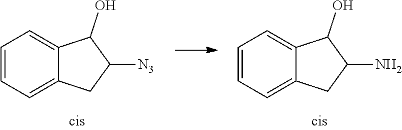 [SMILES: [N-]=[N+]=NC1Cc2ccccc2C1O ; NC1Cc2ccccc2C1O]

A suspension of cis-2-azido-2,3-dihydro-1H-inden-1-ol (Intermediate 32, 0.400 g, 2.283 mmol) and palladium on carbon (10% w/w) (0.243 g, 0.228 mmol) in EtOH (10 mL) was evacuated and purged with nitrogen three times, then stirred under an atmosphere of hydrogen for 2 hours. The suspension was filtered through diatomaceous earth and concentrated in vacuo. The crude product was loaded onto a cation exchange cartridge, washed with methanol and eluted with 2M NH₃/MeOH solution then concentrated in vacuo to afford the title compound.

¹H NMR (400 MHz, DMSO-d₆) δ ppm 2.56-2.65 (m, 1H), 2.84-2.98 (m, 1H), 3.42-3.52 (m, 1H), 4.62-4.71 (m, 1H), 7.11-7.23 (m, 3H), 7.27-7.36 (m, 1H)

### Intermediate 34: 2-(cyclopropylmethoxy)-N-(trans)-[1-(methylsulfanyl)-2,3-dihydro-1H-inden-2-yl]-2-phenylacetamide

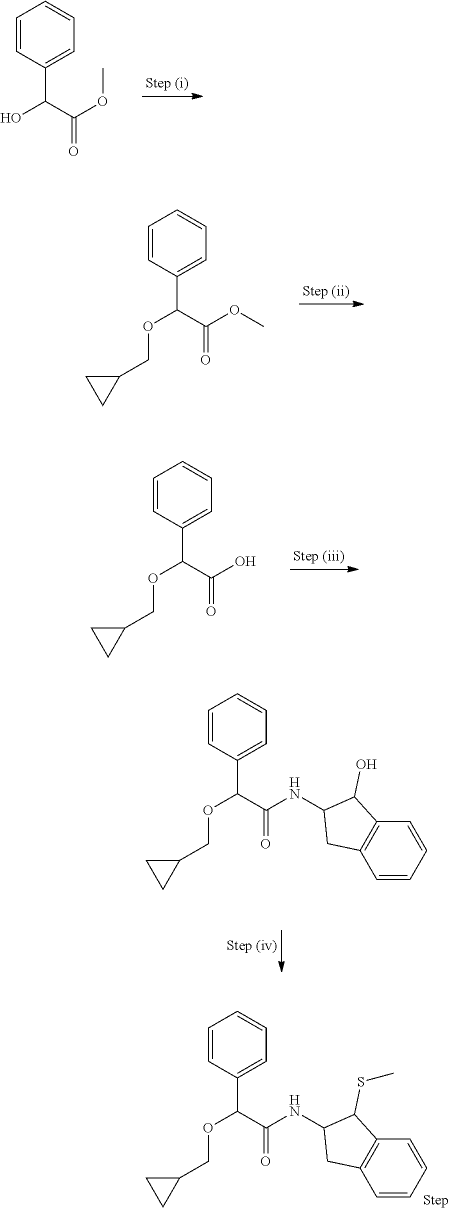 [SMILES: COC(=O)C(O)c1ccccc1 ; COC(=O)C(OCC1CC1)c1ccccc1 ; O=C(O)C(OCC1CC1)c1ccccc1 ; O=C(NC1Cc2ccccc2C1O)C(OCC1CC1)c1ccccc1 ; CSC1c2ccccc2CC1NC(=O)C(OCC1CC1)c1ccccc1]

### (i): methyl-2-(cyclopropylmethoxy)-2-phenylacetate

Sodium hydride (60% dispersion in mineral oil) (144 mg, 3.61 mmol) was added to a stirred solution of (S)-methyl 2-hydroxy-2-phenylacetate (500 mg, 3.01 mmol) in DMF (4 mL), under nitrogen. After 5 minutes, (bromomethyl)cyclopropane (528 mg, 3.91 mmol) was added. After 1 hour the reaction mixture was partitioned between water and EtOAc. The organics were collected, dried (phase separator) and concentrated in vacuo to afford the title compound.

¹H NMR (400 MHz, DMSO-d₆) δ ppm 0.11-0.21 (m, 2H), 0.42-0.53 (m, 2H), 0.96-1.08 (m, 1H), 3.19-3.37 (m, 2H), 3.61-3.64 (m, 3H), 5.03 (s, 1H), 7.32-7.43 (m, 5H)

### Step (ii): 2-(cyclopropylmethoxy)-2-phenylacetic acid

LiOH (420 mg, 17.52 mmol) was added to a stirred solution of methyl-2-(cyclopropylmethoxy)-2-phenylacetate (386 mg, 1.752 mmol) in dioxane (2 mL) and water (2 mL). After 4 hours, the reaction mixture was acidified to approximately pH 1 with concentrated HCl and extracted with EtOAc. The organics were collected, dried (phase separator) and concentrated in vacuo to afford the title compound.

¹H NMR (400 MHz, DMSO-d₆) δ ppm 0.10-0.21 (m, 2H), 0.41-0.51 (m, 2H), 0.97-1.07 (m, 1H), 3.14-3.23 (m, 1H), 3.30-3.40 (m, 1H), 4.84 (s, 1H), 7.29-7.42 (m, 5H)

### Step (iii): 2-(cyclopropylmethoxy)-N-(cis)-(1-hydroxy-2,3-dihydro-1H-inden-2-yl)-2-phenylacetamide

COMU (825 mg, 1.927 mmol) was added to a stirred solution of 2-(cyclopropylmethoxy)-2-phenylacetic acid (361 mg, 1.752 mmol), (cis)-2-amino-2,3-dihydro-1H-inden-1-ol (Intermediate 33, 288 mg, 1.927 mmol) and 2,2,6,6-tetramethylpiperidine (247 mg, 1.752 mmol) in DCM (15 mL) and stirred for 1 hour. The reaction mixture was washed with water, dried (phase separator) and purified by column chromatography on silica, eluted with 0-100% ethyl acetate/petroleum ether. The resulting residue was further purified by reverse phase chromatography on C18 silica eluted with 5-95% acetonitrile/water (with 0.05% ammonia) to afford the title compound.

¹H NMR (400 MHz, DMSO-d₆) δ ppm 0.05-0.27 (m, 2H), 0.37-0.51 (m, 2H), 0.92-1.10 (m, 1H), 2.74-2.89 (m, 1H), 3.01-3.14 (m, 1H), 3.15-3.31 (m, 2H), 4.24-4.40 (m, 1H), 4.77-4.84 (m, 1H), 4.87-4.98 (m, 1H), 5.61-5.70 (m, 1H), 7.17-7.43 (m, 9H), 7.73 (d, J=7.24 Hz, 1H)

### Step (iv): 2-(cyclopropylmethoxy)-N-(trans)-[1-(methylsulfanyl)-2,3-dihydro-1H-inden-2-yl]-2-phenylacetamide

Methanesulfonic anhydride (214 mg, 1.227 mmol) as a solution in THF (2 mL) was added to an acetone/dry ice bath cooled solution of 2-(cyclopropylmethoxy)-N-(cis)-(1-hydroxy-2,3-dihydro-1H-inden-2-yl)-2-phenylacetamide (207 mg, 0.613 mmol) and triethylamine (0.247 mL, 1.840 mmol) in THF (4 mL) under nitrogen. The bath was switched to an ice/water bath and stirred for 30 minutes. Sodium methanethiolate (215 mg, 3.07 mmol) and 15-crown-5 (676 mg, 3.07 mmol) were added to the reaction which was allowed to warm to room temperature for 18 hours. The reaction mixture was partitioned between DCM and water and the organics were collected, dried (phase separator) and concentrated in vacuo to afford the title compound.

MS ES⁺: 368

### Intermediate 35: 2-(cyclopropylformamido)-2-(4-fluorophenyl)acetic acid

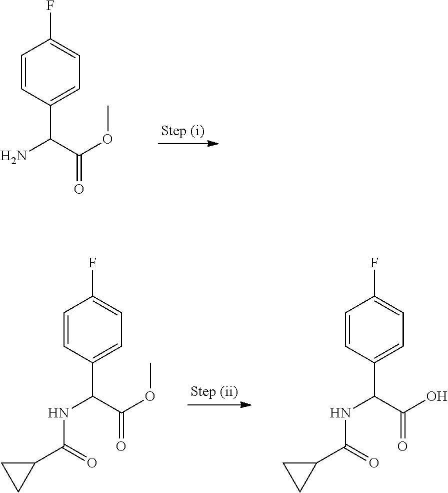 [SMILES: COC(=O)C(N)c1ccc(F)cc1 ; COC(=O)C(NC(=O)C1CC1)c1ccc(F)cc1 ; O=C(NC(C(=O)O)c1ccc(F)cc1)C1CC1]

### Step (i): methyl 2-(cyclopropylformamido)-2-(4-fluorophenyl)acetate

Cyclopropanecarbonyl chloride (0.136 mL, 1.502 mmol) was added to a solution of (S)-methyl 2-amino-2-(4-fluorophenyl)acetate hydrochloride (0.30 g, 1.366 mmol) and triethylamine (0.571 mL, 4.10 mmol) in DCM (10 mL) under nitrogen. The reaction was stirred at room temperature for 1 hour. The mixture was partitioned between DCM and saturated NaHCO₃, dried (phase separator) and concentrated in vacuo. The crude product was purified by column chromatography on silica, eluted with 12-100% ethyl acetate/petroleum ether to afford the title compound.

¹H NMR (400 MHz, DMSO-d₆) δ ppm 0.59-0.80 (m, 4H), 1.76 (s, 1H), 3.63 (s, 3H), 5.46 (d, J=7.24 Hz, 1H), 7.17-7.30 (m, 2H), 7.38-7.49 (m, 2H), 8.95 (d, J=7.24 Hz, 1H)

MS ES⁺: 252

### Step (ii): 2-(cyclopropylformamido)-2-(4-fluorophenyl)acetic acid

LiOH (0.061 g, 2.55 mmol) was added to a solution of methyl 2-(cyclopropylformamido)-2-(4-fluorophenyl)acetate (0.320 g, 1.274 mmol) in acetonitrile (3 mL) and water (3 mL) and stirred for 1.5 hours. The mixture was acidified to pH 2 with 2M HCl and extracted with ethyl acetate. The combined organics were washed with saturated brine, dried (phase separator) and concentrated in vacuo. The product was dried in the vacuum oven to afford the title compound.

¹H NMR (400 MHz, DMSO-d₆) δ ppm 0.55-0.76 (m, 4H), 1.71-1.84 (m, 1H), 5.38 (d, J=7.70 Hz, 1H), 7.17-7.28 (m, 2H), 7.37-7.51 (m, 2H), 8.85 (d, J=7.70 Hz, 1H), 12.88 (br. s, 1H)

MS ES⁻: 236

### Intermediate 36 and 37: (2S)—N-(cis)-(1-hydroxy-2,3-dihydro-1H-inden-2-yl)-2-methoxy-2-phenylacetamide Stereoisomers A and B

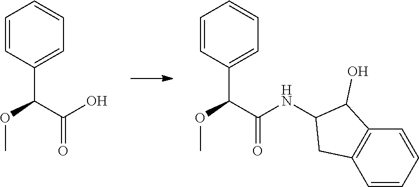 [SMILES: CO[C@H](C(=O)O)c1ccccc1 ; CO[C@H](C(=O)NC1Cc2ccccc2C1O)c1ccccc1]

COMU (1417 mg, 3.31 mmol) was added to a stirred solution of (S)-2-methoxy-2-phenylacetic acid (500 mg, 3.01 mmol), (cis)-2-amino-2,3-dihydro-1H-inden-1-ol (Intermediate 33, 494 mg, 3.31 mmol) and 2,2,6,6-tetramethylpiperidine (425 mg, 3.01 mmol) in DCM (25 mL) and stirred for 1 hour. The reaction mixture was washed with water, dried (phase separator) and purified by column chromatography on silica, eluted with 0-100% ethyl acetate/petroleum ether to afford the title compounds.

### Intermediate 36—Stereoisomer A—First Eluting

¹H NMR (400 MHz, DMSO-d₆) δ ppm 2.83-2.91 (m, 1H), 3.06-3.14 (m, 1H), 3.26 (s, 3H), 4.27-4.36 (m, 1H), 4.70-4.75 (m, 1H), 4.88-4.94 (m, 1H), 5.62 (d, J=6.05 Hz, 1H), 7.18-7.28 (m, 3H), 7.31-7.44 (m, 6H), 7.71-7.77 (m, 1H)

MS ES⁻: 296

### Intermediate 37—Stereoisomer B—Second Eluting

¹H NMR (400 MHz, DMSO-d₆) δ ppm 2.78-2.86 (m, 1H), 3.01-3.09 (m, 1H), 3.30 (s, 3H), 4.29-4.39 (m, 1H), 4.71 (s, 1H), 4.91-4.97 (m, 1H), 5.60 (d, J=6.05 Hz, 1H), 7.19-7.29 (m, 3H), 7.30-7.41 (m, 6H), 7.73-7.78 (m, 1H)

MS ES⁻: 296

### Intermediate 38: (2S)-2-methoxy-N-(trans)-[1-(methylsulfanyl)-2,3-dihydro-1H-inden-2-yl]-2-phenylacetamide and (2S)-2-methoxy-N-(trans)-[1-(ethylsulfanyl)-2,3-dihydro-1H-inden-2-yl]-2-phenylacetamide

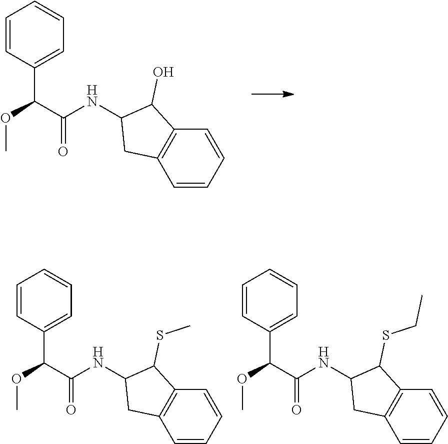

Methanesulfonic anhydride (232 mg, 1.332 mmol) was added as a solution in THF (2 mL) to an acetone/dry ice cooled solution of (2 S)—N-(cis)-(1-hydroxy-2,3-dihydro-1H-inden-2-yl)-2-methoxy-2-phenylacetamide (Intermediate 36, 198 mg, 0.666 mmol) and triethylamine (202 mg, 1.998 mmol) in THF (4 mL) and the cooling bath switched to ice. After 30 minutes, sodium methanethiolate (233 mg, 3.33 mmol) and 15-crown-5 (733 mg, 3.33 mmol) were added and the reaction were stirred for 2 hours. Sodium ethanethiolate (280 mg, 3.33 mmol) was added to the reaction. After a further 3 hours the reaction mixture was partitioned between DCM and water. The organics were collected, dried (phase separator) and concentrated in vacuo to afford a mixture of the title compounds.

MS ES⁺: 350 and 364

### Intermediate 39: lithio 2-[4-(difluoromethoxy)phenyl]propanoate

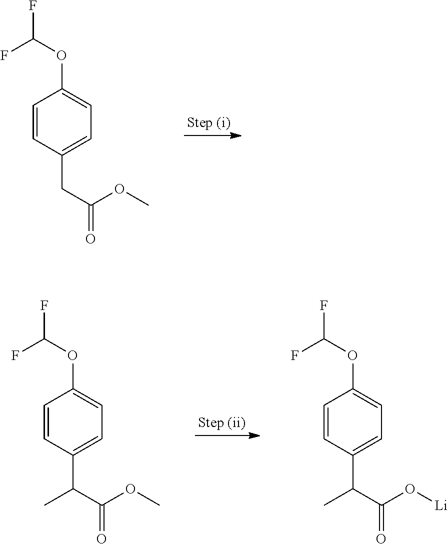

### Step (i): methyl 2-[4-(difluoromethoxy)phenyl]propanoate

NaHMDS (1M in THF, 0.966 mL, 0.966 mmol) was added to a solution of methyl 2-(4-(difluoromethoxy)phenyl)acetate (167 mg, 0.772 mmol) in THF (4 mL) under nitrogen at −78° C. After 30 minutes, methyl iodide (0.051 mL, 0.811 mmol) was added and the reaction was stirred at room temperature for 5 hours. The mixture was partitioned between ethyl acetate and water. The phases were separated and the aqueous extracted with ethyl acetate. The combined organics were dried (phase separator) and concentrated in vacuo. The crude product was purified by column chromatography on silica, eluted with 0-50% ethyl acetate/petroleum ether to afford the title compound.

¹H NMR (400 MHz, DICHLOROMETHANE-d₂) δ ppm 1.47 (d, J=7.15 Hz, 3H), 3.62-3.65 (m, 3H), 5.31-5.33 (m, 1H), 6.31-6.75 (m, 1H), 7.03-7.12 (m, 2H), 7.26-7.39 (m, 2H)

### Step (ii): lithio 2-[4-(difluoromethoxy)phenyl]propanoate

Lithium hydroxide (181 mg, 7.56 mmol) was added to a solution of methyl 2-[4-(difluoromethoxy)phenyl]propanoate (87 mg, 0.378 mmol) in THF (1 mL) and water (1 mL). The reaction was stirred at room temperature for 72 hours. The mixture was concentrated in vacuo to afford the title compound.

MS ES⁻: 215

### Intermediate 40: 2-(4-fluoro-2-methoxyphenyl)propanoic acid

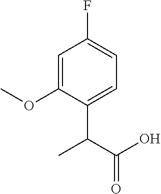

Lithio 2-(4-fluoro-2-methoxyphenyl)propanoate was prepared as described for Intermediate 39 using methyl 2-(4-fluoro-2-methoxyphenyl)acetate (122 mg, 0.616 mmol).

The crude reaction was acidified with 2N HCl and extracted with EtOAc. The organics were collected, dried (phase separator) and concentrated in vacuo to afford the title compound.

¹H NMR (400 MHz, DICHLOROMETHANE-d₂) δ ppm 1.41-1.46 (m, 3H), 3.79-3.83 (m, 3H), 3.95-4.04 (m, 1H), 6.60-6.69 (m, 2H), 7.13-7.21 (m, 1H)

MS ES⁻: 197

### Intermediate 41: 2-(2-chloro-4-fluoro)propanoic acid

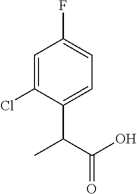

Lithio 2-(2-chloro-4-fluoro)propanoate was prepared as described for Intermediate 39 using methyl 2-(2-chloro-4-fluorophenyl)acetate (150 mg, 0.740 mmol).

The crude reaction was acidified with 2N HCl and extracted with EtOAc. The organics were collected, dried (phase separator) and concentrated in vacuo to afford the title compound.

¹H NMR (400 MHz, DICHLOROMETHANE-d₂) δ ppm 1.55 (d, J=7.24 Hz, 3H), 4.26 (q, J=7.21 Hz, 1H), 7.03-7.09 (m, 1H), 7.18-7.22 (m, 1H), 7.37-7.42 (m, 1H)

MS ES⁻: 201

### Intermediate 42: 2-[4-fluoro-2-(trifluoromethyl)phenyl]propanoic acid

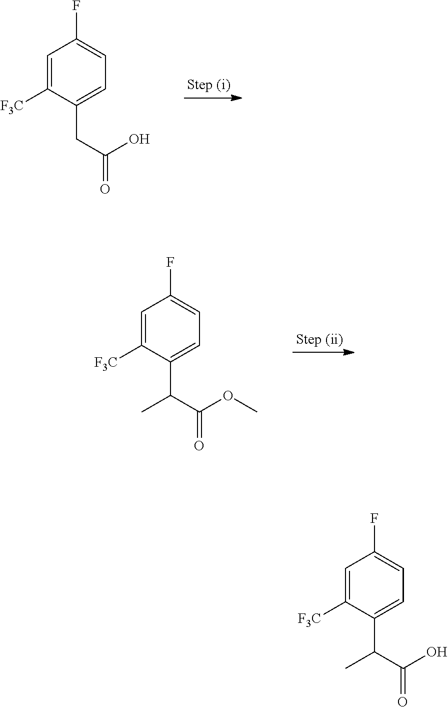 [SMILES: O=C(O)Cc1ccc(F)cc1C(F)(F)F ; COC(=O)C(C)c1ccc(F)cc1C(F)(F)F ; CC(C(=O)O)c1ccc(F)cc1C(F)(F)F]

### Step (i): methyl 2-[4-fluoro-2-(trifluoromethyl)phenyl]propanoate

A solution of HCl (4N in dioxane, 0.17 mL, 0.680 mmol) and 2-(4-fluoro-2-(trifluoromethyl)phenyl)acetic acid (150 mg, 0.675 mmol) in MeOH (2 mL) was heated under microwave irradiation at 120° C. for 20 minutes. The mixture was concentrated in vacuo. A solution of the crude material in THF (4 mL) under nitrogen at −78° C. was treated with sodium bis(trimethylsilyl)amide (0.6 mL, 0.600 mmol). The reaction was stirred at −78° C. for 30 minutes. Methyl iodide (0.052 ml, 0.838 mmol) was added and the reaction stirred for 5 hours. The mixture was partitioned between ethyl acetate and saturated brine. The phases were separated and the aqueous extracted three times with ethyl acetate. The combined organics were dried (phase separator) and concentrated in vacuo. The crude product was purified by column chromatography on silica, eluted with 0-50% ethyl acetate/petroleum ether to afford the title compound.

¹H NMR (400 MHz, DICHLOROMETHANE-d₂) δ ppm 1.48 (d, J=7.06 Hz, 3H), 3.65 (s, 3H), 5.28-5.37 (m, 1H), 7.22-7.31 (m, 1H), 7.33-7.41 (m, 1H), 7.47-7.57 (m, 1H)

### Step (ii): 2-[4-fluoro-2-(trifluoromethyl)phenyl]propanoic acid

Lithium hydroxide (126 mg, 5.28 mmol) was added to a solution of methyl 2-[4-fluoro-2-(trifluoromethyl)phenyl]propanoate (66 mg, 0.264 mmol) in water (1.0 mL) and THF (1.0 mL) under nitrogen. The reaction was stirred at room temperature for 72 hours. The mixture was partitioned between ethyl acetate and 2M HCl. The phases were separated and the aqueous extracted three times with DCM. The combined organics were dried (phase separator) and concentrated in vacuo to afford the title compound.

MS ES⁻: 235

### Intermediate 43: tert-butyl ((1R,2R)-2-amino-2,3-dihydro-1H-inden-1-yl)(methyl)carbamate

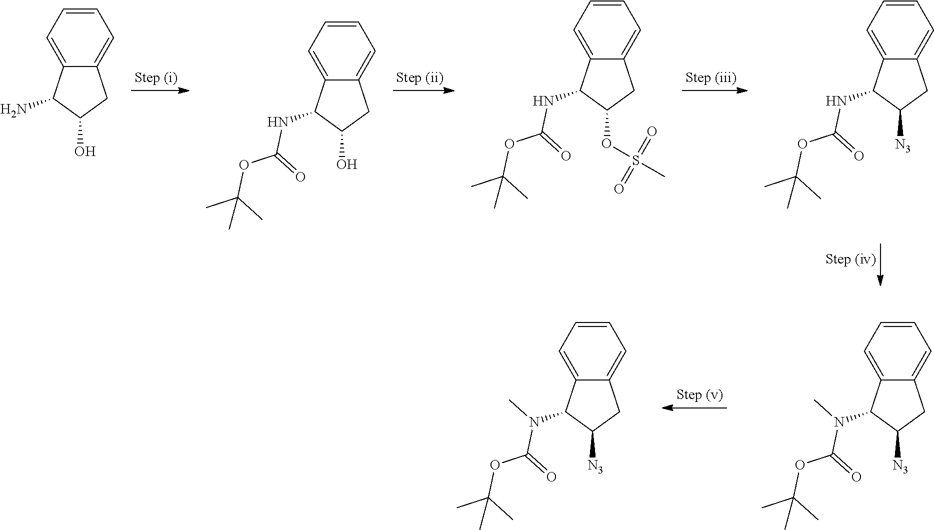 [SMILES: N[C@@H]1c2ccccc2C[C@@H]1O ; CC(C)(C)OC(=O)N[C@@H]1c2ccccc2C[C@@H]1O ; CC(C)(C)OC(=O)N[C@@H]1c2ccccc2C[C@@H]1OS(C)(=O)=O ; CC(C)(C)OC(=O)N[C@@H]1c2ccccc2C[C@H]1N=[N+]=[N-] ; CN(C(=O)OC(C)(C)C)[C@@H]1c2ccccc2C[C@H]1N=[N+]=[N-] ; CN(C(=O)OC(C)(C)C)[C@@H]1c2ccccc2C[C@H]1N=[N+]=[N-]]

### Step (i): tert-butyl N-[(1R,2S)-2-hydroxy-2,3-dihydro-1H-inden-1-yl]carbamate

Di-tert-butyl dicarbonate (3.42 mL, 14.75 mmol) as a solution in THF (4 mL) was added to a stirred suspension of (1R,2S)-1-amino-2,3-dihydro-1H-inden-2-ol (2.0 g, 13.41 mmol) and Na₂CO₃ (2.84 g, 26.8 mmol) in THF (12 mL) and water (12 mL). After stirring for 1.5 hours the reaction mixture was partitioned between water and EtOAc. The organics were separated, dried (phase separator) and concentrated in vacuo to afford the title compound.

¹H NMR (300 MHz, DMSO-d₆) δ ppm 1.45 (s, 9H), 2.69-2.86 (m, 1H), 2.94-3.08 (m, 1H), 4.33-4.47 (m, 1H), 4.82-5.04 (m, 2H), 6.28-6.42 (m, 1H), 7.12-7.24 (m, 4H)

### Step (ii): tert-butyl N-[(1R,2S)-2-(methanesulfonyloxy)-2,3-dihydro-1H-inden-1-yl]carbamate

Methanesulfonic anhydride (2.57 g, 14.75 mmol) as a solution in THF (20 mL) was added to an ice bath cooled solution of tert-butyl ((1R,2S)-2-hydroxy-2,3-dihydro-1H-inden-1-yl)carbamate (3.34 g, 13.41 mmol) and triethylamine (2.056 mL, 14.75 mmol) in THF (40 mL) and allowed to warm to room temp for 1 hour. The reaction mixture was partitioned between water and EtOAc. The organic phase was collected, dried (phase separator) and concentrated in vacuo to afford the title compound.

¹H NMR (300 MHz, DMSO-d₆) δ ppm 1.44 (s, 9H), 3.09-3.27 (m, 5H), 5.16-5.36 (m, 2H), 7.19-7.30 (m, 4H), 7.33-7.44 (m, 1H)

### Step (iii): tert-butyl N-[(1R,2R)-2-azido-2,3-dihydro-1H-inden-1-yl]carbamate

Sodium azide (0.871 g, 13.40 mmol) was added to a solution of tert-butyl N-[(1R,2S)-2-(methanesulfonyloxy)-2,3-dihydro-1H-inden-1-yl]carbamate (4.387 g, 13.40 mmol) in DMSO (40 mL) with stirring and heated to 80° C. under nitrogen for 2 hours. The reaction mixture was partitioned between water and ethyl acetate and the organics concentrated in vacuo. The crude product was purified by column chromatography on silica, eluted with 0-50% ethyl acetate/petrol to afford the title compound.

¹H NMR (300 MHz, DMSO-d₆) δ ppm 1.45 (s, 9H), 2.68-2.83 (m, 1H), 3.14-3.28 (m, 1H), 4.11-4.23 (m, 1H), 4.88-5.00 (m, 1H), 7.07-7.30 (m, 4H), 7.46-7.57 (m, 1H)

### Step (iv): tert-butyl N-[(2R)-2-azido-2,3-dihydro-1H-inden-1-yl]-N-methylcarbamate

tert-butyl ((1R,2R)-2-azido-2,3-dihydro-1H-inden-1-yl)carbamate (3.16 g, 11.52 mmol) as a solution in DMF (20 mL) was added drop wise to an ice cooled, stirred suspension of NaH (0.691 g, 17.28 mmol) in DMF (10 mL). After 30 minutes, methyl iodide (0.936 mL, 14.97 mmol) was added and stirring continued for 30 minutes. The reaction was quenched with water and extracted with EtOAc. The organic phase was collected, dried (phase separator) and concentrated in vacuo to afford the title compound.

¹H NMR (400 MHz, DMSO-d₆) δ ppm 1.36-1.55 (m, 9H), 2.58-2.65 (m, 3H), 2.74-2.85 (m, 1H), 3.20-3.30 (m, 1H), 4.37-4.53 (m, 1H), 5.44-5.66 (m, 1H), 6.99-7.13 (m, 1H), 7.25-7.34 (m, 3H)

### Step (v): tert-butyl N-[(2R)-2-amino-2,3-dihydro-1H-inden-1-yl]-N-methylcarbamate

Pd-C (10 wt. %, 1.224 g, 1.150 mmol) and tert-butyl ((1R,2R)-2-azido-2,3-dihydro-1H-inden-1-yl)(methyl)carbamate (3.32 g, 11.5 mmol) were placed into a flask with ethanol (115 mL) and evacuated/flushed with nitrogen several times. A hydrogen balloon was placed on the reaction and it was stirred overnight. The reaction mixture was filtered through diatomaceous earth, washing with DCM and the filtrate was loaded onto a cation exchange cartridge, washed with methanol and eluted with 2M ammonia/methanol solution then concentrated in vacuo to afford the title compound.

¹H NMR (400 MHz, DMSO-d₆) δ ppm 1.27-1.56 (m, 9H), 1.84 (br. s, 2H), 2.54-2.66 (m, 4H), 2.99-3.11 (m, 1H), 3.43-3.60 (m, 1H), 5.00-5.27 (m, 1H), 6.87-7.03 (m, 1H), 7.12-7.29 (m, 3H)

### Intermediate 44: tert-butyl N-[(1S,2S)-2-amino-2,3-dihydro-1H-inden-1-yl]-N-methylcarbamate

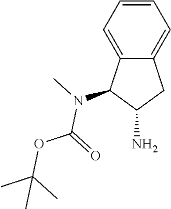 [SMILES: CN(C(=O)OC(C)(C)C)[C@H]1c2ccccc2C[C@@H]1N]

Prepared as described for Intermediate 43 using, (1S,2R)-1-amino-2,3-dihydro-1H-inden-2-ol (5 g, 33.5 mmol) to afford the title compound.

¹H NMR (300 MHz, DMSO-d₆) δ ppm 1.30-1.58 (m, 9H), 1.84 (s, 2H), 2.54-2.70 (m, 4H), 2.96-3.12 (m, 1H), 3.36-3.61 (m, 1H), 4.97-5.30 (m, 1H), 6.86-7.01 (m, 1H), 7.13-7.28 (m, 3H)

### Intermediate 45: tert-butyl N-[(1R,2S)-2-amino-2,3-dihydro-1H-inden-1-yl]-N-methylcarbamate

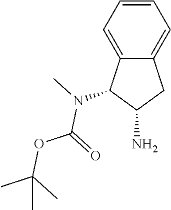 [SMILES: CN(C(=O)OC(C)(C)C)[C@@H]1c2ccccc2C[C@@H]1N]

Prepared as described for Intermediate 43 using, (1R,2R)-1-amino-2,3-dihydro-1H-inden-2-ol (0.50 g, 3.35 mmol) to afford the title compound.

¹H NMR (400 MHz, DMSO) δ ppm 1.46 (s, 9H), 1.63-1.89 (m, 2H), 2.47 (s, 3H), 2.55-2.68 (m, 1H), 3.02-3.13 (m, 1H), 3.69-3.82 (m, 1H), 5.12-5.41 (m, 1H), 7.13-7.31 (m, 4H)

### Intermediate 46 and 47: 2-(cyclopropylmethoxy)-2-(4-fluorophenyl)-N-(cis)-(1-hydroxy-2,3-dihydro-1H-inden-2-yl)acetamide Stereoisomers A and B

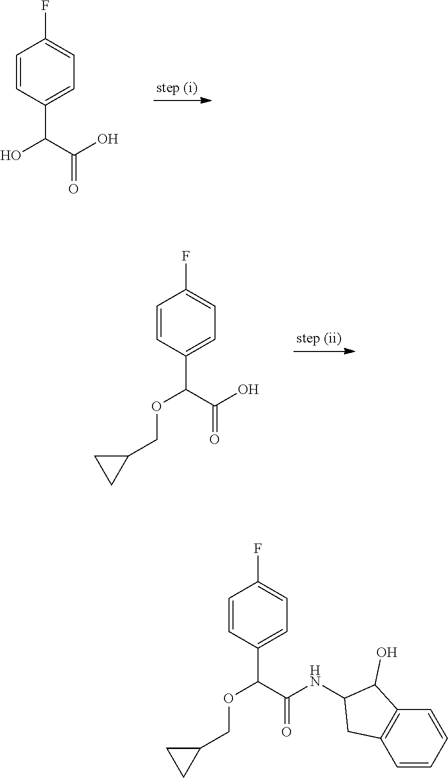 [SMILES: O=C(O)C(O)c1ccc(F)cc1 ; O=C(O)C(OCC1CC1)c1ccc(F)cc1 ; O=C(NC1Cc2ccccc2C1O)C(OCC1CC1)c1ccc(F)cc1]

### Step (i): 2-(cyclopropylmethoxy)-2-(4-fluorophenyl)acetic acid

Sodium hydride (60% dispersion in mineral oil, 5.88 g, 147 mmol) was added to a solution of 2-(4-fluorophenyl)-2-hydroxyacetic acid (10.0 g, 58.8 mmol) and in DMF (180 mL) under nitrogen and stirred for 30 minutes. (Bromomethyl)cyclopropane (14.27 mL, 147 mmol) was added and the reaction was stirred at room temperature for 18 hours. The mixture was diluted with ethyl acetate and washed with saturated sodium bicarbonate solution. The aqueous was acidified to pH 1 with 2M HCl and extracted with EtOAc. The combined organics were dried (phase separator) and concentrated in vacuo to afford the title compound.

¹H NMR (300 MHz, DICHLOROMETHANE-d₂) δ ppm 0.10-0.27 (m, 2H), 0.45-0.62 (m, 2H), 1.00-1.17 (m, 1H), 3.24-3.46 (m, 2H), 4.91 (s, 1H), 6.99-7.14 (m, 2H), 7.39-7.50 (m, 2H), 8.72-9.22 (m, 1H)

MS ES⁻: 223

### Step (ii): 2-(cyclopropylmethoxy)-2-(4-fluorophenyl)-N-(cis)-(1-hydroxy-2,3-dihydro-1H-inden-2-yl)acetamide

HATU (2.447 g, 6.43 mmol) was added to a solution of 2-(cyclopropylmethoxy)-2-(4-fluorophenyl)acetic acid (1.443 g, 6.43 mmol) in DMF (10 mL) under nitrogen. To this was added DIPEA (1.124 mL, 6.43 mmol) and the reaction was stirred at room temperature for 10 mins. 2-Amino-2,3-dihydro-1H-inden-1-ol (0.8 g, 5.36 mmol) was added and the reaction was stirred at room temperature for 24 hours. The mixture was partitioned between ethyl acetate and saturated NaHCO₃. The phases were separated and the aqueous extracted with ethyl acetate. The combined organics were washed with water, dried (phase separator) and concentrated in vacuo. The crude product was purified by column chromatography on silica, eluted with 0-50% ethyl acetate/petroleum ether to afford the title compounds.

### Intermediate 46—Stereoisomer A—First Eluting Stereoisomer

¹H NMR (300 MHz, DMSO-d₆) δ ppm 0.04-0.20 (m, 2H), 0.35-0.48 (m, 2H), 0.90-1.07 (m, 1H), 2.78-2.90 (m, 1H), 3.02-3.16 (m, 1H), 3.19-3.30 (m, 2H), 4.24-4.39 (m, 1H), 4.85 (s, 1H), 4.87-4.97 (m, 1H), 5.60-5.68 (m, 1H), 7.09-7.28 (m, 5H), 7.32-7.50 (m, 3H), 7.67-7.79 (m, 1H)

MS ES⁺: 356

### Intermediate 47—Stereoisomer B—Second Eluting Stereoisomer

¹H NMR (300 MHz, DMSO-d₆) δ ppm 0.06-0.29 (m, 2H), 0.38-0.54 (m, 2H), 0.96-1.11 (m, 1H), 2.71-2.84 (m, 1H), 2.99-3.12 (m, 1H), 3.31-3.36 (m, 2H), 4.34 (s, 1H), 4.85 (s, 1H), 4.90-4.98 (m, 1H), 5.57-5.67 (m, 1H), 7.10-7.31 (m, 5H), 7.33-7.47 (m, 3H), 7.68-7.80 (m, 1H)

MS ES⁺: 356

### Intermediate 48: 2-(cyclopropylmethoxy)-2-(4-fluorophenyl)-N-(trans)-[1-(methylsulfanyl)-2,3-dihydro-1H-inden-2-yl]acetamide

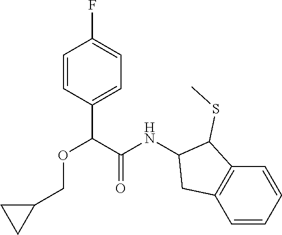 [SMILES: CSC1c2ccccc2CC1NC(=O)C(OCC1CC1)c1ccc(F)cc1]

Prepared as described for Intermediate 34 (step (iv)) using 2-(cyclopropylmethoxy)-2-(4-fluorophenyl)-N-(cis)-(1-hydroxy-2,3-dihydro-1H-inden-2-yl)acetamide (Intermediate 46, 0.550 g, 1.548 mmol) to afford the title compound.

MS ES⁻: 384

### Intermediate 49: 2-(cyclopropylmethoxy)-2-(4-fluorophenyl)-N-(trans)-[1-(methylsulfanyl)-2,3-dihydro-1H-inden-2-yl]acetamide

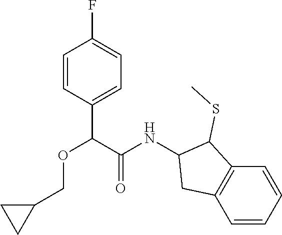 [SMILES: CSC1c2ccccc2CC1NC(=O)C(OCC1CC1)c1ccc(F)cc1]

Prepared as described for Intermediate 34 (step (iv)) using 2-(cyclopropylmethoxy)-2-(4-fluorophenyl)-N-(cis)-(1-hydroxy-2,3-dihydro-1H-inden-2-yl)acetamide (Intermediate 47, 0.660 g, 1.857 mmol) to afford the title compound.

MS ES⁻: 384

### Intermediate 50: tert-butyl 3-{[(1S,2S)-1-{[(tert-butoxy)carbonyl]amino}-2,3-dihydro-1H-inden-2-yl]carbamoyl}-3-phenylpyrrolidine-1-carboxylate

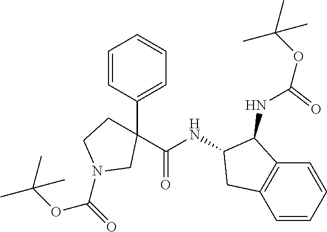 [SMILES: CC(C)(C)OC(=O)N[C@H]1c2ccccc2C[C@@H]1NC(=O)C1(c2ccccc2)CCN(C(=O)OC(C)(C)C)C1]

Prepared as described for Example 1, using 1-(tert-butoxycarbonyl)-3-phenylpyrrolidine-3-carboxylic acid (70 mg, 0.240 mmol) and tert-butyl ((1S,2S)-2-amino-2,3-dihydro-1H-inden-1-yl)carbamate (60 mg, 0.242 mmol). The crude material was purified by column chromatography on silica, eluted with 0-100% ethyl acetate/petroleum ether to afford the title compound.

¹H NMR (400 MHz, DICHLOROMETHANE-d₂) δ ppm 1.38-1.48 (m, 18H), 2.18-2.77 (m, 3H), 3.18-3.63 (m, 4H), 3.97-4.18 (m, 2H), 4.80-5.07 (m, 2H), 6.47-6.72 (m, 1H), 7.11-7.23 (m, 4H), 7.26-7.42 (m, 5H)

MS ES⁺: 522

### Intermediate 51: (2S)-2-(4-fluorophenyl)-N-(trans)-[1-(3-hydroxyazetidin-1-yl)-2,3-dihydro-1H-inden-2-yl]propanamide

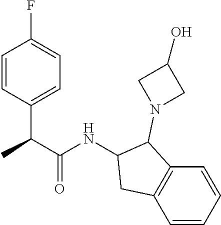 [SMILES: C[C@H](C(=O)NC1Cc2ccccc2C1N1CC(O)C1)c1ccc(F)cc1]

TBAF (1M in THF) (0.300 mL, 0.300 mmol) was added to a solution of (2S)—N-(trans)-(1-{3-[(tert-butyldimethylsilyl)oxy]azetidin-1-yl}-2,3-dihydro-1H-inden-2-yl)-2-(4-fluorophenyl)propanamide (Intermediate 20, 0.128 g, 0.273 mmol) in THF (2 mL) under nitrogen. The reaction was stirred at room temperature for 1 hour. The mixture was partitioned between ethyl acetate and water. The phases were separated and the aqueous extracted twice with ethyl acetate. The combined organics were washed with saturated brine, dried (phase separator) and concentrated in vacuo. The crude product was purified by column chromatography on basic silica, eluted with 0-10% methanol/DCM to afford the title compound.

¹H NMR (400 MHz, DMSO-d₆) δ ppm 1.28 (d, J=6.97 Hz, 3H) 2.96-3.04 (m, 2H) 3.11-3.23 (m, 2H) 3.33-3.40 (m, 1H) 3.49-3.65 (m, 3H) 4.04-4.17 (m, 2H) 5.26 (d, J=6.42 Hz, 1H) 7.04-7.25 (m, 5H) 7.26-7.36 (m, 3H) 8.20 (d, J=7.15 Hz, 1H)

MS ES⁺: 355

### 2. EXAMPLES

### Example 1: (2R)—N-((trans)-1-hydroxy-2,3-dihydro-1H-inden-2-yl)-2-phenylpropanamide

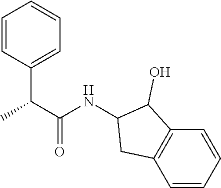 [SMILES: C[C@@H](C(=O)NC1Cc2ccccc2C1O)c1ccccc1]

HATU (133 mg, 0.350 mmol) was added to a solution of (R)-2-phenylpropanoic acid (50 mg, 0.333 mmol) and DIPEA (0.128 mL, 0.732 mmol) in DMF (0.5 mL). The mixture was stirred and allowed to stand. After 5 minutes, (trans)-2-amino-2,3-dihydro-1H-inden-1-ol hydrochloride (61.8 mg, 0.333 mmol) was added. The mixture was stirred and allowed to stand for 5 minutes. The mixture was purified by reverse phase preparative HPLC to afford the title compound.

¹H NMR (400 MHz, DMSO-d₆) δ ppm 1.27-1.41 (m, 3H) 2.42-2.66 (m, 1H) 3.04-3.26 (m, 1H) 3.56-3.71 (m, 1H) 4.01-4.19 (m, 1H) 4.82-4.94 (m, 1H) 5.52 (s, 1H) 7.08-7.40 (m, 9H) 8.35 (d, J=7.07 Hz, 1H)

MS ES⁺: 304 (M+Na)

### Example 2: (2S)—N-((trans)-1-hydroxy-2,3-dihydro-1H-inden-2-yl)-2-phenylpropanamide

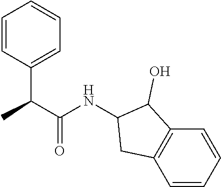 [SMILES: C[C@H](C(=O)NC1Cc2ccccc2C1O)c1ccccc1]

Prepared as described for Example 1 using (S)-2-phenylpropanoic acid (50 mg, 0.333 mmol) to afford the title compound.

¹H NMR (400 MHz, DMSO-d₆) δ ppm 1.28-1.49 (m, 3H) 2.43-2.65 (m, 1H) 3.05-3.25 (m, 1H) 3.59-3.70 (m, 1H) 4.02-4.19 (m, 1H) 4.78-4.96 (m, 1H) 5.41-5.53 (m, 1H) 7.10-7.40 (m, 9H) 8.35 (d, J=7.07 Hz, 1H)

MS ES⁺: 304 (M+Na)

### Examples 3 and 4: (2S)—N-((trans)-1-hydroxy-2,3-dihydro-1H-inden-2-yl)-2-phenylpropanamide Stereoisomers A and B

Example 2 was separated by chiral SFC (AY Daicel CHIRALPAK, 26% isopropanol) to afford the title compounds.

### Example 3—Stereoisomer A—First Eluting Peak

¹H NMR (400 MHz, DMSO-d₆) δ ppm 1.35 (d, J=7.15 Hz, 3H) 2.41-2.48 (m, 1H) 3.05-3.15 (m, 1H) 3.58-3.69 (m, 1H) 4.06-4.18 (m, 1H) 4.85-4.95 (m, 1H) 5.52 (d, J=6.42 Hz, 1H) 7.10-7.25 (m, 4H) 7.26-7.38 (m, 5H) 8.35 (d, J=7.15 Hz, 1H)

MS ES⁺: 304 (M+Na)

### Example 4—Stereoisomer B—Second Eluting Peak

¹H NMR (400 MHz, DMSO-d₆) δ ppm 1.34 (d, J=6.97 Hz, 3H) 2.55-2.66 (m, 1H) 3.14-3.24 (m, 1H) 3.58-3.67 (m, 1H) 4.02-4.14 (m, 1H) 4.79-4.88 (m, 1H) 5.44 (d, J=5.40 Hz, 1H) 7.15-7.39 (m, 9H) 8.36 (d, J=7.15 Hz, 1H)

MS ES⁺: 304 (M+Na)

### Examples 5 and 6: (2S)—N-((cis)-1-hydroxy-2,3-dihydro-1H-inden-2-yl)-2-phenylpropanamide Stereoisomers A and B

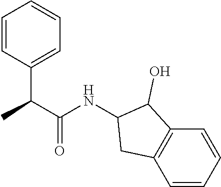 [SMILES: C[C@H](C(=O)NC1Cc2ccccc2C1O)c1ccccc1]

Triethylamine (0.258 mL, 1.850 mmol) was added to a suspension of (S)-2-phenylpropanoic acid (0.102 g, 0.678 mmol), (cis)-2-amino-2,3-dihydro-1H-inden-1-ol (0.092 g, 0.617 mmol), EDC (0.177 g, 0.925 mmol) and HOAt (0.143 g, 0.925 mmol) in DCM (3 mL). The reaction was stirred at room temperature for 4 hours. The reaction was partitioned between DCM and water, passed through a phase separator and concentrated in vacuo. The crude product was purified by column chromatography on silica, eluted with 0-100% ethyl acetate/petroleum.

The product was separated by chiral SFC (AD Daicel CHIRALPAK, 14% Ethanol) to afford the title compounds.

### Example 5—Stereoisomer A—First Eluting Peak

¹H NMR (400 MHz, DMSO-d₆) δ ppm 1.35 (d, J=7.10 Hz, 3H) 2.67-2.77 (m, 1H) 2.90-2.99 (m, 1H) 3.72-3.80 (m, 1H) 4.24-4.35 (m, 1H) 4.84-4.90 (m, 1H) 5.25-5.32 (m, 1H) 7.14-7.24 (m, 4H) 7.25-7.38 (m, 5H) 7.81 (d, J=7.70 Hz, 1H)

MS ES⁻: 280

### Example 6—Stereoisomer B—Second Eluting Peak

¹H NMR (400 MHz, DMSO-d₆) δ ppm 1.34 (d, J=6.97 Hz, 3H) 2.80-2.89 (m, 1H) 3.01-3.09 (m, 1H) 3.74-3.82 (m, 1H) 4.26-4.35 (m, 1H) 4.78-4.84 (m, 1H) 5.24-5.29 (m, 1H) 7.17-7.26 (m, 4H) 7.27-7.40 (m, 5H) 7.75 (d, J=7.34 Hz, 1H)

MS ES⁻: 280

### Example 7: (2S)—N-((trans)-1-methoxy-2,3-dihydro-1H-inden-2-yl)-2-phenylpropanamide

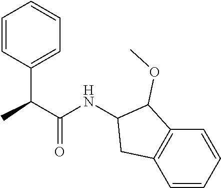 [SMILES: COC1c2ccccc2CC1NC(=O)[C@@H](C)c1ccccc1]

Methyl iodide (0.056 mL, 0.889 mmol) was added to a suspension of (trans)-(2S)—N-(1-hydroxy-2,3-dihydro-1H-inden-2-yl)-2-phenylpropanamide (Example 2, 0.1 g, 0.355 mmol) and silver oxide (0.412 g, 1.777 mmol) in acetonitrile (2 mL) and DMF (1 mL). The reaction was stirred at room temperature for 2 days (in the dark) in a sealed tube. The suspension was filtered and concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 1.28-1.38 (m, 3H) 2.53-2.74 (m, 1H) 3.13-3.40 (m, 4H) 3.54-3.64 (m, 1H) 4.25-4.37 (m, 1H) 4.48-4.68 (m, 1H) 7.16-7.37 (m, 9H) 8.34 (d, J=7.52 Hz, 1H)

MS ES⁺: 318 (M+Na)

### Examples 8 and 9: (2S)—N-((trans)-1-methoxy-2,3-dihydro-1H-inden-2-yl)-2-phenylpropanamide Stereoisomers A and B

Example 7 was separated by chiral SFC (IC Daicel CHIRALPAK, 14% Methanol) to afford the title compounds.

### Example 8—Stereoisomer A—First Eluting Peak

¹H NMR (400 MHz, DMSO-d₆) δ ppm 1.33 (d, J=7.15 Hz, 3H) 2.55-2.63 (m, 1H) 3.14-3.23 (m, 1H) 3.39 (s, 3H) 3.55-3.64 (m, 1H) 4.26-4.34 (m, 1H) 4.66 (d, J=4.22 Hz, 1H) 7.19-7.37 (m, 9H) 8.35 (d, J=7.70 Hz, 1H)

MS ES⁺: 318 (M+Na)

### Example 9—Stereoisomer B—Second Eluting Peak

¹H NMR (400 MHz, DMSO-d₆) δ ppm 2.05-2.10 (m, 3H) 2.65-2.72 (m, 1H) 3.18-3.27 (m, 4H) 3.54-3.62 (m, 1H) 4.26-4.36 (m, 1H) 4.50 (d, J=4.59 Hz, 1H) 7.17-7.36 (m, 9H) 8.34 (d, J=7.70 Hz, 1H)

MS ES⁺: 318 (M+Na)

### Example 10: (2S)—N-((cis)-1-methoxy-2,3-dihydro-1H-inden-2-yl)-2-phenylpropanamide

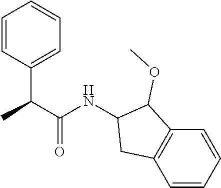 [SMILES: COC1c2ccccc2CC1NC(=O)[C@@H](C)c1ccccc1]

T3P (50% solution in ethyl acetate, 0.201 mL, 0.460 mmol) was added to a solution of (S)-2-phenylpropanoic acid (0.055 g, 0.368 mmol), (cis)-1-methoxy-2,3-dihydro-1H-inden-2-amine (synthesis described in Org. Lett, 2004, 6, 14, 2321, 0.05 g, 0.306 mmol) and triethylamine (0.128 mL, 0.919 mmol) in DCM (2 mL) under nitrogen. The reaction was stirred at room temperature for 1 hour. The mixture was partitioned between DCM and water, passed through a phase separator and concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 1.34 (t, J=7.20 Hz, 3H) 2.71-3.03 (m, 2H) 3.03-3.36 (m, 3H) 3.77 (q, J=7.20 Hz, 1H) 4.38-4.59 (m, 2H) 7.15-7.44 (m, 9H) 7.98-8.11 (m, 1H)

MS ES⁺: 264 (M-OMe)

### Example 11: (2S)—N-[(1S,2S)-1-amino-2,3-dihydro-1H-inden-2-yl]-2-phenylpropanamide hydrochloride

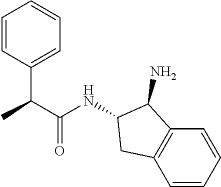 [SMILES: C[C@H](C(=O)N[C@H]1Cc2ccccc2[C@@H]1N)c1ccccc1]

EDC (145 mg, 0.755 mmol), HOAt (125 mg, 0.755 mmol) and triethylamine (0.175 mL, 1.510 mmol) were added to a solution of tert-butyl ((1S,2S)-2-amino-2,3-dihydro-1H-inden-1-yl)carbamate (125 mg, 0.503 mmol) and (S)-2-phenylpropanoic acid (76 mg, 0.503 mmol) in DCM (10 mL) under nitrogen. The reaction was stirred at room temperature for 18 hours. The mixture was washed with saturated NaHCO₃, 2 M HCl and brine, dried (phase separator) and concentrated in vacuo. The crude product was triturated with ether and filtered to afford tert-butyl N-[(1S,2S)-2-[(2S)-2-phenylpropanamido]-2,3-dihydro-1H-inden-1-yl]carbamate. This was treated with HCl (4 M in dioxane, 3 mL) overnight and the solution concentrated in vacuo to afford the title compound.

¹H NMR (400 MHz, DMSO-d₆) δ ppm 1.36-1.39 (m, 3H) 2.89-2.91 (m, 1H) 3.38-3.42 (m, 1H) 3.65-3.69 (m, 1H) 4.32-4.47 (m, 1H) 4.47-4.54 (m, 1H) 7.13-7.43 (m, 8H) 7.55-7.59 (m, 1H) 8.57 (br. s., 2H) 8.73 (d, J=5.87 Hz, 1H)

MS ES⁻: 279

### Example 12: (2S)—N-[(1R,2R)-1-amino-2,3-dihydro-1H-inden-2-yl]-2-phenylpropanamide

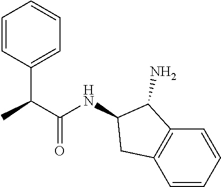 [SMILES: C[C@H](C(=O)N[C@@H]1Cc2ccccc2[C@H]1N)c1ccccc1]

HCl (4 M solution in dioxane, 0.453 mL, 1.813 mmol) was added to a solution of tert-butyl ((1R,2R)-2-((S)-2-phenylpropanamido)-2,3-dihydro-1H-inden-1-yl)carbamate (Example 18, 69 mg, 0.181 mmol) in DCM (1 mL) and stirred over the weekend. The reaction mixture was concentrated in vacuo and purified by reverse phase preparative HPLC eluted with acetonitrile/water (with 0.1% ammonia) to afford the title compound.

¹H NMR (400 MHz, DMSO-d₆) δ ppm 1.37 (d, J=7.06 Hz, 3H) 2.48 (s, 1H) 3.05-3.14 (m, 1H) 3.60-3.70 (m, 1H) 3.90-4.03 (m, 1H) 4.09 (d, J=7.61 Hz, 1H) 7.04-7.25 (m, 4H) 7.27-7.40 (m, 5H) 8.31 (d, J=6.97 Hz, 1H)

MS ES⁺: 281

### Example 13: (2S)—N-[(1S,2S)-1-acetamido-2,3-dihydro-1H-inden-2-yl]-2-phenylpropanamide

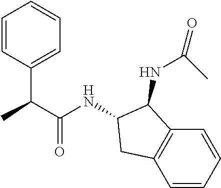 [SMILES: CC(=O)N[C@H]1c2ccccc2C[C@@H]1NC(=O)[C@@H](C)c1ccccc1]

(S)—N-((1S,2S)-1-amino-2,3-dihydro-1H-inden-2-yl)-2-phenylpropanamide hydrochloride (Example 11, 33 mg, 0.118 mmol) and triethylamine (0.092 ml, 0.663 mmol) were added to a solution of acetyl chloride (0.025 mL, 0.354 mmol) in DCM (10 mL). The solution was stirred at room temperature overnight. The reaction was washed with saturated NaHCO₃, 2 M HCl and brine, dried (phase separator) and concentrated in vacuo. The crude product was triturated with ether to afford the title compound.

¹H NMR (400 MHz, DMSO-d₆) δ ppm 1.32-1.37 (m, 3H) 1.81 (s, 3H) 2.66-2.71 (m, 1H) 3.10-3.23 (m, 1H) 3.58-3.62 (m, 1H) 4.28-4.31 (m, 1H) 5.23-5.26 (m, 1H) 7.00-7.38 (m, 9H) 8.22 (d, J=7.98 Hz, 1H) 8.42 (d, J=7.52 Hz, 1H)

MS ES⁻: 321

### Example 14: N-[(1S,2S)-1-amino-2,3-dihydro-1H-inden-2-yl]-2-(2,4-difluorophenyl)butanamide hydrochloride

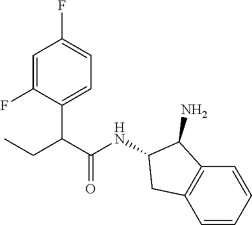 [SMILES: CCC(C(=O)N[C@H]1Cc2ccccc2[C@@H]1N)c1ccc(F)cc1F]

EDC (122 mg, 0.637 mmol), HOAt (105 mg, 0.637 mmol) and triethylamine (0.148 mL, 1.274 mmol) were added to a solution of tert-butyl ((1S,2S)-2-amino-2,3-dihydro-1H-inden-1-yl)carbamate (105 mg, 0.425 mmol) and 2-(2,4-difluorophenyl)butanoic acid (Intermediate 1, 85 mg, 0.425 mmol) in DCM (2 mL) under nitrogen. The reaction was stirred at room temperature for 18 hours. The mixture was washed with saturated NaHCO₃, 2 M HCl and brine, dried (phase separator) and concentrated in vacuo. The crude product was purified by column chromatography on silica, eluted with 0-30% ethyl acetate/petroleum ether to afford tert-butyl ((1S,2S)-2-(2-(2,4-difluorophenyl)butanamido)-2,3-dihydro-1H-inden-1-yl)carbamate. This was treated with HCl (4 M solution in dioxane, 4 mL) for 2 hours. The solution was concentrated in vacuo to afford the title compound.

¹H NMR (400 MHz, DMSO-d₆) δ ppm 0.84 (t, J=7.29 Hz, 3H) 1.56-1.72 (m, 1H) 1.84-2.06 (m, 1H) 2.69-2.93 (m, 1H) 3.34-3.48 (m, 1H) 3.62-3.74 (m, 1H) 4.41 (t, J=6.79 Hz, 1H) 4.52-4.63 (m, 1H) 6.94-7.75 (m, 9H) 8.68-8.89 (m, 1H)

MS ES⁺: 331

### Examples 15 and 16: N-[(1S,2S)-1-amino-2,3-dihydro-1H-inden-2-yl]-2-(2,4-difluorophenyl)butanamide Stereoisomers A and B hydrochlorides

Example 14 was separated by chiral SFC (ID Daicel CHIRALPAK, 40% isopropyl alcohol+0.5% diethylamine) to afford the title compounds.

### Example 15—Stereoisomer A—First Eluting Peak

¹H NMR (400 MHz, DMSO-d₆) δ ppm 0.84 (t, J=7.34 Hz, 3H) 1.57-1.72 (m, 1H) 1.87-2.03 (m, 1H) 2.85-2.89 (m, 1H) 3.40-3.44 (m, 1H) 3.68-3.72 (m, 1H) 4.34-4.44 (m, 1H) 4.53 (d, J=5.59 Hz, 1H) 7.06-7.09 (m, 1H) 7.18-7.23 (m, 1H) 7.28-7.43 (m, 3H) 7.55-7.68 (m, 2H) 8.51 (br. s., 2H) 8.81 (d, J=6.33 Hz, 1H)

MS ES⁺: 331

### Example 16—Stereoisomer B—Second Eluting Peak

¹H NMR (400 MHz, DMSO-d₆) δ ppm 0.84 (t, J=7.34 Hz, 3H) 1.57-1.72 (m, 1H) 1.87-2.03 (m, 1H) 2.85-2.89 (m, 1H) 3.40-3.44 (m, 1H) 3.68-3.72 (m, 1H) 4.34-4.44 (m, 1H) 4.53 (d, J=5.59 Hz, 1H) 7.06-7.09 (m, 1H) 7.18-7.23 (m, 1H) 7.28-7.43 (m, 3H) 7.55-7.68 (m, 2H) 8.51 (br. s., 2H) 8.81 (d, J=6.33 Hz, 1H)

MS ES⁺: 331

### Example 17: N-[(1R,2R)-1-amino-2,3-dihydro-1H-inden-2-yl]-2-(2,4-difluorophenyl)butanamide hydrochloride

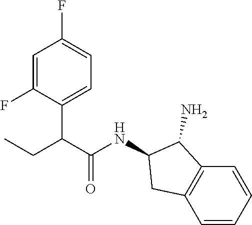 [SMILES: CCC(C(=O)N[C@@H]1Cc2ccccc2[C@H]1N)c1ccc(F)cc1F]

tert-butyl N-[(1R,2R)-2-[2-(2,4-difluorophenyl)butanamido]-2,3-dihydro-1H-inden-1-yl]carbamate (Example 41, 75 mg, 0.174 mmol) was treated with HCl (4 M solution in dioxane, 3 mL). The reaction was stirred for 3 hours then concentrated in vacuo to afford the title compound.

¹H NMR (400 MHz, DMSO-d₆) δ ppm 0.84 (t, J=7.34 Hz, 3H) 1.60-1.73 (m, 1H) 1.89-2.04 (m, 1H) 2.70-2.92 (m, 1H) 3.35-3.50 (m, 1H) 3.64-3.75 (m, 1H) 4.33-4.43 (m, 1H) 4.50-4.64 (m, 1H) 7.01-7.12 (m, 1H) 7.14-7.27 (m, 1H) 7.27-7.39 (m, 3H) 7.46-7.65 (m, 2H) 8.54-8.63 (m, 3H) 8.76-8.81 (m, 1H)

MS ES⁺: 331

### Example 18: tert-butyl N-[(1R,2R)-2-[(2S)-2-phenylpropanamido]-2,3-dihydro-1H-inden-1-yl]carbamate

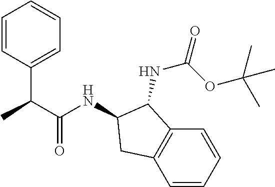 [SMILES: C[C@H](C(=O)N[C@@H]1Cc2ccccc2[C@H]1NC(=O)OC(C)(C)C)c1ccccc1]

Prepared as described in Example 10, using T3P (50% solution in ethyl acetate, 0.479 mL, 0.805 mmol), (S)-2-phenylpropanoic acid (60.5 mg, 0.403 mmol), tert-butyl ((1R,2R)-2-amino-2,3-dihydro-1H-inden-1-yl)carbamate (100 mg, 0.403 mmol) and triethylamine (0.164 mL, 1.208 mmol). The reaction time was 30 minutes and purification was by column chromatography on silica, eluted with 0-100% ethyl acetate/DCM, to afford the title compound.

¹H NMR (400 MHz, DMSO-d₆) δ ppm 1.34 (d, J=7.06 Hz, 3H) 1.45 (s, 9H) 2.53-2.59 (m, 1H) 2.96-3.14 (m, 1H) 3.62 (d, J=7.06 Hz, 1H) 4.19-4.42 (m, 1H) 4.89-5.09 (m, 1H) 6.99-7.41 (m, 10H) 8.28-8.47 (m, 1H)

MS ES⁻: 379

### Example 19: (2S)—N-[(1S,2S)-1-amino-2,3-dihydro-1H-inden-2-yl]-2-methoxy-2-phenylacetamide hydrochloride

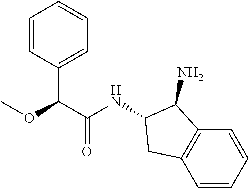 [SMILES: CO[C@H](C(=O)N[C@H]1Cc2ccccc2[C@@H]1N)c1ccccc1]

Prepared as described for Example 11 using tert-butyl ((1S,2S)-2-amino-2,3-dihydro-1H-inden-1-yl)carbamate (100 mg, 0.403 mmol), (S)-2-methoxy-2-phenylacetic acid (66.9 mg, 0.403 mmol), EDC (116 mg, 0.604 mmol), HOAt (100 mg, 0.604 mmol) and triethylamine (0.140 mL, 1.208 mmol). The crude product was purified by column chromatography on silica, eluted with 0-30% ethyl acetate/petroleum ether to afford tert-butyl ((1S,2S)-2-((S)-2-methoxy-2-phenylacetamido)-2,3-dihydro-1H-inden-1-yl)carbamate. This was treated with HCl (4 M solution in dioxane, 4 mL) for 4 hours. The solution was concentrated in vacuo and triturated with ether to afford the title compound.

¹H NMR (400 MHz, DMSO-d₆) δ ppm 2.90-3.01 (m, 1H) 3.20-3.31 (m, 1H) 3.34 (s, 3H) 3.44-3.53 (m, 1H) 3.64-3.79 (m, 1H) 4.48-4.58 (m, 1H) 7.17-7.48 (m, 8H) 7.54-7.64 (m, 1H) 8.54-8.66 (m, 3H) 8.68-8.77 (m, 1H)

MS ES⁺: 297

### Example 20: N-[(1S,2S)-1-amino-2,3-dihydro-1H-inden-2-yl]-2-(4-chlorophenyl)-3-methylbutanamide hydrochloride

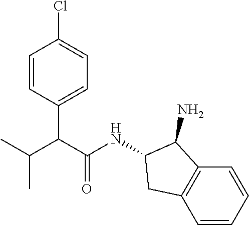 [SMILES: CC(C)C(C(=O)N[C@H]1Cc2ccccc2[C@@H]1N)c1ccc(Cl)cc1]

Prepared as described for Example 14 using tert-butyl ((1S,2S)-2-amino-2,3-dihydro-1H-inden-1-yl)carbamate (100 mg, 0.403 mmol), 2-(4-chlorophenyl)-3-methylbutanoic acid (86 mg, 0.403 mmol), EDC (116 mg, 0.604 mmol), HOAt (100 mg, 0.604 mmol) and triethylamine (0.140 mL, 1.208 mmol). The crude product was purified by column chromatography on silica, eluted with 0-30% ethyl acetate/petroleum ether to afford tert-butyl ((1S,2S)-2-(2-(4-chlorophenyl)-3-methylbutanamido)-2,3-dihydro-1H-inden-1-yl)carbamate. This was treated with HCl (4 M solution in dioxane, 4 mL) for 4 hours. The solution was concentrated in vacuo and triturated with ether to afford the title compound.

¹H NMR (400 MHz, DMSO-d₆) δ ppm 0.58-0.71 (m, 3H) 0.98 (t, J=6.65 Hz, 3H) 2.74-2.88 (m, 1H) 3.08-3.11 (m, 1H) 3.38-3.43 (m, 1H) 3.60-3.78 (m, 1H) 4.20-4.40 (m, 2H) 7.26-7.43 (m, 8H) 7.47-7.67 (m, 1H) 8.77-8.93 (m, 3H)

MS ES⁺: 343

### Example 21: tert-butyl N-[(1S,2S)-2-[(2S)-2-(4-fluorophenyl)propanamido]-2,3-dihydro-1H-inden-1-yl]carbamate

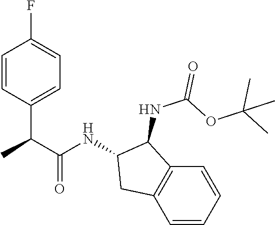 [SMILES: C[C@H](C(=O)N[C@H]1Cc2ccccc2[C@@H]1NC(=O)OC(C)(C)C)c1ccc(F)cc1]

Triethylamine (0.337 mL, 2.416 mmol) was added to a solution of (S)-2-(4-fluorophenyl)propanoic acid (Intermediate 3, 0.149 g, 0.886 mmol), tert-butyl ((1S,2S)-2-amino-2,3-dihydro-1H-inden-1-yl)carbamate (0.2 g, 0.805 mmol), EDC (0.232 g, 1.208 mmol) and HOAt (0.186 g, 1.208 mmol) in DCM (5 mL) under nitrogen. The reaction was stirred at room temperature overnight. The mixture was partitioned between DCM and saturated NaHCO₃. The phases were separated and the aqueous extracted with DCM. The combined organics were washed with water, dried (phase separator) and concentrated in vacuo. The crude product was purified by column chromatography on silica, eluted with 0-50% ethyl acetate/petroleum ether to afford the title compound.

¹H NMR (400 MHz, DMSO-d₆) δ ppm 1.27-1.41 (m, 12H) 2.59-2.70 (m, 1H) 3.07-3.18 (m, 1H) 3.62 (q, J=6.97 Hz, 1H) 4.22-4.34 (m, 1H) 4.94 (t, J=8.80 Hz, 1H) 7.03-7.26 (m, 7H) 7.32-7.39 (m, 2H) 8.38 (d, J=8.07 Hz, 1H)

MS ES⁺: 399

### Example 22: (2S)—N-[(1S,2S)-1-(methylamino)-2,3-dihydro-1H-inden-2-yl]-2-phenylpropanamide

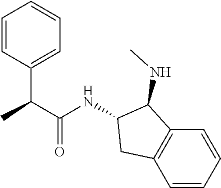 [SMILES: CN[C@H]1c2ccccc2C[C@@H]1NC(=O)[C@@H](C)c1ccccc1]

EDC (145 mg, 0.755 mmol), HOAt (125 mg, 0.755 mmol) and triethylamine (0.175 mL, 1.510 mmol) were added to a solution of tert-butyl ((1S,2S)-2-amino-2,3-dihydro-1H-inden-1-yl)carbamate (125 mg, 0.503 mmol) and (S)-2-phenylpropanoic acid (76 mg, 0.503 mmol) in DCM (10 mL) under nitrogen. The reaction was stirred at room temperature for 18 hours. The mixture was washed with saturated NaHCO₃, 2 M HCl and brine, dried (phase separator) and concentrated in vacuo. The crude product was triturated with ether and filtered to afford tert-butyl N-[(1S,2S)-2-[(2S)-2-phenylpropanamido]-2,3-dihydro-1H-inden-1-yl]carbamate. Lithium aluminium hydride (1 M solution in THF, 99 μl, 0.099 mmol) was added to a solution of tert-butyl N-[(1S,2S)-2-[(2S)-2-phenylpropanamido]-2,3-dihydro-1H-inden-1-yl]carbamate (25 mg, 0.066 mmol) in THF (0.2 mL) and stirred for 30 minutes at room temperature and then heated to 60° C. for 1 hour. After allowing to cool to room temperature, a further portion of lithium aluminium hydride (1 M in THF, 99 μl, 0.099 mmol) was added and the reaction was heated to 60° C. for 1 hour. A saturated solution of sodium sulphate was added dropwise and the mixture was extracted with ethyl acetate. The organics were concentrated in vacuo and the crude product was purified by reverse phase preparative HPLC eluted with acetonitrile/water (with 0.1% ammonia) to afford the title compound.

¹H NMR (400 MHz, DMSO-d₆) δ ppm 1.34 (d, J=7.06 Hz, 3H) 2.16 (s, 3H) 2.57-2.73 (m, 1H) 3.21 (s, 1H) 3.52-3.66 (m, 1H) 3.77-3.90 (m, 1H) 4.15-4.33 (m, 1H) 7.08-7.39 (m, 9H) 8.26 (d, J=7.89 Hz, 1H)

MS ES⁺: 295

### Example 23: tert-butyl N-[(1S,2S)-2-[(2S)-2-(2,4-difluorophenyl)propanamido]-2,3-dihydro-1H-inden-1-yl]carbamate

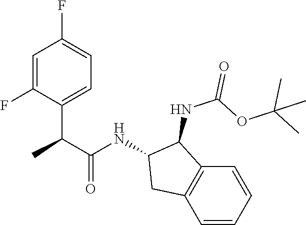 [SMILES: C[C@H](C(=O)N[C@H]1Cc2ccccc2[C@@H]1NC(=O)OC(C)(C)C)c1ccc(F)cc1F]

Prepared as described for Example 21 using triethylamine (0.337 mL, 2.416 mmol), (S)-2-(2,4-difluorophenyl)propanoic acid (Intermediate 2, 0.165 g, 0.886 mmol), tert-butyl ((1S,2S)-2-amino-2,3-dihydro-1H-inden-1-yl)carbamate (0.2 g, 0.805 mmol), EDC (0.232 g, 1.208 mmol) and HOAt (0.186 g, 1.208 mmol). The crude product was purified by column chromatography on silica, eluted with 0-50% ethyl acetate/petroleum ether to afford the title compound.

¹H NMR (400 MHz, DMSO-d₆) δ ppm 1.27-1.41 (m, 12H) 2.61-2.70 (m, 1H) 3.07-3.19 (m, 1H) 3.82-3.90 (m, 1H) 4.26-4.37 (m, 1H) 4.91-4.98 (m, 1H) 6.97-7.09 (m, 2H) 7.10-7.28 (m, 5H) 7.43-7.52 (m, 1H) 8.37-8.42 (m, 1H)

MS ES⁺: 417

### Example 24: (2S)—N-[(1S,2S)-1-amino-2,3-dihydro-1H-inden-2-yl]-2-(4-fluorophenyl)propanamide

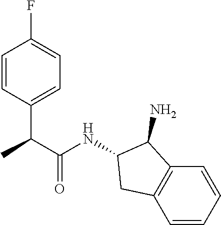 [SMILES: C[C@H](C(=O)N[C@H]1Cc2ccccc2[C@@H]1N)c1ccc(F)cc1]

HCl (4M solution in dioxane, 0.596 mL, 2.384 mmol) was added to a solution of tert-butyl ((1S,2S)-2-((S)-2-(4-fluorophenyl)propanamido)-2,3-dihydro-1H-inden-1-yl)carbamate (Example 21, 0.19 g, 0.477 mmol) in methanol (10 mL). The reaction was stirred at room temperature overnight. The solution was concentrated in vacuo and azeotroped with toluene. The crude product was loaded onto a cation exchange cartridge, washed with methanol and eluted with 2M ammonia/methanol solution then concentrated in vacuo. The product was recrystallised from ethyl acetate/heptane to afford the title compound.

¹H NMR (400 MHz, DMSO-d₆) δ ppm 1.34 (d, J=6.97 Hz, 3H) 1.88 (br. s., 2H) 2.57-2.65 (m, 1H) 3.14-3.22 (m, 1H) 3.65 (q, J=6.97 Hz, 1H) 3.87-4.01 (m, 2H) 7.10-7.21 (m, 5H) 7.24-7.30 (m, 1H) 7.35-7.42 (m, 2H) 8.30 (d, J=6.79 Hz, 1H)

MS ES⁻: 297

### Example 25: (2S)-2-(2,4-difluorophenyl)-N-((trans)-1-methoxy-2,3-dihydro-1H-inden-2-yl)propanamide

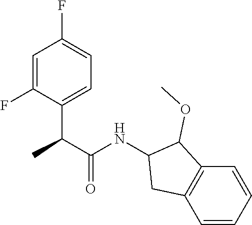 [SMILES: COC1c2ccccc2CC1NC(=O)[C@@H](C)c1ccc(F)cc1F]

Prepared as described for Example 21 using triethylamine (0.168 mL, 1.209 mmol), (S)-2-(2,4-difluorophenyl)propanoic acid (Intermediate 2, 0.075 g, 0.403 mmol), (trans)-1-methoxy-2,3-dihydro-1H-inden-2-amine (Intermediate 4, 0.069 g, 0.423 mmol), EDC (0.116 g, 0.604 mmol) and HOAt (0.093 g, 0.604 mmol). The reaction was diluted with DCM and washed with saturated NaHCO₃, dried (phase separator) and concentrated in vacuo. The crude product was purified by column chromatography on silica, eluted with 0-50% ethyl acetate/petroleum ether, then further purified by column chromatography on silica, eluted with 0-25% ethyl acetate/petroleum ether to afford the title compound.

¹H NMR (400 MHz, DMSO-d₆) δ ppm 1.31-1.37 (m, 3H) 2.57-2.74 (m, 1H) 3.16-3.28 (m, 1H) 3.29-3.41 (m, 3H) 3.79-3.87 (m, 1H) 4.29-4.38 (m, 1H) 4.55-4.69 (m, 1H) 7.03-7.10 (m, 1H) 7.13-7.35 (m, 5H) 7.42-7.52 (m, 1H) 8.37-8.46 (m, 1H)

MS ES⁺: 354 (M+Na)

### Examples 26 and 27: (2S)-2-(2,4-difluorophenyl)-N-((trans)-1-methoxy-2,3-dihydro-1H-inden-2-yl)propanamide Stereoisomers A and B

Example 25 was separated by chiral SFC (Lux-C4 Phenomenex, 10% isopropyl alcohol+0.5% diethylamine) to afford the title compounds.

### Example 26—Stereoisomer A—First Eluting Peak

¹H NMR (400 MHz, DMSO-d₆) δ ppm 1.35 (d, J=7.15 Hz, 3H) 2.57-2.66 (m, 1H) 3.16-3.25 (m, 1H) 3.40 (s, 3H) 3.84 (q, J=7.15 Hz, 1H) 4.29-4.38 (m, 1H) 4.65-4.71 (m, 1H) 7.03-7.11 (m, 1H) 7.14-7.37 (m, 5H) 7.44-7.52 (m, 1H) 8.44 (d, J=7.89 Hz, 1H)

MS ES⁻: 330

### Example 27—Stereoisomer B—Second Eluting Peak

¹H NMR (400 MHz, DMSO-d₆) δ ppm 1.30-1.39 (m, 3H) 2.64-2.75 (m, 1H) 3.18-3.28 (m, 1H) 3.28-3.37 (m, 3H) 3.77-3.87 (m, 1H) 4.30-4.39 (m, 1H) 4.53-4.59 (m, 1H) 7.02-7.10 (m, 1H) 7.13-7.33 (m, 5H) 7.41-7.50 (m, 1H) 8.40 (d, J=7.89 Hz, 1H)

MS ES⁻: 330

### Example 28: (2S)-2-(2,4-difluorophenyl)-N-((trans)-1-ethoxy-2,3-dihydro-1H-inden-2-yl)propanamide

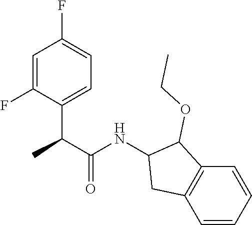 [SMILES: CCOC1c2ccccc2CC1NC(=O)[C@@H](C)c1ccc(F)cc1F]

Prepared as described for Example 21 using triethylamine (0.168 mL, 1.209 mmol), (S)-2-(2,4-difluorophenyl)propanoic acid (Intermediate 2, 0.075 g, 0.403 mmol), (trans)-1-ethoxy-2,3-dihydro-1H-inden-2-amine (Intermediate 5, 0.075 g, 0.423 mmol), EDC (0.116 g, 0.604 mmol) and HOAt (0.093 g, 0.604 mmol). The reaction was diluted with DCM and washed with saturated NaHCO₃, dried (phase separator) and concentrated in vacuo. The crude products were purified by column chromatography on silica, eluted with 0-50% ethyl acetate/petroleum ether, then further purified by column chromatography on silica, eluted with 0-20% ethyl acetate/petroleum ether to afford the title compound.

¹H NMR (400 MHz, DMSO-d₆) δ ppm 1.01-1.17 (m, 3H) 1.34 (d, J=6.97 Hz, 3H) 2.56-2.73 (m, 1H) 3.14-3.26 (m, 1H) 3.43-3.74 (m, 2H) 3.78-3.87 (m, 1H) 4.25-4.36 (m, 1H) 4.62-4.79 (m, 1H) 7.00-7.10 (m, 1H) 7.13-7.34 (m, 5H) 7.41-7.52 (m, 1H) 8.35-8.45 (m, 1H)

MS ES⁺: 368 (M+Na)

### Examples 29 and 30: (2S)-2-(2,4-difluorophenyl)-N-((trans)-1-ethoxy-2,3-dihydro-1H-1-inden-2-yl)propanamide Stereoisomers A and B

Example 28 was separated by chiral SFC (Lux-C4 Phenomenex, 14% methanol) to afford the title compounds.

### Example 29—Stereoisomer A—First Eluting Peak

¹H NMR (400 MHz, DMSO-d₆) δ ppm 1.06 (t, J=7.06 Hz, 3H) 1.34 (d, J=7.15 Hz, 3H) 2.64-2.72 (m, 1H) 3.17-3.26 (m, 1H) 3.42-3.62 (m, 2H) 3.83 (q, J=7.15 Hz, 1H) 4.27-4.35 (m, 1H) 4.62-4.67 (m, 1H) 7.01-7.10 (m, 1H) 7.12-7.30 (m, 5H) 7.40-7.48 (m, 1H) 8.38 (d, J=7.89 Hz, 1H)

MS ES⁻: 344

### Example 30—Stereoisomer B—Second Eluting Peak

¹H NMR (400 MHz, DMSO-d₆) δ ppm 1.14 (t, J=6.97 Hz, 3H) 1.34 (d, J=7.15 Hz, 3H) 2.56-2.65 (m, 1H) 3.15-3.24 (m, 1H) 3.56-3.74 (m, 2H) 3.78-3.87 (m, 1H) 4.25-4.34 (m, 1H) 4.72-4.79 (m, 1H) 7.02-7.10 (m, 1H) 7.13-7.28 (m, 4H) 7.29-7.34 (m, 1H) 7.43-7.52 (m, 1H) 8.37-8.45 (m, 1H)

MS ES⁻: 344

### Example 31: (2S)—N-((trans)-1-ethoxy-2,3-dihydro-1H-inden-2-yl)-2-(4-fluorophenyl)propanamide

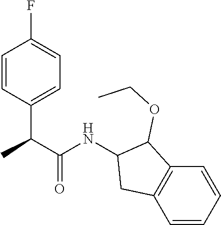 [SMILES: CCOC1c2ccccc2CC1NC(=O)[C@@H](C)c1ccc(F)cc1]

Prepared as described for Example 21 using triethylamine (0.186 mL, 1.338 mmol), (S)-2-(4-fluorophenyl)propanoic acid (Intermediate 3, 0.075 g, 0.446 mmol), (trans)-1-ethoxy-2,3-dihydro-1H-inden-2-amine (Intermediate 5, 0.083 g, 0.468 mmol), EDC (0.128 g, 0.669 mmol) and HOAt (0.103 g, 0.669 mmol). The mixture was diluted with DCM and washed with 5% citric acid, dried (phase separator) and concentrated in vacuo. The crude product was purified by column chromatography on silica, eluted with 0-30% ethyl acetate/petroleum ether. The product was further purified by reverse phase chromatography on C18 silica eluted with 5-95% acetonitrile/water (with 0.05% ammonia) to afford the title compound.

¹H NMR (300 MHz, DMSO-d₆) δ ppm 0.93-1.18 (m, 3H) 1.27-1.37 (m, 3H) 2.53-2.73 (m, 1H) 3.11-3.27 (m, 1H) 3.35-3.47 (m, 1H) 3.53-3.73 (m, 2H) 4.19-4.35 (m, 1H) 4.55-4.76 (m, 1H) 7.06-7.28 (m, 6H) 7.29-7.39 (m, 2H) 8.32-8.39 (m, 1H)

MS ES⁻: 326

### Examples 32 and 33: (2S)—N-((trans)-1-ethoxy-2,3-dihydro-1H-inden-2-yl)-2-(4-fluorophenyl)propanamide Stereoisomers A and B

Example 31 was separated by chiral SFC (Lux-C4 Phenomenex, 14% methanol) to afford the title compounds.

### Example 32—Stereoisomer A—First Eluting Peak

¹H NMR (400 MHz, METHANOL-d₄) δ ppm 1.07 (t, J=6.97 Hz, 3H) 1.43 (d, J=6.97 Hz, 3H) 2.68-2.77 (m, 1H) 3.32-3.36 (m, 1H) 3.39-3.57 (m, 2H) 3.63 (q, J=6.97 Hz, 1H) 4.41-4.51 (m, 1H) 4.65 (d, J=5.14 Hz, 1H) 6.98-7.06 (m, 2H) 7.16-7.30 (m, 4H) 7.32-7.39 (m, 2H)

MS ES⁺: 350 (M+Na)

### Example 33—Stereoisomer B—Second Eluting Peak

¹H NMR (400 MHz, METHANOL-d₄) δ ppm 1.22 (t, J=7.06 Hz, 3H) 1.43 (d, J=7.15 Hz, 3H) 2.60-2.69 (m, 1H) 3.23-3.29 (m, 1H) 3.63 (q, J=7.06 Hz, 1H) 3.66-3.82 (m, 2H) 4.40-4.47 (m, 1H) 4.80-4.83 (m, 1H) 6.98-7.07 (m, 2H) 7.17-7.29 (m, 3H) 7.31-7.39 (m, 3H)

MS ES⁺: 350 (M+Na)

### Example 34: (2S)—N-[(1S,2S)-1-amino-2,3-dihydro-1H-inden-2-yl]-2-(2,4-difluorophenyl)propanamide

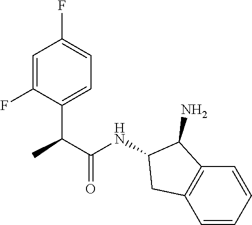 [SMILES: C[C@H](C(=O)N[C@H]1Cc2ccccc2[C@@H]1N)c1ccc(F)cc1F]

Prepared as described for Example 24 using HCl (4 M solution in dioxane) (0.630 mL, 2.52 mmol) and tert-butyl ((1S,2S)-2-((S)-2-(2,4-difluorophenyl)propanamido)-2,3-dihydro-1H-inden-1-yl)carbamate (Example 23, 0.21 g, 0.504 mmol) to afford the title compound.

¹H NMR (400 MHz, DMSO-d₆) δ ppm 1.36 (d, J=7.15 Hz, 3H) 1.87-2.04 (m, 2H) 2.58-2.66 (m, 1H) 3.12-3.23 (m, 1H) 3.89 (q, J=7.20 Hz, 1H) 3.93-4.05 (m, 2H) 7.02-7.11 (m, 1H) 7.13-7.24 (m, 4H) 7.26-7.33 (m, 1H) 7.47-7.57 (m, 1H) 8.34 (d, J=6.60 Hz, 1H)

MS ES⁺: 317

### Example 35: (2S)—N-[(1R,2R)-1-amino-2,3-dihydro-1H-inden-2-yl]-2-(2,4-difluorophenyl)propanamide

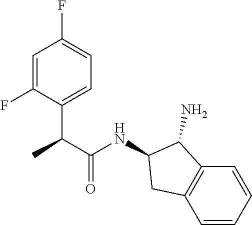 [SMILES: C[C@H](C(=O)N[C@@H]1Cc2ccccc2[C@H]1N)c1ccc(F)cc1F]

Triethylamine (0.126 mL, 0.906 mmol) was added to a suspension of (S)-2-(2,4-difluorophenyl)propanoic acid (Intermediate 2, 0.062 g, 0.332 mmol), tert-butyl ((1R,2R)-2-amino-2,3-dihydro-1H-inden-1-yl)carbamate (0.075 g, 0.302 mmol), EDC (0.087 g, 0.453 mmol) and HOAt (0.070 g, 0.453 mmol) in DCM (2 mL) under nitrogen. The reaction was stirred at room temperature overnight. The mixture was partitioned between DCM and saturated NaHCO₃, passed through a phase separator and concentrated in vacuo. The crude product was purified by column chromatography on silica, eluted with 0-50% ethyl acetate/petroleum ether to afford tert-butyl ((1R,2R)-2-((S)-2-(2,4-difluorophenyl)propanamido)-2,3-dihydro-1H-inden-1-yl)carbamate. This was treated with HCl (4 M solution in dioxane, 0.150 mL, 0.600 mmol) in methanol (2 mL). The reaction was stirred at room temperature overnight. The solution was concentrated in vacuo and azeotroped with toluene. The crude product was loaded onto a cation exchange cartridge, washed with methanol and eluted with 2M ammonia/methanol solution then concentrated in vacuo to afford the title compound.

¹H NMR (400 MHz, DMSO-d₆) δ ppm 1.37 (d, J=7.15 Hz, 3H) 2.50-2.57 (m, 1H) 2.89 (br. s., 2H) 3.09-3.19 (m, 1H) 3.83-3.93 (m, 1H) 3.94-4.05 (m, 1H) 4.08-4.15 (m, 1H) 7.02-7.10 (m, 1H) 7.12-7.24 (m, 4H) 7.33 (d, J=6.60 Hz, 1H) 7.43-7.52 (m, 1H) 8.36 (d, J=6.97 Hz, 1H)

MS ES⁺: 317

### Example 36: (2S)-2-(4-fluorophenyl)-N-((trans)-1-methoxy-2,3-dihydro-1H-inden-2-yl)propanamide

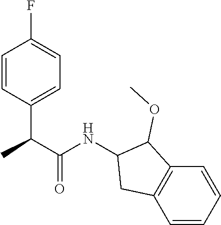 [SMILES: COC1c2ccccc2CC1NC(=O)[C@@H](C)c1ccc(F)cc1]

Prepared as described for Example 21 using triethylamine (0.186 mL, 1.338 mmol), (S)-2-(4-fluorophenyl)propanoic acid (Intermediate 3, 0.075 g, 0.446 mmol), (trans)-1-methoxy-2,3-dihydro-1H-inden-2-amine (Intermediate 4, 0.076 g, 0.468 mmol), EDC (0.128 g, 0.669 mmol) and HOAt (0.103 g, 0.669 mmol). The mixture was diluted with DCM and washed with 5% citric acid, dried (phase separator) and concentrated in vacuo. The crude product was purified by column chromatography on silica, eluted with 0-30% ethyl acetate/petroleum ether. The product was further purified by reverse phase chromatography on C18 silica eluted with 5-95% acetonitrile/water (with 0.05% ammonia) to afford the title compound.

¹H NMR (300 MHz, DMSO-d₆) δ ppm 1.32 (s, 3H) 2.52-2.74 (m, 1H) 3.10-3.40 (m, 4H) 3.53-3.65 (m, 1H) 4.23-4.38 (m, 1H) 4.45-4.68 (m, 1H) 7.04-7.18 (m, 2H) 7.18-7.39 (m, 6H) 8.31-8.42 (m, 1H)

MS ES⁻: 312

### Examples 37 and 38: (2S)-2-(4-fluorophenyl)-N-((trans)-1-methoxy-2,3-dihydro-1H-inden-2-yl)propanamide Stereoisomers A and B

Example 36 was separated by chiral SFC (Lux-C4 Phenomenex 20% isopropyl alcohol) to afford the title compounds.

### Example 37—Stereoisomer A—First Eluting Peak

¹H NMR (400 MHz, METHANOL-d₄) δ ppm 1.43 (d, J=7.20 Hz, 3H) 2.59-2.70 (m, 1H) 3.24-3.30 (m, 1H) 3.50 (s, 3H) 3.63 (q, J=7.20 Hz, 1H) 4.43-4.51 (m, 1H) 4.70-4.76 (m, 1H) 6.99-7.07 (m, 2H) 7.18-7.30 (m, 3H) 7.32-7.40 (m, 3H)

MS ES⁻: 312

### Example 38—Stereoisomer B—Second Eluting Peak

¹H NMR (400 MHz, METHANOL-d₄) δ ppm 1.44 (d, J=7.15 Hz, 3H) 2.70-2.79 (m, 1H) 3.32-3.39 (m, 4H) 3.63 (q, J=7.15 Hz, 1H) 4.45-4.51 (m, 1H) 4.54-4.58 (m, 1H) 6.98-7.06 (m, 2H) 7.17-7.31 (m, 4H) 7.33-7.38 (m, 2H)

MS ES⁻: 312

### Example 39: (2S)-2-(4-fluorophenyl)-N-(cis)-(1-methoxy-2,3-dihydro-1H-inden-2-yl)propanamide

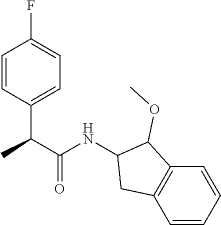 [SMILES: COC1c2ccccc2CC1NC(=O)[C@@H](C)c1ccc(F)cc1]

Triethylamine (0.279 mL, 2.003 mmol) was added to a solution of (S)-2-(4-fluorophenyl)propanoic acid (Intermediate 3, 0.118 g, 0.701 mmol), (cis)-1-methoxy-2,3-dihydro-1H-inden-2-amine ((synthesis described in Org. Lett, 2004, 6, 14, 2321) 0.109 g, 0.668 mmol), EDC (0.192 g, 1.002 mmol) and HOAt (0.136 g, 1.002 mmol) in DCM (5 mL). The reaction was stirred at room temperature for 4 hours. The mixture was partitioned between DCM and saturated NaHCO₃. The phases were separated, dried (phase separator) and concentrated in vacuo. The crude product was purified by column chromatography on silica, eluted with 0-50% ethyl acetate/petroleum ether. The product was further purified by reverse phase preparative HPLC eluted with acetonitrile/water (with 0.1% ammonia) to afford the title compound.

¹H NMR (400 MHz, DMSO-d₆) δ ppm 1.28-1.37 (m, 3H) 2.71-3.04 (m, 2H) 3.04-3.32 (m, 3H) 3.74-3.83 (m, 1H) 4.39-4.58 (m, 2H) 7.07-7.16 (m, 2H) 7.18-7.32 (m, 3H) 7.33-7.43 (m, 3H) 8.04-8.15 (m, 1H)

MS ES⁻: 312

### Example 40: (2S)-2-(2,4-difluorophenyl)-N-[(1S,2S)-1-{[(oxan-4-yl)methyl]amino}-2,3-dihydro-1H-inden-2-yl]propanamide

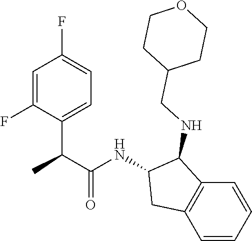 [SMILES: C[C@H](C(=O)N[C@H]1Cc2ccccc2[C@@H]1NCC1CCOCC1)c1ccc(F)cc1F]

Tetrahydro-2H-pyran-4-carbaldehyde (76 mg, 0.666 mmol) was added to a suspension of (S)—N-((1S,2S)-1-amino-2,3-dihydro-1H-inden-2-yl)-2-(2,4-difluorophenyl)propanamide hydrochloride (the hydrochloride salt of the compound of Example 34, 196 mg, 0.555 mmol) and triethylamine (0.075 mL, 0.555 mmol) in THF (2 mL) and stirred for 45 mins. Sodium triacetoxyhydroborate (141 mg, 0.666 mmol) was added and the reaction was stirred for 30 minutes at room temperature. The reaction was quenched with water and extracted with DCM. The combined organics were purified by column chromatography on silica, eluted with 0-100% ethyl acetate/petroleum ether to afford the title compound.

¹H NMR (400 MHz, METHANOL-d₄) δ ppm 1.05-1.31 (m, 2H) 1.49 (d, J=7.06 Hz, 3H) 1.54-1.66 (m, 2H) 1.68-1.79 (m, 1H) 2.51-2.66 (m, 2H) 2.78-2.89 (m, 1H) 3.34-3.46 (m, 3H) 3.84-4.01 (m, 3H) 4.17 (d, J=5.96 Hz, 1H) 4.43-4.54 (m, 1H) 6.85-7.02 (m, 2H) 7.16-7.29 (m, 3H) 7.33-7.42 (m, 1H) 7.53 (m, 1H)

MS ES⁺: 415

### Example 41: tert-butyl N-[(1R,2R)-2-[2-(2,4-difluorophenyl)butanamido]-2,3-dihydro-1H-inden-1-yl]carbamate

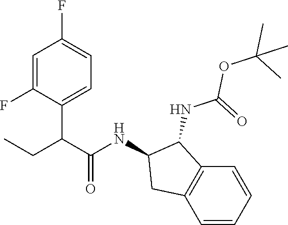 [SMILES: CCC(C(=O)N[C@@H]1Cc2ccccc2[C@H]1NC(=O)OC(C)(C)C)c1ccc(F)cc1F]

Prepared as described for Example 21 using tert-butyl ((1R,2R)-2-amino-2,3-dihydro-1H-inden-1-yl)carbamate (75 mg, 0.302 mmol), 2-(2,4-difluorophenyl)butanoic acid (Intermediate 1, 60.5 mg, 0.302 mmol), EDC (87 mg, 0.453 mmol), HOAt (74.8 mg, 0.453 mmol) and triethylamine (0.105 mL, 0.906 mmol). The mixture was washed with 2 M HCl, saturated NaHCO₃ and brine, dried (phase separator) and concentrated in vacuo. The crude product was purified by column chromatography on silica, eluted with 0-30% ethyl acetate/petroleum ether to afford the title compound.

¹H NMR (400 MHz, DMSO-d₆) δ ppm 0.79-0.93 (m, 3H) 1.31-1.47 (m, 9H) 1.54-1.68 (m, 1H) 1.86-2.00 (m, 1H) 2.54-2.72 (m, 1H) 2.99-3.20 (m, 1H) 3.59-3.70 (m, 1H) 4.25-4.45 (m, 1H) 4.95-5.06 (m, 1H) 6.99-7.33 (m, 7H) 7.52-7.62 (m, 1H) 8.49-8.57 (m, 1H)

MS ES⁺: 431

### Example 42: tert-butyl N-[(1S,2S)-2-[(2S)-2-(4-fluorophenyl)butanamido]-2,3-dihydro-1H-inden-1-yl]carbamate

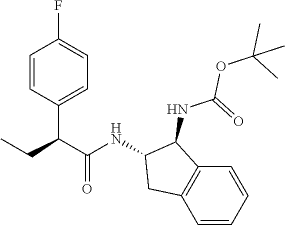 [SMILES: CC[C@H](C(=O)N[C@H]1Cc2ccccc2[C@@H]1NC(=O)OC(C)(C)C)c1ccc(F)cc1]

Prepared as described for Example 21 using EDC (275 mg, 1.436 mmol), HOAt (195 mg, 1.436 mmol), (S)-2-(4-fluorophenyl)butanoic acid (Intermediate 6, 218 mg, 1.197 mmol), tert-butyl ((1S,2S)-2-amino-2,3-dihydro-1H-inden-1-yl)carbamate (297 mg, 1.197 mmol) and 4-methylmorpholine (242 mg, 2.393 mmol). The crude product was purified by column chromatography on silica, eluted with 0-70% ethyl acetate/petroleum ether to afford the title compound.

¹H NMR (300 MHz, DMSO-d₆) δ ppm 0.83 (t, J=7.22 Hz, 3H) 1.36 (s, 9H) 1.53-1.70 (m, 1H) 1.84-2.04 (m, 1H) 2.57-2.79 (m, 1H) 3.06-3.20 (m, 1H) 3.33-3.46 (m, 1H) 4.13-4.37 (m, 1H) 4.83-5.09 (m, 1H) 7.01-7.25 (m, 7H) 7.30-7.48 (m, 2H) 8.30-8.51 (m, 1H)

MS ES⁺: 413

### Example 43: tert-butyl N-[(1R,2R)-2-[(2S)-2-(4-fluorophenyl)butanamido]-2,3-dihydro-1H-inden-1-yl]carbamate

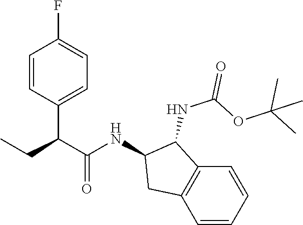 [SMILES: CC[C@H](C(=O)N[C@@H]1Cc2ccccc2[C@H]1NC(=O)OC(C)(C)C)c1ccc(F)cc1]

Prepared as described for Example 21 using EDC (275 mg, 1.436 mmol), HOAt (195 mg, 1.436 mmol), (S)-2-(4-fluorophenyl)butanoic acid (Intermediate 6, 218 mg, 1.197 mmol), tert-butyl ((1R,2R)-2-amino-2,3-dihydro-1H-inden-1-yl)carbamate (297 mg, 1.197 mmol) and 4-methylmorpholine (242 mg, 2.393 mmol). The crude product was purified by column chromatography on silica, eluted with 0-70% ethyl acetate/petroleum ether to afford the title compound.

¹H NMR (300 MHz, DMSO-d₆) δ ppm 0.84 (t, J=7.22 Hz, 3H) 1.44 (s, 9H) 1.53-1.68 (m, 1H) 1.84-2.07 (m, 1H) 2.54-2.59 (m, 1H) 2.91-3.21 (m, 1H) 3.34-3.43 (m, 1H) 4.24-4.51 (m, 1H) 4.87-5.07 (m, 1H) 7.00-7.46 (m, 9H) 8.36-8.53 (m, 1H)

MS ES⁺: 413

### Example 44: (2S)-2-(4-fluorophenyl)-N-((trans)-1-methoxy-2,3-dihydro-1H-inden-2-yl)butanamide

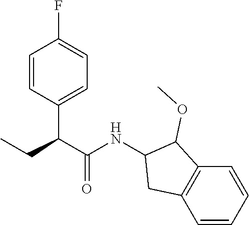 [SMILES: CC[C@H](C(=O)NC1Cc2ccccc2C1OC)c1ccc(F)cc1]

Prepared as described for Example 21 using (S)-2-(4-fluorophenyl)butanoic acid (Intermediate 6, 0.15 g, 0.823 mmol), (trans)-1-methoxy-2,3-dihydro-1H-inden-2-amine (Intermediate 4, 0.148 g, 0.906 mmol), EDC (0.237 g, 1.235 mmol), HOAt (0.168 g, 1.235 mmol) and triethylamine (0.344 mL, 2.470 mmol). The mixture was partitioned between DCM and 5% citric acid, passed through a phase separator and concentrated in vacuo. The crude product was purified by column chromatography on silica, eluted with 5-40% ethyl acetate/petroleum ether to afford the title compound.

¹H NMR (300 MHz, DMSO-d₆) δ ppm 0.74-0.88 (m, 3H) 1.51-2.04 (m, 2H) 2.53-2.74 (m, 1H) 3.12-3.42 (m, 5H) 4.24-4.37 (m, 1H) 4.49-4.67 (m, 1H) 7.05-7.18 (m, 2H) 7.19-7.39 (m, 6H) 8.34-8.47 (m, 1H)

MS ES⁻: 326

### Examples 45 and 46: (2S)-2-(4-fluorophenyl)-N-((trans)-1-methoxy-2,3-dihydro-1H-inden-2-yl)butanamide Stereoisomers A and B

Example 44 was separated by chiral SFC (IC Daicel CHIRALPAK, 10% methanol) to afford the title compounds.

### Example 45—Stereoisomer A—First Eluting Peak

¹H NMR (400 MHz, DMSO-d₆) δ ppm 0.80 (t, J=7.24 Hz, 3H) 1.53-1.66 (m, 1H) 1.88-2.01 (m, 1H) 2.53-2.62 (m, 1H) 3.13-3.22 (m, 1H) 3.33-3.36 (m, 1H) 3.39 (s, 3H) 4.26-4.36 (m, 1H) 4.63 (d, J=4.22 Hz, 1H) 7.07-7.16 (m, 2H) 7.18-7.30 (m, 3H) 7.31-7.38 (m, 3H) 8.41 (d, J=7.52 Hz, 1H)

MS ES⁻: 326

### Example 46—Stereoisomer B—Second Eluting Peak

¹H NMR (400 MHz, DMSO-d₆) δ ppm 0.81 (t, J=7.24 Hz, 3H) 1.54-1.67 (m, 1H) 1.88-2.02 (m, 1H) 2.63-2.71 (m, 1H) 3.17-3.29 (m, 4H) 3.33-3.38 (m, 1H) 4.26-4.35 (m, 1H) 4.51 (d, J=4.58 Hz, 1H) 7.07-7.15 (m, 2H) 7.17-7.29 (m, 4H) 7.30-7.37 (m, 2H) 8.42 (d, J=7.70 Hz, 1H)

MS ES⁻: 326

### Example 47: (2S)—N-[(1S,2S)-1-[(cyclopropylmethyl)amino]-2,3-dihydro-1H-inden-2-yl]-2-(4-fluorophenyl)propanamide

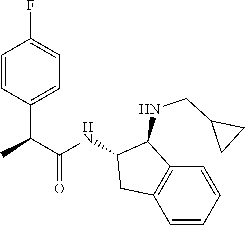 [SMILES: C[C@H](C(=O)N[C@H]1Cc2ccccc2[C@@H]1NCC1CC1)c1ccc(F)cc1]

Lithium bis(trimethylsilyl)amide (1M in THF, 0.15 mL, 0.151 mmol) was added to a solution of tert-butyl ((1S,2S)-2-((S)-2-(4-fluorophenyl)propanamido)-2,3-dihydro-1H-inden-1-yl)carbamate (Example 21, 50 mg, 0.125 mmol) in DMF (1 mL). The reaction was stirred at room temperature for 15 minutes under nitrogen. (Bromomethyl)cyclopropane (20.33 mg, 0.151 mmol) was added and stirred for 4 hours. The mixture was partitioned between ethyl acetate and saturated brine. The phases were separated and the aqueous extracted with ethyl acetate. The combined organics were washed with half saturated brine, dried (phase separator) and concentrated in vacuo. The crude product was purified by column chromatography on silica, eluted with 0-50% ethyl acetate/petroleum ether to afford tert-butyl (cyclopropylmethyl)((1S,2S)-2-((S)-2-(4-fluorophenyl)propanamido)-2,3-dihydro-1H-inden-1-yl)carbamate. This was treated with HCl (4 M solution in dioxane, 0.055 mL, 0.221 mmol) in methanol (2 mL). The reaction was stirred at room temperature overnight. A further portion of HCl (4 M in dioxane, 0.055 mL, 0.221 mmol) was added and the reaction stirred for 5 hours. The solution was concentrated and azeotroped with toluene. The crude product was purified by reverse phase preparative HPLC eluted with acetonitrile/water (with 0.1% ammonia) to afford the title compound.

¹H NMR (300 MHz, DICHLOROMETHANE-d₂) δ ppm −0.05-0.10 (m, 2H) 0.34-0.48 (m, 2H) 0.76-0.97 (m, 1H) 1.45 (d, J=7.01 Hz, 3H) 2.41-2.49 (m, 2H) 2.55-2.67 (m, 1H) 3.28-3.42 (m, 1H) 3.42-3.55 (m, 1H) 3.92 (d, J=5.50 Hz, 1H) 4.31-4.46 (m, 1H) 5.61-5.72 (m, 1H) 6.94-7.07 (m, 2H) 7.12-7.34 (m, 6H)

MS ES⁺: 353

### Examples 48 and 49: tert-butyl N-[(1S,2S)-2-[2-(2,4-difluorophenyl)-2-(2-oxo-1,2-dihydropyridin-1-yl)acetamido]-2,3-dihydro-1H-inden-1-yl]carbamate Stereoisomers A and B

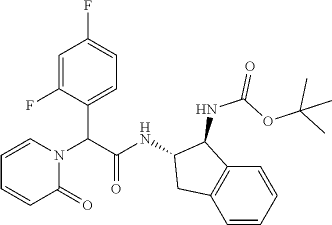 [SMILES: CC(C)(C)OC(=O)N[C@H]1c2ccccc2C[C@@H]1NC(=O)C(c1ccc(F)cc1F)n1ccccc1=O]

HOAt (186 mg, 1.208 mmol) and EDC (232 mg, 1.208 mmol) were added to a solution of 2-(2,4-difluorophenyl)-2-(2-oxopyridin-1(2H)-yl)acetic acid (Intermediate 7, 235 mg, 0.886 mmol), tert-butyl ((1S,2S)-2-amino-2,3-dihydro-1H-inden-1-yl)carbamate (200 mg, 0.805 mmol) and triethylamine (244 mg, 2.416 mmol) in DCM (8 mL). The reaction was stirred at room temperature for 26 hours. The reaction mixture was washed with saturated NaHCO₃ and concentrated in vacuo. The crude product was purified by column chromatography on silica, eluted with 0-50% ethyl acetate/petroleum ether to afford the title compounds.

### Example 48 Stereoisomer A—First Eluting Peak

¹H NMR (300 MHz, DICHLOROMETHANE-d₂) δ ppm 1.46 (s, 9H) 2.68-2.81 (m, 1H) 3.44-3.56 (m, 1H) 4.24 (s, 1H) 5.00-5.15 (m, 2H) 6.21 (t, J=6.33 Hz, 1H) 6.63 (d, J=8.67 Hz, 1H) 6.83 (s, 1H) 6.86-7.05 (m, 2H) 7.17-7.33 (m, 6H) 7.34-7.43 (m, 1H) 7.50-7.62 (m, 1H)

MS ES⁺: 496

### Example 49—Stereoisomer B—Second Eluting Peak

¹H NMR (300 MHz, DICHLOROMETHANE-d₂) δ ppm 1.41 (s, 9H) 2.70-2.83 (m, 1H) 3.36-3.51 (m, 1H) 4.23-4.39 (m, 1H) 4.98-5.14 (m, 2H) 6.10-6.19 (m, 1H) 6.53 (d, J=9.35 Hz, 1H) 6.84-6.94 (m, 2H) 6.99 (t, J=8.84 Hz, 1H) 7.16-7.28 (m, 5H) 7.30-7.41 (m, 2H) 7.53-7.66 (m, 1H)

MS ES⁺: 496

### Example 50: N-[(1S,2S)-1-amino-2,3-dihydro-1H-inden-2-yl]-2-(2,4-difluorophenyl)-2-(2-oxo-1,2-dihydropyridin-1-yl)acetamide

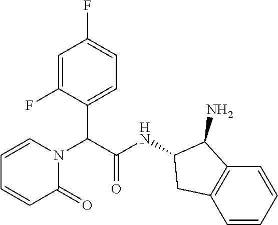 [SMILES: N[C@H]1c2ccccc2C[C@@H]1NC(=O)C(c1ccc(F)cc1F)n1ccccc1=O]

HCl (4 M solution in dioxane, 0.75 mL, 3.00 mmol) was added to a solution of tert-butyl ((1S,2S)-2-(2-(2,4-difluorophenyl)-2-(2-oxopyridin-1(2H)-yl)acetamido)-2,3-dihydro-1H-inden-1-yl)carbamate (Example 48 (first eluting peak), 150 mg, 0.303 mmol) in dioxane (1.5 mL). The reaction was stirred at room temperature for 3 hours. Further HCl (4 M solution in dioxane, 0.75 mL, 3.00 mmol) was added to the reaction mixture. The reaction was stirred at room temperature for a further 72 hours then concentrated in vacuo. The crude product was purified by reverse phase preparative HPLC eluted with acetonitrile/water (with 0.1% ammonia) to afford the title compound.

¹H NMR (300 MHz, DICHLOROMETHANE-d₂) δ ppm 2.56-2.67 (m, 1H) 3.32-3.42 (m, 1H) 4.31-4.49 (m, 2H) 6.10-6.18 (m, 1H) 6.40 (d, J=9.08 Hz, 1H) 6.85-6.95 (m, 2H) 6.98-7.07 (m, 1H) 7.10-7.25 (m, 4H) 7.28-7.36 (m, 2H) 7.43 (d, J=6.42 Hz, 1H) 7.54-7.63 (m, 1H)

MS ES⁺: 396

### Example 51: N-[(1S,2S)-1-amino-2,3-dihydro-1H-inden-2-yl]-2-(2,4-difluorophenyl)-2-(2-oxo-1,2-dihydropyridin-1-yl)acetamide

Prepared as described for Example 50 using tert-butyl ((1S,2S)-2-(2-(2,4-difluorophenyl)-2-(2-oxopyridin-1(2H)-yl)acetamido)-2,3-dihydro-1H-inden-1-yl)carbamate (Example 49 (second eluting peak), 196 mg, 0.396 mmol) to afford the title compound.

¹H NMR (300 MHz, DICHLOROMETHANE-d₂) δ ppm 2.79-2.92 (m, 1H) 3.21-3.30 (m, 1H) 4.18-4.28 (m, 1H) 4.28-4.34 (m, 1H) 6.12-6.19 (m, 1H) 6.46 (d, J=9.26 Hz, 1H) 6.85-6.95 (m, 2H) 6.98-7.05 (m, 1H) 7.13-7.27 (m, 4H) 7.30-7.40 (m, 2H) 7.54 (d, J=6.24 Hz, 1H) 7.63-7.72 (m, 1H)

MS ES⁺: 396

### Example 52: (2S)—N-[(1S,2S)-1-amino-2,3-dihydro-1H-inden-2-yl]-2-(4-fluorophenyl)butanamide

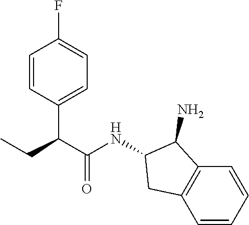 [SMILES: CC[C@H](C(=O)N[C@H]1Cc2ccccc2[C@@H]1N)c1ccc(F)cc1]

HCl (4 M solution in dioxane, 3 mL, 12.00 mmol) was added to a stirred solution of tert-butyl ((1S,2S)-2-((S)-2-(4-fluorophenyl)butanamido)-2,3-dihydro-1H-inden-1-yl)carbamate (Example 42, 450 mg, 1.091 mmol) in DCM (7 mL) and stirred for 6 hours. The solvent was removed in vacuo. The crude product was partitioned between DCM and saturated NaHCO₃ and the organics concentrated in vacuo. The crude product was recrystallised from EtOAc/heptane to afford the title compound.

¹H NMR (400 MHz, DMSO-d₆) δ ppm 0.84 (t, J=7.29 Hz, 3H) 1.49-1.70 (m, 1H) 1.87-2.06 (m, 3H) 2.55-2.66 (m, 1H) 3.15-3.25 (m, 1H) 3.35-3.45 (m, 1H) 3.82-4.04 (m, 2H) 7.05-7.21 (m, 5H) 7.25-7.31 (m, 1H) 7.35-7.47 (m, 2H) 8.37 (d, J=6.88 Hz, 1H)

MS ES⁺: 313

### Example 53: (2S)—N-[(1R,2R)-1-amino-2,3-dihydro-1H-inden-2-yl]-2-(4-fluorophenyl)butanamide

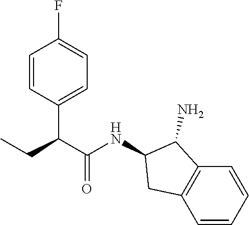 [SMILES: CC[C@H](C(=O)N[C@@H]1Cc2ccccc2[C@H]1N)c1ccc(F)cc1]

HCl (4 M solution in dioxane, 2.485 mL, 9.94 mmol) was added to a solution of tert-butyl ((1R,2R)-2-((S)-2-(4-fluorophenyl)butanamido)-2,3-dihydro-1H-inden-1-yl)carbamate (Example 43, 410 mg, 0.994 mmol) in DCM (5 mL) and stirred overnight. An additional portion of HCl (4 M in dioxane, 1 mL) was added and the reaction stirred for 6 hours. The reaction mixture was partitioned between DCM and saturated NaHCO₃, adding methanol to aid solubilisation. The organics were dried (phase separator), concentrated in vacuo and the crude product purified by column chromatography on basic silica, eluted with 0-100% ethyl acetate/petroleum. The product was recrystallised from ethyl acetate/heptane to afford the title compound.

¹H NMR (300 MHz, DMSO-d₆) δ ppm 0.85 (t, J=7.36 Hz, 3H) 1.49-1.70 (m, 1H) 1.89-2.06 (m, 3H) 2.38-2.47 (m, 1H) 2.99-3.18 (m, 1H) 3.34-3.44 (m, 1H) 3.87-4.11 (m, 2H) 7.03-7.52 (m, 8H) 8.34 (d, J=7.22 Hz, 1H)

MS ES⁺: 313

### Example 54: (2S)-2-amino-2-(4-fluorophenyl)-N-(trans)-(1-methoxy-2,3-dihydro-1H-inden-2-yl)acetamide

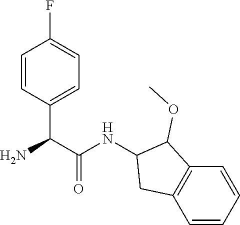 [SMILES: COC1c2ccccc2CC1NC(=O)[C@@H](N)c1ccc(F)cc1]

HCl (4 M solution in dioxane, 5.91 mL, 23.64 mmol) was added to a solution of tert-butyl ((1S)-1-(4-fluorophenyl)-2-((trans)-(1-methoxy-2,3-dihydro-1H-inden-2-yl)amino)-2-oxoethyl)carbamate (Intermediate 8, 1.96 g, 4.73 mmol) in methanol (40 mL). The reaction was stirred at room temperature for 7.5 hours. The solution was concentrated in vacuo and azeotroped with toluene to afford the HCl salt of the title compound. This was purified by reverse phase preparative HPLC eluted with acetonitrile/water (with 0.1% ammonia) to afford the title compound.

¹H NMR (400 MHz, DMSO-d₆) δ ppm 2.24 (br. s., 2H) 2.62-2.76 (m, 1H) 3.14-3.34 (m, 4H) 4.26-4.41 (m, 2H) 4.57-4.73 (m, 1H) 7.13 (t, J=8.71 Hz, 2H) 7.18-7.36 (m, 4H) 7.38-7.47 (m, 2H) 8.43 (t, J=7.61 Hz, 1H)

MS ES⁻: 313

### Examples 55 and 56: (2S)-2-[(cyclopropylmethyl)amino]-2-(4-fluorophenyl)-N-(trans)-(1-methoxy-2,3-dihydro-1H-inden-2-yl)acetamide Stereoisomers A and B

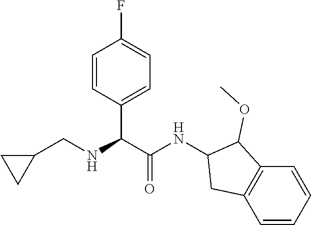 [SMILES: COC1c2ccccc2CC1NC(=O)[C@@H](NCC1CC1)c1ccc(F)cc1]

Cyclopropanecarbaldehyde (0.045 mL, 0.599 mmol) was added to a solution of (2S)-2-amino-2-(4-fluorophenyl)-N-(trans)-(1-methoxy-2,3-dihydro-1H-inden-2-yl)acetamide hydrochloride (the hydrochloride salt of the compound of Example 54, 0.2 g, 0.570 mmol) and triethylamine (0.079 mL, 0.570 mmol) in DCM (5 mL) under nitrogen. The reaction was stirred at room temperature for 45 minutes. Sodium triacetoxyborohydride (0.242 g, 1.140 mmol) was added and the reaction stirred at room temperature for 2 hours. The mixture was partitioned between DCM and water, dried (phase separator) and concentrated in vacuo. The crude product was purified by column chromatography on basic silica, eluted with 12-100% ethyl acetate/petroleum ether to afford the title compound as a mixture of the 2 trans diastereomers. The mixture was separated by chiral SFC (Lux-C4 Phenomenex, 16% methanol) to afford the title compounds.

### Example 55—Stereoisomer A, First Eluting Peak

¹H NMR (300 MHz, DMSO-d₆) δ ppm −0.02-0.07 (m, 2H) 0.32-0.42 (m, 2H) 0.79-0.94 (m, 1H) 2.20-2.39 (m, 2H) 2.66-2.77 (m, 1H) 3.14-3.26 (m, 4H) 4.16-4.40 (m, 2H) 4.56-4.64 (m, 1H) 7.08-7.33 (m, 6H) 7.37-7.47 (m, 2H) 8.44 (d, J=8.12 Hz, 1H), NH not observed, too broad

MS ES⁺: 369

### Example 56—Stereoisomer B, Second Eluting Peak

¹H NMR (400 MHz, DMSO-d₆) δ ppm 0.00-0.08 (m, 2H) 0.35-0.42 (m, 2H) 0.83-0.92 (m, 1H) 2.22-2.37 (m, 2H) 2.61-2.69 (m, 1H) 3.13-3.22 (m, 1H) 3.29 (s, 3H) 4.16-4.23 (m, 1H) 4.28-4.37 (m, 1H) 4.69 (d, J=4.58 Hz, 1H) 7.09-7.18 (m, 2H) 7.19-7.35 (m, 4H) 7.39-7.46 (m, 2H) 8.45 (d, J=8.25 Hz, 1H), NH not observed, too broad

MS ES⁺: 369

### Example 57: N-[(1S,2S)-1-amino-2,3-dihydro-1H-inden-2-yl]-2-(4-methoxyphenyl)propanamide hydrochloride

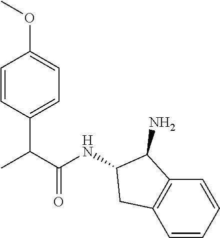 [SMILES: COc1ccc(C(C)C(=O)N[C@H]2Cc3ccccc3[C@@H]2N)cc1]

Triethylamine (0.140 mL, 1.208 mmol) was added to a solution of EDC (116 mg, 0.604 mmol), tert-butyl ((1S,2S)-2-amino-2,3-dihydro-1H-inden-1-yl)carbamate (100 mg, 0.403 mmol), 2-(4-methoxyphenyl)propanoic acid (72.6 mg, 0.403 mmol) and HOAt (100 mg, 0.604 mmol) in DCM (20 mL) under nitrogen. The reaction was stirred at room temperature for 36 hours. The mixture was partitioned between DCM and water, the organic layer was separated, washed with 1M HCl, and saturated NaHCO₃, dried (phase separator) and concentrated in vacuo. The solid was crystallized from diethyl ether, filtered and dried to afford tert-butyl ((1S,2S)-2-(2-(4-methoxyphenyl)propanamido)-2,3-dihydro-1H-inden-1-yl)carbamate. This was taken up in dioxane (5 mL) and HCl (4 M solution in dioxane, 1 mL, 4.00 mmol) was added. The reaction was stirred at room temperature for 18 hours. The reaction was concentrated in vacuo and triturated with diethyl ether to afford the title compound.

¹H NMR (400 MHz, DMSO-d₆) δ ppm 1.29-1.40 (m, 3H) 2.70-2.81 (m, 1H) 3.25-3.31 (m, 1H) 3.54-3.65 (m, 1H) 3.73 (s, 3H) 4.31-4.43 (m, 1H) 4.55-4.65 (m, 1H) 6.83-6.93 (m, 2H) 7.22-7.39 (m, 5H) 7.59-7.64 (m, 1H) 8.53-8.71 (m, 4H)

MS ES⁺: 311

### Example 58: N-[(1S,2S)-1-amino-2,3-dihydro-1H-inden-2-yl]-2-[4-(trifluoromethyl)phenyl]propanamide hydrochloride

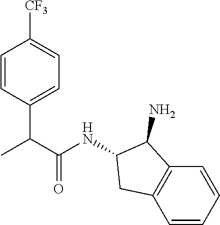 [SMILES: CC(C(=O)N[C@H]1Cc2ccccc2[C@@H]1N)c1ccc(C(F)(F)F)cc1]

Prepared as described for Example 57 using triethylamine (0.140 mL, 1.208 mmol), EDC (116 mg, 0.604 mmol), tert-butyl ((1S,2S)-2-amino-2,3-dihydro-1H-inden-1-yl)carbamate (100 mg, 0.403 mmol), 2-(4-(trifluoromethyl)phenyl)propanoic acid (88 mg, 0.403 mmol) and HOAt (100 mg, 0.604 mmol). This step was followed by the deprotection using HCl (4 M solution in dioxane, 1 mL, 4.00 mmol) to afford the title compound.

¹H NMR (400 MHz, DMSO-d₆) δ ppm 1.25-1.39 (m, 3H) 2.59-2.71 (m, 1H) 3.08-3.17 (m, 1H) 3.75 (d, J=6.97 Hz, 1H) 4.23-4.37 (m, 1H) 4.87-5.00 (m, 1H) 7.01-7.10 (m, 1H) 7.19 (d, J=4.86 Hz, 5H) 7.47-7.69 (m, 5H) 8.38-8.56 (m, 1H)

MS ES⁺: 349

### Example 59: (2S)-2-(2,4-difluorophenyl)-N-[(1S,2S)-1-[(2,2,2-trifluoroethyl)amino]-2,3-dihydro-1H-inden-2-yl]propanamide

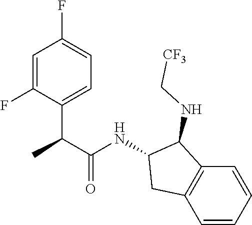 [SMILES: C[C@H](C(=O)N[C@H]1Cc2ccccc2[C@@H]1NCC(F)(F)F)c1ccc(F)cc1F]

2,2,2-Trifluoroethyl trichloromethanesulphonate (310 mg, 1.100 mmol) was added to a mixture of (S)—N-((1S,2S)-1-amino-2,3-dihydro-1H-inden-2-yl)-2-(2,4-difluorophenyl)propanamide hydrochloride (the hydrochloride salt of the compound of Example 34, 194 mg, 0.55 mmol) and K₂CO₃ (380 mg, 2.75 mmol) in acetone (3.5 mL) and treated with microwaves at 120° C. for 30 mins. The reaction mixture was partitioned between DCM and water and the organics concentrated in vacuo prior to purification by reverse phase preparative HPLC eluted with acetonitrile/water (with 0.1% ammonia) to afford the title compound.

¹H NMR (300 MHz, DMSO-d₆) δ ppm 1.35 (d, J=7.08 Hz, 3H) 2.58-2.85 (m, 2H) 3.11-3.29 (m, 2H) 3.78-3.90 (m, 1H) 3.96-4.06 (m, 1H) 4.15-4.29 (m, 1H) 6.97-7.29 (m, 7H) 7.38-7.54 (m, 1H) 8.40 (d, J=7.63 Hz, 1H)

MS ES⁺: 399

### Example 60: (2S)—N-(trans)-(1-ethoxy-2,3-dihydro-1H-inden-2-yl)-2-(4-fluorophenyl)-2-{[(2-methyl-1,3-thiazol-4-yl)methyl]amino}acetamide

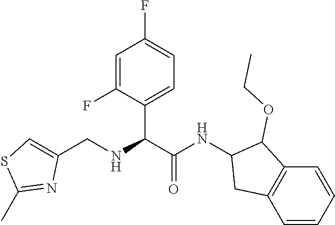 [SMILES: CCOC1c2ccccc2CC1NC(=O)[C@@H](NCc1csc(C)n1)c1ccc(F)cc1F]

2-Methylthiazole-4-carbaldehyde (81 mg, 0.640 mmol) was added to a solution of (2S)-2-amino-N-(trans)-(1-ethoxy-2,3-dihydro-1H-inden-2-yl)-2-(4-fluorophenyl)acetamide (Intermediate 9, 200 mg, 0.609 mmol) in DCM (6 mL) under nitrogen and stirred for 45 minutes. Sodium triacetoxyborohydride (258 mg, 1.218 mmol) was added the reaction was stirred for 45 minutes. The reaction mixture was washed with water and the organics purified by column chromatography on silica, eluted with 0-70% ethyl acetate/petroleum to afford impure product. The product was further purified by reverse phase preparative HPLC eluted with acetonitrile/water (with 0.1% ammonia) to afford the title compound.

¹H NMR (300 MHz, DMSO-d₆) δ ppm 0.92-1.13 (m, 3H) 2.61 (s, 3H) 2.64-3.26 (m, 3H) 3.35-3.71 (m, 3H) 4.18-4.38 (m, 2H) 4.68-4.84 (m, 1H) 7.10-7.33 (m, 8H) 7.39-7.51 (m, 2H) 8.50 (d, J=8.12 Hz, 1H)

MS ES⁺: 440

### Example 61: (2S)-2-(4-fluorophenyl)-N-(trans)-(1-hydroxy-2,3-dihydro-1H-inden-2-yl)propanamide

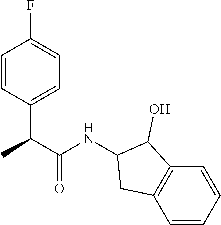 [SMILES: C[C@H](C(=O)NC1Cc2ccccc2C1O)c1ccc(F)cc1]

Prepared as described for Example 21 using triethylamine (0.140 mL, 1.005 mmol), (S)-2-(4-fluorophenyl)propanoic acid (Intermediate 3, 0.059 g, 0.352 mmol), (trans)-2-amino-2,3-dihydro-1H-inden-1-ol (0.05 g, 0.335 mmol), EDC (0.096 g, 0.503 mmol) and HOAt (0.068 g, 0.503 mmol). The crude product was purified by reverse phase preparative HPLC eluted with acetonitrile/water (with 0.1% ammonia) to afford the title compound.

¹H NMR (300 MHz, DMSO-d₆) δ ppm 1.29-1.38 (m, 3H) 2.40-2.66 (m, 1H) 3.04-3.25 (m, 1H) 3.59-3.71 (m, 1H) 4.02-4.18 (m, 1H) 4.79-4.94 (m, 1H) 5.39-5.55 (m, 1H) 7.06-7.43 (m, 8H) 8.36 (d, J=7.15 Hz, 1H)

MS ES⁻: 298

### Examples 62 and 63: (2S)-2-(4-fluorophenyl)-N-(cis)-(1-hydroxy-2,3-dihydro-1H-inden-2-yl)propanamide Stereoisomers A and B

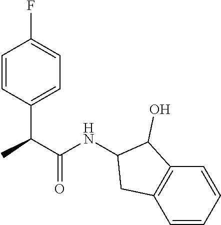 [SMILES: C[C@H](C(=O)NC1Cc2ccccc2C1O)c1ccc(F)cc1]

Triethylamine (2.486 mL, 17.84 mmol) was added to a suspension of (S)-2-(4-fluorophenyl)propanoic acid (Intermediate 3, 1 g, 5.95 mmol), (cis)-2-amino-2,3-dihydro-1H-inden-1-ol (0.932 g, 6.24 mmol, synthesis described in Tett, let, 1993, 34, 52, 8399), EDC (1.710 g, 8.92 mmol) and HOAt (1.214 g, 8.92 mmol) in DCM (50 mL). The reaction was stirred at room temperature overnight. The mixture was partitioned between DCM and 5% citric acid. The phases were separated and the organics were washed with saturated NaHCO₃, water, dried (phase separator) and concentrated in vacuo. The crude product was purified by column chromatography on silica, eluted with 0-50% ethyl acetate/petroleum ether to afford the separated diastereomers of the title compound.

### Example 62—Stereoisomer A (1R,2S)—First Eluting Peak

¹H NMR (400 MHz, DMSO-d₆) δ ppm 1.32 (d, J=6.97 Hz, 3H) 2.79-2.87 (m, 1H) 3.00-3.08 (m, 1H) 3.75-3.83 (m, 1H) 4.25-4.35 (m, 1H) 4.76-4.84 (m, 1H) 5.25 (d, J=5.69 Hz, 1H) 7.06-7.15 (m, 2H) 7.16-7.25 (m, 3H) 7.32 (d, J=6.79 Hz, 1H) 7.36-7.42 (m, 2H) 7.79 (d, J=7.52 Hz, 1H)

MS ES⁻: 298

### Example 63—Stereoisomer B (1S,2R)—Second Eluting Peak

¹H NMR (400 MHz, DMSO-d₆) δ ppm 1.33 (d, J=6.97 Hz, 3H) 2.68-2.76 (m, 1H) 2.90-2.98 (m, 1H) 3.74-3.81 (m, 1H) 4.24-4.34 (m, 1H) 4.84-4.90 (m, 1H) 5.29 (d, J=5.69 Hz, 1H) 7.07-7.14 (m, 2H) 7.16-7.25 (m, 3H) 7.32-7.41 (m, 3H) 7.85 (d, J=7.70 Hz, 1H)

MS ES⁻: 298

### Example 64: (2S)-2-(4-fluorophenyl)-N-[(1S,2S)-1-(methylamino)-2,3-dihydro-1H-inden-2-yl]propanamide

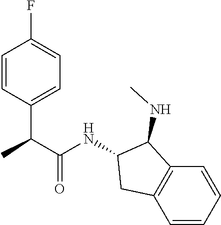 [SMILES: CN[C@H]1c2ccccc2C[C@@H]1NC(=O)[C@@H](C)c1ccc(F)cc1]

Prepared as described for Example 47 using lithium bis(trimethylsilyl)amide (1 M solution in THF, 0.602 mL, 0.602 mmol), tert-butyl ((1S,2S)-2-((S)-2-(4-fluorophenyl)propanamido)-2,3-dihydro-1H-inden-1-yl)carbamate (Example 21, 0.2 g, 0.502 mmol) and methyl iodide (0.038 mL, 0.602 mmol). Reaction time was 1 hour. The crude product was purified by reverse phase chromatography on C18 silica eluted with 5-95% water (with 0.05% ammonia)/acetonitrile. The product was further purified by reverse phase preparative HPLC eluted with acetonitrile/water (with 0.1% ammonia) to afford tert-butyl ((1S,2S)-2-((S)-2-(4-fluorophenyl)propanamido)-2,3-dihydro-1H-inden-1-yl)(methyl)carbamate. This was followed by the deprotection step with HCl (4 M solution in dioxane, 0.161 mL, 0.642 mmol). The crude product was purified by reverse phase preparative HPLC eluted with acetonitrile/water (with 0.1% ammonia). The product was loaded onto a cation exchange cartridge, washed with methanol and eluted with 2M ammonia/methanol solution then concentrated in vacuo. The product was triturated with diethyl ether to afford the title compound.

¹H NMR (300 MHz, DMSO-d₆) δ ppm 1.32 (d, J=6.88 Hz, 3H) 1.90 (br. s., 1H) 2.15 (s, 3H) 2.58-2.70 (m, 1H) 3.15-3.26 (m, 1H) 3.54-3.66 (m, 1H) 3.80-3.88 (m, 1H) 4.15-4.30 (m, 1H) 7.05-7.29 (m, 6H) 7.30-7.41 (m, 2H) 8.26 (d, J=7.57 Hz, 1H)

MS ES⁺: 313

### Example 65: (2S)-2-(4-fluorophenyl)-N-(trans)-(1-methoxy-2,3-dihydro-1H-inden-2-yl)-2-[(1-methyl-1H-pyrazol-4-yl)formamido]acetamide

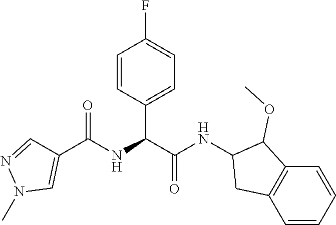 [SMILES: COC1c2ccccc2CC1NC(=O)[C@@H](NC(=O)c1cnn(C)c1)c1ccc(F)cc1]

Triethylamine (0.060 mL, 0.428 mmol) was added to a suspension of (2S)-2-amino-2-(4-fluorophenyl)-N-(trans)-(1-methoxy-2,3-dihydro-1H-inden-2-yl)acetamide hydrochloride (the hydrochloride salt of the compound of Example 54, 0.05 g, 0.143 mmol) and 1-methyl-1H-pyrazole-4-carbonyl chloride (0.023 g, 0.157 mmol) in DCM (2 mL). The reaction was stirred at room temperature for 2 hours. The mixture was partitioned between DCM and saturated NaHCO₃, passed through a phase separator and concentrated in vacuo. The crude product was purified by reverse phase preparative HPLC eluted with acetonitrile/water (with 0.1% ammonia) to afford the title compound.

¹H NMR (300 MHz, DMSO-d₆) δ ppm 2.55-2.79 (m, 1H) 3.16-3.41 (m, 4H) 3.85 (s, 3H) 4.26-4.40 (m, 1H) 4.51-4.72 (m, 1H) 5.61-5.69 (m, 1H) 7.13-7.39 (m, 6H) 7.46-7.56 (m, 2H) 7.95 (s, 1H) 8.29 (s, 1H) 8.48 (d, J=7.98 Hz, 1H) 8.66-8.77 (m, 1H)

MS ES⁻: 421

### Example 66: (2S)-2-(cyclopropylformamido)-2-(4-fluorophenyl)-N-(trans)-(1-methoxy-2,3-dihydro-1H-inden-2-yl)acetamide

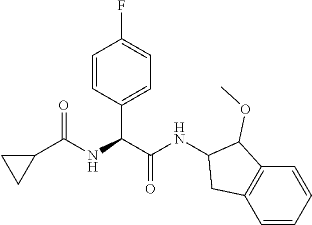 [SMILES: COC1c2ccccc2CC1NC(=O)[C@@H](NC(=O)C1CC1)c1ccc(F)cc1]

Prepared as described for Example 65 using cyclopropanecarbonyl chloride (0.014 mL, 0.157 mmol).

¹H NMR (300 MHz, DMSO-d₆) δ ppm 0.54-0.73 (m, 4H) 1.79-1.93 (m, 1H) 2.55-2.79 (m, 1H) 3.12-3.43 (m, 4H) 4.22-4.38 (m, 1H) 4.44-4.70 (m, 1H) 5.50 (d, J=8.25 Hz, 1H) 7.11-7.52 (m, 8H) 8.64-8.88 (m, 2H)

MS ES⁻: 381

### Example 67: (2S)-2-(4-fluorophenyl)-N-(trans)-[1-(pyrrolidin-1-yl)-2,3-dihydro-1H-inden-2-yl]propanamide

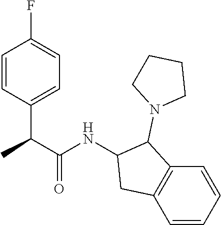 [SMILES: C[C@H](C(=O)NC1Cc2ccccc2C1N1CCCC1)c1ccc(F)cc1]

A solution of methanesulphonic anhydride (0.116 g, 0.668 mmol) in THF (1 mL) was added dropwise to a solution of (2S)-2-(4-fluorophenyl)-N-(cis)-(1-hydroxy-2,3-dihydro-1H-inden-2-yl)propanamide (Example 62 (first eluting peak), 0.1 g, 0.334 mmol) and triethylamine (0.140 mL, 1.002 mmol) in THF (2 mL) at −78° C. under nitrogen. The reaction was stirred at −78° C. for 30 minutes then at 0° C. for 30 minutes. Pyrrolidine (0.138 mL, 1.670 mmol) was added and the reaction was stirred at 0° C. for 1 hour. The reaction was allowed to warm to room temperature and stirred for 4.5 hours. The mixture was partitioned between ethyl acetate and water. The phases were separated and the aqueous extracted with ethyl acetate. The combined organics were washed with saturated brine, dried (phase separator) and concentrated in vacuo. The crude product was loaded onto a cation exchange cartridge, washed with methanol and eluted with 2M ammonia/methanol solution then concentrated in vacuo. The crude product was purified by column chromatography on basic silica, eluted with 0-50% ethyl acetate/petroleum ether. The product was further purified by reverse phase preparative HPLC eluted with acetonitrile/water (with 0.1% ammonia) to afford the title compound.

¹H NMR (400 MHz, DMSO-d₆) δ ppm 1.26-1.33 (m, 3H) 1.45-1.61 (m, 4H) 2.30-2.45 (m, 4H) 2.61-2.69 (m, 1H) 3.17-3.26 (m, 1H) 3.52-3.60 (m, 1H) 3.98 (d, J=4.77 Hz, 1H) 4.43-4.52 (m, 1H) 7.07-7.25 (m, 6H) 7.28-7.35 (m, 2H) 8.26 (d, J=8.07 Hz, 1H)

MS ES⁺: 353

### Example 68: (2S)-2-(4-fluorophenyl)-N-(trans)-[1-(pyrrolidin-1-yl)-2,3-dihydro-1H-inden-2-yl]propanamide

Prepared as described for Example 67, using (2S)-2-(4-fluorophenyl)-N-(cis)-(1-hydroxy-2,3-dihydro-1H-inden-2-yl)propanamide (Example 63 (second eluting peak), 0.1 g, 0.334 mmol). However, the mesylation was performed at between −15 and −5° C. and after pyrrolidine addition the reaction was stirred for 3 hours at −15 to −5° C. followed by 2 hours at room temperature. The crude product was loaded onto a cation exchange cartridge, washed with methanol and eluted with 2M ammonia/methanol solution then concentrated in vacuo. The crude product was purified by column chromatography on basic silica, eluted with 0-50% ethyl acetate/petroleum to afford the title compound.

¹H NMR (400 MHz, DMSO-d₆) δ ppm 1.25-1.33 (m, 3H) 1.59-1.69 (m, 4H) 2.51-2.64 (m, 5H) 3.13-3.23 (m, 1H) 3.52-3.61 (m, 1H) 4.08 (d, J=4.03 Hz, 1H) 4.40-4.50 (m, 1H) 7.07-7.14 (m, 2H) 7.15-7.24 (m, 3H) 7.25-7.37 (m, 3H) 8.26 (d, J=7.70 Hz, 1H)

MS ES⁺: 353

### Examples 69 and 70: N-[(1S,2S)-1-amino-2,3-dihydro-1H-inden-2-yl]-2-(4-chlorophenyl)propanamide Stereoisomers A and B

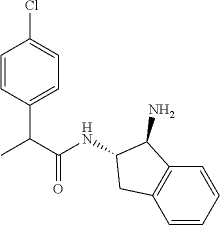 [SMILES: CC(C(=O)N[C@H]1Cc2ccccc2[C@@H]1N)c1ccc(Cl)cc1]

Triethylamine (1.684 mL, 12.08 mmol) was added to a suspension of 2-(4-chlorophenyl)propanoic acid (0.781 g, 4.23 mmol), tert-butyl ((1S,2S)-2-amino-2,3-dihydro-1H-inden-1-yl)carbamate (1 g, 4.03 mmol), EDC (1.158 g, 6.04 mmol) and HOAt (0.822 g, 6.04 mmol) in DCM (30 mL) under nitrogen. The reaction was stirred at room temperature overnight. The mixture was partitioned between DCM and 5% citric acid. The organics were dried (phase separator) and concentrated in vacuo. The crude product was purified by column chromatography on silica, eluted with 0-50% ethyl acetate/petroleum to afford tert-butyl ((1S,2S)-2-(2-(4-chlorophenyl)propanamido)-2,3-dihydro-1H-inden-1-yl)carbamate. This was taken up in methanol (10 mL) and treated with HCl (4 M solution in dioxane, 0.904 mL, 3.62 mmol). The reaction was stirred at room temperature overnight. A further portion of HCl (4 M solution in dioxane, 0.2 mL) was added and the reaction stirred for 1 hour. The solution was concentrated and azeotroped with toluene. The crude product was loaded onto a cation exchange cartridge, washed with methanol and eluted with 2M ammonia/methanol solution then concentrated in vacuo. The crude product was purified by reverse phase preparative HPLC eluted with acetonitrile/water (with 0.1% ammonia) to afford the separated diastereomers of the title compound.

### Example 69—Stereoisomer A—First Eluting Peak

¹H NMR (400 MHz, DMSO-d₆) δ ppm 1.31-1.37 (m, 3H) 1.84 (br. s., 2H) 2.56-2.65 (m, 1H) 3.14-3.21 (m, 1H) 3.61-3.69 (m, 1H) 3.87-4.01 (m, 2H) 7.12-7.21 (m, 3H) 7.24-7.30 (m, 1H) 7.33-7.42 (m, 4H) 8.31 (d, J=6.79 Hz, 1H)

MS ES⁻: 313

### Example 70—Stereoisomer B—Second Eluting Peak

¹H NMR (400 MHz, DMSO-d₆) δ ppm 1.30-1.38 (m, 3H) 1.97 (br. s., 2H) 2.39-2.48 (m, 1H) 3.04-3.14 (m, 1H) 3.61-3.70 (m, 1H) 3.89-3.98 (m, 1H) 4.02-4.08 (m, 1H) 7.09-7.23 (m, 3H) 7.28-7.41 (m, 5H) 8.31 (d, J=6.97 Hz, 1H)

MS ES⁻: 313

### Example 71: tert-butyl N—[(S)-(4-fluorophenyl)[(trans)-(1-methyl-2,3-dihydro-1H-inden-2-yl)carbamoyl]methyl]carbamate

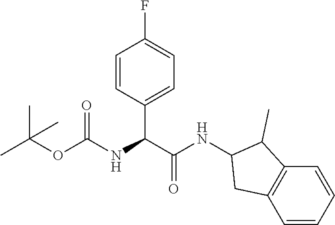 [SMILES: CC1c2ccccc2CC1NC(=O)[C@@H](NC(=O)OC(C)(C)C)c1ccc(F)cc1]

T3P (50% solution in ethyl acetate, 0.404 mL, 0.679 mmol) was added to a solution of triethylamine (0.138 mL, 1.019 mmol), (S)-2-((tert-butoxycarbonyl)amino)-2-(4-fluorophenyl)acetic acid (Intermediate 8 step (i), 91 mg, 0.340 mmol) and (trans)-1-methyl-2,3-dihydro-1H-inden-2-amine (50 mg, 0.340 mmol) in DCM (2 mL) and stirred for 30 minutes. The reaction mixture was washed with saturated NaHCO₃ and concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 0.64-1.09 (m, 3H) 1.38 (s, 9H) 2.64-2.85 (m, 1H) 2.99-3.30 (m, 2H) 4.39-4.60 (m, 1H) 5.12-5.29 (m, 1H) 7.05-7.52 (m, 9H) 8.16-8.35 (m, 1H)

MS ES⁺: 399

### Example 72: (2S)-2-(4-fluorophenyl)-2-methanesulphonamido-N-(trans)-(1-methoxy-2,3-dihydro-1H-inden-2-yl)acetamide

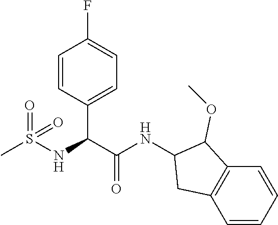 [SMILES: COC1c2ccccc2CC1NC(=O)[C@@H](NS(C)(=O)=O)c1ccc(F)cc1]

Methanesulphonic anhydride (0.041 g, 0.235 mmol) was added to a solution of (2S)-2-amino-2-(4-fluorophenyl)-N-(trans)-(1-methoxy-2,3-dihydro-1H-inden-2-yl)acetamide hydrochloride (the hydrochloride salt of the compound of Example 54, 0.075 g, 0.214 mmol) and triethylamine (0.089 mL, 0.641 mmol) in DCM (2 mL). The reaction was stirred at room temperature overnight. Further portions of methanesulphonic anhydride (0.041 g, 0.235 mmol) and triethylamine (0.089 mL, 0.641 mmol) were added and the reaction stirred for 1 hour. The mixture was partitioned between ethyl acetate and 5% citric acid. The phases were separated and the organic was washed with saturated NaHCO₃, saturated brine, dried (phase separator) and concentrated in vacuo. The crude product was purified by column chromatography on silica, eluted with 0-50% ethyl acetate/petroleum ether to afford the title compound.

¹H NMR (400 MHz, DMSO-d₆) δ ppm 2.53-2.78 (m, 4H) 3.13-3.41 (m, 4H) 4.22-4.35 (m, 1H) 4.47-4.68 (m, 1H) 5.02 (br. s., 1H) 7.16-7.40 (m, 6H) 7.45-7.55 (m, 2H) 8.04 (br. s., 1H) 8.70-8.79 (m, 1H)

MS ES⁻: 391

### Example 73: (2S)-2-(4-fluorophenyl)-N-(trans)-[1-(morpholin-4-yl)-2,3-dihydro-1H-inden-2-yl]propanamide

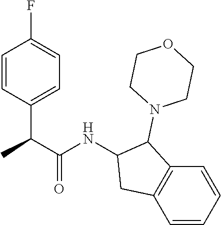 [SMILES: C[C@H](C(=O)NC1Cc2ccccc2C1N1CCOCC1)c1ccc(F)cc1]

Prepared as described for Example 67 using (2S)-2-(4-fluorophenyl)-N-(trans)-(1-hydroxy-2,3-dihydro-1H-inden-2-yl)propanamide (Example 62 (first eluting peak), 0.1 g, 0.334 mmol), methanesulphonic anhydride (0.116 g, 0.668 mmol), triethylamine (0.140 mL, 1.002 mmol) and morpholine (0.146 mL, 1.670 mmol), except the mesylation step was performed between −15 and −5° C. and after morpholine addition the reaction was warmed to room temperature for 5 hours. The crude product was purified by reverse phase preparative HPLC eluted with acetonitrile/water (with 0.1% ammonia) to afford the title compound.

¹H NMR (400 MHz, DMSO-d₆) δ ppm 1.34 (d, J=6.97 Hz, 3H) 2.14-2.27 (m, 4H) 2.63-2.73 (m, 1H) 3.09-3.19 (m, 1H) 3.33-3.39 (m, 2H) 3.42-3.50 (m, 2H) 3.54-3.63 (m, 1H) 4.01 (d, J=6.60 Hz, 1H) 4.50-4.60 (m, 1H) 7.09-7.25 (m, 6H) 7.31-7.40 (m, 2H) 8.33 (d, J=8.62 Hz, 1H)

MS ES⁺: 369

### Example 74: (2S)—N-(trans)-[1-(dimethylamino)-2,3-dihydro-1H-inden-2-yl]-2-(4-fluorophenyl)propanamide

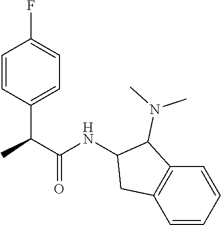 [SMILES: C[C@H](C(=O)NC1Cc2ccccc2C1N(C)C)c1ccc(F)cc1]

Prepared as described for Example 73 using dimethylamine (2 M solution in THF) (0.835 mL, 1.670 mmol). The crude product was loaded onto a cation exchange cartridge, washed with methanol and eluted with 2M ammonia/methanol solution then concentrated in vacuo. The crude product was purified by column chromatography on basic silica, eluted with 0-50% ethyl acetate/petroleum. The product was further purified by reverse phase preparative HPLC eluted with acetonitrile/water (with 0.1% ammonia) to afford the title compound.

¹H NMR (400 MHz, DMSO-d₆) δ ppm 1.34 (d, J=7.15 Hz, 3H) 1.99 (s, 6H) 2.62-2.72 (m, 1H) 3.11-3.20 (m, 1H) 3.53-3.62 (m, 1H) 3.99-4.05 (m, 1H) 4.47-4.57 (m, 1H) 7.07-7.24 (m, 6H) 7.31-7.39 (m, 2H) 8.31 (d, J=8.44 Hz, 1H)

MS ES⁺: 327

### Example 75: N-[(1S,2S)-1-amino-2,3-dihydro-1H-inden-2-yl]-2-fluoro-2-(4-fluorophenyl)propanamide

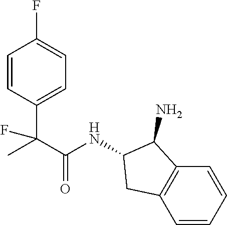 [SMILES: CC(F)(C(=O)N[C@H]1Cc2ccccc2[C@@H]1N)c1ccc(F)cc1]

HATU (399 mg, 1.050 mmol) was added to a solution of 2-fluoro-2-(4-fluorophenyl)propanoic acid (Intermediate 10, 186 mg, 1 mmol) and DIPEA (0.192 mL, 1.100 mmol) in DMF (1 mL) at room temperature. The mixture was stirred for 5 minutes then tert-butyl ((1S,2S)-2-amino-2,3-dihydro-1H-inden-1-yl)carbamate (248 mg, 1.000 mmol) was added. The mixture was stirred at room temperature for 1 hour. The reaction was partitioned between water and ethyl acetate. The organic phase was washed with water, dried and concentrated in vacuo. The crude material was taken up in DCM (5 mL) and treated with HCl (4 M solution in dioxane, 1.250 mL, 5.00 mmol) for 3 hours at room temperature. A further portion of HCl (4 M solution in dioxane, 1.250 mL, 5.00 mmol) was added and the reaction stirred for 1 hour, then concentrated in vacuo. The residue was suspended in MTBE and the mixture filtered. The crude product was purified by reverse phase preparative HPLC eluted with acetonitrile/water (with 0.1% ammonia) to afford the title compound.

¹H NMR (400 MHz, DMSO-d₆) δ ppm 1.79-1.93 (m, 5H) 2.59-2.80 (m, 1H) 2.96-3.17 (m, 1H) 3.95-4.12 (m, 1H) 4.15-4.28 (m, 1H) 7.08-7.36 (m, 6H) 7.51-7.68 (m, 2H) 8.62 (d, J=6.60 Hz, 1H)

MS ES⁺: 317

### Examples 76 and 77: N-[(1S,2S)-1-amino-2,3-dihydro-1H-inden-2-yl]-2-fluoro-2-(4-fluorophenyl)propanamide Stereoisomers A and B

Example 75 was separated by chiral SFC (AI Daicel CHIRALPAK, 23% IPA) to afford the title compounds.

### Example 76—Stereoisomer A—First Eluting Peak

¹H NMR (400 MHz, DMSO-d₆) δ ppm 1.77-1.95 (m, 5H) 2.69-2.81 (m, 1H) 3.05-3.19 (m, 1H) 3.95-4.10 (m, 1H) 4.19 (d, J=8.25 Hz, 1H) 7.11-7.35 (m, 6H) 7.55-7.67 (m, 2H) 8.62 (d, J=5.32 Hz, 1H)

MS ES⁺: 317

### Example 77—Stereoisomer B—Second Eluting Peak

¹H NMR (400 MHz, DMSO-d₆) δ ppm 1.77-2.06 (m, 5H) 2.56-2.70 (m, 1H) 2.95-3.07 (m, 1H) 3.97-4.10 (m, 1H) 4.23 (d, J=8.44 Hz, 1H) 7.05-7.36 (m, 6H) 7.49-7.65 (m, 2H) 8.61 (d, J=5.69 Hz, 1H)

MS ES⁺: 317

### Example 78: (2S)-2-phenyl-N-(trans)-[1-(pyrrolidin-1-yl)-2,3-dihydro-1H-inden-2-yl]propanamide

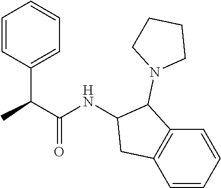 [SMILES: C[C@H](C(=O)NC1Cc2ccccc2C1N1CCCC1)c1ccccc1]

Prepared as described for Example 67 using methanesulphonic anhydride (0.097 g, 0.554 mmol), (2S)—N-((cis)-1-hydroxy-2,3-dihydro-1H-inden-2-yl)-2-phenylpropanamide (the mixture of compounds of Examples 5 and 6 prior to their separation by SFC, 0.078 g, 0.277 mmol), triethylamine (0.116 mL, 0.832 mmol) and pyrrolidine (0.115 mL, 1.386 mmol). The reaction time was overnight. The crude product was purified by column chromatography on basic silica, eluted with 0-100% ethyl acetate/petroleum. The product was further purified by reverse phase preparative HPLC eluted with acetonitrile/water (with 0.1% ammonia) to afford the title compound.

¹H NMR (400 MHz, DMSO-d₆) δ ppm 1.26-1.36 (m, 3H) 1.48-1.71 (m, 4H) 2.31-2.46 (m, 2H) 2.53-2.71 (m, 3H) 3.13-3.27 (m, 1H) 3.52-3.61 (m, 1H) 3.96-4.14 (m, 1H) 4.42-4.54 (m, 1H) 7.13-7.35 (m, 9H) 8.21-8.30 (m, 1H)

MS ES⁺: 335

### Example 79: N-[(1S,2S)-1-amino-2,3-dihydro-1H-inden-2-yl]-1-(2-chlorophenyl)cyclopropane-1-carboxamide

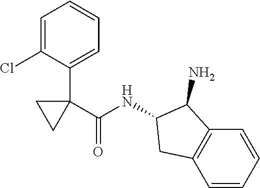 [SMILES: N[C@H]1c2ccccc2C[C@@H]1NC(=O)C1(c2ccccc2Cl)CC1]

HATU (59.9 mg, 0.158 mmol) was added to a solution of 1-(2-chlorophenyl)cyclopropanecarboxylic acid (29.5 mg, 0.15 mmol) and DIPEA (0.029 mL, 0.165 mmol) in DMF (0.5 mL) at room temperature. The mixture was stirred for 5 minutes then tert-butyl ((1S,2S)-2-amino-2,3-dihydro-1H-inden-1-yl)carbamate (37.2 mg, 0.150 mmol) was added. The mixture was stirred for 30 minutes, then HCl (4 M solution in dioxane, 0.375 mL, 1.500 mmol) was added. The mixture was heated under microwave irradiation at 60° C. for 2 hours. The reaction was partitioned between DCM and NaOH (2 M). The organic phase was concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 0.95-1.09 (m, 2H) 1.46-1.59 (m, 2H) 1.84 (br. s., 2H) 2.56-2.71 (m, 1H) 2.92-3.07 (m, 1H) 3.96-4.12 (m, 2H) 6.85-6.97 (m, 1H) 7.06-7.20 (m, 3H) 7.24 (d, J=6.42 Hz, 1H) 7.29-7.41 (m, 2H) 7.42-7.54 (m, 2H)

MS ES⁺: 327

### Example 80: (2S)-2-(2,4-difluorophenyl)-N-[(1S,2S)-1-acetamido-2,3-dihydro-1H-inden-2-yl]propanamide

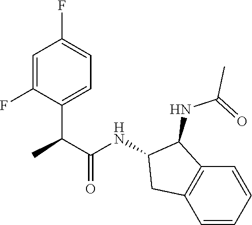 [SMILES: CC(=O)N[C@H]1c2ccccc2C[C@@H]1NC(=O)[C@@H](C)c1ccc(F)cc1F]

Acetyl chloride (0.006 mL, 0.084 mmol) was added to a solution of the hydrochloride salt of (S)—N-((1S,2S)-1-amino-2,3-dihydro-1H-inden-2-yl)-2-(2,4-difluorophenyl)propanamide (Example 34, 0.025 g, 0.071 mmol) and DIPEA (0.025 mL, 0.142 mmol) in DCM (0.5 mL). The reaction was stirred at room temperature for 1 hour. The reaction mixture was concentrated under a stream of dry nitrogen, then purified (three times) by reverse phase preparative HPLC eluted with acetonitrile/water (with 0.1% ammonia) to afford the title compound.

¹H NMR (400 MHz, DMSO-d₆) δ ppm 1.26-1.40 (m, 3H), 1.74-1.91 (m, 3H), 2.56-2.77 (m, 1H), 3.09-3.22 (m, 1H), 3.77-4.04 (m, 1H), 4.25-4.42 (m, 1H), 5.14-5.29 (m, 1H), 6.98-7.32 (m, 6H), 7.36-7.58 (m, 1H), 8.16-8.47 (m, 2H)

MS ES⁺: 359

### Example 81: tert-butyl N-[(1S,2S)-2-[2-(2,4-difluorophenyl)-2-(1H-pyrazol-1-yl)acetamido]-2,3-dihydro-1H-inden-1-yl]carbamate (Diastereomeric Mixture)

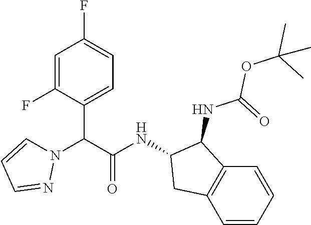 [SMILES: CC(C)(C)OC(=O)N[C@H]1c2ccccc2C[C@@H]1NC(=O)C(c1ccc(F)cc1F)n1cccn1]

Prepared as described for Example 1 using lithio 2-(2,4-difluorophenyl)-2-(1H-pyrazol-1-yl)acetate (Intermediate 11, 100 mg, 0.410 mmol) and tert-butyl ((1S,2S)-2-amino-2,3-dihydro-1H-inden-1-yl)carbamate (112 mg, 0.451 mmol). The crude product was purified by reverse phase chromatography on C18 silica eluted with 0-100% methanol/water (with 0.05% ammonia) then by reverse phase preparative HPLC eluted with acetonitrile/water (with 0.1% ammonia) to afford the title compound.

¹H NMR (400 MHz, DICHLOROMETHANE-d₂) δ ppm 1.38-1.47 (m, 9H), 2.61-2.85 (m, 1H), 3.31-3.58 (m, 1H), 4.15-4.50 (m, 1H), 4.99-5.28 (m, 2H), 6.28-6.34 (m, 1H), 6.82-7.05 (m, 2H), 7.12-7.32 (m, 5H), 7.41-7.63 (m, 3H), 7.94-8.15 (m, 1H)

MS ES⁺: 469

### Example 82: tert-butyl N-[(1S,2S)-2-[2-(2,4-difluorophenyl)-2-(2-methyl-1H-imidazol-1-yl)acetamido]-2,3-dihydro-1H-inden-1-yl]carbamate (Diastereomeric Mixture)

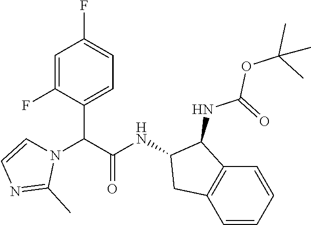 [SMILES: Cc1nccn1C(C(=O)N[C@H]1Cc2ccccc2[C@@H]1NC(=O)OC(C)(C)C)c1ccc(F)cc1F]

Prepared as described for Example 1 using lithio 2-(2,4-difluorophenyl)-2-(2-methyl-1H-imidazol-1-yl)acetate (Intermediate 12, 65 mg, 0.252 mmol) and tert-butyl ((1S,2S)-2-amino-2,3-dihydro-1H-inden-1-yl)carbamate (69 mg, 0.278 mmol). The crude product was purified by reverse phase chromatography on C18 silica eluted with 0-100% methanol/water (with 0.05% ammonia) to afford the title compound.

¹H NMR (400 MHz, DICHLOROMETHANE-d₂) δ ppm 1.39-1.47 (m, 9H) 2.54 (d, J=4.03 Hz, 3H) 2.67-2.81 (m, 1H) 3.44-3.57 (m, 1H) 4.15-4.33 (m, 1H) 5.03-5.12 (m, 2H) 6.07 (d, J=6.79 Hz, 1H) 6.88-7.04 (m, 4H) 7.18-7.44 (m, 6H)

MS ES⁺: 483

### Example 83: (2S)-2-(3,5-dimethyl-1,2-isoxazole-4-sulfonamido)-2-(4-fluorophenyl)-N-((trans)-1-methoxy-2,3-dihydro-1H-inden-2-yl)acetamide

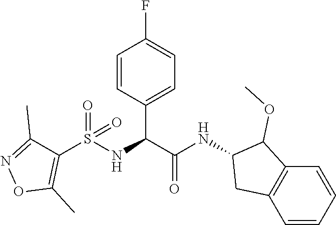 [SMILES: COC1c2ccccc2C[C@@H]1NC(=O)[C@@H](NS(=O)(=O)c1c(C)noc1C)c1ccc(F)cc1]

Triethylamine (0.119 mL, 0.855 mmol) was added to a suspension of the hydrochloride salt of (2S)-2-amino-2-(4-fluorophenyl)-N-(trans)-(1-methoxy-2,3-dihydro-1H-inden-2-yl)acetamide (Example 54, 0.1 g, 0.285 mmol) and 3,5-dimethylisoxazole-4-sulfonyl chloride (0.061 g, 0.314 mmol) in DCM (2 mL) under nitrogen. The reaction was stirred at room temperature for 18 hours. The mixture was diluted with DCM and washed with water, dried (phase separator) and concentrated in vacuo. The crude product was purified by reverse phase preparative HPLC eluted with acetonitrile/water (with 0.1% formic acid) to afford the title compound.

¹H NMR (400 MHz, DMSO-d₆) δ ppm 2.23-2.49 (m, 6H), 3.09-3.32 (m, 4H), 4.07-4.16 (m, 1H), 4.23-4.42 (m, 2H), 4.90-4.98 (m, 1H), 7.12-7.48 (m, 8H), 8.53-8.70 (m, 1H), 8.89-9.01 (m, 1H)

MS ES⁻: 472

### Example 84: (2S)—N-{(trans)-1-[(2,2-difluoroethyl)amino]-2,3-dihydro-1H-inden-2-yl}-2-(4-fluorophenyl)propanamide Single Stereoisomer

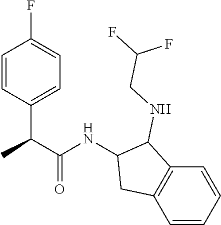 [SMILES: C[C@H](C(=O)NC1Cc2ccccc2C1NCC(F)F)c1ccc(F)cc1]

Prepared as described for Example 67 using (2S)-2-(4-fluorophenyl)-N-(cis)-(1-hydroxy-2,3-dihydro-1H-inden-2-yl)propanamide (Example 62, stereoisomer A, 0.150 g, 0.501 mmol) and 2,2-difluoroethanamine (0.203 g, 2.506 mmol). The crude product was purified by reverse phase preparative HPLC eluted with acetonitrile/water (with 0.1% ammonia) to afford the title compound.

¹H NMR (300 MHz, DMSO-d₆) δ ppm 1.33 (d, J=7.02 Hz, 3H) 2.23-2.35 (m, 1H) 2.59-2.88 (m, 3H) 3.13-3.24 (m, 1H) 3.56-3.67 (m, 1H) 3.88-4.00 (m, 1H) 4.13-4.27 (m, 1H) 5.60-6.04 (m, 1H) 7.03-7.29 (m, 6H) 7.30-7.40 (m, 2H) 8.31 (d, J=7.84 Hz, 1H)

MS ES⁺: 363

### Example 85: (2S)-2-(4-fluorophenyl)-2-methanesulfonamido-N-((trans)-1-methyl-2,3-dihydro-1H-inden-2-yl)acetamide

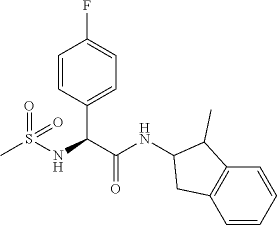 [SMILES: CC1c2ccccc2CC1NC(=O)[C@@H](NS(C)(=O)=O)c1ccc(F)cc1]

Methanesulfonic anhydride (57 mg, 0.326 mmol) was added to a stirred solution of (2S)-2-amino-2-(4-fluorophenyl)-N-(trans)-(1-methyl-2,3-dihydro-1H-inden-2-yl)acetamide (Intermediate 13, 49 mg, 0.163 mmol) and triethylamine (0.114 mL, 0.815 mmol) in THF (2 mL). The reaction was stirred for 30 minutes under nitrogen. The reaction mixture was partitioned between DCM and water and the organics were collected, dried (phase separator) and concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 0.67-1.18 (m, 3H) 2.58-2.85 (m, 4H) 2.97-3.27 (m, 2H) 4.34-4.67 (m, 1H) 5.03-5.17 (m, 1H) 7.04-7.27 (m, 6H) 7.44-7.58 (m, 2H) 7.89-8.09 (m, 1H) 8.43 (d, J=8.25 Hz, 1H)

MS ES⁺: 377

### Example 86: N-[(1S,2S)-1-amino-2,3-dihydro-1H-inden-2-yl]-2-cyclopropyl-2-(4-fluorophenyl)acetamide (Diastereomeric Mixture)

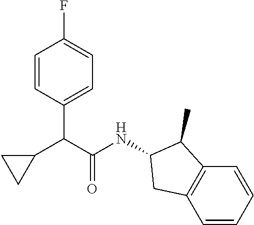 [SMILES: C[C@H]1c2ccccc2C[C@@H]1NC(=O)C(c1ccc(F)cc1)C1CC1]

T3P (50% in ethyl acetate, 1.839 mL, 3.09 mmol) was added to a stirred solution of 2-cyclopropyl-2-(4-fluorophenyl)acetic acid (300 mg, 1.545 mmol), tert-butyl ((1S,2S)-2-amino-2,3-dihydro-1H-inden-1-yl)carbamate (384 mg, 1.545 mmol) and triethylamine (0.626 mL, 4.63 mmol) in DCM (5 mL). After stirring for 20 minutes, the reaction mixture was washed with saturated aq. NaHCO₃, dried (phase separator) and the organics were purified by column chromatography on silica, eluted with 0-60% ethyl acetate/petroleum ether to afford tert-butyl ((1S,2S)-2-(2-cyclopropyl-2-(4-fluorophenyl)acetamido)-2,3-dihydro-1H-inden-1-yl)carbamate. This was treated with HCl (4 M solution in dioxane, 6.4 mL) for 4 hours. The reaction mixture was washed with saturated aq. NaHCO₃ solution and concentrated in vacuo to afford the title compound.

¹H NMR (400 MHz, DMSO-d₆) δ ppm 0.06-0.19 (m, 1H) 0.29-0.41 (m, 1H) 0.42-0.66 (m, 2H) 1.28-1.47 (m, 1H) 1.83-2.20 (m, 2H) 2.55-2.85 (m, 2H) 3.06-3.26 (m, 1H) 3.89-4.15 (m, 2H) 7.03-7.22 (m, 5H) 7.26-7.34 (m, 1H) 7.37-7.50 (m, 2H) 8.18-8.36 (m, 1H)

MS ES⁺: 325

### Example 87: tert-butyl N-[(1S,2S)-2-[2-(4-fluorophenyl)-2-methylpropanamido]-2,3-dihydro-1H-inden-1-yl]carbamate

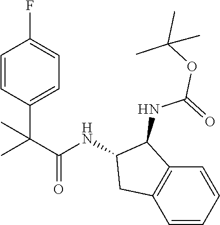 [SMILES: CC(C)(C)OC(=O)N[C@H]1c2ccccc2C[C@@H]1NC(=O)C(C)(C)c1ccc(F)cc1]

Prepared as described for Example 1 using tert-butyl ((1S,2S)-2-amino-2,3-dihydro-1H-inden-1-yl)carbamate (55 mg, 0.221 mmol) and 2-(4-fluorophenyl)-2-methylpropanoic acid (40 mg, 0.220 mmol). The crude product was purified by column chromatography on silica eluted with 0-100% EtOAc/petroleum ether to afford the title compound.

¹H NMR (400 MHz, DICHLOROMETHANE-d₂) δ ppm 1.46 (s, 9H), 1.54-1.57 (m, 6H), 2.45-2.58 (m, 1H), 3.36-3.50 (m, 1H), 4.05-4.22 (m, 2H), 4.90-5.07 (m, 1H), 6.35-6.54 (m, 1H), 6.98-7.09 (m, 2H), 7.14-7.27 (m, 4H), 7.32-7.42 (m, 2H)

MS ES⁻: 411

### Example 88: tert-butyl N-[(1S,2S)-2-(3-phenyloxetane-3-amido)-2,3-dihydro-1H-inden-1-yl]carbamate

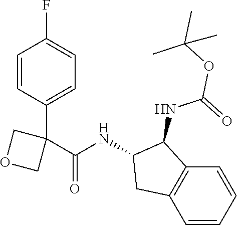 [SMILES: CC(C)(C)OC(=O)N[C@H]1c2ccccc2C[C@@H]1NC(=O)C1(c2ccc(F)cc2)COC1]

Prepared as described for Example 71 using 3-phenyloxetane-3-carboxylic acid (100 mg, 0.561 mmol) and tert-butyl ((1S,2S)-2-amino-2,3-dihydro-1H-inden-1-yl)carbamate (139 mg, 0.561 mmol). The crude product was purified by column chromatography on silica, eluted with 0-70% ethyl acetate/petroleum ether to afford the title compound.

¹H NMR (400 MHz, DMSO-d₆) δ ppm 1.44 (s, 9H), 2.57-2.72 (m, 1H), 2.94-3.16 (m, 1H), 4.35-4.48 (m, 1H), 4.69-4.84 (m, 2H), 4.97-5.19 (m, 1H), 7.00-7.24 (m, 4H), 7.27-7.54 (m, 6H), 8.23-8.47 (m, 1H)

MS ES⁺: 409

### Example 89: (2S)-2-(2,4-difluorophenyl)-N-[(1S,2S)-1-methanesulfonamido-2,3-dihydro-1H-inden-2-yl]propanamide

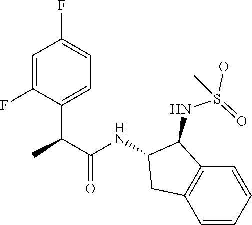

Methanesulfonyl chloride (7 μL, 0.090 mmol) was added to a solution of the hydrochloride salt of (S)—N-((1S,2S)-1-amino-2,3-dihydro-1H-inden-2-yl)-2-(2,4-difluorophenyl)propanamide (Example 34, 25 mg, 0.071 mmol) and DIPEA (25 μL, 0.143 mmol) in DCM (0.5 mL). The reaction was stirred at room temperature for 30 minutes. The mixture was partitioned between DCM and water. The phases were separated and the aqueous extracted twice with DCM. The combined organics were concentrated in vacuo. The crude product was purified by column chromatography on silica, eluted with 0-50% ethyl acetate/petroleum ether then by reverse phase preparative HPLC eluted with acetonitrile/water (with 0.1% ammonia) to afford the title compound.

¹H NMR (400 MHz, DICHLOROMETHANE-d₂) δ ppm 1.50 (d, J=7.15 Hz, 3H), 2.69-2.79 (m, 1H), 2.81 (s, 3H), 3.30-3.41 (m, 1H), 3.81-3.90 (m, 1H), 4.30-4.42 (m, 1H), 4.64-4.73 (m, 1H), 5.02 (d, J=8.07 Hz, 1H), 6.17 (d, J=5.04 Hz, 1H), 6.79-6.88 (m, 1H), 6.88-6.96 (m, 1H), 7.17-7.30 (m, 3H), 7.33-7.39 (m, 1H), 7.39-7.47 (m, 1H)

MS ES⁺: 395

### Example 90: (2S)—N-[(1S,2S)-1-[(cyclobutylmethyl)amino]-2,3-dihydro-1H-inden-2-yl]-2-(2,4-difluorophenyl)propanamide

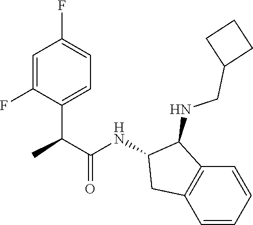 [SMILES: C[C@H](C(=O)N[C@H]1Cc2ccccc2[C@@H]1NCC1CCC1)c1ccc(F)cc1F]

Sodium triacetoxyborohydride (60 mg, 0.283 mmol) was added to a solution of the hydrochloride salt of (S)—N-((1S,2S)-1-amino-2,3-dihydro-1H-inden-2-yl)-2-(2,4-difluorophenyl)propanamide (Example 34, 50 mg, 0.142 mmol), cyclobutanecarbaldehyde (14 mg, 0.166 mmol) and glacial acetic acid (10 μL, 0.175 mmol) in DCM (1 mL) under nitrogen. The reaction was stirred at room temperature for 18 hours. The reaction mixture was partitioned between DCM and 50% saturated aq. NaHCO₃. The phases were separated and the aqueous extracted twice with DCM. The combined organics were concentrated in vacuo and the crude product was purified by reverse phase preparative HPLC eluted with acetonitrile/water (with 0.1% ammonia) to afford the title compound.

¹H NMR (300 MHz, DICHLOROMETHANE-d₂) δ ppm 1.37-1.50 (m, 3H), 1.52-1.68 (m, 3H), 1.72-2.10 (m, 4H), 2.24-2.44 (m, 1H), 2.53-2.74 (m, 3H), 3.28-3.45 (m, 1H), 3.68-3.84 (m, 1H), 3.86-3.98 (m, 1H), 4.33-4.51 (m, 1H), 5.66-5.84 (m, 1H), 6.73-6.98 (m, 2H), 7.11-7.32 (m, 4H), 7.35-7.52 (m, 1H)

MS ES⁺: 385

### Example 91: (2S)—N-[(1S,2S)-1-(cyclobutylamino)-2,3-dihydro-1H-inden-2-yl]-2-(2,4-difluorophenyl)propanamide

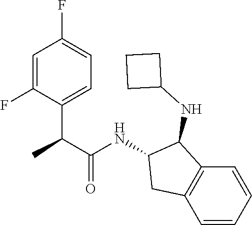 [SMILES: C[C@H](C(=O)N[C@H]1Cc2ccccc2[C@@H]1NC1CCC1)c1ccc(F)cc1F]

Prepared as described for Example 90 using (S)—N-((1S,2S)-1-amino-2,3-dihydro-1H-inden-2-yl)-2-(2,4-difluorophenyl)propanamide hydrochloride (Example 34, 50 mg, 0.142 mmol) and cyclobutanone (13 μL, 0.173 mmol). The crude product was purified by reverse phase preparative HPLC eluted with acetonitrile/water (with 0.1% ammonia) to afford the title compound.

¹H NMR (300 MHz, DICHLOROMETHANE-d₂) δ ppm 1.38-1.71 (m, 7H), 1.99-2.19 (m, 3H), 2.57-2.70 (m, 1H), 3.28-3.48 (m, 2H), 3.68-3.83 (m, 1H), 3.84-3.98 (m, 1H), 4.21-4.39 (m, 1H), 5.66-5.83 (m, 1H), 6.70-7.01 (m, 2H), 7.09-7.33 (m, 4H), 7.36-7.51 (m, 1H)

MS ES⁺: 371

### Example 92: (2S)-2-(2,4-difluorophenyl)-N-[(1S,2S)-1-{[(3-fluoropyridin-2-yl)methyl]amino}-2,3-dihydro-1H-inden-2-yl]propanamide

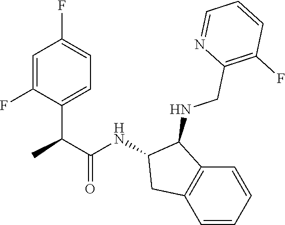 [SMILES: C[C@H](C(=O)N[C@H]1Cc2ccccc2[C@@H]1NCc1ncccc1F)c1ccc(F)cc1F]

Prepared as described for Example 90 using (S)—N-((1S,2S)-1-amino-2,3-dihydro-1H-inden-2-yl)-2-(2,4-difluorophenyl)propanamide hydrochloride (Example 34, 50 mg, 0.142 mmol) and 3-fluoropicolinaldehyde (22 mg, 0.176 mmol). The crude product was purified by reverse phase preparative HPLC eluted with acetonitrile/water (with 0.1% ammonia) to afford the title compound.

¹H NMR (400 MHz, DICHLOROMETHANE-d₂) δ ppm 1.46 (d, J=7.15 Hz, 4H), 2.62-2.71 (m, 1H), 3.45-3.56 (m, 1H), 3.77-3.86 (m, 1H), 3.97-4.04 (m, 1H), 4.06-4.13 (m, 2H), 4.36-4.47 (m, 1H), 6.38 (d, J=5.69 Hz, 1H), 6.72-6.80 (m, 1H), 6.81-6.89 (m, 1H), 7.16-7.27 (m, 4H), 7.31 (d, J=6.33 Hz, 1H), 7.35-7.45 (m, 2H), 8.30-8.37 (m, 1H)

MS ES⁺: 426

### Example 93: tert-butyl N-[(1S,2S)-2-[2-(2,4-difluorophenyl)-2-(3-fluoroazetidin-1-yl)acetamido]-2,3-dihydro-1H-inden-1-yl]carbamate Diastereomeric Mixture

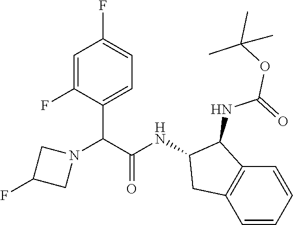 [SMILES: CC(C)(C)OC(=O)N[C@H]1c2ccccc2C[C@@H]1NC(=O)C(c1ccc(F)cc1F)N1CC(F)C1]

Prepared as described for Example 1 using lithio 2-(2,4-difluorophenyl)-2-(3-fluoroazetidin-1-yl)acetate (Intermediate 14, 270 mg, 1.075 mmol) and tert-butyl ((1S,2S)-2-amino-2,3-dihydro-1H-inden-1-yl)carbamate (294 mg, 1.183 mmol). The crude product was purified by column chromatography on silica, eluted with 0-100% ethyl acetate/petroleum ether to afford the title compound.

¹H NMR (400 MHz, DICHLOROMETHANE-d₂) δ ppm 1.37-1.51 (m, 9H) 2.57-2.79 (m, 1H) 3.06-3.97 (m, 5H) 4.17-4.41 (m, 2H) 4.95-5.28 (m, 3H) 6.83-6.98 (m, 2H) 7.15-7.29 (m, 4H) 7.41 (br. s., 1H) 7.75 (d, J=6.69 Hz, 1H)

MS ES⁺: 476

### Example 94: tert-butyl N-[(1S,2S)-2-[4-(4-fluorophenyl)oxane-4-amido]-2,3-dihydro-1H-inden-1-yl]carbamate

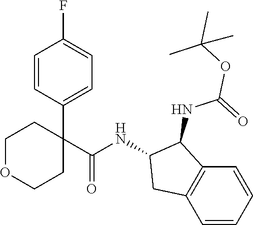 [SMILES: CC(C)(C)OC(=O)N[C@H]1c2ccccc2C[C@@H]1NC(=O)C1(c2ccc(F)cc2)CCOCC1]

Prepared as described for Example 1 using tert-butyl ((1S,2S)-2-amino-2,3-dihydro-1H-inden-1-yl)carbamate (55 mg, 0.221 mmol) and 4-(4-fluorophenyl)tetrahydro-2H-pyran-4-carboxylic acid (40 mg, 0.178 mmol) after purification by column chromatography on silica, eluted with 0-100% ethyl acetate/petroleum ether to afford the title compound.

¹H NMR (400 MHz, DICHLOROMETHANE-d₂) δ ppm 1.45 (s, 9H), 1.92-2.14 (m, 2H), 2.33-2.54 (m, 3H), 3.45-3.56 (m, 1H), 3.58-3.86 (m, 4H), 3.95-4.08 (m, 1H), 4.93-5.13 (m, 2H), 6.80-6.90 (m, 1H), 7.00-7.10 (m, 2H), 7.16-7.28 (m, 4H), 7.35-7.45 (m, 2H)

MS ES⁻: 453

### Example 95: (2S)-2-(4-fluorophenyl)-N-[(1R,2R)-1-(methylamino)-2,3-dihydro-1H-inden-2-yl]propanamide

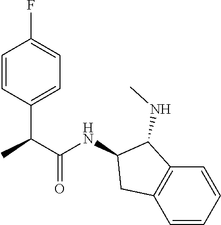 [SMILES: CN[C@@H]1c2ccccc2C[C@H]1NC(=O)[C@@H](C)c1ccc(F)cc1]

Prepared as described for Example 67 using (2S)-2-(4-fluorophenyl)-N-(cis)-(1-hydroxy-2,3-dihydro-1H-inden-2-yl)propanamide (Example 63 (second eluting peak), 0.300 g, 1.00 mmol) and methylamine (2M in THF, 2.50 mL, 5.01 mmol). The crude product was purified by reverse phase chromatography on C18 silica eluted with 5-95% acetonitrile/water (with 0.1% formic acid). The crude product was loaded onto a cation exchange cartridge, washed with methanol and eluted with 2M ammonia/methanol solution then concentrated in vacuo. The resulting residue was triturated with diethyl ether to afford the title compound.

¹H NMR (400 MHz, DMSO-d₆) δ ppm 1.32 (d, J=6.97 Hz, 3H) 1.97-2.19 (m, 1H) 2.33 (s, 3H) 2.50-2.58 (m, 1H) 3.07-3.20 (m, 1H) 3.55-3.67 (m, 1H) 3.97 (d, J=5.87 Hz, 1H) 4.18-4.31 (m, 1H) 7.06-7.22 (m, 5H) 7.25-7.41 (m, 3H) 8.28 (d, J=7.70 Hz, 1H)

MS ES⁺: 313

### Examples 96 and 97: N-[(1S,2S)-1-amino-2,3-dihydro-1H-inden-2-yl]-2-cyclopropyl-2-(4-fluorophenyl)acetamide Stereoisomers A and B

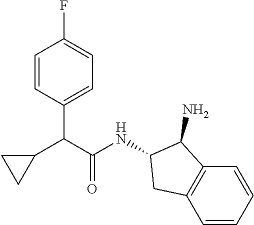 [SMILES: N[C@H]1c2ccccc2C[C@@H]1NC(=O)C(c1ccc(F)cc1)C1CC1]

Example 86 was separated by chiral SFC (IA Diacel CHIRALPAK, 23% IPA+0.5% DEA) to afford the title compounds.

### Example 96—Stereoisomer A—First Eluting Peak

¹H NMR (400 MHz, DMSO-d₆) δ ppm 0.05-0.17 (m, 1H) 0.31-0.38 (m, 1H) 0.42-0.65 (m, 2H) 1.29-1.48 (m, 1H) 1.70-1.99 (m, 2H) 2.55-2.65 (m, 1H) 2.75 (d, J=9.90 Hz, 1H) 3.13-3.27 (m, 1H) 3.78-4.05 (m, 2H) 7.04-7.23 (m, 5H) 7.26-7.33 (m, 1H) 7.37-7.57 (m, 2H) 8.28 (d, J=6.88 Hz, 1H)

MS ES⁺: 325

### Example 97—Stereoisomer B—Second Eluting Peak

¹H NMR (400 MHz, DMSO-d₆) δ ppm −0.06-0.20 (m, 1H) 0.30-0.40 (m, 1H) 0.43-0.64 (m, 2H) 1.27-1.47 (m, 1H) 1.85-2.14 (m, 2H) 2.36-2.48 (m, 1H) 2.71-2.79 (m, 1H) 3.05-3.17 (m, 1H) 3.81-3.99 (m, 1H) 4.02-4.11 (m, 1H) 7.05-7.23 (m, 5H) 7.27-7.34 (m, 1H) 7.38-7.47 (m, 2H) 8.19-8.34 (m, 1H)

MS ES⁺: 325

### Example 98: (2S)-2-(4-fluorophenyl)-N-(trans)-(1-methoxy-2,3-dihydro-1H-inden-2-yl)-2-[(oxan-4-yl)formamido]acetamide

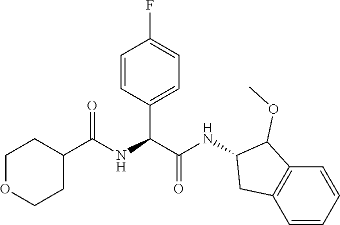 [SMILES: COC1c2ccccc2C[C@@H]1NC(=O)[C@@H](NC(=O)C1CCOCC1)c1ccc(F)cc1]

Prepared as described for Example 42 using (2S)-2-amino-2-(4-fluorophenyl)-N-(trans)-1-(methoxy-2,3-dihydro-1H-inden-2-yl)acetamide (Example 54, 0.2 g, 0.570 mmol) and tetrahydro-2H-pyran-4-carboxylic acid (0.082 g, 0.627 mmol) with purification by column chromatography on silica, eluted with 0-100% ethyl acetate/petroleum ether and reverse phase preparative HPLC eluted with acetonitrile/water (with 0.1% ammonia) to afford the title compound.

¹H NMR (400 MHz, DMSO-d₆) δ ppm 1.42-1.62 (m, 4H) 2.53-2.75 (m, 2H) 3.11-3.40 (m, 6H) 3.77-3.90 (m, 2H) 4.22-4.36 (m, 1H) 4.46-4.69 (m, 1H) 5.44 (d, J=8.07 Hz, 1H) 7.09-7.38 (m, 6H) 7.40-7.50 (m, 2H) 8.42-8.54 (m, 1H) 8.63-8.74 (m, 1H)

MS ES⁻: 425

### Example 99: N-[(1S,2S)-1-amino-2,3-dihydro-1H-inden-2-yl]-2-(4-fluorophenyl)-2-methylpropanamide

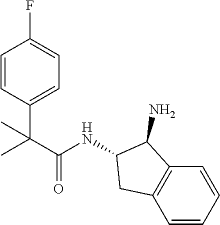 [SMILES: CC(C)(C(=O)N[C@H]1Cc2ccccc2[C@@H]1N)c1ccc(F)cc1]

To a solution of Example 87 (153 mg, 0.371 mmol) in DCM (5 mL) was added TFA (0.150 mL, 1.947 mmol). The reaction was stirred at room temperature for 18 hours. Another portion of TFA (0.150 mL, 1.947 mmol) was added to the reaction mixture and stirred at room temperature for a further 4 hours. The reaction mixture was concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 1.45-1.55 (m, 6H), 1.87 (br. s., 2H), 2.55-2.66 (m, 1H), 3.01-3.14 (m, 1H), 3.98-4.13 (m, 2H), 7.08-7.21 (m, 5H), 7.24-7.32 (m, 1H), 7.35-7.44 (m, 2H), 7.52-7.61 (m, 1H)

MS ES⁺: 313

### Example 100: tert-butyl N-[(1S,2S)-2-[2-(azetidin-1-yl)-2-(2,4-difluorophenyl)acetamido]-2,3-dihydro-1H-inden-1-yl]carbamate

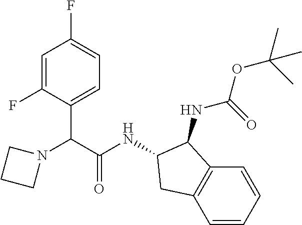 [SMILES: CC(C)(C)OC(=O)N[C@H]1c2ccccc2C[C@@H]1NC(=O)C(c1ccc(F)cc1F)N1CCC1]

Prepared as described for Example 1 using lithio 2-(azetidin-1-yl)-2-(2,4-difluorophenyl)acetate (Intermediate 17, 247 mg, 1.059 mmol) and tert-butyl ((1S,2S)-2-amino-2,3-dihydro-1H-inden-1-yl)carbamate (105 mg, 0.424 mmol) to afford the title compound. The crude product was purified by reverse phase preparative HPLC eluted with acetonitrile/water (with 0.1% ammonia) to afford the title compound.

¹H NMR (400 MHz, DMSO-d₆) δ ppm 1.33-1.47 (m, 9H) 1.91-2.04 (m, 2H) 2.68-3.11 (m, 4H) 3.13-3.26 (m, 2H) 4.14 (d, J=9.90 Hz, 1H) 4.24-4.42 (m, 1H) 5.13-5.26 (m, 1H) 6.93-7.34 (m, 7H) 7.45-7.70 (m, 1H) 8.32-8.44 (m, 1H)

MS ES⁺: 458

### Examples 101 and 102: (2S)-2-(cyclopropylformamido)-2-(4-fluorophenyl)-N-(trans)-(1-methoxy-2,3-dihydro-1H-inden-2-yl)acetamide Stereoisomers A and B

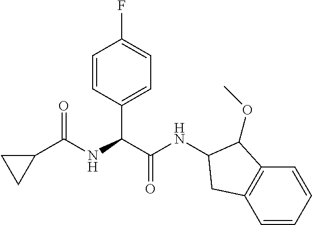 [SMILES: COC1c2ccccc2CC1NC(=O)[C@@H](NC(=O)C1CC1)c1ccc(F)cc1]

Example 66 was separated by chiral SFC (IA Diacel CHIRALPAK, 30% IPA) to afford the title compounds.

### Example 101—Stereoisomer A—First Eluting Peak

¹H NMR (400 MHz, DMSO-d₆) δ ppm 0.56-0.72 (m, 4H) 1.79-1.92 (m, 1H) 2.53-2.63 (m, 1H) 3.13-3.24 (m, 1H) 3.38 (s, 3H) 4.22-4.34 (m, 1H) 4.62-4.69 (m, 1H) 5.49 (d, J=8.25 Hz, 1H) 7.10-7.32 (m, 5H) 7.32-7.38 (m, 1H) 7.41-7.50 (m, 2H) 8.71 (d, J=7.52 Hz, 1H) 8.84 (d, J=8.25 Hz, 1H)

MS ES⁺: 383

### Example 102—Stereoisomer B—Second Eluting Peak

¹H NMR (400 MHz, DMSO-d₆) δ ppm 0.57-0.72 (m, 4H) 1.81-1.91 (m, 1H) 2.65-2.76 (m, 1H) 3.16 (s, 3H) 3.19-3.28 (m, 1H) 4.25-4.35 (m, 1H) 4.47-4.53 (m, 1H) 5.50 (d, J=8.25 Hz, 1H) 7.12-7.32 (m, 6H) 7.39-7.51 (m, 2H) 8.74 (d, J=8.07 Hz, 1H) 8.84 (d, J=8.25 Hz, 1H)

MS ES⁺: 383

### Example 103: N-[(1S,2S)-1-amino-2,3-dihydro-1H-inden-2-yl]-2-(3,3-difluoroazetidin-1-yl)-2-(2,4-difluorophenyl)acetamide formate

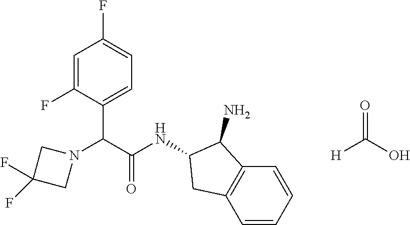 [SMILES: N[C@H]1c2ccccc2C[C@@H]1NC(=O)C(c1ccc(F)cc1F)N1CC(F)(F)C1 ; O=CO]

Prepared as described for Example 75 using lithio 2-(azetidin-1-yl)-2-(2,4-difluorophenyl)acetate (Intermediate 18, 152 mg, 0.565 mmol) and tert-butyl ((1S,2S)-2-amino-2,3-dihydro-1H-inden-1-yl)carbamate (154 mg, 0.621 mmol). The crude product was purified by reverse phase preparative HPLC eluted with acetonitrile/water (with 0.1% ammonia) then again by reverse phase preparative HPLC eluted with acetonitrile/water (with 0.1% formic acid) to afford the title compound as the formate salt.

¹H NMR (400 MHz, DMSO-d₆) δ ppm 2.62-2.72 (m, 1H) 3.04-3.15 (m, 2H) 3.60-3.74 (m, 5H) 4.00-4.13 (m, 1H) 4.24 (d, J=7.79 Hz, 1H) 4.47 (d, J=3.21 Hz, 1H) 7.10-7.36 (m, 5H) 7.60-7.69 (m, 1H) 8.21 (s, 1H) 8.39-8.46 (m, 1H)

MS ES⁺: 394

### Example 104: tert-butyl N-[(1S,2S)-2-[2-(2,4-difluorophenyl)-2-(3-methoxyazetidin-1-yl)acetamido]-2,3-dihydro-1H-inden-1-yl]carbamate

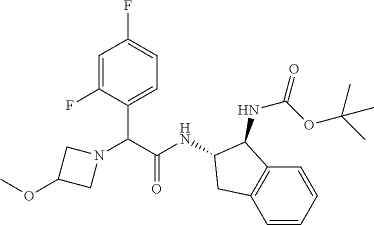 [SMILES: COC1CN(C(C(=O)N[C@H]2Cc3ccccc3[C@@H]2NC(=O)OC(C)(C)C)c2ccc(F)cc2F)C1]

Prepared as described for Example 1 using lithio 2-(2,4-difluorophenyl)-2-(3-methoxyazetidin-1-yl)acetate (Intermediate 19, 442 mg, 1.680 mmol) and tert-butyl ((1S,2S)-2-amino-2,3-dihydro-1H-inden-1-yl)carbamate (167 mg, 0.672 mmol). The crude product was purified by column chromatography on silica, eluted with 0-50% ethyl acetate/petroleum ether, then by reverse phase preparative HPLC eluted with acetonitrile/water (with 0.1% ammonia) to afford the title compound.

¹H NMR (400 MHz, DMSO-d₆) δ ppm 1.32-1.47 (m, 9H), 2.69-3.06 (m, 4H), 3.12 (s, 3H), 3.22-3.30 (m, 1H), 3.56-3.73 (m, 1H), 3.92-4.04 (m, 1H), 4.18 (d, J=17.51 Hz, 1H), 4.25-4.43 (m, 1H), 5.12-5.25 (m, 1H), 6.97-7.33 (m, 7H), 7.44-7.68 (m, 1H), 8.42 (d, J=8.80 Hz, 1H)

MS ES⁺: 488

### Example 105: N-[(1S,2S)-1-amino-2,3-dihydro-1H-inden-2-yl]-2-(2,4-difluorophenyl)-2-(3-fluoroazetidin-1-yl)acetamide

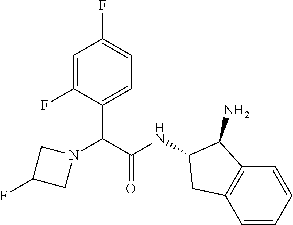 [SMILES: N[C@H]1c2ccccc2C[C@@H]1NC(=O)C(c1ccc(F)cc1F)N1CC(F)C1]

Prepared as described for Example 50 using tert-butyl N-[(1S,2S)-2-[2-(2,4-difluorophenyl)-2-(3-fluoroazetidin-1-yl)acetamido]-2,3-dihydro-1H-inden-1-yl]carbamate (Example 93, 354 mg, 0.744 mmol). The crude product was purified by reverse phase preparative HPLC eluted with acetonitrile/water (with 0.1% ammonia) to afford the title compound.

¹H NMR (300 MHz, DMSO-d₆) δ ppm 1.90 (br. s., 2H) 2.60-2.72 (m, 1H) 3.00-3.29 (m, 3H) 3.39-3.56 (m, 1H) 3.58-3.75 (m, 1H) 3.91-4.07 (m, 1H) 4.12-4.23 (m, 1H) 4.34 (s, 1H) 5.04-5.35 (m, 1H) 7.07-7.34 (m, 6H) 7.56-7.70 (m, 1H) 8.33 (d, J=7.84 Hz, 1H)

MS ES⁺: 376

### Examples 106 and 107: N-[(1S,2S)-1-amino-2,3-dihydro-1H-inden-2-yl]-2-(2,4-difluorophenyl)-2-(3-fluoroazetidin-1-yl)acetamide Stereoisomers A and B

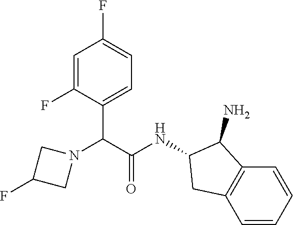 [SMILES: N[C@H]1c2ccccc2C[C@@H]1NC(=O)C(c1ccc(F)cc1F)N1CC(F)C1]

Example 105 was separated by chiral SFC (ID Diacel CHIRALPAK, 34% IPA+0.2% DEA) to afford the title compounds.

### Example 106—Stereoisomer A—First Eluting Peak

¹H NMR (400 MHz, DMSO-d₆) δ ppm 1.93 (br. s., 2H), 2.59-2.72 (m, 1H), 3.00-3.10 (m, 1H), 3.12-3.31 (m, 2H), 3.40-3.52 (m, 1H), 3.61-3.73 (m, 1H), 3.92-4.05 (m, 1H), 4.16 (d, J=8.16 Hz, 1H), 4.33 (s, 1H), 5.06-5.32 (m, 1H), 7.08-7.33 (m, 6H), 7.57-7.67 (m, 1H), 8.34 (d, J=7.89 Hz, 1H)

MS ES⁺: 376

### Example 107—Stereoisomer B—Second Eluting Peak

¹H NMR (400 MHz, DMSO-d₆) δ ppm 1.89 (br. s., 2H), 2.60-2.73 (m, 1H), 3.01-3.29 (m, 3H), 3.39-3.54 (m, 1H), 3.57-3.74 (m, 1H), 3.91-4.06 (m, 1H), 4.17 (d, J=8.07 Hz, 1H), 4.33 (s, 1H), 5.06-5.32 (m, 1H), 7.08-7.33 (m, 6H), 7.58-7.68 (m, 1H), 8.34 (d, J=7.79 Hz, 1H)

MS ES⁺: 376

### Example 108: N-[(1S,2S)-1-amino-2,3-dihydro-1H-inden-2-yl]-2-(azetidin-1-yl)-2-(2,4-difluorophenyl)acetamide

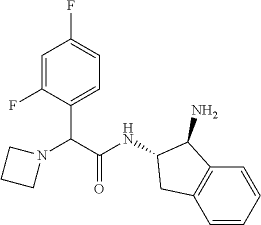 [SMILES: N[C@H]1c2ccccc2C[C@@H]1NC(=O)C(c1ccc(F)cc1F)N1CCC1]

Prepared as described for Example 50 using tert-butyl N-[(1S,2S)-2-[2-(azetidin-1-yl)-2-(2,4-difluorophenyl)acetamido]-2,3-dihydro-1H-inden-1-yl]carbamate (Example 100, 24 mg, 0.052 mmol). The crude product was purified by reverse phase preparative HPLC eluted with acetonitrile/water (with 0.1% ammonia) to afford the title compound.

¹H NMR (400 MHz, DICHLOROMETHANE-d₂) δ ppm 1.97-2.16 (m, 4H), 2.70-2.84 (m, 1H), 3.08-3.19 (m, 2H), 3.22-3.40 (m, 3H), 4.14-4.30 (m, 3H), 6.81-6.96 (m, 2H), 7.17-7.28 (m, 3H), 7.30-7.47 (m, 2H), 7.48-7.61 (m, 1H)

MS ES⁺: 358

### Examples 109 and 110: N-[(1S,2S)-1-amino-2,3-dihydro-1H-inden-2-yl]-2-(2,4-difluorophenyl)-2-(6-oxo-1,6-dihydropyridazin-1-yl)acetamide Stereoisomers A and B

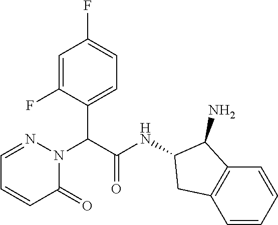 [SMILES: N[C@H]1c2ccccc2C[C@@H]1NC(=O)C(c1ccc(F)cc1F)n1ncccc1=O]

Prepared as described for Example 50 using tert-butyl N-[(1S,2S)-2-[2-(2,4-difluorophenyl)-2-(6-oxo-1,6-dihydropyridazin-1-yl)acetamido]-2,3-dihydro-1H-inden-1-yl]carbamate (Intermediate 22, 450 mg, 0.906 mmol). The crude product was purified by column chromatography on basic silica, eluted with 0-100% ethyl acetate/petroleum ether then 0-10% methanol (with 0.1% ammonia)/DCM, then twice by reverse phase preparative HPLC eluted with acetonitrile/water (with 0.1% ammonia) to afford the title compounds.

### Example 109—Stereoisomer A—First Eluting Peak

¹H NMR (300 MHz, DICHLOROMETHANE-d₂) δ ppm 1.93-2.10 (m, 2H), 2.66-2.82 (m, 1H), 3.22-3.42 (m, 1H), 4.10-4.33 (m, 2H), 6.26-6.50 (m, 1H), 6.80-7.02 (m, 4H), 7.13-7.26 (m, 4H), 7.29-7.38 (m, 1H), 7.59-7.69 (m, 1H), 7.73-7.80 (m, 1H)

MS ES⁺: 397

### Example 110—Stereoisomer B—Second Eluting Peak

¹H NMR (400 MHz, DMSO-d₆) δ ppm 1.93-2.10 (m, 2H), 2.53-2.63 (m, 1H), 3.19-3.26 (m, 1H), 4.01-4.17 (m, 2H), 6.69 (s, 1H), 6.97-7.04 (m, 1H), 7.10-7.49 (m, 7H), 7.82-7.89 (m, 1H), 8.20 (s, 1H), 8.74 (d, J=6.51 Hz, 1H)

MS ES⁺: 397

### Example 111: N-[(1S,2S)-1-amino-2,3-dihydro-1H-inden-2-yl]-2-(2,4-difluorophenyl)-2-(3-methoxyazetidin-1-yl)acetamide

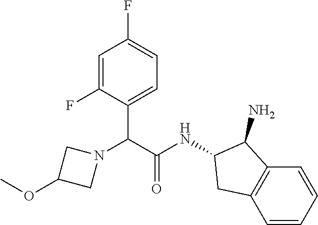 [SMILES: COC1CN(C(C(=O)N[C@H]2Cc3ccccc3[C@@H]2N)c2ccc(F)cc2F)C1]

Prepared as described for Example 50 using tert-butyl N-[(1S,2S)-2-[2-(2,4-difluorophenyl)-2-(3-methoxyazetidin-1-yl)acetamido]-2,3-dihydro-1H-inden-1-yl]carbamate (Example 104, 71 mg, 0.146 mmol). The crude product was purified by reverse phase preparative HPLC eluted with acetonitrile/water (with 0.1% ammonia) to afford the title compound.

¹H NMR (400 MHz, DMSO-d₆) δ ppm 2.15 (br. s., 2H), 2.61-2.73 (m, 1H), 2.78-2.86 (m, 1H), 2.91-3.16 (m, 6H), 3.57-3.67 (m, 1H), 3.92-4.05 (m, 2H), 4.14-4.20 (m, 1H), 4.23 (d, J=2.11 Hz, 1H), 7.05-7.33 (m, 6H), 7.54-7.67 (m, 1H), 8.31 (d, J=7.89 Hz, 1H)

MS ES⁺: 488

### Example 112: (2S)—N-[(1R,2R)-1-(3-fluoroazetidin-1-yl)-2,3-dihydro-1H-inden-2-yl]-2-(4-fluorophenyl)propanamide

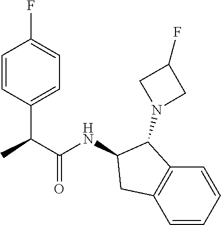 [SMILES: C[C@H](C(=O)N[C@@H]1Cc2ccccc2[C@H]1N1CC(F)C1)c1ccc(F)cc1]

DAST (0.037 mL, 0.282 mmol) was added slowly to a suspension of (2S)-2-(4-fluorophenyl)-N-[(1R,2R)-1-(3-hydroxyazetidin-1-yl)-2,3-dihydro-1H-inden-2-yl]propanamide (enantiomer of Intermediate 51, 0.05 g, 0.141 mmol) in DCM (1 mL) under nitrogen at −78° C. The reaction was stirred at −78° C. for 90 minutes. The reaction was quenched with saturated aq. NaHCO₃, diluted with DCM and allowed to warm to room temperature. The phases were separated and the organic layer was concentrated in vacuo. The crude product was purified by reverse phase preparative HPLC eluted with acetonitrile/water (with 0.1% ammonia) to afford the title compound.

¹H NMR (400 MHz, METHANOL-d₄) δ ppm 1.43 (d, J=7.15 Hz, 3H) 2.62-2.72 (m, 1H) 3.35-3.44 (m, 1H) 3.47-3.64 (m, 3H) 3.66-3.91 (m, 3H) 4.26-4.33 (m, 1H) 5.03-5.26 (m, 1H) 6.99-7.07 (m, 2H) 7.18-7.40 (m, 6H)

MS ES⁺: 357

### Example 113: (2S)-2-(4-fluorophenyl)-N-[(1R,2R)-1-methanesulfonyl-2,3-dihydro-1H-inden-2-yl]propanamide

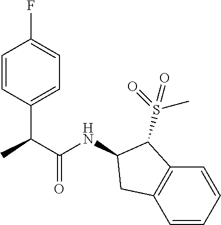 [SMILES: C[C@H](C(=O)N[C@@H]1Cc2ccccc2[C@H]1S(C)(=O)=O)c1ccc(F)cc1]

mCPBA (0.170 g, 0.759 mmol) was added to a solution of (2S)-2-(4-fluorophenyl)-N-(trans)-[1-(methylsulfanyl)-2,3-dihydro-1H-inden-2-yl]propanamide (Intermediate 23, 0.1 g, 0.304 mmol) in DCM (2 mL) under nitrogen. The reaction was stirred at room temperature for 1 hour. Calcium hydroxide (0.084 g, 1.138 mmol) was added and then stirred for 20 minutes. MgSO₄ was added and the suspension filtered and concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 1.31 (d, J=6.90 Hz, 3H) 2.66-2.76 (m, 1H) 3.12 (s, 3H) 3.33-3.42 (m, 1H) 3.56 (q, J=6.90 Hz, 1H) 4.64-4.70 (m, 1H) 4.75-4.82 (m, 1H) 7.07-7.17 (m, 2H) 7.26-7.40 (m, 5H) 7.45-7.53 (m, 1H) 8.61 (d, J=7.15 Hz, 1H)

MS ES⁺: 362

### Example 114: (2S)—N-[(1S,2S)-1-{bis[(1,3-oxazol-2-yl)methyl]amino}-2,3-dihydro-1H-inden-2-yl]-2-(2,4-difluorophenyl)propanamide

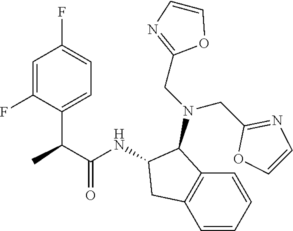 [SMILES: C[C@H](C(=O)N[C@H]1Cc2ccccc2[C@@H]1N(Cc1ncco1)Cc1ncco1)c1ccc(F)cc1F]

Sodium triacetoxyborohydride (100 mg, 0.474 mmol) was added to a solution of (S)—N-((1S,2S)-1-amino-2,3-dihydro-1H-inden-2-yl)-2-(2,4-difluorophenyl)propanamide (Example 34, 75 mg, 0.237 mmol), oxazole-2-carbaldehyde (28 mg, 0.288 mmol) and glacial acetic acid (0.016 mL, 0.284 mmol) in DCM (1 mL) under nitrogen. The reaction was stirred at room temperature for 6 hours. The reaction mixture was partitioned between DCM and saturated aq. NaHCO₃. The phases were separated and the aqueous extracted twice with DCM. The combined organics were concentrated in vacuo. The crude product was purified by reverse phase preparative HPLC eluted with acetonitrile/water (with 0.1% ammonia) to afford the title compound.

¹H NMR (400 MHz, DMSO-d₆) δ ppm 1.38 (d, J=7.15 Hz, 3H), 2.71-2.83 (m, 1H), 3.10-3.23 (m, 1H), 3.47-3.60 (m, 2H), 3.63-3.76 (m, 2H), 3.79-3.93 (m, 1H), 4.22 (d, J=6.60 Hz, 1H), 4.52-4.70 (m, 1H), 6.79 (s, 2H), 6.98-7.12 (m, 1H), 7.14-7.33 (m, 5H), 7.43-7.58 (m, 1H), 8.18 (s, 2H), 8.49 (d, J=8.25 Hz, 1H)

MS ES⁺: 479.3

### Example 115: N-[(1S,2S)-1-amino-2,3-dihydro-1H-inden-2-yl]-2-(3-fluoroazetidin-1-yl)-2-(4-fluorophenyl)acetamide

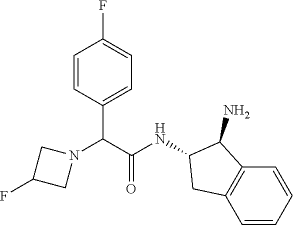 [SMILES: N[C@H]1c2ccccc2C[C@@H]1NC(=O)C(c1ccc(F)cc1)N1CC(F)C1]

TFA (0.168 mL, 2.186 mmol) was added to a suspension of tert-butyl N-[(1S,2S)-2-[2-(3-fluoroazetidin-1-yl)-2-(4-fluorophenyl)acetamido]-2,3-dihydro-1H-inden-1-yl]carbamate (Intermediate 25, 100 mg, 0.219 mmol) in DCM (1.0 mL) under nitrogen. The reaction was stirred at room temperature for 24 hours. The reaction mixture was loaded onto a cation exchange cartridge, washed with methanol and eluted with 2M ammonia/methanol solution then concentrated in vacuo to afford the title compound.

¹H NMR (400 MHz, DMSO-d₆) δ ppm 2.63-2.75 (m, 1H), 2.99-3.09 (m, 1H), 3.13-3.28 (m, 2H), 3.33-3.44 (m, 3H), 3.64-3.79 (m, 1H), 3.94-4.12 (m, 2H), 4.19-4.29 (m, 1H), 5.08-5.33 (m, 1H), 7.11-7.26 (m, 5H), 7.29-7.38 (m, 1H), 7.44-7.53 (m, 2H), 8.27-8.36 (m, 1H)

MS ES⁺: 358

### Examples 116 and 117: N-[(1S,2S)-1-amino-2,3-dihydro-1H-inden-2-yl]-2-(3-fluoroazetidin-1-yl)-2-(4-fluorophenyl)acetamide Single Stereoisomers A and B

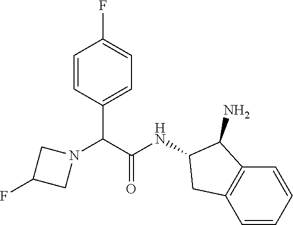 [SMILES: N[C@H]1c2ccccc2C[C@@H]1NC(=O)C(c1ccc(F)cc1)N1CC(F)C1]

Example 115 was separated by chiral SFC (AD Diacel CHIRALPAK, 18% EtOH+0.2% DEA) to afford the title compounds.

### Example 116—Stereoisomer A—First Eluting Peak

¹H NMR (400 MHz, DMSO-d₆) δ ppm 1.89 (br. s., 2H), 2.60-2.71 (m, 1H), 2.97-3.12 (m, 2H), 3.14-3.21 (m, 1H), 3.44 (t, J=6.92 Hz, 1H), 3.68 (t, J=6.92 Hz, 1H), 3.93-4.04 (m, 1H), 4.17 (d, J=7.98 Hz, 1H), 4.32 (s, 1H), 4.67-4.78 (m, 1H), 6.42-6.89 (m, 1H), 7.05-7.33 (m, 6H), 7.56-7.67 (m, 1H), 8.34 (d, J=7.70 Hz, 1H)

MS ES⁺: 424

### Example 117—Stereoisomer B—Second Eluting Peak

¹H NMR (400 MHz, DMSO-d₆) δ ppm 1.91 (d, J=4.58 Hz, 2H), 2.59-2.72 (m, 1H), 2.97-3.11 (m, 2H), 3.13-3.22 (m, 1H), 3.43 (t, J=6.56 Hz, 1H), 3.70 (t, J=6.69 Hz, 1H), 3.90-4.03 (m, 1H), 4.16 (d, J=8.16 Hz, 1H), 4.31 (s, 1H), 4.65-4.79 (m, 1H), 6.41-6.91 (m, 1H), 7.04-7.37 (m, 6H), 7.52-7.68 (m, 1H), 8.34 (d, J=7.79 Hz, 1H)

MS ES⁺: 424

### Example 118: N-[(1S,2S)-1-amino-2,3-dihydro-1H-inden-2-yl]-2-(3-fluoroazetidin-1-yl)-2-(4-fluorophenyl)acetamide Single Stereoisomer

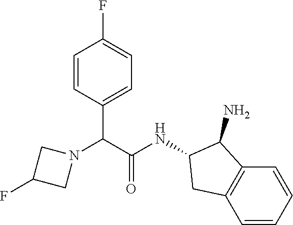 [SMILES: N[C@H]1c2ccccc2C[C@@H]1NC(=O)C(c1ccc(F)cc1)N1CC(F)C1]

Prepared as described for Example 115 using tert-butyl N-[(1S,2S)-2-[2-(3-fluoroazetidin-1-yl)-2-(4-fluorophenyl)acetamido]-2,3-dihydro-1H-inden-1-yl]carbamate (Intermediate 26, 240 mg, 0.458 mmol) and TFA (0.353 mL, 4.58 mmol). The crude product was purified by cation exchange cartridge to afford the title compound.

¹H NMR (400 MHz, DMSO-d₆) δ ppm 2.56-3.01 (m, 3H), 3.04-3.27 (m, 3H), 3.34-3.43 (m, 1H), 3.59-3.72 (m, 1H), 3.93-4.04 (m, 2H), 4.21 (d, J=7.89 Hz, 1H), 5.09-5.31 (m, 1H), 7.12-7.25 (m, 5H), 7.31 (d, J=6.60 Hz, 1H), 7.46-7.52 (m, 2H), 8.28 (d, J=7.70 Hz, 1H)

MS ES⁺: 358

### Example 119: N-[(1S,2S)-1-amino-2,3-dihydro-1H-inden-2-yl]-2-[3-(difluoromethoxy)azetidin-1-yl]-2-(2,4-difluorophenyl)acetamide

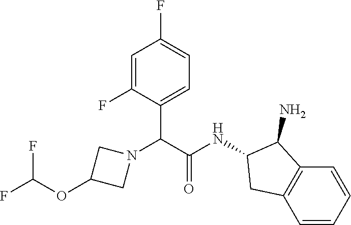 [SMILES: N[C@H]1c2ccccc2C[C@@H]1NC(=O)C(c1ccc(F)cc1F)N1CC(OC(F)F)C1]

Prepared as described for Example 115 using tert-butyl N-[(1S,2S)-2-{2-[3-(difluoromethoxy)azetidin-1-yl]-2-(2,4-difluorophenyl)acetamido}-2,3-dihydro-1H-inden-1-yl]carbamate (Intermediate 28, 240 mg, 0.458 mmol) and TFA (0.353 mL, 4.58 mmol). The crude product was purified by cation exchange cartridge to afford the title compound.

¹H NMR (400 MHz, DMSO-d₆) δ ppm 2.33 (br. s., 2H), 2.62-2.76 (m, 1H), 2.96-3.22 (m, 3H), 3.39-3.50 (m, 1H), 3.63-3.74 (m, 1H), 3.93-4.08 (m, 1H), 4.13-4.22 (m, 1H), 4.32 (d, J=2.57 Hz, 1H), 4.65-4.80 (m, 1H), 6.43-6.91 (m, 1H), 7.06-7.37 (m, 6H), 7.52-7.70 (m, 1H), 8.36 (d, J=7.79 Hz, 1H)

MS ES⁺: 424

### Example 120: N-[(1S,2S)-1-amino-2,3-dihydro-1H-inden-2-yl]-3-phenylpyrrolidine-3-carboxamide

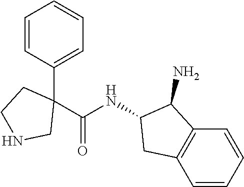 [SMILES: N[C@H]1c2ccccc2C[C@@H]1NC(=O)C1(c2ccccc2)CCNC1]

Prepared as described for Example 115 using tert-butyl 3-(((1S,2S)-1-((tert-butoxycarbonyl)amino)-2,3-dihydro-1H-inden-2-yl)carbamoyl)-3-phenylpyrrolidine-1-carboxylate (Intermediate 50, 90 mg, 0.173 mmol) and TFA (0.130 mL, 1.687 mmol). The crude product was purified by reverse phase preparative HPLC eluted with acetonitrile/water (with 0.1% ammonia) to afford the title compound.

¹H NMR (400 MHz, DMSO-d₆) δ ppm 1.83-2.29 (m, 2H), 2.53-2.63 (m, 1H), 2.65-3.17 (m, 5H), 3.21-3.40 (m, 1H), 3.86 (d, J=11.10 Hz, 1H), 3.98-4.17 (m, 2H), 7.10-7.41 (m, 10H), 7.86-8.01 (m, 1H)

MS ES⁺: 322

### Example 121: tert-butyl N-[(1S,2S)-2-(5-oxo-3-phenylpyrrolidine-3-amido)-2,3-dihydro-1H-1-inden-1-yl]carbamate

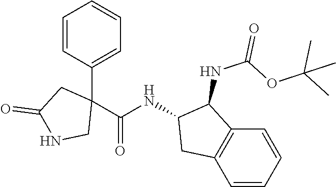 [SMILES: CC(C)(C)OC(=O)N[C@H]1c2ccccc2C[C@@H]1NC(=O)C1(c2ccccc2)CNC(=O)C1]

Prepared as described for Example 1 using 5-oxo-3-phenylpyrrolidine-3-carboxylic acid (50 mg, 0.244 mmol) and tert-butyl ((1S,2S)-2-amino-2,3-dihydro-1H-inden-1-yl)carbamate (60 mg, 0.242 mmol). The crude product was purified by reverse phase preparative HPLC eluted with acetonitrile/water (with 0.1% ammonia) to afford the title compound.

¹H NMR (400 MHz, DICHLOROMETHANE-d₂) δ ppm 1.37-1.52 (m, 9H), 2.45-2.61 (m, 1H), 2.64-2.85 (m, 1H), 3.02-3.25 (m, 1H), 3.36-3.49 (m, 1H), 3.56-3.76 (m, 1H), 4.04-4.29 (m, 2H), 4.88-4.96 (m, 1H), 5.03-5.12 (m, 1H), 5.61-5.72 (m, 1H), 6.56-6.67 (m, 1H), 7.12-7.25 (m, 4H), 7.28-7.46 (m, 5H)

MS ES⁺: 436

### Example 122: N-[(1S,2S)-1-amino-2,3-dihydro-1H-inden-2-yl]-5-oxo-3-phenylpyrrolidine-3-carboxamide

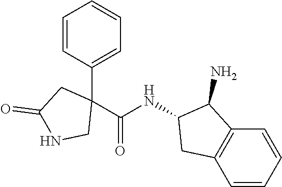 [SMILES: N[C@H]1c2ccccc2C[C@@H]1NC(=O)C1(c2ccccc2)CNC(=O)C1]

Prepared as described for Example 115 using tert-butyl N-[(1S,2S)-2-(5-oxo-3-phenylpyrrolidine-3-amido)-2,3-dihydro-1H-inden-1-yl]carbamate (Example 121, 20 mg, 0.046 mmol) and TFA (0.034 mL, 0.439 mmol). The crude material was purified by reverse phase preparative HPLC eluted with acetonitrile/water (with 0.1% ammonia) to afford the title compound.

¹H NMR (400 MHz, DICHLOROMETHANE-d₂) δ ppm 1.76-1.83 (m, 2H), 2.40-2.55 (m, 1H), 2.71-2.81 (m, 1H), 3.04-3.30 (m, 2H), 3.59-3.69 (m, 1H), 3.91-4.04 (m, 1 H), 4.08-4.27 (m, 2H), 5.57-5.72 (m, 1H), 5.74-5.89 (m, 1H), 7.09-7.35 (m, 7H), 7.37-7.44 (m, 2H)

MS ES⁺: 336

### Example 123: tert-butyl N-[(1S,2S)-2-(3-oxo-1-phenylcyclobutaneamido)-2,3-dihydro-1H-inden-1-yl]carbamate

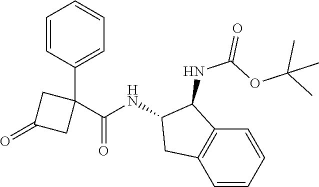 [SMILES: CC(C)(C)OC(=O)N[C@H]1c2ccccc2C[C@@H]1NC(=O)C1(c2ccccc2)CC(=O)C1]

Prepared as described for Example 1 using 3-oxo-1-phenylcyclobutanecarboxylic acid (77 mg, 0.403 mmol) and tert-butyl ((1S,2S)-2-amino-2,3-dihydro-1H-inden-1-yl)carbamate (100 mg, 0.403 mmol). The crude product was purified by column chromatography on silica, eluted with 0-100% ethyl acetate/petroleum ether to afford the title compound.

¹H NMR (400 MHz, DICHLOROMETHANE-d₂) δ ppm 1.50 (s, 9H), 2.47-2.62 (m, 1H), 3.35-3.61 (m, 3H), 3.78-4.04 (m, 2H), 4.10-4.27 (m, 1H), 4.86-5.10 (m, 2H), 6.30-6.45 (m, 1H), 7.14-7.29 (m, 4H), 7.36-7.54 (m, 5H)

MS ES⁺: 421

### Examples 124, 125, 126 and 127: 2-(2,4-difluorophenyl)-N-(trans)-(1-methoxy-2,3-dihydro-1H-inden-2-yl)-2-(6-oxo-1,6-dihydropyridazin-1-yl)acetamide Stereoisomers A, B, C and D

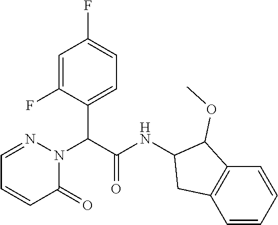 [SMILES: COC1c2ccccc2CC1NC(=O)C(c1ccc(F)cc1F)n1ncccc1=O]

Prepared as described for Example 1 using lithio 2-(2,4-difluorophenyl)-2-(6-oxo-1,6-dihydropyridazin-1-yl)acetate (Intermediate 21, 351 mg, 1.290 mmol) and (trans)-1-methoxy-2,3-dihydro-1H-inden-2-amine (Intermediate 4, 221 mg, 1.354 mmol). The crude material was purified by column chromatography on silica, eluted with 0-100% ethyl acetate/petroleum ether. The stereoisomers were separated by chiral SFC (ID Diacel CHIRALPAK, 21% IPA) to afford 3 peaks. The first was a mixture of two stereoisomers that were further separated by chiral SFC (AD Diacel CHIRALPAK, 20% MeOH) to afford the title compounds.

### Example 124—Stereoisomer A—First Eluting Peak from the First Eluting Peak

¹H NMR (400 MHz, DICHLOROMETHANE-d₂) δ ppm 2.59-2.76 (m, 1H), 3.38-3.54 (m, 4H), 4.54-4.68 (m, 2H), 6.03 (d, J=5.69 Hz, 1H), 6.79 (s, 1H), 6.82-6.99 (m, 3H), 7.16-7.38 (m, 5H), 7.52-7.63 (m, 1H), 7.71-7.77 (m, 1H)

MS ES⁺: 412

### Example 125—Stereoisomer B—Second Eluting Peak from the First Eluting Peak

¹H NMR (400 MHz, DICHLOROMETHANE-d₂) δ ppm 2.59-2.69 (m, 1H), 3.39-3.51 (m, 4H), 4.56-4.68 (m, 2H), 6.02 (d, J=6.88 Hz, 1H), 6.79 (s, 1H), 6.83-6.99 (m, 3H), 7.16-7.40 (m, 5H), 7.52-7.64 (m, 1H), 7.70-7.78 (m, 1H)

MS ES⁺: 412

### Example 126—Stereoisomer C—Second Eluting Peak

¹H NMR (400 MHz, DICHLOROMETHANE-d₂) δ ppm 2.57-2.70 (m, 1H), 3.37-3.53 (m, 4H), 4.55-4.64 (m, 1H), 4.66 (d, J=3.76 Hz, 1H), 6.06 (d, J=7.43 Hz, 1H), 6.79 (s, 1H), 6.83-6.99 (m, 3H), 7.14-7.31 (m, 4H), 7.36 (d, J=6.97 Hz, 1H), 7.52-7.64 (m, 1H), 7.68-7.78 (m, 1H)

MS ES⁺: 412

### Example 127—Stereoisomer D—Third Eluting Peak

¹H NMR (400 MHz, DICHLOROMETHANE-d₂) δ ppm 2.61-2.76 (m, 1H), 3.37-3.51 (m, 4H), 4.54-4.69 (m, 2H), 6.02 (d, J=6.88 Hz, 1H), 6.79 (s, 1H), 6.83-7.00 (m, 3H), 7.16-7.38 (m, 5H), 7.53-7.62 (m, 1H), 7.71-7.77 (m, 1H)

MS ES⁺: 412

### Examples 128, 129, 130 and 131: 2-(4-fluorophenyl)-N-(trans)-(1-methoxy-2,3-dihydro-1H-inden-2-yl)-2-(6-oxo-1,6-dihydropyridazin-1-yl)acetamide Stereoisomers A, B, C and D

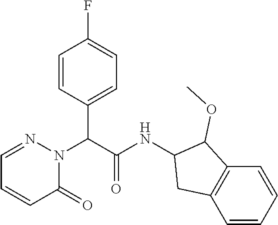 [SMILES: COC1c2ccccc2CC1NC(=O)C(c1ccc(F)cc1)n1ncccc1=O]

Prepared as described for Example 1 using lithio 2-(4-fluorophenyl)-2-(6-oxo-1,6-dihydropyridazin-1-yl)acetate (Intermediate 31, 351 mg, 1.290 mmol) and (trans)-1-methoxy-2,3-dihydro-1H-inden-2-amine (Intermediate 4, 221 mg, 1.354 mmol). The crude material was purified by column chromatography on silica, eluted with 0-100% ethyl acetate/petroleum ether. The stereoisomers were separated by chiral SFC (Lux-C4 Diacel CHIRALPAK, 34% MeOH) to afford 2 peaks. Both were mixtures of two stereoisomers that were further separated by chiral SFC (AD Diacel CHIRALPAK, 18% MeOH, Peak 1) to afford stereoisomers A and B or by chiral SFC (IC Diacel CHIRALPAK, 20% EtOH, Peak 2) to afford the stereoisomers C and D.

### Example 128—Stereoisomer A—First Eluting Peak from the First Eluting Peak

¹H NMR (400 MHz, DICHLOROMETHANE-d₂) δ ppm 2.60-2.73 (m, 1H), 3.37-3.51 (m, 4H), 4.53-4.65 (m, 2H), 6.06 (d, J=5.87 Hz, 1H), 6.63 (s, 1H), 6.86-6.95 (m, 1H), 7.02-7.12 (m, 2H), 7.15-7.36 (m, 5H), 7.42-7.51 (m, 2H), 7.74-7.80 (m, 1H)

MS ES⁺: 416 (M+Na)

### Example 129—Stereoisomer B—Second Eluting Peak from the First Eluting Peak

¹H NMR (400 MHz, DICHLOROMETHANE-d₂) δ ppm 2.58-2.68 (m, 1H), 3.36-3.50 (m, 4H), 4.53-4.67 (m, 2H), 6.06 (d, J=7.06 Hz, 1H), 6.63 (s, 1H), 6.87-6.94 (m, 1H), 7.03-7.12 (m, 2H), 7.15-7.31 (m, 4H), 7.35 (d, J=7.15 Hz, 1H), 7.42-7.51 (m, 2H), 7.73-7.80 (m, 1H)

MS ES⁺: 416 (M+Na)

### Example 130—Stereoisomer C—First Eluting Peak from the Second Eluting Peak

¹H NMR (400 MHz, DICHLOROMETHANE-d₂) δ ppm 2.57-2.68 (m, 1H), 3.38-3.51 (m, 4H), 4.55-4.63 (m, 1H), 4.63-4.65 (m, 1H), 6.06 (d, J=7.15 Hz, 1H), 6.63 (s, 1H), 6.86-6.94 (m, 1H), 7.02-7.12 (m, 2H), 7.15-7.31 (m, 4H), 7.35 (d, J=6.69 Hz, 1H), 7.42-7.52 (m, 2H), 7.72-7.81 (m, 1H)

MS ES⁺: 416 (M+Na)

### Example 131—Stereoisomer D—Second Eluting Peak from the Second Eluting Peak

¹H NMR (400 MHz, DICHLOROMETHANE-d₂) δ ppm 2.61-2.73 (m, 1H), 3.38-3.50 (m, 4H), 4.55-4.65 (m, 2H), 6.06 (d, J=5.96 Hz, 1H), 6.63 (s, 1H), 6.87-6.94 (m, 1H), 7.01-7.11 (m, 2H), 7.15-7.30 (m, 4H), 7.33 (d, J=7.52 Hz, 1H), 7.42-7.51 (m, 2H), 7.73-7.79 (m, 1H)

MS ES⁺: 416 (M+Na)

### Example 132: (2S)-2-(4-fluorophenyl)-N-[(1S,2S)-1-methanesulfonyl-2,3-dihydro-1H-inden-2-yl]propanamide

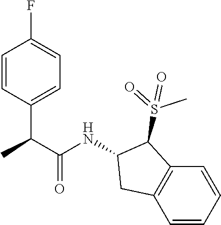 [SMILES: C[C@H](C(=O)N[C@H]1Cc2ccccc2[C@@H]1S(C)(=O)=O)c1ccc(F)cc1]

A solution of methanesulfonic anhydride (0.419 g, 2.405 mmol) in THF (3 mL) was added drop wise to a solution of (S)-2-(4-fluorophenyl)-N-((1R,2S)-1-hydroxy-2,3-dihydro-1H-inden-2-yl)propanamide (Example 62, stereoisomer A, 0.36 g, 1.203 mmol) and triethylamine (0.503 mL, 3.61 mmol) in THF (3 mL) at −78° C. under nitrogen. The reaction was stirred in a salt/ice bath for 20 minutes. A suspension of sodium methanethiolate (0.253 g, 3.61 mmol) and 15-crown-5 (0.714 mL, 3.61 mmol) in THF (1 mL) was added. The reaction was stirred in the ice bath for 1 hour. Further sodium methanethiolate (168 mg, 2.406 mmol) and 15-crown-5 (0.476 mL, 2.406 mmol) in THF (1 mL) was added and the reaction stirred in the ice bath for a further hour. The mixture was partitioned between ethyl acetate and water. The phases were separated and the aqueous extracted with ethyl acetate. The combined organics were washed with saturated brine, dried (phase separator) and concentrated in vacuo to afford crude (2S)-2-(4-fluorophenyl)-N-[(1S,2S)-1-(methylsulfanyl)-2,3-dihydro-1H-inden-2-yl]propanamide. mCPBA (0.673 g, 3.01 mmol) was added to a solution of ((2S)-2-(4-fluorophenyl)-N-[(1S,2S)-1-(methylsulfanyl)-2,3-dihydro-1H-inden-2-yl]propanamide (0.396 g, 1.202 mmol) in DCM (6 mL) under nitrogen. The reaction was stirred at room temperature for 30 minutes. Calcium hydroxide (0.334 g, 4.51 mmol) was added and then stirred for 20 minutes. MgSO₄ was added and the suspension filtered and concentrated in vacuo. The crude product was purified by column chromatography on silica, eluted with 17-70% ethyl acetate/petroleum ether. The product was recrystallised from ethyl acetate/heptanes to afford the title compound.

¹H NMR (300 MHz, DMSO-d₆) δ ppm 1.32 (d, J=7.02 Hz, 3H) 2.78-2.91 (m, 1H) 3.02 (s, 3H) 3.35-3.50 (m, 1H) 3.53-3.65 (m, 1H) 4.51 (d, J=2.06 Hz, 1H) 4.78-4.90 (m, 1H) 7.04-7.17 (m, 2H) 7.21-7.41 (m, 5H) 7.46 (d, J=7.57 Hz, 1H) 8.59 (d, J=7.01 Hz, 1H)

MS ES⁺: 362

### Example 133: (2S)-2-(2,4-difluorophenyl)-N-[(1S,2S)-1-[(pyrimidin-2-yl)amino]-2,3-dihydro-1H-inden-2-yl]propanamide

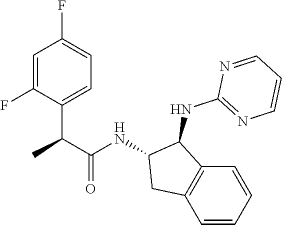 [SMILES: C[C@H](C(=O)N[C@H]1Cc2ccccc2[C@@H]1Nc1ncccn1)c1ccc(F)cc1F]

DIPEA (0.110 mL, 0.632 mmol) was added to a solution of (S)—N-((1S,2S)-1-amino-2,3-dihydro-1H-inden-2-yl)-2-(2,4-difluorophenyl)propanamide (Example 34, 100 mg, 0.316 mmol) and 2-chloropyrimidine (44 mg, 0.384 mmol) in ethanol (1.6 mL) under nitrogen. The reaction was stirred at room temperature for 4 days. The mixture was heated under microwave irradiation at 120° C. for 6.5 hours. The reaction mixture was concentrated under a stream of dry nitrogen. The crude product was purified by reverse phase preparative HPLC eluted with acetonitrile/water (with 0.1% ammonia) to afford the title compound.

¹H NMR (400 MHz, DICHLOROMETHANE-d₂) δ ppm 1.41 (d, J=7.06 Hz, 3H), 2.65-2.77 (m, 1H), 3.55-3.65 (m, 1H), 3.78-3.87 (m, 1H), 4.11-4.23 (m, 1H), 5.45-5.53 (m, 1H), 5.59 (d, J=8.07 Hz, 1H), 6.55 (t, J=4.81 Hz, 1H), 6.66-6.77 (m, 2H), 7.16-7.33 (m, 6H), 8.15 (br. s., 2H)

MS ES⁺: 395

### Example 134: (2S)—N-[(1S,2S)-1-(ethylamino)-2,3-dihydro-1H-inden-2-yl]-2-(4-fluorophenyl)propanamide

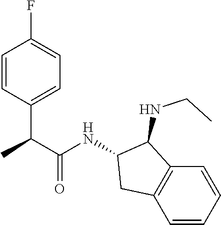 [SMILES: CCN[C@H]1c2ccccc2C[C@@H]1NC(=O)[C@@H](C)c1ccc(F)cc1]

Prepared as described for Example 67 using (2S)-2-(4-fluorophenyl)-N-(cis)-(1-hydroxy-2,3-dihydro-1H-inden-2-yl)propanamide (Example 62, stereoisomer A, 0.100 g, 0.334 mmol) and ethanamine (0.835 mL, 1.670 mmol). The crude product was purified by reverse phase preparative HPLC eluted with acetonitrile/water (with 0.1% ammonia) to afford the title compound.

¹H NMR (400 MHz, DMSO-d₆) δ ppm 0.86 (t, J=7.11 Hz, 3H) 1.32 (d, J=7.06 Hz, 3H) 2.27-2.47 (m, 2H) 2.57-2.69 (m, 1H) 3.09-3.26 (m, 1H) 3.54-3.65 (m, 1H) 3.85-3.95 (m, 1H) 4.12-4.30 (m, 1H) 5.76 (s, 1H) 7.06-7.21 (m, 5H) 7.22-7.27 (m, 1H) 7.31-7.41 (m, 2H) 8.29 (d, J=8.07 Hz, 1H)

MS ES⁺: 327

### Example 135: 2-(cyclopropylmethoxy)-N-(trans)-(1-methanesulfonyl-2,3-dihydro-1H-inden-2-yl)-2-phenylacetamide

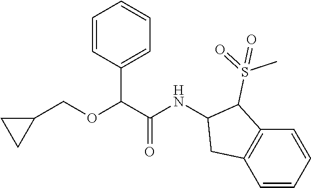 [SMILES: CS(=O)(=O)C1c2ccccc2CC1NC(=O)C(OCC1CC1)c1ccccc1]

Prepared as described for Example 113 using 2-(cyclopropylmethoxy)-N-(trans)-[1-(methylsulfanyl)-2,3-dihydro-1H-inden-2-yl]-2-phenylacetamide (Intermediate 34, 225 mg, 0.613 mmol). The crude material was purified by reverse phase preparative HPLC eluted with acetonitrile/water (with 0.1% formic acid) to afford the title compound.

¹H NMR (400 MHz, DMSO-d₆) δ ppm 0.02-0.23 (m, 2H) 0.32-0.54 (m, 2H) 1.04 (d, J=7.70 Hz, 1H) 2.79-3.04 (m, 4H) 3.16-3.48 (m, 3H) 4.67-4.98 (m, 3H) 7.19-7.46 (m, 8H) 7.48-7.59 (m, 1H) 8.55-8.79 (m, 1H)

MS ES⁺: 400

### Examples 136, 137, 138 and 139: 2-(2,4-difluorophenyl)-2-(3-fluoroazetidin-1-yl)-N-(trans)-(1-methoxy-2,3-dihydro-1H-inden-2-yl)acetamide Stereoisomers A, B, C and D

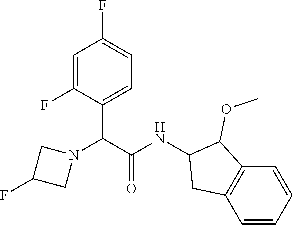 [SMILES: COC1c2ccccc2CC1NC(=O)C(c1ccc(F)cc1F)N1CC(F)C1]

Prepared as described for Example 1 using lithio 2-(2,4-difluorophenyl)-2-(3-fluoroazetidin-1-yl)acetate (Intermediate 14, 153 mg, 0.609 mmol) and (trans)-1-methoxy-2,3-dihydro-1H-inden-2-amine (Intermediate 4, 119 mg, 0.731 mmol). The crude product was purified by column chromatography on silica, eluted with 0-50% ethyl acetate/petroleum ether to afford a mixture of the four stereoisomers. The four stereoisomers were purified by chiral SFC (AD Diacel CHIRALPAK, 12% EtOH) to afford three peaks (stereoisomers A, B and a mixture of stereoisomers C and D). Peak three (stereoisomers C and D) was further purified by chiral SFC (Lux-C4 Diacel CHIRALPAK, 24% MeOH) to afford stereoisomers C and D.

### Example 136—Stereoisomer A—First Eluting Peak

¹H NMR (400 MHz, DMSO-d₆) δ ppm 2.71-2.83 (m, 1H), 3.07-3.29 (m, 3H), 3.36 (s, 3H), 3.40-3.52 (m, 1H), 3.55-3.70 (m, 1H), 4.26-4.43 (m, 2H), 4.78 (d, J=5.41 Hz, 1H), 5.05-5.31 (m, 1H), 7.06-7.36 (m, 6H), 7.48-7.66 (m, 1H), 8.53 (d, J=8.44 Hz, 1H)

MS ES⁺: 391

### Example 137—Stereoisomer B—Second Eluting Peak

¹H NMR (400 MHz, DMSO-d₆) δ ppm 2.70-2.81 (m, 1H), 3.07-3.30 (m, 6H), 3.40-3.52 (m, 1H), 3.54-3.68 (m, 1H), 4.26-4.44 (m, 2H), 4.80 (d, J=5.23 Hz, 1H), 5.05-5.32 (m, 1H), 7.08-7.17 (m, 1H), 7.18-7.33 (m, 5H), 7.51-7.65 (m, 1H), 8.53 (d, J=8.53 Hz, 1H)

MS ES⁺: 391

### Example 138—Stereoisomer C—First Eluting Peak from the Third Eluting Peak

¹H NMR (400 MHz, DMSO-d₆) δ ppm 2.72-2.83 (m, 1H), 3.10-3.28 (m, 3H), 3.36 (s, 3H), 3.39-3.52 (m, 1H), 3.55-3.68 (m, 1H), 4.28-4.40 (m, 2H), 4.78 (d, J=5.50 Hz, 1H), 5.07-5.31 (m, 1H), 7.08-7.17 (m, 1H), 7.18-7.33 (m, 5H), 7.51-7.63 (m, 1H), 8.53 (d, J=8.62 Hz, 1H)

MS ES⁺: 391

### Example 139—Stereoisomer D—Second Eluting Peak from the Third Eluting Peak

¹H NMR (400 MHz, DMSO-d₆) δ ppm 2.70-2.81 (m, 1H), 3.06-3.29 (m, 6H), 3.39-3.52 (m, 1H), 3.55-3.68 (m, 1H), 4.27-4.42 (m, 2H), 4.79 (d, J=5.41 Hz, 1H), 5.05-5.31 (m, 1H), 7.05-7.35 (m, 6H), 7.52-7.64 (m, 1H), 8.53 (d, J=8.53 Hz, 1H)

MS ES⁺: 391

### Examples 140, 141, 142 and 143: 2-(4-fluorophenyl)-2-(3-fluoroazetidin-1-yl)-N-(trans)-(1-methoxy-2,3-dihydro-1H-inden-2-yl)acetamide Stereoisomers A, B, C and D

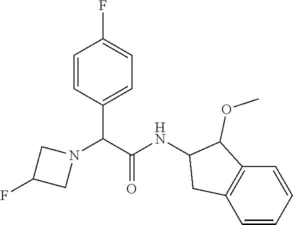 [SMILES: COC1c2ccccc2CC1NC(=O)C(c1ccc(F)cc1)N1CC(F)C1]

Prepared as described for Example 1 using lithio 2-(3-fluoroazetidin-1-yl)-2-(4-fluorophenyl)acetate (Intermediate 24, 124 mg, 0.532 mmol) and (trans)-1-methoxy-2,3-dihydro-1H-inden-2-amine (Intermediate 4, 104 mg, 0.638 mmol). The crude product was purified by column chromatography on silica, eluted with 0-50% ethyl acetate/petroleum ether to afford a mixture of the four stereoisomers. The four stereoisomers were purified by chiral SFC (IC Diacel CHIRALPAK, 36% IPA) to afford two peaks (a mixture of stereoisomers A, B and C and stereoisomer D). Peak 1 (stereoisomers A, B and C) was further purified by chiral SFC (AD Diacel CHIRALPAK, 18% EtOH) to afford stereoisomer A and a mixture of stereoisomers B and C. Peak 2 of the second run (stereoisomers B and C) was further purified by chiral SFC (Lux-C4 Diacel CHIRALPAK, 14% MeOH) to afford stereoisomers B and C.

### Example 140—Stereoisomer A—First Eluting Peak from First Eluting Peak

¹H NMR (400 MHz, DMSO-d₆) δ ppm 2.69-2.79 (m, 1H), 3.08-3.24 (m, 6H), 3.33-3.43 (m, 1H), 3.56-3.69 (m, 1H), 3.98 (s, 1H), 4.27-4.39 (m, 1H), 4.77 (d, J=5.59 Hz, 1H), 5.08-5.30 (m, 1H), 7.12-7.30 (m, 6H), 7.41-7.50 (m, 2H), 8.44 (d, J=8.71 Hz, 1H)

MS ES⁺: 373

### Example 141—Stereoisomer B—First Eluting Peak from Second Eluting Peak from First Eluting Peak

¹H NMR (400 MHz, DMSO-d₆) δ ppm 2.70-2.80 (m, 1H), 3.03-3.24 (m, 3H), 3.32-3.48 (m, 4H), 3.56-3.68 (m, 1H), 3.98 (s, 1H), 4.27-4.39 (m, 1H), 4.76 (d, J=5.69 Hz, 1H), 5.05-5.33 (m, 1H), 7.13-7.31 (m, 6H), 7.40-7.50 (m, 2H), 8.44 (d, J=8.53 Hz, 1H)

MS ES⁺: 373

### Example 142—Stereoisomer C—Second Eluting Peak from Second Eluting Peak from First Eluting Peak

¹H NMR (400 MHz, DMSO-d₆) δ ppm 2.69-2.79 (m, 1H), 3.08-3.23 (m, 6H), 3.33-3.43 (m, 1H), 3.56-3.69 (m, 1H), 3.98 (s, 1H), 4.28-4.39 (m, 1H), 4.77 (d, J=5.59 Hz, 1H), 5.07-5.32 (m, 1H), 7.12-7.31 (m, 6H), 7.40-7.51 (m, 2H), 8.45 (d, J=8.71 Hz, 1H)

MS ES⁺: 373

### Example 143—Stereoisomer D—Second Eluting Peak

¹H NMR (400 MHz, DMSO-d₆) δ ppm 2.70-2.79 (m, 1H), 3.03-3.21 (m, 3H), 3.32 (s, 3H), 3.33-3.40 (m, 1H), 3.55-3.67 (m, 1H), 3.97 (s, 1H), 4.26-4.39 (m, 1H), 4.75 (d, J=5.50 Hz, 1H), 5.06-5.29 (m, 1H), 7.11-7.31 (m, 6H), 7.39-7.49 (m, 2H), 8.44 (d, J=8.62 Hz, 1H)

MS ES⁺: 373

### Example 144: (2R)-2-(cyclopropylformamido)-2-(4-fluorophenyl)-N-(trans)-(1-methoxy-2,3-dihydro-1H-inden-2-yl)acetamide

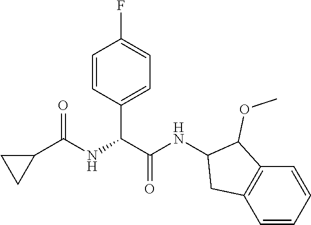 [SMILES: COC1c2ccccc2CC1NC(=O)[C@H](NC(=O)C1CC1)c1ccc(F)cc1]

Prepared as described for Example 11 using 2-(cyclopropylformamido)-2-(4-fluorophenyl)acetic acid (Intermediate 35, 0.275 g, 1.159 mmol) and (trans)-1-methoxy-2,3-dihydro-1H-inden-2-amine (Intermediate 4, 0.208 g, 1.275 mmol). The crude material was purified by column chromatography on silica eluted with 18-75% ethyl acetate/petroleum ether to afford the product as a mixture of diastereomers. The crude material was purified by chiral SFC (AD Diacel CHIRALPAK, 32% EtOH) to afford 3 peaks with peak 2 corresponding to the title compound.

¹H NMR (400 MHz, DMSO-d₆) δ ppm 0.58-0.71 (m, 4H) 1.85-1.92 (m, 1H) 2.53-2.76 (m, 1H) 3.14-3.41 (m, 4H) 4.24-4.34 (m, 1H) 4.47-4.68 (m, 1H) 5.50 (d, J=8.25 Hz, 1H) 7.13-7.50 (m, 8H) 8.67-8.87 (m, 2H)

MS ES⁻: 381

### Examples 145, 146, 147 and 148: 2-(cyclopropylmethoxy)-N-(trans)-(1-methanesulfonyl-2,3-dihydro-1H-inden-2-yl)-2-phenylacetamide Stereoisomers A, B, C and D

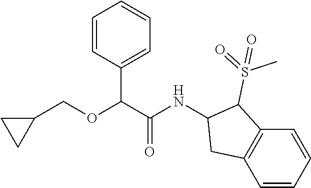 [SMILES: CS(=O)(=O)C1c2ccccc2CC1NC(=O)C(OCC1CC1)c1ccccc1]

Example 135 was separated by chiral SFC (Lux-C4 Diacel CHIRALPAK, 32% EtOH) to afford three peaks. Peak 1 was a mixture of two stereoisomers (C and D) and peaks 2 and 3 were separate stereoisomers (A and B respectively). Peak 1 was further purified by chiral SFC (IC Diacel CHIRALPAK, 40% IPA) to afford peaks 1 and 2 as stereoisomers C and D respectively.

### Example 145—Stereoisomer A—Second Eluting Peak

¹H NMR (400 MHz, DMSO-d₆) δ ppm 0.08-0.15 (m, 2H) 0.39-0.46 (m, 2H) 0.95-1.08 (m, 1H) 2.86-2.95 (m, 1H) 3.00 (s, 3H) 3.16-3.24 (m, 1H) 3.34-3.43 (m, 1H) 4.70-4.77 (m, 1H) 4.80 (s, 1H) 4.84-4.93 (m, 1H) 7.24-7.43 (m, 8H) 7.47-7.57 (m, 1H) 8.57-8.68 (m, 1H)

MS ES⁺: 400

### Example 146—Stereoisomer B—Third Eluting Peak

¹H NMR (400 MHz, DMSO-d₆) δ ppm 0.07-0.24 (m, 2H), 0.38-0.50 (m, 2H), 0.95-1.11 (m, 1H), 2.77-2.95 (m, 4H), 3.15-3.25 (m, 1H), 3.34-3.49 (m, 1H), 4.76-4.94 (m, 3H), 7.24-7.46 (m, 8H), 7.50-7.56 (m, 1H), 8.66-8.74 (m, 1H)

MS ES⁺: 400

### Example 147—Stereoisomer C—First Eluting Peak from First Eluting Peak

¹H NMR (400 MHz, DMSO-d₆) δ ppm 0.07-0.17 (m, 2H), 0.39-0.47 (m, 2H), 0.97-1.08 (m, 1H), 2.87-2.96 (m, 1H), 3.00 (s, 3H), 3.16-3.25 (m, 1H), 3.34-3.43 (m, 1H), 4.71-4.76 (m, 1H), 4.81 (s, 1H), 4.85-4.93 (m, 1H), 7.24-7.43 (m, 8H), 7.47-7.54 (m, 1H), 8.59-8.67 (m, 1H)

MS ES⁺: 400

### Example 148—Stereoisomer D—Second Eluting Peak from First Eluting Peak

¹H NMR (400 MHz, DMSO-d₆) δ ppm 0.10-0.21 (m, 2H), 0.42-0.49 (m, 2H), 0.99-1.09 (m, 1H), 2.82-2.90 (m, 1H), 2.92 (s, 3H), 3.15-3.25 (m, 1H), 3.35-3.46 (m, 1H), 4.77-4.84 (m, 2H), 4.85-4.94 (m, 1H), 7.24-7.45 (m, 8H), 7.50-7.56 (m, 1H), 8.65-8.74 (m, 1H)

MS ES⁺: 400

### Examples 149 and 150: (2S)—N-(trans)-(1-methanesulfonyl-2,3-dihydro-1H-inden-2-yl)-2-methoxy-2-phenylacetamide and (2S)—N-(trans)-[1-(ethanesulfonyl)-2,3-dihydro-1H-inden-2-yl]-2-methoxy-2-phenylacetamide

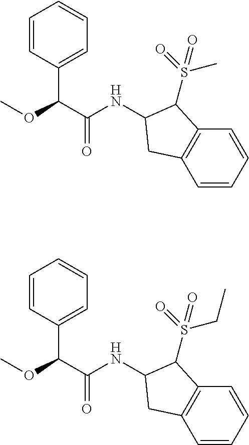 [SMILES: CO[C@H](C(=O)NC1Cc2ccccc2C1S(C)(=O)=O)c1ccccc1 ; CCS(=O)(=O)C1c2ccccc2CC1NC(=O)[C@@H](OC)c1ccccc1]

A mixture of (2S)-2-methoxy-N-(trans)-[1-(methylsulfanyl)-2,3-dihydro-1H-inden-2-yl]-2-phenylacetamide and (2S)-2-methoxy-N-(trans)-[1-(ethylsulfanyl)-2,3-dihydro-1H-inden-2-yl]-2-phenylacetamide (Intermediate 38, 218 mg, 0.666 mmol) was dissolved in DCM (10 mL) and mCPBA (287 mg, 1.665 mmol) was added. The reaction was stirred at room temperature for 2 hours. The reaction mixture was washed with saturated aq. NaHCO₃, dried (phase separator) and concentrated in vacuo. The crude product was purified by reverse phase preparative HPLC eluted with acetonitrile/water (with 0.1% ammonia) to afford the title compounds.

### Example 149: (2S)—N-(trans)-(1-methanesulfonyl-2,3-dihydro-1H-inden-2-yl)-2-methoxy-2-phenylacetamide

¹H NMR (300 MHz, DMSO-d₆) δ ppm 2.81-2.96 (m, 4H), 3.31 (s, 3H), 3.35-3.47 (m, 1H), 4.67 (s, 1H), 4.78-4.97 (m, 2H), 7.23-7.45 (m, 8H), 7.53 (d, J=7.57 Hz, 1H), 8.79 (d, J=7.29 Hz, 1H)

MS ES⁺: 360

### Example 150: (2S)—N-(trans)-(1-ethanesulfonyl-2,3-dihydro-1H-inden-2-yl)-2-methoxy-2-phenylacetamide

¹H NMR (300 MHz, DMSO-d₆) δ ppm 1.10 (t, J=7.43 Hz, 3H), 2.80-3.18 (m, 3H), 3.28 (s, 3H), 3.40 (s, 1H), 4.68 (s, 1H), 4.76-4.94 (m, 2H), 7.19-7.47 (m, 8H), 7.53 (d, J=7.63 Hz, 1H), 8.79 (d, J=7.70 Hz, 1H)

MS ES⁺: 374

### Example 151: tert-butyl N-[(1S,2S)-2-{2-[4-(difluoromethoxy)phenyl]propanamido}-2,3-dihydro-1H-inden-1-yl]carbamate

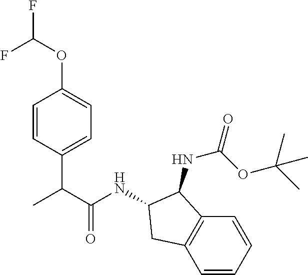 [SMILES: CC(C(=O)N[C@H]1Cc2ccccc2[C@@H]1NC(=O)OC(C)(C)C)c1ccc(OC(F)F)cc1]

Prepared as described for Example 1 using lithio 2-[4-(difluoromethoxy)phenyl]propanoate (Intermediate 39, 84 mg, 0.378 mmol) and tert-butyl ((1S,2S)-2-amino-2,3-dihydro-1H-inden-1-yl)carbamate (94 mg, 0.378 mmol). The crude material was purified by reverse phase preparative HPLC eluted with acetonitrile/water (with 0.1% ammonia) to afford the title compound.

¹H NMR (400 MHz, DICHLOROMETHANE-d₂) δ ppm 1.36-1.51 (m, 12H), 2.49-2.67 (m, 1H), 3.30-3.48 (m, 1H), 3.49-3.62 (m, 1H), 4.03-4.24 (m, 1H), 4.92-5.07 (m, 2H), 6.30-6.73 (m, 2H), 7.02-7.11 (m, 2H), 7.13-7.26 (m, 4H), 7.29-7.37 (m, 2H)

MS ES⁺: 447

### Example 152: tert-butyl N-[(1S,2S)-2-[2-(4-fluoro-2-methoxyphenyl)propanamido]-2,3-dihydro-1H-inden-1-yl]carbamate

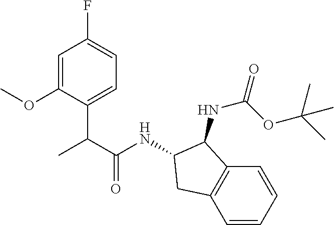 [SMILES: COc1cc(F)ccc1C(C)C(=O)N[C@H]1Cc2ccccc2[C@@H]1NC(=O)OC(C)(C)C]

Prepared as described for Example 1 using 2-(4-fluoro-2-methoxyphenyl)propanoic acid (Intermediate 40, 64 mg, 0.323 mmol) and tert-butyl ((1S,2S)-2-amino-2,3-dihydro-1H-inden-1-yl)carbamate (120 mg, 0.484 mmol). The crude material was purified by reverse phase preparative HPLC eluted with acetonitrile/water (with 0.1% ammonia) to afford the title compound.

¹H NMR (400 MHz, DICHLOROMETHANE-d₂) δ ppm 1.36-1.50 (m, 12H), 2.45-2.70 (m, 1H), 3.26-3.48 (m, 1H), 3.80-3.97 (m, 4H), 4.11-4.28 (m, 1H), 4.83-5.09 (m, 2H), 6.41-6.55 (m, 1H), 6.60-6.73 (m, 2H), 7.11-7.31 (m, 5H)

MS ES⁺: 429

### Example 153: tert-butyl N-[(1S,2S)-2-[2-(2-chloro-4-fluorophenyl)propanamido]-2,3-dihydro-1H-inden-1-yl]carbamate

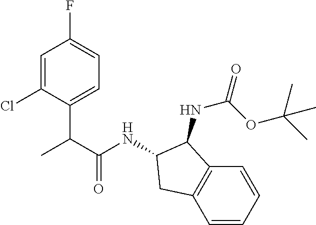 [SMILES: CC(C(=O)N[C@H]1Cc2ccccc2[C@@H]1NC(=O)OC(C)(C)C)c1ccc(F)cc1Cl]

Prepared as described for Example 1 using 2-(2-chloro-4-fluorophenyl)propanoic acid (Intermediate 41, 90 mg, 0.444 mmol) and tert-butyl ((1S,2S)-2-amino-2,3-dihydro-1H-inden-1-yl)carbamate (110 mg, 0.444 mmol). The crude material was purified by reverse phase preparative HPLC eluted with acetonitrile/water (with 0.1% ammonia) to afford the title compound.

¹H NMR (400 MHz, DICHLOROMETHANE-d₂) δ ppm 1.36-1.52 (m, 12H), 2.51-2.71 (m, 1H), 3.32-3.50 (m, 1H), 3.94-4.06 (m, 1H), 4.09-4.29 (m, 1H), 4.93-5.08 (m, 2H), 6.50-6.63 (m, 1H), 6.98-7.08 (m, 1H), 7.11-7.27 (m, 5H), 7.40-7.49 (m, 1H)

MS ES⁺: 433

### Example 154: tert-butyl N-[(1S,2S)-2-{2-[4-fluoro-2-(trifluoromethyl)phenyl]propanamido}-2,3-dihydro-1H-inden-1-yl]carbamate

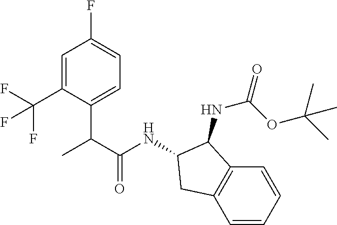 [SMILES: CC(C(=O)N[C@H]1Cc2ccccc2[C@@H]1NC(=O)OC(C)(C)C)c1ccc(F)cc1C(F)(F)F]

Prepared as described for Example 1 using 2-[4-fluoro-2-(trifluoromethyl)phenyl]propanoic acid (Intermediate 42, 35 mg, 0.148 mmol) and tert-butyl ((1S,2S)-2-amino-2,3-dihydro-1H-inden-1-yl)carbamate (37 mg, 0.149 mmol). The crude material was purified by reverse phase preparative HPLC eluted with acetonitrile/water (with 0.1% ammonia) to afford the title compound.

¹H NMR (400 MHz, DICHLOROMETHANE-d₂) δ ppm 1.31-1.53 (m, 12H), 2.46-2.70 (m, 1H), 3.29-3.57 (m, 1H), 3.85-3.98 (m, 1H), 4.03-4.23 (m, 1H), 4.92-5.08 (m, 2H), 6.43-6.62 (m, 1H), 7.13-7.32 (m, 5H), 7.33-7.42 (m, 1H), 7.66-7.75 (m, 1H)

MS ES⁺: 467

### Example 155: (2R)-2-(4-fluorophenyl)-N-[(1R,2R)-1-(methylamino)-2,3-dihydro-1H-inden-2-yl]propanamide

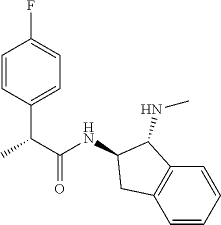 [SMILES: CN[C@@H]1c2ccccc2C[C@H]1NC(=O)[C@H](C)c1ccc(F)cc1]

Prepared as described for Example 11 using (R)-2-(4-fluorophenyl)propanoic acid (60 mg, 0.357 mmol) and tert-butyl N-[(2R)-2-amino-2,3-dihydro-1H-inden-1-yl]-N-methylcarbamate (Intermediate 43, 94 mg, 0.357 mmol). The crude material was purified by reverse phase preparative HPLC eluted with acetonitrile/water (with 0.1% ammonia) to afford the title compound.

¹H NMR (400 MHz, DMSO-d₆) δ ppm 1.33 (d, J=6.88 Hz, 3H), 2.15 (s, 3H), 2.58-2.69 (m, 1H), 3.14-3.28 (m, 1H), 3.53-3.66 (m, 1H), 3.80-3.88 (m, 1H), 4.15-4.29 (m, 1H), 7.05-7.28 (m, 6H), 7.31-7.42 (m, 2H), 8.28 (d, J=7.43 Hz, 1H)

MS ES⁺: 313

### Example 156: tert-butyl N-[(1S,2S)-2-[2-phenyl-3-(pyrrolidin-1-yl)propanamido]-2,3-dihydro-1H-inden-1-yl]carbamate Diastereomeric Mixture

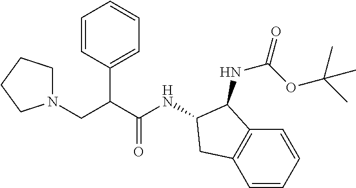 [SMILES: CC(C)(C)OC(=O)N[C@H]1c2ccccc2C[C@@H]1NC(=O)C(CN1CCCC1)c1ccccc1]

Methanesulfonic anhydride (43.9 mg, 0.252 mmol) was added as a solution in THF (0.5 mL) to an ice bath cooled solution of tert-butyl ((1S,2S)-2-(3-hydroxy-2-phenylpropanamido)-2,3-dihydro-1H-inden-1-yl)carbamate (Intermediate 15, 50 mg, 0.126 mmol) and triethylamine (0.051 mL, 0.378 mmol) in THF (1 mL). After 30 minutes, pyrrolidine (44.8 mg, 0.631 mmol) was added and the reaction left for 18 hours. The reaction mixture was partitioned between DCM and water and the organics was collected, dried (phase separator) and concentrated in vacuo. The resulting residue was purified by reverse phase preparative HPLC eluted with acetonitrile/water (with 0.1% ammonia) to afford the title compound as a diastereomeric mixture, as racemisation was observed during the reaction.

¹H NMR (400 MHz, METHANOL-d₄) δ ppm 1.38-1.55 (m, 9H), 1.74-1.87 (m, 4H), 2.53-2.84 (m, 7H), 3.11-3.30 (m, 1H), 3.35-3.43 (m, 1H), 3.74-3.86 (m, 1H), 4.30-4.47 (m, 1H), 4.94-5.07 (m, 1H), 7.11-7.44 (m, 10H)

MS ES⁺: 450

### Example 157: (2R)-2-(4-fluorophenyl)-N-[(1S,2S)-1-(methylamino)-2,3-dihydro-1H-inden-2-yl]propanamide

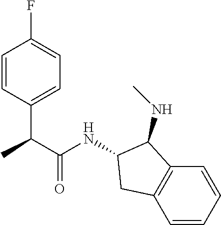 [SMILES: CN[C@H]1c2ccccc2C[C@@H]1NC(=O)[C@@H](C)c1ccc(F)cc1]

Prepared as described for Example 11 using (R)-2-(4-fluorophenyl)propanoic acid (60 mg, 0.357 mmol) and tert-butyl ((1S,2S)-2-amino-2,3-dihydro-1H-inden-1-yl)(methyl)carbamate (84 mg, 0.321 mmol) (Intermediate 44, 94 mg, 0.357 mmol). The crude material was purified by cation exchange cartridge, loading with MeOH and eluting with 2M NH₃ in MeOH to afford the title compound.

¹H NMR (300 MHz, CD₂Cl₂) δ ppm 1.39-1.50 (m, 3H), 2.45-2.59 (m, 4H), 3.27-3.41 (m, 1H), 3.45-3.57 (m, 1H), 3.98-4.12 (m, 1H), 4.35-4.53 (m, 1H), 5.78-6.01 (m, 1H), 6.95-7.09 (m, 2H), 7.11-7.40 (m, 6H)

MS ES⁺: 313

### Examples 158 and 159: (2R)—N-(trans)-(1-methanesulfonyl-2,3-dihydro-1H-inden-2-yl)-2-methoxy-2-phenylacetamide and (2S)—N-(trans)-(1-methanesulfonyl-2,3-dihydro-1H-inden-2-yl)-2-methoxy-2-phenylacetamide Stereoisomers A and B

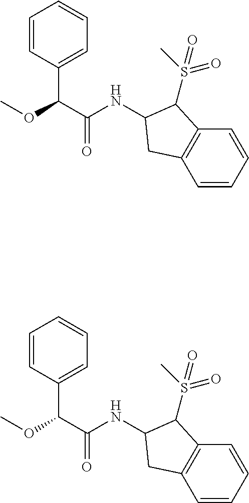 [SMILES: CO[C@H](C(=O)NC1Cc2ccccc2C1S(C)(=O)=O)c1ccccc1 ; CO[C@@H](C(=O)NC1Cc2ccccc2C1S(C)(=O)=O)c1ccccc1]

Methanesulfonic anhydride (0.232 g, 1.332 mmol) was added as a solution in THF (2 mL) to an acetone/dry ice cooled solution of (2 S)—N-(trans)-(1-hydroxy-2,3-dihydro-1H-inden-2-yl)-2-methoxy-2-phenylacetamide (Intermediate 37 (which had been left standing for about 6 weeks and had epimerised at the OMe position), 0.198 g, 0.666 mmol) and triethylamine (0.271 mL, 1.998 mmol) in THF (4 mL) and the reaction was warmed to 0° C. for 30 mins. Sodium methanethiolate (0.233 mg, 3.33 mmol) and 15-crown-5 (733 mg, 3.33 mmol) were added and the reaction was warmed to room temperature for 18 hours. The reaction was partitioned between DCM and water. The organic phase was dried (phase separator) and concentrated in vacuo. The residue was taken up in DCM (2 mL). mCPBA (287 mg, 1.665 mmol) was added and the reaction was stirred at room temperature for 2 hours. The reaction mixture was washed with saturated aq. NaHCO₃ solution and purified by column chromatography on silica, eluted with 0-100% ethyl acetate/petroleum ether to afford a mixture of products. The residue was purified by chiral SFC (Lux-C4 Diacel CHIRALPAK, 23% EtOH) to afford the title compounds.

### Example 158: (2R)—N-(trans)-(1-methanesulfonyl-2,3-dihydro-1H-inden-2-yl)-2-methoxy-2-phenylacetamide—Stereoisomer A—First Eluting Peak

¹H NMR (300 MHz, DMSO-d₆) δ ppm 2.70-3.06 (m, 4H), 3.27 (s, 3H), 3.35-3.47 (m, 1H), 4.67 (s, 1H), 4.76-4.98 (m, 2H), 7.23-7.42 (m, 7H), 7.49-7.58 (m, 1H), 8.71-8.85 (m, 1H)

MS ES⁺: 360

### Example 159: (2S)—N-(trans)-(1-methanesulfonyl-2,3-dihydro-1H-inden-2-yl)-2-methoxy-2-phenylacetamide—Stereoisomer B—Second Eluting Peak

¹H NMR (400 MHz, DMSO-d₆) δ ppm 2.87-2.95 (m, 1H), 3.03 (s, 3H), 3.27 (s, 3H), 3.35-3.44 (m, 1H), 4.67 (s, 1H), 4.74-4.82 (m, 1H), 4.83-4.96 (m, 1H), 7.22-7.41 (m, 7H), 7.47-7.55 (m, 1H), 8.67-8.80 (m, 1H)

MS ES⁺: 360

### Example 160: (2S)-2-(4-fluorophenyl)-N-[(1R,2S)-1-(methylamino)-2,3-dihydro-1H-inden-2-yl]propanamide

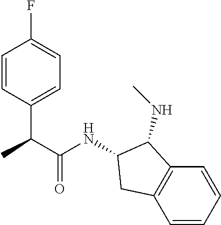 [SMILES: CN[C@@H]1c2ccccc2C[C@@H]1NC(=O)[C@@H](C)c1ccc(F)cc1]

Prepared as described for Example 11 using (R)-2-(4-fluorophenyl)propanoic acid (27 mg, 0.240 mmol) and tert-butyl N-[(1R,2S)-2-amino-2,3-dihydro-1H-inden-1-yl]-N-methylcarbamate (42 mg, 0.160 mmol) (Intermediate 45, 94 mg, 0.357 mmol). The crude material was purified by cation exchange cartridge, loading with MeOH and eluting with 2M NH₃ in MeOH to afford the title compound.

¹H NMR (400 MHz, DMSO-d₆) δ ppm 1.25-1.34 (m, 3H), 1.99 (s, 3H), 2.74-2.85 (m, 1H), 2.98-3.12 (m, 1H), 3.62-3.74 (m, 1H), 3.89-4.02 (m, 1H), 4.44-4.58 (m, 1H), 7.02-7.45 (m, 8H), 7.82-7.95 (m, 1H)

MS ES⁺: 313

### Example 161: 2-(cyclopropylmethoxy)-2-(4-fluorophenyl)-N-(trans)-(1-methanesulfonyl-2,3-dihydro-1H-inden-2-yl)acetamide

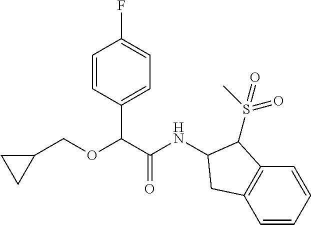 [SMILES: CS(=O)(=O)C1c2ccccc2CC1NC(=O)C(OCC1CC1)c1ccc(F)cc1]

Prepared as described for Example 113 using 2-(cyclopropylmethoxy)-2-(4-fluorophenyl)-N-(trans)-(1-(methylthio)-2,3-dihydro-1H-inden-2-yl)acetamide (Intermediate 48, 0.600 g, 1.556 mmol). The crude material was purified by reverse phase preparative HPLC eluted with acetonitrile/water (with 0.1% ammonia) to afford the title compound.

¹H NMR (400 MHz, DMSO-d₆) δ ppm 0.02-0.20 (m, 2H), 0.36-0.51 (m, 2H), 0.95-1.09 (m, 1H), 2.78-2.90 (m, 1H), 2.92-3.05 (m, 3H), 3.13-3.24 (m, 1H), 3.29-3.32 (m, 1H), 3.35-3.48 (m, 1H), 4.79-4.93 (m, 2H), 7.08-7.56 (m, 8H), 8.68-8.79 (m, 1H)

MS ES⁺: 418

### Example 162: 2-(cyclopropylmethoxy)-2-(4-fluorophenyl)-N-(trans)-(1-methanesulfonyl-2,3-dihydro-1H-inden-2-yl)acetamide

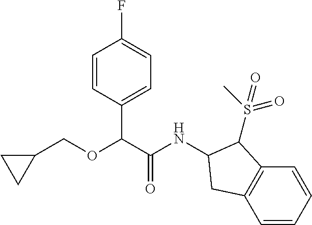 [SMILES: CS(=O)(=O)C1c2ccccc2CC1NC(=O)C(OCC1CC1)c1ccc(F)cc1]

Prepared as described for Example 113 using 2-(cyclopropylmethoxy)-2-(4-fluorophenyl)-N-(trans)-(1-(methylthio)-2,3-dihydro-1H-inden-2-yl)acetamide (Intermediate 49, 0.716 g, 1.857 mmol). The crude material was purified by reverse phase preparative HPLC eluted with acetonitrile/water (with 0.1% ammonia) to afford the title compound.

¹H NMR (400 MHz, DMSO-d₆) δ ppm 0.03-0.18 (m, 2H), 0.35-0.53 (m, 2H), 0.92-1.10 (m, 1H), 2.83-3.04 (m, 4H), 3.13-3.22 (m, 1H), 3.27-3.31 (m, 1H), 3.34-3.46 (m, 1H), 4.70-4.76 (m, 1H), 4.79-4.91 (m, 2H), 7.05-7.57 (m, 8H), 8.61-8.69 (m, 1H)

MS ES⁺: 418

### 3. BIOLOGICAL EFFICACY OF COMPOUNDS OF THE INVENTION MGLUR7 ASSAY

The ability of the test compounds to activate mGluR7 was determined by their ability to reduce forskolin stimulated cAMP production. Compounds were assessed in a CRE-directed luciferase reporter gene assay, using a stable CHO cell line expressing the CRE-luc reporter and human mGluR7 genes. In this cell line, production of cAMP stimulated the transcription of the luciferase gene and luciferase activity was then measured in a luminescent enzyme assay (Steady Glo assay; Promega E2550). Activation of mGluR7 decreased the forskolin stimulated luminescence signal.

The day prior to the assay, compounds were serially diluted in DMSO (100× final assay concentration (FAC)), in 384-well plates which were then stored in the dark at room temperature (RT) until use. Cells were seeded at 12.5 k/well in white, clear bottom 384-well plates (Corning 3707) and left for one hour at RT followed by an overnight incubation (37° C.). The following day, the DMSO compound plate was diluted 1:20 (5× FAC) in Opti-MEM I (Life Technologies 11058021). The growth media was removed from the cell plate and replaced with 15 μl Opti-MEM I, followed by a 5 μl addition from the 5× compound plate and a fifteen minute incubation (37° C.). Forskolin (Sigma F3917) was then added to the wells (5 μl of 2.5 μM) and the plate was incubated for five hours (37° C.). During this incubation, the Steady Glo Substrate reagent was warmed to 37° C. Aliquots (11 ml; stored at −20° C.) of this reagent were prepared by dissolving the contents of 1 vial of lyophilised substrate in 100 ml Steady-Glo buffer. A 25 μl addition of the substrate was made to all wells and the plate was incubated for thirty minutes at RT, on a plate shaker (300 rpm; in the dark). Luminescence was then measured using the EnVision Multilabel Reader (Perkin Elmer).

Compound activity was examined using a 10-point, half log concentration-response range and each concentration was tested in duplicate wells. Luminescence values were normalised to ‘maximum’ (forskolin alone) and ‘minimum’ (forskolin in the presence of tool mGluR7 agonist) controls. EC₅₀ values were derived from this data using non-linear regression and a four parameter curve fit. The EC₅₀ values for the compounds of the Examples are shown in Table 1.

### Results

TABLE 1

| Ex No. | EC₅₀ (nM) | Ex No. | EC₅₀ (nM) | Ex No. | EC₅₀ (nM) |
|---|---|---|---|---|---|
| 1 | 559 | 2 | 107 | 3 | 349 |
| 4 | 121 | 5 | 654 | 6 | 1556 |
| 7 | 29 | 8 | 57 | 9 | 37 |
| 10 | 1030 | 11 | 119 | 12 | 290 |
| 13 | 1203 | 14 | 139 | 15 | 25 |
| 16 | 747 | 17 | 195 | 18 | 150 |
| 19 | 973 | 20 | 104 | 21 | 28 |
| 22 | 70 | 23 | 18 | 24 | 66 |
| 25 | 24 | 26 | 64 | 27 | 21 |
| 28 | 28 | 29 | 10 | 30 | 22 |
| 31 | 19 | 32 | 14 | 33 | 39 |
| 34 | 95 | 35 | 233 | 36 | 36 |
| 37 | 38 | 38 | 16 | 39 | 633 |
| 40 | 23 | 41 | 216 | 42 | 13 |
| 43 | 130 | 44 | 9 | 45 | 17 |
| 46 | 11 | 47 | 21 | 48 | 336 |
| 49 | 133 | 50 | 3922 | 51 | 565 |
| 52 | 53 | 53 | 126 | 54 | 66 |
| 55 | 1 | 56 | 1 | 57 | 2209 |
| 58 | 1785 | 59 | 7 | 60 | 3 |
| 61 | 56 | 62 | 704 | 63 | 767 |
| 64 | 45 | 65 | 4 | 66 | 4 |
| 67 | 16 | 68 | 28 | 69 | 47 |
| 70 | 956 | 71 | 2 | 72 | 11 |
| 73 | 45 | 74 | 94 | 75 | 320 |
| 76 | 288 | 77 | 2626 | 78 | 322 |
| 79 | 440 | 80 | 814 | 81 | 83 |
| 82 | 772 | 83 | 26 | 84 | 8 |
| 85 | 26 | 86 | 721 | 87 | 582 |
| 88 | 53 | 89 | 1607 | 90 | 10 |
| 91 | 38 | 92 | 7 | 93 | 23 |
| 94 | 46 | 95 | 9 | 96 | 54 |
| 97 | 807 | 98 | 4 | 99 | 2188 |
| 100 | 27 | 101 | 6 | 102 | 6 |
| 103 | 218 | 104 | 12 | 105 | 146 |
| 106 | 81 | 107 | 1216 | 108 | 190 |
| 109 | 573 | 110 | 1152 | 111 | 135 |
| 112 | 11 | 113 | 38 | 114 | 101 |
| 115 | 181 | 116 | 241 | 117 | 10 |
| 118 | 2871 | 119 | 63 | 120 | 3318 |
| 121 | 42 | 122 | 485 | 123 | 68 |
| 124 | 1050 | 125 | 1068 | 126 | 280 |
| 127 | 82 | 128 | 1324 | 129 | 3898 |
| 130 | 148 | 131 | 89 | 132 | 5 |
| 133 | 178 | 134 | 86 | 135 | 4 |
| 136 | 1976 | 137 | 741 | 138 | 14 |
| 139 | 61 | 140 | 1267 | 141 | 39 |
| 142 | 62 | 143 | 1247 | 144 | 191 |
| 145 | 0.6 | 146 | 4 | 147 | 39 |
| 148 | 16 | 149 | 225 | 150 | 156 |
| 151 | 441 | 152 | 183 | 153 | 63 |
| 154 | 326 | 155 | 2218 | 156 | not |
| 157 | 1381 | 158 | 56 | | determined |
| 160 | 5944 | 161 | 2 | 159 | 25 |
| | | | | 162 | 2 |

### REFERENCES

1. O'Connor R. M., Finger B. C., Flor P. J. and Cryan J. F., 2010. Metabotropic glutamate receptor 7: at the interface of cognition and emotion. Eur J Pharmacol., 639 (1-3), 123-31.
2. Konieczny J. and Lenda T., 2013 Contribution of the mGluR7 receptor to antiparkinsonian-like effects in rats: a behavioral study with the selective agonist AMN082. Pharmacol Rep., 65 (5), 1194-1203.
3. Greco B., Lopez S., van der Putten H. and Flor P. J., 2010. Amalric M. Metabotropic glutamate 7 receptor subtype modulates motor symptoms in rodent models of Parkinson's disease. J Pharmacol Exp Ther., 332 (3), 1064-71.
4. Bradley S. R., Standaert D. G., Levey A. I. and Conn P. J., 1999. Distribution of group III mGluRs in rat basal ganglia with subtype-specific antibodies. Ann N Y Acad Sci., 868, 531-4.
5. Conn P. J. and Niswender C. M., 2006. mGluR7's lucky number. Proceedings of the National Academy of Sciences of the United States of America, 103 (2), 251-2.
6. Hovelso N., Sotty F., Montezinho L., Pinheiro P., Herrik K. and Mork A., 2012. Therapeutic Potential of Metabotropic Glutamate Receptor Modulators. Curr Neuropharmacol., 10 (1), 12-48.
7. Kandaswamy R., McQuillin A., Curtis D. and Gurling H., 2014. Allelic Association, DNA Resequencing and Copy Number Variation at the Metabotropic Glutamate Receptor GRM7 Gene Locus in Bipolar Disorder. Am J Med Genet B Neuropsychiatr Genet., 165 (4), 365-72.
8. Palucha-Poniewiera A., Szewczyk B. and Pilc A, 2014. Activation of the mTOR signaling pathway in the antidepressant-like activity of the mGlu5 antagonist MTEP and the mGlu7 agonist AMN082 in the FST in rats. Neuropharmacology, 82, 59-68.
9. Palucha A., Klak K., Branski P., van der Putten H., Flor P. J. and Pile A., 2007. Activation of the mGlu7 receptor elicits antidepressant-like effects in mice. Psychopharmacology (Berl)., 194 (4), 555-62.
10. Kalinichev M., Rouillier M., Girard F., Royer-Urios I., Bournique B., Finn T. et al, 2013. ADX71743, a potent and selective negative allosteric modulator of metabotropic glutamate receptor 7: in vitro and in vivo characterization. J Pharmacol Exp Ther., 344 (3), 624-36.
11. Bolonna A. A., Kerwin R. W., Munro J., Arranz M. J., Makoff A. J., 2001. Polymorphisms in the genes for mGluR types 7 and 8: association studies with schizophrenia. Schizophr Res., 47 (1), 99-103.

1. A compound of formula (I) 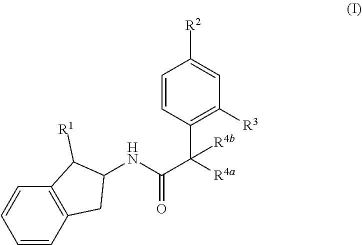
wherein
R¹ represents hydroxyl, —CH₂OH, cyano, —SO₂R^{1a}, —(CH₂)ₘ—(O)ₙ—R⁵ or —(CH₂)ₚNR⁶R⁷;
m is 0 or 1;
n is 0 or 1;
p is 0 or 1;
R^{1a} represents C₁-C₆ alkyl, C₃-C₆ cycloalkyl or C₃-C₆ cycloalkylmethyl;
R² and R³ each independently represent hydrogen, halogen, fluoromethyl, difluoromethyl, trifluoromethyl, methoxy, fluoromethoxy, difluoromethoxy or trifluoromethoxy;
either R^{4a} represents (X)ₜ—(CH₂)ᵥ—R¹⁶ or —CH₂O—R¹⁷ and R^{4b} represents hydrogen, methyl or fluorine, or
R^{4a} and R^{4b} together with the carbon atom to which they are attached form a saturated 3- to 6-membered carbocyclic or heterocyclic ring, the heterocyclic ring comprising at least one ring heteroatom selected from nitrogen and oxygen atoms, wherein the carbocyclic or heterocyclic ring is unsubstituted or substituted with at least one substituent selected from halogen, oxo, C₁-C₃ alkyl, C₁-C₃ alkoxy, amino, methylamino, dimethylamino and C₁-C₃ haloalkyl;
R⁵ represents a C₃-C₆ cycloalkyl group, a saturated 4- to 6-membered heterocyclic ring containing a single ring heteroatom being a nitrogen atom wherein the heterocyclic ring is unsubstituted or substituted with at least one substituent selected from halogen, C₁-C₃ alkyl and C₁-C₃ haloalkyl, or a C₁-C₆ alkyl group which is unsubstituted or substituted with at least one substituent selected from C₃-C₆ cycloalkyl, —NR²²R²³ and a saturated 4- to 6-membered heterocyclic ring comprising at least one ring heteroatom selected from nitrogen and oxygen atoms, which heterocyclic ring is unsubstituted or substituted by halogen;
R⁶ and R⁷ each independently represent hydrogen, —(CH₂)_{q}—R⁸, —SO₂R⁹, C₁-C₆ alkyl, C₁-C₆ alkylcarbonyl, C₃-C₆ cycloalkylcarbonyl or C₁-C₆ alkoxycarbonyl, wherein each of the alkyl, cycloalkyl or alkoxy moieties in the latter four substituents is unsubstituted or substituted with at least one substituent selected from halogen, C₁-C₄ alkoxy and —NR¹⁰R¹¹, or
R⁶ and R⁷ together with the nitrogen atom to which they are attached form a saturated or unsaturated 4- to 7-membered heterocyclic ring optionally comprising a further ring heteroatom selected from nitrogen, oxygen and sulphur, the heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹²R¹³;
q is 0, 1 or 2;
R⁸ represents a saturated or unsaturated 3- to 6-membered carbocyclic or heterocyclic ring wherein the heterocyclic ring comprises from 1 to 4 ring heteroatoms independently selected from nitrogen, oxygen and sulphur, the carbocyclic or heterocyclic ring being unsubstituted or substituted with at least one substituent selected from halogen, cyano, C₁-C₆ alkyl, C₃-C₆ cycloalkyl, C₃-C₆ cycloalkylmethyl, C₁-C₆ haloalkyl, C₁-C₆ alkoxy, C₃-C₆ cycloalkyloxy, C₃-C₆ cycloalkylmethyloxy and —NR¹⁴R¹⁵;
R⁹ represents C₁-C₆ alkyl, C₃-C₆ cycloalkyl or C₃-C₆ cycloalkylmethyl, each of which is unsubstituted or substituted with at least one halogen atom;
R¹⁰ and R¹¹ each independently represent hydrogen, C₁-C₆ alkyl, C₃-C₆ cycloalkyl or C₃-C₆ cycloalkylmethyl, or
R¹⁰ and R¹¹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;
R¹² and R¹³ each independently represent hydrogen, C₁-C₆ alkyl, C₃-C₆ cycloalkyl or C₃-C₆ cycloalkylmethyl, or
R¹² and R¹³ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;
R¹⁴ and R¹⁵ each independently represent hydrogen, C₁-C₆ alkyl, C₃-C₆ cycloalkyl or C₃-C₆ cycloalkylmethyl, or
R¹⁴ and R¹⁵ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;
t is 0 or 1;
v is 0, 1 or 2;
R¹⁶ represents —R¹⁷, —NR¹⁸R¹⁹ or a saturated or unsaturated 4- to 6-membered heterocyclic ring comprising from 1 to 4 ring heteroatoms independently selected from nitrogen, oxygen and sulphur, the heterocyclic ring being unsubstituted or substituted with at least one substituent selected from oxo, halogen, cyano, fluoromethoxy, difluoromethoxy, trifluoromethoxy, C₁-C₆ alkyl, C₁-C₆ alkoxy and C₁-C₆ haloalkyl;
X is O, NH, —NHC(O)—, —NHC(O)—, —NHC(O)O—, —C(O)NH—, —NHSO₂— or —SO₂NH—, provided that when X is O, NH, —C(O)NH— or —SO₂NH— and R¹⁶ represents —NR¹⁸R¹⁹, then v is 2;
R¹⁷ represents C₁-C₆ alkyl, C₃-C₆ cycloalkyl or C₃-C₆ cycloalkylmethyl, each of which is unsubstituted or substituted with at least one substituent selected from hydroxyl, halogen and —NR²⁰R²¹;
R¹⁸ and R¹⁹ each independently represent hydrogen, C₁-C₆ alkyl, C₁-C₆ alkylcarbonyl, C₃-C₆ cycloalkylcarbonyl, C₁-C₆ alkylsulphonyl or C₃-C₆ cycloalkylsulphonyl, wherein each of the alkyl or cycloalkyl moieties in the latter five substituents is unsubstituted or substituted with at least one substituent selected from halogen and C₁-C₄ alkoxy, or
R¹⁸ and R¹⁹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;
R²⁰ and R²¹ each independently represent hydrogen, C₁-C₆ alkyl, C₃-C₆ cycloalkyl or C₃-C₆ cycloalkylmethyl, or
R²⁰ and R²¹ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl; and
R²² and R²³ each independently represent hydrogen, C₁-C₆ alkyl, C₃-C₆ cycloalkyl or C₃-C₆ cycloalkylmethyl, or
R²² and R²³ together with the nitrogen atom to which they are attached form a saturated 4- to 6-membered heterocyclic ring optionally comprising a further ring heteroatom selected from nitrogen and oxygen atoms, the heterocyclic ring being unsubstituted or substituted by at least one substituent selected from halogen and C₁-C₃ alkyl;
provided that the compound of formula (I) is not N-[(1S,2S)-1-amino-2,3-dihydro-1H-inden-2-yl]-1-(4-fluorophenyl)cycl ° propane-1-carboxamide;
or a pharmaceutically acceptable salt thereof.

2. The compound according to claim 1, wherein R¹ represents —(CH₂)ₚNR⁶R⁷.

3. The compound according to claim 1, wherein p is 0.

4. The compound according to claim 1, wherein R⁶ and R⁷ each independently represent hydrogen, —(CH₂)_{q}—R⁸, C₁-C₂ alkyl, C₁-C₂ alkylcarbonyl or C₁-C₄ alkoxycarbonyl, wherein each of the alkyl or alkoxy moieties in the latter three substituents is unsubstituted or substituted with at least one substituent selected from fluorine, chlorine, C₁-C₂ alkoxy and —NR¹⁰R¹¹.

5. The compound according to claim 1, wherein R⁸ represents a saturated 3- to 6-membered carbocyclic ring or a saturated or unsaturated 5- to 6-membered heterocyclic ring comprising one or two ring heteroatoms independently selected from nitrogen and oxygen, the carbocyclic or heterocyclic ring being unsubstituted or substituted with at least one halogen atom.

6. The compound according to claim 1, wherein q is 1.

7. The compound according to claim 1, wherein R² and R³ each independently represent hydrogen, halogen, trifluoromethyl or methoxy.

8. The compound according to claim 7, wherein R² and R³ each independently represent hydrogen or fluorine.

9. The compound according to claim 1, wherein R^{4a} represents (X)ₜ—(CH₂)ᵥ—R¹⁶ and R^{4b} represents hydrogen, methyl or fluorine.

10. The compound according to claim 1, wherein t is 0.

11. The compound according to claim 1, wherein t is 1 and X is NH, —NHC(O)—, or —NHSO₂.

12. The compound according to claim 1, wherein v is 0 or 1.

13. The compound according to claim 1, wherein R¹⁶ represents R¹⁷ and R¹⁷ represents a C₁-C₆ alkyl or C₃-C₆ cycloalkyl group.

14. The compound according to claim 1, wherein R¹⁶ represents —NR¹⁸R¹⁹ in which R¹⁸ and R¹⁹ each independently represent hydrogen, C₁-C₂ alkyl, C₁-C₂ alkylcarbonyl, cyclopropylcarbonyl, C₁-C₂ alkylsulphonyl or cyclopropylsulphonyl, wherein each of the alkyl or cyclopropyl moieties in the latter five substituents is unsubstituted or substituted with at least one substituent selected from fluorine and methoxy.

15. The compound according to claim 1, wherein R¹⁶ represents an unsaturated 5- to 6-membered heterocyclic ring system comprising one or two ring heteroatoms independently selected from nitrogen, oxygen and sulphur, the ring system being unsubstituted or substituted as defined in claim 1.

16. The compound according to claim 1 selected from:
(2R)—N-((trans)-1-hydroxy-2,3-dihydro-1H-inden-2-yl)-2-phenylpropanamide;
(2 S)—N-((trans)-1-hydroxy-2,3-dihydro-1H-inden-2-yl)-2-phenylpropanamide;
(2 S)—N-((cis)-1-hydroxy-2,3-dihydro-1H-inden-2-yl)-2-phenylpropanamide;
(2 S)—N-((trans)-1-methoxy-2,3-dihydro-1H-inden-2-yl)-2-phenylpropanamide;
(2 S)—N-((cis)-1-methoxy-2,3-dihydro-1H-inden-2-yl)-2-phenylpropanamide;
(2S)—N-[(1S,2S)-1-amino-2,3-dihydro-1H-inden-2-yl]-2-phenylpropanamide;
(2S)—N-[(1R,2R)-1-amino-2,3-dihydro-1H-inden-2-yl]-2-phenylpropanamide;
(2S)—N-[(1S,2S)-1-acetamido-2,3-dihydro-1H-inden-2-yl]-2-phenylpropanamide;
N-[(1S,2S)-1-amino-2,3-dihydro-1H-inden-2-yl]-2-(2,4-difluorophenyl)butanamide;
N-[(1R,2R)-1-amino-2,3-dihydro-1H-inden-2-yl]-2-(2,4-difluorophenyl)butanamide;
tert-butyl N-[(1R,2R)-2-[(2S)-2-phenylpropanamido]-2,3-dihydro-1H-inden-1-yl]carbamate;
(2S)—N-[(1S,2S)-1-amino-2,3-dihydro-1H-inden-2-yl]-2-methoxy-2-phenylacetamide;
N-[(1S,2S)-1-amino-2,3-dihydro-1H-inden-2-yl]-2-(4-chlorophenyl)-3-methylbutanamide;
tert-butyl N-[(1S,2S)-2-[(2S)-2-(4-fluorophenyl)propanamido]-2,3-dihydro-1H-inden-1-yl]carbamate;
(2S)—N-[(1S,2S)-1-(methylamino)-2,3-dihydro-1H-inden-2-yl]-2-phenylpropanamide;
tert-butyl N-[(1S,2S)-2-[(2S)-2-(2,4-difluorophenyl)propanamido]-2,3-dihydro-1H-inden-1-yl]carbamate;
(2S)—N-[(1S,2S)-1-amino-2,3-dihydro-1H-inden-2-yl]-2-(4-fluorophenyl)propanamide;
(2S)-2-(2,4-difluorophenyl)-N-((trans)-1-methoxy-2,3-dihydro-1H-inden-2-yl)propanamide;
(2S)-2-(2,4-difluorophenyl)-N-((trans)-1-ethoxy-2,3-dihydro-1H-inden-2-yl)propanamide;
(2 S)—N-((trans)-1-ethoxy-2,3-dihydro-1H-inden-2-yl)-2-(4-fluorophenyl)propanamide;
(2S)—N-[(1S,2S)-1-amino-2,3-dihydro-1H-inden-2-yl]-2-(2,4-difluorophenyl)propanamide;
(2S)—N-[(1R,2R)-1-amino-2,3-dihydro-1H-inden-2-yl]-2-(2,4-difluorophenyl)propanamide;
(2S)-2-(4-fluorophenyl)-N-((trans)-1-methoxy-2,3-dihydro-1H-inden-2-yl)propanamide;
(2S)-2-(4-fluorophenyl)-N-(cis)-(1-methoxy-2,3-dihydro-1H-inden-2-yl)propanamide;
(2S)-2-(2,4-difluorophenyl)-N-[(1S,2S)-1-{[(oxan-4-yl)methyl]amino}-2,3-dihydro-1H-inden-2-yl]propanamide;
tert-butyl N-[(1R,2R)-2-[2-(2,4-difluorophenyl)butanamido]-2,3-dihydro-1H-inden-1-yl]carbamate;
tert-butyl N-[(1S,2S)-2-[(2S)-2-(4-fluorophenyl)butanamido]-2,3-dihydro-1H-inden-1-yl]carbamate;
tert-butyl N-[(1R,2R)-2-[(2S)-2-(4-fluorophenyl)butanamido]-2,3-dihydro-1H-inden-1-yl]carbamate;
(2S)-2-(4-fluorophenyl)-N-((trans)-1-methoxy-2,3-dihydro-1H-inden-2-yl)butanamide;
(2S)—N-[(1S,2S)-1-[(cyclopropylmethyl)amino]-2,3-dihydro-1H-inden-2-yl]-2-(4-fluorophenyl)propanamide;
tert-butyl N-[(1S,2S)-2-[2-(2,4-difluorophenyl)-2-(2-oxo-1,2-dihydropyridin-1-yl)acetamido]-2,3-dihydro-1H-inden-1-yl]carbamate;
N-[(1S,2S)-1-amino-2,3-dihydro-1H-inden-2-yl]-2-(2,4-difluorophenyl)-2-(2-oxo-1,2-dihydropyridin-1-yl)acetamide;
(2S)—N-[(1S,2S)-1-amino-2,3-dihydro-1H-inden-2-yl]-2-(4-fluorophenyl)butanamide;
(2S)—N-[(1R,2R)-1-amino-2,3-dihydro-1H-inden-2-yl]-2-(4-fluorophenyl)butanamide;
(2S)-2-amino-2-(4-fluorophenyl)-N-(trans)-(1-methoxy-2,3-dihydro-1H-inden-2-yl)acetamide;
(2S)-2-[(cyclopropylmethyl)amino]-2-(4-fluorophenyl)-N-(trans)-(1-methoxy-2,3-dihydro-1H-inden-2-yl)acetamide;
N-[(1S,2S)-1-amino-2,3-dihydro-1H-inden-2-yl]-2-(4-methoxyphenyl)propanamide;
N-[(1S,2S)-1-amino-2,3-dihydro-1H-inden-2-yl]-2-[4-(trifluoromethyl)phenyl]propanamide;
(2S)-2-(2,4-difluorophenyl)-N-[(1S,2S)-1-[(2,2,2-trifluoroethyl)amino]-2,3-dihydro-1H-inden-2-yl]propanamide;
(2 S)—N-(trans)-(1-ethoxy-2,3-dihydro-1H-inden-2-yl)-2-(4-fluorophenyl)-2-{[(2-methyl-1,3-thiazol-4-yl)methyl]amino}acetamide;
(2S)-2-(4-fluorophenyl)-N-(trans)-(1-hydroxy-2,3-dihydro-1H-inden-2-yl)propanamide;
(2S)-2-(4-fluorophenyl)-N-(cis)-(1-hydroxy-2,3-dihydro-1H-inden-2-yl)propanamide;
(2S)-2-(4-fluorophenyl)-N-[(1S,2S)-1-(methylamino)-2,3-dihydro-1H-inden-2-yl]propanamide;
(2S)-2-(4-fluorophenyl)-N-(trans)-(1-methoxy-2,3-dihydro-1H-inden-2-yl)-2-[(1-methyl-1H-pyrazol-4-yl)formamido]acetamide;
(2S)-2-(cyclopropylformamido)-2-(4-fluorophenyl)-N-(trans)-(1-methoxy-2,3-dihydro-1H-inden-2-yl)acetamide;
(2S)-2-(4-fluorophenyl)-N-(trans)-[1-(pyrrolidin-1-yl)-2,3-dihydro-1H-inden-2-yl]propanamide;
N-[(1S,2S)-1-amino-2,3-dihydro-1H-inden-2-yl]-2-(4-chlorophenyl)propanamide;
tert-butyl N—[(S)-(4-fluorophenyl)[(trans)-(1-methyl-2,3-dihydro-1H-inden-2-yl)carbamoyl]methyl]carbamate;
(2S)-2-(4-fluorophenyl)-2-methanesulphonamido-N-(trans)-(1-methoxy-2,3-dihydro-1H-inden-2-yl)acetamide;
(2S)-2-(4-fluorophenyl)-N-(trans)-[1-(morpholin-4-yl)-2,3-dihydro-1H-inden-2-yl]propanamide;
(2S)—N-(trans)-[1-(dimethylamino)-2,3-dihydro-1H-inden-2-yl]-2-(4-fluorophenyl)propanamide;
N-[(1S,2S)-1-amino-2,3-dihydro-1H-inden-2-yl]-2-fluoro-2-(4-fluorophenyl)propanamide;
(2S)-2-phenyl-N-(trans)-[1-(pyrrolidin-1-yl)-2,3-dihydro-1H-inden-2-yl]propanamide;
N-[(1S,2S)-1-amino-2,3-dihydro-1H-inden-2-yl]-1-(2-chlorophenyl)cyclopropane-1-carboxamide;
(2S)-2-(2,4-difluorophenyl)-N-[(1S,2S)-1-acetamido-2,3-dihydro-1H-inden-2-yl]propanamide;
tert-butyl N-[(1S,2S)-2-[2-(2,4-difluorophenyl)-2-(1H-pyrazol-1-yl)acetamido]-2,3-dihydro-1H-inden-1-yl]carbamate;
tert-butyl N-[(1S,2S)-2-[2-(2,4-difluorophenyl)-2-(2-methyl-1H-imidazol-1-yl)acetamido]-2,3-dihydro-1H-inden-1-yl]carbamate;
(2S)-2-(3,5-dimethyl-1,2-isoxazole-4-sulfonamido)-2-(4-fluorophenyl)-N-((trans)-1-methoxy-2,3-dihydro-1H-inden-2-yl)acetamide;
(2 S)—N-{(trans)-1-[(2,2-difluoroethyl)amino]-2,3-dihydro-1H-inden-2-yl}-2-(4-fluorophenyl)propanamide;
(2S)-2-(4-fluorophenyl)-2-methanesulfonamido-N-((trans)-1-methyl-2,3-dihydro-1H-inden-2-yl)acetamide;
N-[(1S,2S)-1-amino-2,3-dihydro-1H-inden-2-yl]-2-cyclopropyl-2-(4-fluorophenyl)acetamide;
tert-butyl N-[(1S,2S)-2-[2-(4-fluorophenyl)-2-methylpropanamido]-2,3-dihydro-1H-inden-1-yl]carbamate;
tert-butyl N-[(1S,2S)-2-(3-phenyloxetane-3-amido)-2,3-dihydro-1H-inden-1-yl]carbamate;
(2S)-2-(2,4-difluorophenyl)-N-[(1S,2S)-1-methanesulfonamido-2,3-dihydro-1H-inden-2-yl]propanamide;
(2S)—N-[(1S,2S)-1-[(cyclobutylmethyl)amino]-2,3-dihydro-1H-inden-2-yl]-2-(2,4-difluorophenyl)propanamide;
(2S)—N-[(1S,2S)-1-(cyclobutylamino)-2,3-dihydro-1H-inden-2-yl]-2-(2,4-difluorophenyl)propanamide;
(2S)-2-(2,4-difluorophenyl)-N-[(1S,2S)-1-{[(3-fluoropyridin-2-yl)methyl]amino}-2,3-dihydro-1H-inden-2-yl]propanamide;
tert-butyl N-[(1S,2S)-2-[2-(2,4-difluorophenyl)-2-(3-fluoroazetidin-1-yl)acetamido]-2,3-dihydro-1H-inden-1-yl]carbamate;
tert-butyl N-[(1S,2S)-2-[4-(4-fluorophenyl)oxane-4-amido]-2,3-dihydro-1H-inden-1-yl]carbamate;
(2S)-2-(4-fluorophenyl)-N-[(1R,2R)-1-(methylamino)-2,3-dihydro-1H-inden-2-yl]propanamide;
(2S)-2-(4-fluorophenyl)-N-(trans)-(1-methoxy-2,3-dihydro-1H-inden-2-yl)-2-[(oxan-4-yl)formamido]acetamide;
N-[(1S,2S)-1-amino-2,3-dihydro-1H-inden-2-yl]-2-(4-fluorophenyl)-2-methylpropanamide;
tert-butyl N-[(1S,2S)-2-[2-(azetidin-1-yl)-2-(2,4-difluorophenyl)acetamido]-2,3-dihydro-1H-inden-1-yl]carbamate;
N-[(1S,2S)-1-amino-2,3-dihydro-1H-inden-2-yl]-2-(3,3-difluoroazetidin-1-yl)-2-(2,4-difluorophenyl)acetamide;
tert-butyl N-[(1S,2S)-2-[2-(2,4-difluorophenyl)-2-(3-methoxyazetidin-1-yl)acetamido]-2,3-dihydro-1H-inden-1-yl]carbamate;
N-[(1S,2S)-1-amino-2,3-dihydro-1H-inden-2-yl]-2-(2,4-difluorophenyl)-2-(3-fluoroazetidin-1-yl)acetamide;
N-[(1S,2S)-1-amino-2,3-dihydro-1H-inden-2-yl]-2-(azetidin-1-yl)-2-(2,4-difluorophenyl)acetamide;
N-[(1S,2S)-1-amino-2,3-dihydro-1H-inden-2-yl]-2-(2,4-difluorophenyl)-2-(6-oxo-1,6-dihydropyridazin-1-yl)acetamide;
N-[(1S,2S)-1-amino-2,3-dihydro-1H-inden-2-yl]-2-(2,4-difluorophenyl)-2-(3-methoxyazetidin-1-yl)acetamide;
(2 S)—N-[(1R,2R)-1-(3-fluoroazetidin-1-yl)-2,3-dihydro-1H-inden-2-yl]-2-(4-fluorophenyl)propanamide;
(2S)-2-(4-fluorophenyl)-N-[(1R,2R)-1-methanesulfonyl-2, 3-dihydro-1H-inden-2-yl]propanamide;
(2S)—N-[(1S,2S)-1-{bis[(1,3-oxazol-2-yl)methyl]amino}-2,3-dihydro-1H-inden-2-yl]-2-(2,4-difluorophenyl)propanamide;
N-[(1S,2S)-1-amino-2,3-dihydro-1H-inden-2-yl]-2-(3-fluoroazetidin-1-yl)-2-(4-fluorophenyl)acetamide;
N-[(1S,2S)-1-amino-2,3-dihydro-1H-inden-2-yl]-2-(3-fluoroazetidin-1-yl)-2-(4-fluorophenyl)acetamide;
N-[(1S,2S)-1-amino-2,3-dihydro-1H-inden-2-yl]-2-[3-(difluoromethoxy)azetidin-1-yl]-2-(2,4-difluorophenyl)acetamide;
N-[(1S,2S)-1-amino-2,3-dihydro-1H-inden-2-yl]-3-phenylpyrrolidine-3-carboxamide;
tert-butyl N-[(1S,2S)-2-(5-oxo-3-phenylpyrrolidine-3-amido)-2,3-dihydro-1H-inden-1-yl]carbamate;
N-[(1S,2S)-1-amino-2,3-dihydro-1H-inden-2-yl]-5-oxo-3-phenylpyrrolidine-3-carboxamide;
tert-butyl N-[(1S,2S)-2-(3-oxo-1-phenylcyclobutaneamido)-2,3-dihydro-1H-inden-1-yl]carbamate;
2-(2,4-difluorophenyl)-N-(trans)-(1-methoxy-2,3-dihydro-1H-inden-2-yl)-2-(6-oxo-1,6-dihydropyridazin-1-yl)acetamide;
2-(4-fluorophenyl)-N-(trans)-(1-methoxy-2,3-dihydro-1H-inden-2-yl)-2-(6-oxo-1,6-dihydropyridazin-1-yl)acetamide;
(2S)-2-(4-fluorophenyl)-N-[(1S,2S)-1-methanesulfonyl-2,3-dihydro-1H-inden-2-yl]propanamide;
(2S)-2-(2,4-difluorophenyl)-N-[(1S,2S)-1-[(pyrimidin-2-yl)amino]-2,3-dihydro-1H-inden-2-yl]propanamide;
(2S)—N-[(1S,2S)-1-(ethylamino)-2,3-dihydro-1H-inden-2-yl]-2-(4-fluorophenyl)propanamide;
2-(cyclopropylmethoxy)-N-(trans)-(1-methanesulfonyl-2,3-dihydro-1H-inden-2-yl)-2-phenylacetamide;
2-(2,4-difluorophenyl)-2-(3-fluoroazetidin-1-yl)-N-(trans)-(1-methoxy-2,3-dihydro-1H-inden-2-yl)acetamide;
2-(4-fluorophenyl)-2-(3-fluoroazetidin-1-yl)-N-(trans)-(1-methoxy-2,3-dihydro-1H-inden-2-yl)acetamide;
(2R)-2-(cyclopropylformamido)-2-(4-fluorophenyl)-N-(trans)-(1-methoxy-2,3-dihydro-1H-inden-2-yl)acetamide;
(2 S)—N-(trans)-(1-methanesulfonyl-2,3-dihydro-1H-inden-2-yl)-2-methoxy-2-phenylacetamide;
(2S)—N-(trans)-[1-(ethanesulfonyl)-2,3-dihydro-1H-inden-2-yl]-2-methoxy-2-phenylacetamide;
tert-butyl N-[(1S,2S)-2-{2-[4-(difluoromethoxy)phenyl]propanamido}-2,3-dihydro-1H-inden-1-yl]carbamate;
tert-butyl N-[(1S,2S)-2-[2-(4-fluoro-2-methoxyphenyl)propanamido]-2,3-dihydro-1H-inden-1-yl]carbamate;
tert-butyl N-[(1S,2S)-2-[2-(2-chloro-4-fluorophenyl)propanamido]-2,3-dihydro-1H-inden-1-yl]carbamate;
tert-butyl N-[(1S,2S)-2-{2-[4-fluoro-2-(trifluoromethyl)phenyl]propanamido}-2,3-dihydro-1H-inden-1-yl]carbamate;
(2R)-2-(4-fluorophenyl)-N-[(1R,2R)-1-(methylamino)-2,3-dihydro-1H-inden-2-yl]propanamide;
tert-butyl N-[(1S,2S)-2-[2-phenyl-3-(pyrrolidin-1-yl)propanamido]-2,3-dihydro-1H-inden-1-yl]carbamate;
(2R)-2-(4-fluorophenyl)-N-[(1S,2S)-1-(methylamino)-2,3-dihydro-1H-inden-2-yl]propanamide;
(2R)—N-(trans)-(1-methanesulfonyl-2,3-dihydro-1H-inden-2-yl)-2-methoxy-2-phenylacetamide;
(2 S)—N-(trans)-(1-methanesulfonyl-2,3-dihydro-1H-inden-2-yl)-2-methoxy-2-phenylacetamide;
(2S)-2-(4-fluorophenyl)-N-[(1R,2S)-1-(methylamino)-2,3-dihydro-1H-inden-2-yl]propanamide;
2-(cyclopropylmethoxy)-2-(4-fluorophenyl)-N-(trans)-(1-methanesulfonyl-2,3-dihydro-1H-inden-2-yl)acetamide;
2-(cyclopropylmethoxy)-2-(4-fluorophenyl)-N-(trans)-(1-methanesulfonyl-2,3-dihydro-1H-inden-2-yl)acetamide; and
enantiomers, diastereoisomers and mixtures thereof; and
pharmaceutically acceptable salts of any of the foregoing.

17. A process for the preparation of a compound of formula (I) or a pharmaceutically acceptable salt thereof as defined in claim 1 which comprises reacting a compound of formula (II), or a salt thereof, 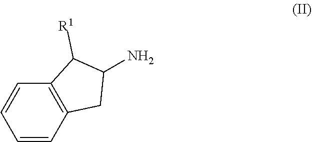
in which R¹ is as defined in formula (I), with a compound of formula (III), or a salt thereof, 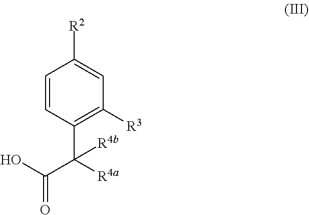
in which R², R³, R^{4a} and R^{4b} are as defined in formula (I);
and optionally thereafter carrying out one or more of the following procedures:
converting a compound of formula (I) into another compound of formula (I) removing any protecting groups forming a pharmaceutically acceptable salt.

18. A pharmaceutical composition comprising a compound of formula (I) or a pharmaceutically acceptable salt thereof, as claimed in claim 1, in association with a pharmaceutically acceptable adjuvant, diluent or carrier, and optionally one or more other therapeutic agents.

19. The compound of formula (I) or a pharmaceutically acceptable salt thereof, as claimed in claim 1, for use in therapy.

20. The compound of formula (I) or a pharmaceutically acceptable salt thereof, as claimed in claim 1, for use in treating alcohol, drug or nicotine addiction.

21. The compound of formula (I) or a pharmaceutically acceptable salt thereof, as claimed in claim 1, for use in treating hearing loss or tinnitus.

22. The compound of formula (I) or a pharmaceutically acceptable salt thereof, as claimed in claim 1, for use in treating schizophrenia.
